SEQUENCE LISTING (1 sequence)

SEQ ID NO: 1 - DNA, 4073 bp, synthetic construct
  source 1..4073: mol_type = other DNA; note = SARS-CoV-2 prefusion-stabilized two-proline S2P spike mRNA sequence was designed in-house
agacagatcgcctggagacgccatccacgctgttttgacctccatagaagacaccgggaccgatccagcctccgcggccgggaacggtgcattggaacgcggattccccgtgccaagagtgactcaccgtccttgacacgatgttcgtattcctcgtattgctgccactggtgagcagccagtgcgtgaacctgaggaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcatcgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacagaagctacctgacccccggcgacagcagcagcggctggaccgccggcgccgccgcctactacgtgggctacctgcagccaagaaccttcctgctgaagtacaacgagaacggcaccatcaccgacgccgtggactgcgccctggaccccctgagcgagacaaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagcaggagaagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggacattcggcgccggcgccgccctgcagatcccattcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacagactgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacatctgatagtaacgggtggcatccctgtgacccctccccagtgcctctcctggccctggaagttgccactccagtgcccaccagccttgtcctaataaaattaagttgcatcaagctctctag

(19) United States
(12) Patent Application Publication
(10) Pub. No.: US 2025/0205158 A1
(43) Pub. Date: Jun. 26, 2025

(54) BIODEGRADABLE LIPIDS AND FORMULATIONS FOR DELIVERY OF MRNA

(71) Applicants: Massachusetts Institute of Technology, Cambridge, MA (US); Children's Medical Center Corporation, Boston, MA (US)

(72) Inventors: Daniel Griffith Anderson, Framingham, MA (US); Arnab Rudra, Brookline, MA (US); Akash Gupta, Arlington, MA (US); Kaelan Reed, Cambridge, MA (US)

(73) Assignees: Massachusetts Institute of Technology, Cambridge, MA (US); Children's Medical Center Corporation, Boston, MA (US)

(21) Appl. No.: 19/002,347

(22) Filed: Dec. 26, 2024

Related U.S. Application Data
(60) Provisional application No. 63/614,873, filed on Dec. 26, 2023.

(51) Int. Cl. A61K 9/1272; A61K 9/51; C07D 243/08; C07D 255/02; C07D 259/00; C07D 471/10

(52) U.S. Cl. CPC A61K 9/1272; A61K 9/5123; C07D 243/08; C07D 255/02; C07D 259/00; C07D 471/10

(57) ABSTRACT

Provided herein are compounds, such as compounds of Formulae (I), (I′), (XI), (XII), and (XIII), and pharmaceutically acceptable salts, solvates, tautomers, stereoisomers, and isotopically labeled derivatives thereof, and compositions, methods, uses, and kits thereof. The compounds provided herein are lipids useful for delivery of agents, including polynucleotides such as mRNA, for the treatment and/or prevention of various diseases and conditions (e.g., genetic diseases, proliferative diseases, hematological diseases, neurological diseases, liver diseases, spleen diseases, lung diseases, painful conditions, psychiatric disorders, musculoskeletal diseases, metabolic disorders, inflammatory diseases, and autoimmune diseases). Also provided herein are methods of synthesis of compounds of Formulae (I′), (XI), (XII), (XIII), and (VIII).

### CROSS-REFERENCE TO RELATED APPLICATIONS

This application claims the benefit of and priority under 35 U.S.C. § 119(e) to U.S. Provisional Application, U.S. Ser. No. 63/614,873, filed Dec. 26, 2023, titled “Biodegradable Lipids and Formulations for Delivery of mRNA,” which is incorporated herein by reference.

### REFERENCE TO AN ELECTRONIC SEQUENCE LISTING

The contents of the electronic sequence listing (M123770152US01-SEQ-CLL.xml; Size: 6,276 bytes; and Date of Creation: Dec. 24, 2024) are herein incorporated by reference in its entirety.

### BACKGROUND

mRNA therapeutics have significant potential to treat various diseases through protein replacement, immunomodulation, and gene editing. Non-viral nanoparticles are promising mRNA delivery vehicles to target cells in vivo. For example, mRNA-based vaccines against SARS-CoV-2 has highlighted the utility of lipid nanoparticle formulations for the effective delivery of mRNA. New compounds capable of forming delivery vehicles may be useful for mRNA-based vaccines and chimeric antigen receptor T (CAR-T) cell therapy.

Provided herein are ionizable lipids and their formulations that are highly efficient for intramuscular (IM) and intravenous (IV) administration of mRNA.

### SUMMARY OF THE INVENTION

Provided herein are biodegradable ionizable lipids that, when incorporated in formulations containing mRNA, demonstrate efficient in vivo transfection, for example, via either intramuscular (IM) or intravenous (IV) administration. The lipids provided herein are also suitable for in vitro and ex vivo transfection of mRNA to hard-to-transfect cells.

In one aspect, provided herein is a compound of Formula (I):

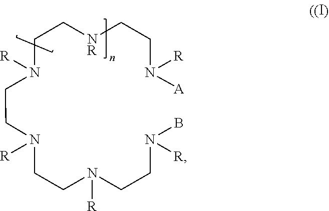

or a pharmaceutically acceptable salt, solvate, tautomer, stereoisomer, or isotopically labeled derivative thereof, wherein n, A, B, and R are as defined herein.

In another aspect, provided herein is a compound of Formula (XI):

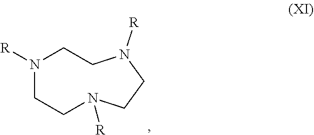

or a pharmaceutically acceptable salt, solvate, tautomer, stereoisomer, or isotopically labeled derivative thereof, wherein R is as defined herein.

In another aspect, provided herein is a compound of Formula (XII):

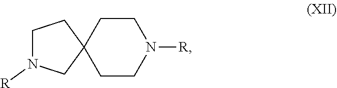

or a pharmaceutically acceptable salt, solvate, tautomer, stereoisomer, or isotopically labeled derivative thereof, wherein R is as defined herein.

In another aspect, provided herein is a compound of Formula (XIII):

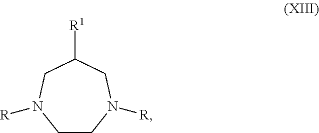

or a pharmaceutically acceptable salt, solvate, tautomer, stereoisomer, or isotopically labeled derivative thereof, wherein R¹ and R are as defined herein.

In another aspect, provided herein is a composition comprising a compound provided herein, or a pharmaceutically acceptable salt thereof, and an agent.

In another aspect, provided herein is a composition comprising a compound provided herein, or a pharmaceutically acceptable salt thereof, or a composition provided herein, and a pharmaceutically acceptable excipient. In some embodiments, the composition is a pharmaceutical composition.

In another aspect, provided herein is a method of delivering an agent to a subject, tissue, or a cell, comprising administering to the subject or contacting the tissue or the cell with a composition comprising an agent and a compound provided herein, or a pharmaceutically acceptable salt thereof.

In another aspect, provided herein is a method of treating and/or preventing a disease, disorder, or condition in a subject, comprising administering to the subject a composition comprising an agent and a compound provided herein, or a pharmaceutically acceptable salt thereof.

In another aspect, provided herein is a method of preparing a compound of Formula (I′):

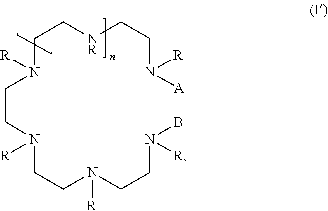

or a salt, solvate, tautomer, stereoisomer, or isotopically labeled derivative thereof, the method comprising reacting a compound of Formula (VII):

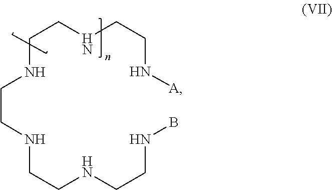

or a salt, solvate, tautomer, stereoisomer, or isotopically labeled derivative thereof, with a compound of Formula (VIII):

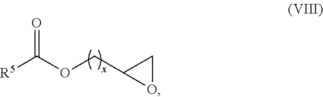

or a salt, solvate, tautomer, stereoisomer, or isotopically labeled derivative thereof, wherein n, A, B, R, R⁵, and x are as defined herein.

In another aspect, provided herein is a method of preparing a compound of Formula (XI′):

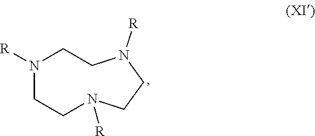

or a salt, solvate, tautomer, stereoisomer, or isotopically labeled derivative thereof, the method comprising reacting a compound of Formula (XI″):

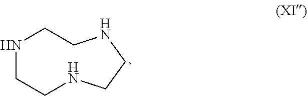

or a salt, solvate, tautomer, stereoisomer, or isotopically labeled derivative thereof, with a compound of Formula (VIII):

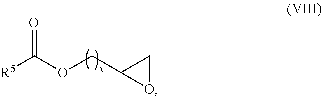

or a salt, solvate, tautomer, stereoisomer, or isotopically labeled derivative thereof, wherein R, R⁵, and x are as defined herein.

In another aspect, provided herein is a method of preparing a compound of Formula (XII′):

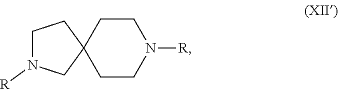

or a salt, solvate, tautomer, stereoisomer, or isotopically labeled derivative thereof, the method comprising reacting a compound of Formula (XII″):

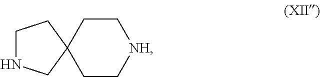

or a salt, solvate, tautomer, stereoisomer, or isotopically labeled derivative thereof, with a compound of Formula (VIII):

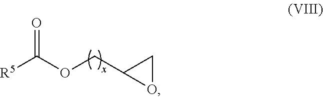

or a salt, solvate, tautomer, stereoisomer, or isotopically labeled derivative thereof, wherein R, R⁵, and x are as defined herein.

In another aspect, provided herein is a method of preparing a compound of Formula (XIII′):

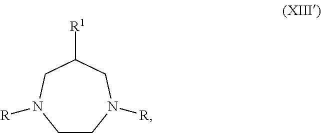

or a salt, solvate, tautomer, stereoisomer, or isotopically labeled derivative thereof, the method comprising reacting a compound of Formula (XIII″):

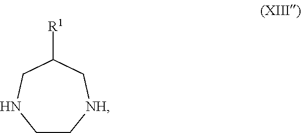

or a salt, solvate, tautomer, stereoisomer, or isotopically labeled derivative thereof, with a compound of Formula (VIII):

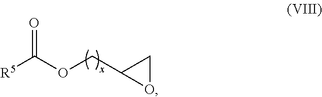

or a salt, solvate, tautomer, stereoisomer, or isotopically labeled derivative thereof, wherein R¹, R, R⁵, and x are as defined herein.

In another aspect, provided herein is a method of preparing a compound of Formula (VIII):

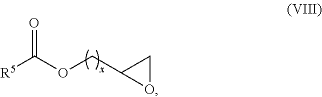

or a salt, solvate, tautomer, stereoisomer, or isotopically labeled derivative thereof, the method comprising reacting a compound of Formula (IX):

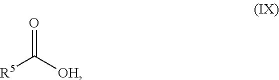

or a salt, solvate, tautomer, stereoisomer, or isotopically labeled derivative thereof, with a carbodiimide coupling agent, or a salt, solvate, tautomer, stereoisomer, or isotopically labeled derivative thereof, and a compound of Formula (X):

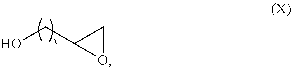

or a salt, solvate, tautomer, stereoisomer, or isotopically labeled derivative thereof, wherein x and R⁵ are as defined herein.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIGS. 1A-1D show structures of head groups used for the library of ionizable lipids (FIG. 1A), structures of tails from an alkene walking strategy (FIG. 1B), the structure of AMG1041 (FIG. 1C), and the structure of AMG1541 (FIG. 1D).

FIGS. 2A-2B show a comparison between in vitro performance of the ionizable lipids screened in HeLa cells (FIG. 2A) and DC2.4 cells (FIG. 2B).

FIG. 3 shows the Flue expression profile for AMG1041, AMG1241, AMG1541, and AMG1641.

FIG. 4 shows endpoint titers at day 35 after prime (day 0) and boost (day 21) doses.

FIG. 5 shows pseudovirus neutralization IC50 values.

FIGS. 6A-6B show in vitro (FIG. 4A) and in vivo (FIG. 4B) protein expression via intramuscular administration for LNPs made with compounds of the present disclosure.

FIG. 7 shows total bioluminescent flux in liver.

FIGS. 8A-8D show evaluation of AMG1541 for intravenous hEPO protein production.

FIGS. 9A-9D show Cas9 mediated gene editing efficiency using AMG1541 LNPs.

FIGS. 10A and 10B show structures of additional tails.

FIGS. 11A-11F show a combinatorial lipid library. FIG. 11A shows a schematic illustrating synthetic scheme for combinatorial lipid library that was screened using batch-based analysis to identify potent ionizable lipids for mRNA delivery. Subsequent, iterative development of hit lipids through medicinal chemistry, computational chemistry, and in vivo screening yielded potent vaccine delivery systems inducing stronger immune response. FIG. 11B shows structures of amine headgroups (Am). FIG. 11C shows structures of ester tails (G) used to synthesize AMG ionizable lipids. FIG. 11D shows results of batch-based analysis where the screen 1 identified potent tail groups (15 μg, n=3). FIG. 11E shows results of batch-based analysis using identified potent smaller batches of headgroups from the tails groups down-selected from the screen 1 (5 μg, n=3). FIG. 11F shows individually tested top lipids (1 μg, n=3) identified from screen 2. All the mice were imaged 24 hours post injection. Statistical significance was analyzed using a two-way analysis of variance with Šídák's multiple comparisons test with a single pooled variance.

FIGS. 12A-12F show evaluation of a lipids comprising cyclic head groups. FIG. 12A shows structures of different tails and quantification of Fluc protein expression upon intramuscular injection in mice for tail derivatives (5 μg, 24 hours). FIG. 12B shows structures of different headgroups and quantification of Fluc protein expression upon intramuscular injection in mice for head group analogues (1 μg, 6 hours). FIG. 12C shows representative images and graph comparing intramuscular Fluc protein expression upon intramuscular injection (1 μg, 6 hours) of AMG lipids and to lipid controls (SM-102 and ckk-e12). FIG. 12D shows the structure of AMG1541 ionizable lipid. FIG. 12E shows AMG lipid concentration (nanograms per gram) after IM administration of modified mRNA encoding luciferase in muscles. FIG. 12F shows Fluc expression in liver and spleen at 6 hours post-injection (n=3 per group per time point), showing rapid degradation of AMG1541 with minimal accumulation in liver and spleen. Statistical significance was analyzed using a two-way analysis of variance with Šídák's multiple comparisons test with a single pooled variance.

FIGS. 13A-13H show AMG1541 LNPs induce strong humoral and cellular vaccine responses. FIG. 13A shows the vaccination protocol used for all vaccine experiments. The same indicated mRNA dose was used for both vaccine doses. FIG. 13B shows a correlation of firefly luciferase luminescent flux (dose=1 μg, n=3) with day 35 binding antibody titers following vaccination with spike mRNA (dose=1 μg, n=5). Data are presented as mean±±s.d. A linear best fit line and 95% confidence interval is shown. FIG. 13C shows day 35 binding antibody titers following vaccination with HA mRNA (dose=1 μg, n=5). FIG. 13D shows fold-change cytokine levels in the cell culture supernatant of peptide-stimulated splenocytes following vaccination with HA mRNA (dose=1 μg, n=5). FIG. 13E BALB/cJ serum cytokines (pg/mL) 6 hrs. after boost with HA mRNA (dose=1 g). FIG. 13F shows day 35 female C57B1/6J serum binding antibody titers after vaccination with HA mRNA (dose=1 μg, n=5). FIG. 13G shows day 35 peptide pool-stimulated splenocyte IFN-γ ELISpot SFU (top) and representative images (bottom) from female C57B1/6J mice vaccinated with HA mRNA (dose=1 g). FIG. 13H shows day 35 serum neutralizing antibody titers from female BALB/cJ mice vaccinated with HA mRNA (n=5). Statistical significance was analyzed using a two-way analysis of variance with Šídák's multiple comparisons test with a single pooled variance. (FIGS. 13C, 13G, and 13H) repeated measures two-way analysis of variance with Tukey's multiple comparisons test with individual variances (FIGS. 13D and 13E) or an unpaired, two-tailed t-test (FIG. 13F).

FIGS. 14A-14K shows that AMG1541 enhances mRNA expression in immune cell populations and induces robust germinal center reactions in response to vaccination. FIG. 14A shows representative flow cytometry plots of muscle cells showing DiI-labeled LNP uptake. FIG. 14B shows DiI-labeled LNP uptake in cells isolated from the muscle (n=5). FIG. 14C shows DiI-labeled LNP uptake in cells isolated from LDLN (n=5). FIG. 14D shows representative flow cytometry plots of muscle cells showing tdTomato expression. FIG. 14E shows tdTomato mRNA expression in cells isolated from the muscle. FIG. 14F shows tdTomato mRNA expression in cells isolated from the local draining lymph node. FIG. 14G shows IHC images showing tdTomato expression in the muscle following LNP injection. FIG. 14H shows representative flow cytometry plots showing GC B cells (n=5). FIG. 14I shows representative flow cytometry plots showing number of GCB cells from the LDLN (n=5). FIG. 14J shows representative flow cytometry plots showing antigen positive GC B cells (n=5). FIG. 14K shows representative flow cytometry plots showing number of antigen positive GC B cells in the local draining lymph node following vaccination (n=5). Statistical analysis was performed using a two-way analysis of variance with Tukey's multiple comparisons test with a single pooled variance (FIGS. 14B, 14C, 14E, and 14F) or a one-way analysis of variance with a Dunnett's multiple comparisons test with a single pooled variance (FIGS. 141 and 14K).

FIGS. 15A-15G show how structural features of AMG1541 impact its properties. FIG. 15A shows functional groups present in ionizable lipid AMG1541. FIG. 15B shows TNS measurement of LNP pKa for AMG1541 and SM-102. FIG. 15C shows lipid pKa versus fold increase in mRNA transfection IM at (1 μg, 6 hour) IM for AMG lipids. FIG. 15D shows ³¹P NMR spectra of AMG1541 lipids showing spectral shift indicating bilayer membrane to hexagonal H_{II} transformation at acidic pH. FIG. 15E shows in vivo FLuc mRNA transfection of AMG1541 and linear counterpart AMG2041 at 0.1 g. FIG. 15F shows in vivo FLuc mRNA transfection of AMG1041 and —OH deleted derivative AMG1041OHD at 0.1 μg, 6 hour post IM injection. FIG. 15G shows the structure of —OH deleted derivative AMG1041OHD. Statistical significance was analyzed using a two-way analysis of variance with Šídák's multiple comparisons test with a single pooled variance (FIGS. 15E, 15F, and 15G).

FIGS. 16A and 16B show in vitro transfection data for AMG lipid library in C2C12 myoblast cells after treatment with FLuc encapsulating LNPs dosed at 40 ng/well. FIG. 16A shows the data in bar graphs. FIG. 16B shows the data in heat maps. Mean±s.d., n=3 wells per group. RLU, relative light unit.

FIGS. 17A and 17B show the effect of various lipid tails. FIG. 17A shows the structures of different tails. FIG. 17B shows quantification of Fluc protein expression upon intramuscular injection in mice for AMG1541 tail derivatives (0.1 μg, 6 hours).

FIGS. 18A and 18B show quantification of EPO expression in blood, 6 hours post intravenous administration of EPO encapsulating LNPs. FIG. 18A shows a comparison of AMG1541 and cKK-E12 in a dose response study. FIG. 18B shows a comparison of delivery efficacy with Lipid 5 and LP01. Statistical significance was analyzed using a two-way analysis of variance with Šídák's multiple comparisons test with a single pooled variance (n=3 per group).

FIGS. 19A-19E show comparative intramuscular Flue protein expression 6-hour post intramuscular injection. FIG. 19A shows a comparison of AMG1041 and 1041 OHD (0.1 and 1 g). FIG. 19B shows a comparison of AMG1041, 1041R, and 1041 S (1 g). FIG. 19C shows a comparison of AMG1541, 1541R, and 1541 S (0.1 g). FIG. 19D shows a comparison of AMG1541 and its analogue without β-amino alcohol (AMG1559). FIG. 19E shows the structures of AMG1041OHD and AMG1041. Statistical significance was analyzed using a two-way analysis of variance with Šídák's multiple comparisons test with a single pooled variance (n=3-5 per group).

FIGS. 20A-20C show additional vaccine response characterization. FIG. 20A shows day 35 peptide pool-stimulated splenocyte IFN-γ ELISpot SFU (left) and representative images (right) from female BALB/cJ mice vaccinated with HA mRNA (dose=1 μg, n=5). FIG. 28B shows day 35 dose-response serum antibody titers in female BALB/cJ mice following H3 HA vaccination (n=5). FIG. 20C shows serum cytokines 6 hrs. after boosting with HA mRNA in BALB/cJ mice (dose=1 μg, n=5). Statistical analysis was performed using an unpaired t test with Welch correction with the two-stage linear step-up procedure of Benjamini, Krieger, and Yekutieli (FIG. 20A), an ordinary two-way analysis of variance with Šídák's multiple comparisons tests (FIG. 20B), or a repeated measurement two-way analysis of variances with the Geisser-Greenhouse correction and Šídák's multiple comparisons with individual variances for each comparison (FIG. 20C).

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In a formula, the bond [symbol] is a single bond, the dashed line [symbol] is a single bond or absent, and the bond [symbol] or [symbol] is a single or double bond.

The term “aliphatic” refers to alkyl, alkenyl, alkynyl, and carbocyclic groups.

Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”).

In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

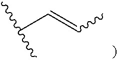

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has Ito 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁. 8 alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₅). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR—, —SC(═O)OR—, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{aa})₂, —P(OR^{cc})₂, —P(R^{aa})₃⁺X⁻, —P(OR)₃⁺X⁻, —P(R^{aa})₄, —P(OR^{cc})₄, —OP(R^{aa})₂, —OP(R^{aa})₃⁺X⁻, —OP(OR^{cc})₂, —OP(OR)₃⁺X⁻, —OP(R^{aa})₄, —OP(OR^{cc})₄, —B(R′)₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd}groups; wherein X⁻ is a counterion;
or two geminal hydrogens on a carbon atom are replaced with the group ═O, ═S, ═NN(R^{bb})₂, ═NNR^{bb}C(═O)R^{aa}, =NNR^{bb}C(═O)OR^{aa}, =NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{cc}, —C(═O)N(R^{aa})₂, —CO₂R^{aa}, —SO₂R^{aa}, —C(═NR^{cc})OR^{aa}, —C(═NR^{cc})N(R^{cc})₂, —SO₂N(R^{aa})₂, —SO₂R^{aa}, —SO₂OR^{cc}, —SOR^{aa}, —C(═S)N(R^{aa})₂, —C(═O)SR^{cc}, —C(═S)SR^{cc}, —P(═O)(R′)₂, —P(═O)(OR^{cc})₂, —P(═O)(N(R^{aa})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{f})₂, —N(R^{f})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ee} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻is a counterion.

In certain embodiments, each carbon atom substituent is independently halogen, substituted (e.g., substituted with one or more halogen) or unsubstituted C₁₋₆ alkyl, —OR^{aa}, —SR—, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{a}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR—, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{cc} and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR—, —C(═O)—O—C(═O)R—, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂—CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR—, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2—(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3—(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR)₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{cc})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

### Other Definitions

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·×H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “isotopes” refers to variants of a particular chemical element such that, while all isotopes of a given element share the same number of protons in each atom of the element, those isotopes differ in the number of neutrons.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₅ alkenyl, C₂-C₅ alkynyl, aryl, C₇-C₁₂ substituted aryl, and C₇-C₁₂ arylalkyl esters of the compounds described herein may be preferred.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms.

The terms “phosphorylethanolamine” and “phosphoethanolamine” are used interchangeably.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid). Exemplary amino acid side chains are depicted herein.

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “apolipoprotein” refers to a protein that binds a lipid (e.g., triacylglycerol or cholesterol) to form a lipoprotein. Apolipoproteins also serve as enzyme cofactors, receptor ligands, and lipid transfer carriers that regulate the metabolism of lipoproteins and their uptake in tissues. Major types of apolipoproteins include integral and non-integral apolipoproteins. Exemplary apolipoproteins include apoA (e.g., apoA-I, apoA-II, apoA-IV, and apoA-V); apoB (e.g., apoB48 and apoB 100); apoC (e.g., apoC-I, apoC-II, apoC-III, and apoC-IV); apoD; apoE; apoH; and apoJ.

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. The oligonucleotide may comprise one or more locked nucleic acid (LNA) moieties. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids.

Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), a provirus, a lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), a polyinosinic acid, a ribozyme, a flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Such promoters include, but are not limited to: the SV40 early promoter region (Bernoist et al., Nature, 290, 304-310, (1981); Yamamoto et al., Cell, 22, 787-797, (1980); Wagner et al., Proc. Natl. Acad. Sci. U.S.A. 78, 1441-1445, (1981); Brinster et al., Nature 296, 39-42, (1982)). Any type of plasmid, cosmid, yeast artificial chromosome, or viral vector can be used to prepare the recombinant DNA construct that can be introduced directly into the tissue site. Alternatively, viral vectors can be used which selectively infect the desired tissue, in which case administration may be accomplished by another route (e.g., systemically).

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design. Examples of recombinant nucleic acid molecule include recombinant vectors, such as cloning or expression vectors which contain DNA sequences encoding Ror family proteins or immunoglobulin proteins which are in a 5′ to 3′ (sense) orientation or in a 3′ to 5′ (antisense) orientation.

The term “pDNA,” “plasmid DNA,” or “plasmid” refers to a small DNA molecule that is physically separate from, and can replicate independently of, chromosomal DNA within a cell. Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer. Artificial plasmids are widely used as vectors in molecular cloning, serving to drive the replication of recombinant DNA sequences within host subjects. Plasmid sizes may vary from 1 to over 1,000 kbp. Plasmids are considered replicons, capable of replicating autonomously within a suitable host.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “gene silencing” refers to an epigenetic process of gene regulation where a gene is “switched off” by a mechanism other than genetic modification. That is, a gene which would be expressed (i.e., “turned on”) under normal circumstances is switched off by machinery in the cell. Gene silencing occurs when RNA is unable to make a protein during translation. Genes are regulated at either the transcriptional or post-transcriptional level. Transcriptional gene silencing is the result of histone modifications, creating an environment of heterochromatin around a gene that makes it inaccessible to transcriptional machinery (e.g., RNA polymerase and transcription factors). Post-transcriptional gene silencing is the result of mRNA of a particular gene being destroyed or blocked. The destruction of the mRNA prevents translation and thus the formation of a gene product (e.g., a protein). A common mechanism of post-transcriptional gene silencing is RNAi.

As used herein, a “chimeric receptor” or “chimeric antigen receptor” refers to a non-naturally occurring molecule that can be expressed on the surface of a host cell and comprises binding domain that provides specificity of the chimeric receptor (e.g., an antigen-binding fragment that binds to an epitope of a cell surface protein). In general, chimeric receptors comprise at least two domains that are derived from different molecules. In addition to the epitope-binding fragment described herein, the chimeric receptor may further comprise one or more of the following: a hinge domain, a transmembrane domain, a co-stimulatory domain, a cytoplasmic signaling domain, and combinations thereof. In some embodiments, the chimeric receptor comprises from N terminus to C terminus, an antigen-binding fragment that binds to a cell surface protein, a hinge domain, a transmembrane domain, and a cytoplasmic signaling domain. In some embodiments, the chimeric receptor further comprises at least one co-stimulatory domain. See, e.g., Marin-Acevedo et al. J. Hematol. Oncol.(2018)11: 8.

In some embodiments, the chimeric receptors described herein comprise one or more hinge domain(s). In some embodiments, the hinge domain may be located between the antigen-binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the antigen-binding fragment relative to another domain of the chimeric receptor can be used.

In some embodiments, the chimeric receptors described herein may comprise one or more transmembrane domain(s). The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, the transmembrane domain may be a synthetic, non-naturally occurring protein segment, e.g., a hydrophobic protein segment that is thermodynamically stable in a cell membrane.

In some embodiments, the chimeric receptors described herein comprise one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher-than-normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound or agent described herein refers to an amount sufficient to elicit the desired biological response. An effective amount of a compound or agent described herein may vary depending on such factors as the desired biological endpoint, severity of side effects, disease, or disorder, the identity, pharmacokinetics, and pharmacodynamics of the particular compound or agent, the condition being treated, the mode, route, and desired or required frequency of administration, the species, age and health or general condition of the subject. In certain embodiments, an effective amount is a therapeutically effective amount. In certain embodiments, an effective amount is a prophylactically effective amount. In certain embodiments, an effective amount is the amount of a compound or agent described herein in a single dose. In certain embodiments, an effective amount is the combined amounts of a compound or agent described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound or agent described herein is an amount sufficient to provide a therapeutic benefit in the treatment of a condition or to delay or minimize one or more symptoms associated with the condition. A therapeutically effective amount of a compound or agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for delivering an agent to a subject or a cell. In certain embodiments, a therapeutically effective amount is an amount sufficient for delivering a polynucleotide to a subject or a cell. In certain embodiments, a therapeutically effective amount is an amount sufficient for delivering mRNA to a subject or a cell. In certain embodiments, a therapeutically effective amount is an amount sufficient for treating a disease, disorder, or condition. In certain embodiments, a therapeutically effective amount is an amount sufficient for delivering an agent to a subject or a cell and treating a disease, disorder, or condition. In certain embodiments, a therapeutically effective amount is an amount sufficient for delivering a polynucleotide to a subject or a cell and treating a disease, disorder, or condition. In certain embodiments, a therapeutically effective amount is an amount sufficient for delivering mRNA to a subject or a cell and treating a disease, disorder, or condition. In certain embodiments, a therapeutically effective amount is an amount effective for chimeric antigen receptor T cell therapy.

A “prophylactically effective amount” of a compound or agent described herein is an amount sufficient to prevent a condition, or one or more symptoms associated with the condition or prevent its recurrence. A prophylactically effective amount of a compound or agent means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for delivering an agent to a subject or a cell. In certain embodiments, a prophylactically effective amount is an amount sufficient for delivering a polynucleotide to a subject or a cell. In certain embodiments, a prophylactically effective amount is an amount sufficient for delivering mRNA to a subject or a cell. In certain embodiments, a prophylactically effective amount is an amount sufficient for preventing a disease, disorder, or condition. In certain embodiments, a prophylactically effective amount is an amount sufficient for delivering an agent to a subject or a cell and preventing a disease, disorder, or condition. In certain embodiments, a prophylactically effective amount is an amount sufficient for delivering a polynucleotide to a subject or a cell and preventing a disease, disorder, or condition. In certain embodiments, a prophylactically effective amount is an amount sufficient for delivering mRNA to a subject or a cell and preventing a disease, disorder, or condition. In certain embodiments, a prophylactically effective amount is an amount effective for chimeric antigen receptor T cell therapy.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculoskeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g., nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transsection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia). [00449] In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

In certain embodiments, the metabolic disorder is a wasting condition. A “wasting condition” includes but is not limited to, anorexia and cachexias of various natures (e.g., weight loss associated with cancer, weight loss associated with other general medical conditions, weight loss associated with failure to thrive, and the like). In certain embodiments, the metabolic disorder is an obesity-related condition or a complication thereof. An “obesity-related condition” includes, but is not limited to, obesity, undesired weight gain (e.g., from medication-induced weight gain, from cessation of smoking) and an over-eating disorder (e.g., binge eating, bulimia, compulsive eating, or a lack of appetite control each of which can optionally lead to undesired weight gain or obesity). “Obesity” and “obese” refers to class I obesity, class II obesity, class III obesity and pre-obesity (e.g., being “over-weight”) as defined by the World Health Organization.

Reduction of storage fat is expected to provide various primary and/or secondary benefits in a subject (e.g., in a subject diagnosed with a complication associated with obesity) such as, for example, an increased insulin responsiveness (e.g., in a subject diagnosed with Type II diabetes mellitus); a reduction in elevated blood pressure; a reduction in elevated cholesterol levels; and/or a reduction (or a reduced risk or progression) of ischemic heart disease, arterial vascular disease, angina, myocardial infarction, stroke, migraines, congestive heart failure, deep vein thrombosis, pulmonary embolism, gall stones, gastroesophagael reflux disease, obstructive sleep apnea, obesity hypoventilation syndrome, asthma, gout, poor mobility, back pain, erectile dysfunction, urinary incontinence, liver injury (e.g., fatty liver disease, liver cirrhosis, alcoholic cirrhosis, endotoxin mediated liver injury) or chronic renal failure.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes.

Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formula (I), (XI), (XII), and (XIII)), and pharmaceutically acceptable salts, solvates, tautomers, stereoisomers, and isotopically labeled derivatives thereof, and compositions and kits thereof. The compounds provided herein can form particles and may therefore be used to deliver agents (e.g., a polynucleotide) to a subject, tissue, or cell. Also provided herein are methods of delivery and methods of treating a disease, disorder, or condition, comprising administering to the subject a composition comprising a compound provided herein (e.g., a compound of Formula (I), (XI), (XII), or (XIII)), or a pharmaceutically acceptable salt thereof.

### Compounds

Provided herein is a compound of Formula (I):

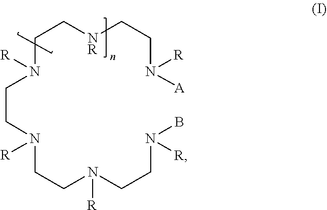

or a pharmaceutically acceptable salt, solvate, tautomer, stereoisomer, or isotopically labeled derivative thereof, wherein:
n is 1, 2, or 3;
A and B are independently optionally substituted saturated or unsaturated aliphatic; or
A and B combine to form optionally substituted C₂-C₆ alkylene;
each R is independently hydrogen, a nitrogen protecting group, or a group of the Formula (i), (ii), (iii), (iv) or (v):
   (i)

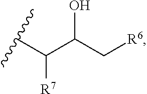

wherein one of R⁶ and R⁷ is hydrogen, and the other is

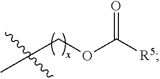

(ii)

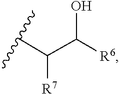

wherein one of R⁶ and R⁷ is hydrogen, and the other is

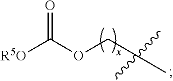

(iii)

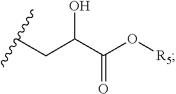

(iv)

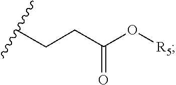

and
(v)

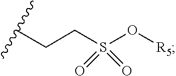

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In a particular embodiment, provided herein is a compound of Formula (I), or a pharmaceutically acceptable salt thereof.

Also provided herein is a compound of the formula:

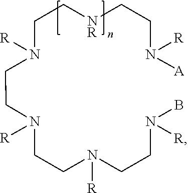

or a pharmaceutically acceptable salt thereof, wherein:
n is 1, 2, or 3;
A and B are independently optionally substituted saturated or unsaturated aliphatic; or
A and B combine to form optionally substituted C₂-C₆ alkylene;
each R is independently hydrogen, a nitrogen protecting group, or a group of the Formula (i), (ii), (iii), (iv) or (v):
   (i)

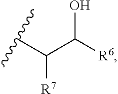

wherein one of R⁶ and R⁷ is hydrogen, and the other is

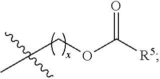

(ii)

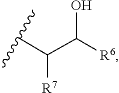

wherein one of R⁶ and R⁷ is hydrogen, and the other is

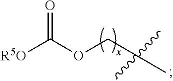

(iii)

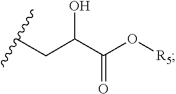

(iv)

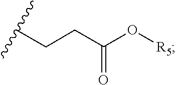

and
(v)

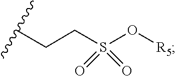

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In some embodiments of Formula (I), each R is independently hydrogen, a nitrogen protecting group, or a group of the Formula (i), provided that at least one R is of formula (i).

In some embodiments of Formula (I), each R is independently hydrogen, a nitrogen protecting group, or a group of the Formula (ii), provided that at least one R is of Formula (ii).

In some embodiments of Formula (I), each R is independently hydrogen, a nitrogen protecting group, or a group of the Formula (iii), provided that at least one R is of Formula (iii).

In some embodiments of Formula (I), each R is independently hydrogen, a nitrogen protecting group, or a group of the Formula (iv), provided that at least one R is of Formula (iv).

In some embodiments of Formula (I), each R is independently hydrogen, a nitrogen protecting group, or a group of the Formula (v), provided that at least one R is of Formula (v).

Also provided herein is a compound of Formula (I′):

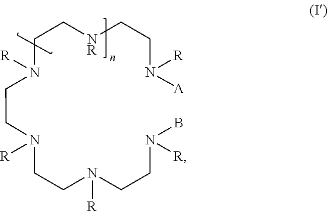

or a pharmaceutically acceptable salt, solvate, tautomer, stereoisomer, or isotopically labeled derivative thereof, wherein:
n is 1, 2, or 3;
A and B are independently optionally substituted saturated or unsaturated aliphatic; or
A and B combine to form optionally substituted C₂-C₆ alkylene;
each R is independently hydrogen, a nitrogen protecting group, or

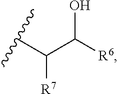

wherein one of R⁶ and R⁷ is hydrogen, and the other is

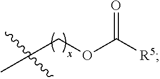

each instance of R⁵ is independently optionally substituted, C₄-C₃₅ saturated or unsaturated aliphatic or optionally substituted, C₄-C₃₅ saturated or unsaturated heteroaliphatic; and
each x independently is an integer from 1 to 10, inclusive;
provided that at least one R is

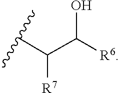

In a particular embodiment, provided herein is a compound of Formula (I′), or a pharmaceutically acceptable salt thereof.

Also provided herein is a compound of the formula:

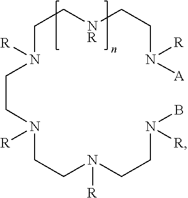

or a pharmaceutically acceptable salt thereof, wherein:
n is 1, 2, or 3;
A and B are independently optionally substituted saturated or unsaturated aliphatic; or
A and B combine to form optionally substituted C₂-C₆ alkylene;
each R is independently hydrogen, a nitrogen protecting group, or

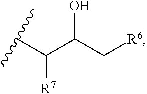

wherein one of R⁶ and R⁷ is hydrogen, and the other is

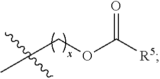

each instance of R⁵ is independently optionally substituted, C₄-C₃₅ saturated or unsaturated aliphatic or optionally substituted, C₄-C₃₅ saturated or unsaturated heteroaliphatic; and
each x independently is an integer from 1 to 10, inclusive;
provided that at least one R is

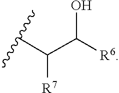

Provided herein is a compound of Formula (XI):

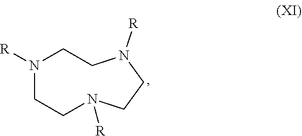

or a pharmaceutically acceptable salt, solvate, tautomer, stereoisomer, or isotopically labeled derivative thereof, wherein:
each R is independently hydrogen, a nitrogen protecting group, or a group of the formula (i), (ii), (iii), (iv) or (v):
   (i)

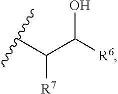

wherein one of R⁶ and R⁷ is hydrogen, and the other is

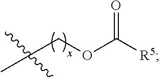

(ii)

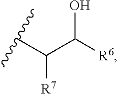

wherein one of R⁶ and R⁷ is hydrogen, and the other is

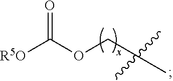

(iii)

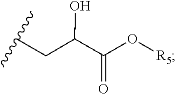

(iv)

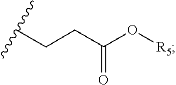

and

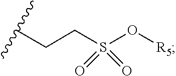

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In a particular embodiment, provided herein is a compound of Formula (XI), or a pharmaceutically acceptable salt thereof.

In some embodiments of Formula (XI), each R is independently hydrogen, a nitrogen protecting group, or a group of the Formula (i), provided that at least one R is of formula (i).

In some embodiments of Formula (XI), each R is independently hydrogen, a nitrogen protecting group, or a group of the Formula (ii), provided that at least one R is of Formula (ii).

In some embodiments of Formula (XI), each R is independently hydrogen, a nitrogen protecting group, or a group of the Formula (iii), provided that at least one R is of Formula (iii).

In some embodiments of Formula (XI), each R is independently hydrogen, a nitrogen protecting group, or a group of the Formula (iv), provided that at least one R is of Formula (iv).

In some embodiments of Formula (XI), each R is independently hydrogen, a nitrogen protecting group, or a group of the Formula (v), provided that at least one R is of Formula (v).

Provided herein is a compound of Formula (XII):

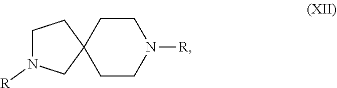

or a pharmaceutically acceptable salt, solvate, tautomer, stereoisomer, or isotopically labeled derivative thereof, wherein:
each R is independently hydrogen, a nitrogen protecting group, or a group of the formula (i), (ii), (iii), (iv) or (v):
   (i)

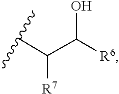

wherein one of R⁶ and R⁷ is hydrogen, and the other is

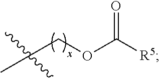

(ii)

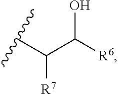

wherein one of R⁶ and R⁷ is hydrogen, and the other is

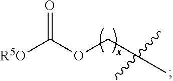

(iii)

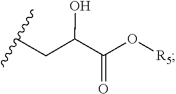

(iv)

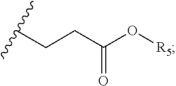

and
(v)

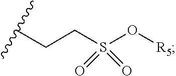

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In a particular embodiment, provided herein is a compound of Formula (XII), or a pharmaceutically acceptable salt thereof.

In some embodiments of Formula (XII), each R is independently hydrogen, a nitrogen protecting group, or a group of the Formula (i), provided that at least one R is of formula (i).

In some embodiments of Formula (XII), each R is independently hydrogen, a nitrogen protecting group, or a group of the Formula (ii), provided that at least one R is of Formula (ii).

In some embodiments of Formula (XII), each R is independently hydrogen, a nitrogen protecting group, or a group of the Formula (iii), provided that at least one R is of Formula (iii).

In some embodiments of Formula (XII), each R is independently hydrogen, a nitrogen protecting group, or a group of the Formula (iv), provided that at least one R is of Formula (iv).

In some embodiments of Formula (XII), each R is independently hydrogen, a nitrogen protecting group, or a group of the Formula (v), provided that at least one R is of Formula (v).

Provided herein is a compound of Formula (XIII):

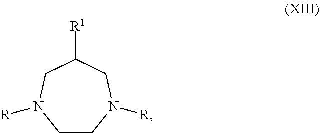

or a pharmaceutically acceptable salt, solvate, tautomer, stereoisomer, or isotopically labeled derivative thereof, wherein:
R¹ is hydrogen, —OR^{A}, —SR^{A}, or —N(R^{A})₂,
each instance of R^{A} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, an oxygen protecting group when attached to an oxygen atom, a nitrogen protecting group when attached to a nitrogen atom, or a sulfur protecting group when attached to a sulfur atom, or wherein two instances of R^{A} attached to the same nitrogen atom are joined together with the intervening atoms to form optionally substituted heterocyclyl or optionally substituted heteroaryl;
each R is independently hydrogen, a nitrogen protecting group, or a group of the formula (i), (ii), (iii), (iv) or (v):
   (i)

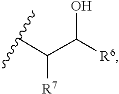

wherein one of R⁶ and R⁷ is hydrogen, and the other is

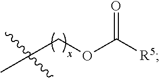

(ii)

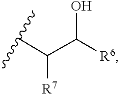

wherein one of R⁶ and R⁷ is hydrogen, and the other is

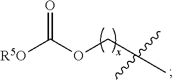

(iii)

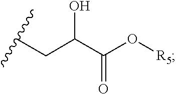

(iv)

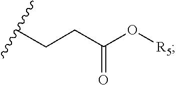

and
(v)

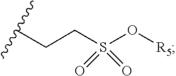

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In a particular embodiment, provided herein is a compound of Formula (XIII), or a pharmaceutically acceptable salt thereof.

In some embodiments of Formula (XIII), each R is independently hydrogen, a nitrogen protecting group, or a group of the Formula (i), provided that at least one R is of formula (i).

In some embodiments of Formula (XIII), each R is independently hydrogen, a nitrogen protecting group, or a group of the Formula (ii), provided that at least one R is of Formula (ii).

In some embodiments of Formula (XIII), each R is independently hydrogen, a nitrogen protecting group, or a group of the Formula (iii), provided that at least one R is of Formula (iii).

In some embodiments of Formula (XIII), each R is independently hydrogen, a nitrogen protecting group, or a group of the Formula (iv), provided that at least one R is of Formula (iv).

In some embodiments of Formula (XIII), each R is independently hydrogen, a nitrogen protecting group, or a group of the Formula (v), provided that at least one R is of Formula (v).

As defined herein, R¹ is hydrogen, —OR^{A}, —SR^{A}, or —N(R^{A})₂. In some embodiments, R¹ is hydrogen or —OR^{A}. In some embodiments, R¹ is hydrogen or —OH. In some embodiments, R¹ is hydrogen or —SR^{A}. In some embodiments, R¹ is hydrogen or —SH. In some embodiments, R¹ is hydrogen or —N(R^{A})₂. In some embodiments, R¹ is hydrogen or —NH₂. In some embodiments, R¹ is hydrogen, —OR^{A}, or —SR^{A}. In some embodiments, R¹ is hydrogen, —OH, or —SH. In some embodiments, R¹ is —OR^{A}, —SR^{A}, or —N(R^{A})₂. In some embodiments, R¹ is hydrogen. In some embodiments, R¹ is —OR^{A}. In some embodiments, R¹ is —OH. In some embodiments, R¹ is —SR^{A}. In some embodiments, R¹ is —SH. In some embodiments, R¹ is —N(R^{A})₂. In some embodiments, R¹ is —NH₂.

As defined herein, each instance of R^{A} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, an oxygen protecting group when attached to an oxygen atom, a nitrogen protecting group when attached to a nitrogen atom, or a sulfur protecting group when attached to a sulfur atom, or wherein two instances of R^{A} attached to the same nitrogen atom are joined together with the intervening atoms to form optionally substituted heterocyclyl or optionally substituted heteroaryl. In some embodiments, R^{A} is —H. In some embodiments, R^{A} is optionally substituted C₁₋₁₀ aliphatic. In some embodiments, R^{A} is optionally substituted C₁₋₁₀ alkyl. In some embodiments, R^{A} is optionally substituted C₁₋₁₀ alkenyl. In some embodiments, R^{A} is optionally substituted C₁₋₁₀ alkynyl. In some embodiments, R^{A} is optionally substituted C₁₋₁₀ heteroaliphatic. In some embodiments, R^{A} is optionally substituted C₁₋₁₀ heteroalkyl. In some embodiments, R^{A} is optionally substituted C₁₋₁₀ heteroalkenyl. In some embodiments, R^{A} is optionally substituted C₁₋₁₀ heteroalkynyl. In some embodiments, R^{A} is optionally substituted acyl. In some embodiments, R^{A} is optionally substituted aryl. In some embodiments, R^{A} is optionally substituted phenyl. In some embodiments, R^{A} is optionally substituted heteroaryl. In some embodiments, R^{A} is optionally substituted 5- or 6-membered heteroaryl. In some embodiments, R^{A} is optionally substituted carbocyclyl. In some embodiments, R^{A} is optionally substituted 3- to 8-membered carbocyclyl. In some embodiments, R^{A} is optionally substituted heterocyclyl. In some embodiments, R^{A} is optionally substituted 3- to 8-membered heterocyclyl. In some embodiments, R^{A} is an oxygen protecting group. In some embodiments, R^{A} is a sulfur protecting group. In some embodiments, R^{A} is a nitrogen protecting group. In some embodiments, two instances of R^{A} attached to the same nitrogen atom are joined together with the intervening atoms to form optionally substituted heterocyclyl. In some embodiments, two instances of R^{A} attached to the same nitrogen atom are joined together with the intervening atoms to form optionally substituted heteroaryl.

As defined herein, n is 1, 2, or 3. In some embodiments, n is 1 or 2. In some embodiments, n is 1 or 3. In some embodiments, n is 2 or 3. In some embodiments, n is 1. In some embodiments, n is 2. In some embodiments, n is 3.

As defined herein, A and B are independently optionally substituted saturated or unsaturated aliphatic; or A and B combine to form optionally substituted C₂-C₆ alkylene. In some embodiments, A and B are the same. In some embodiments, A and B are independently optionally substituted saturated or unsaturated aliphatic. In some embodiments, A and B are independently substituted saturated or unsaturated aliphatic. In some embodiments, A and B are independently substituted saturated aliphatic. In some embodiments, A and B are independently substituted unsaturated aliphatic. In some embodiments, A and B are independently unsubstituted saturated or unsaturated aliphatic. In some embodiments, A and B are independently unsubstituted saturated aliphatic. In some embodiments, A and B are independently unsubstituted unsaturated aliphatic.

In some embodiments, A and B are independently optionally substituted C₁-C₂₀ saturated or unsaturated aliphatic. In some embodiments, A and B are independently substituted C₁-C₂₀ saturated or unsaturated aliphatic. In some embodiments, A and B are independently substituted C₁-C₂₀ saturated aliphatic. In some embodiments, A and B are independently substituted C₁-C₂₀ unsaturated aliphatic. In some embodiments, A and B are independently unsubstituted C₁-C₂₀ saturated or unsaturated aliphatic. In some embodiments, A and B are independently unsubstituted C₁-C₂₀ saturated aliphatic. In some embodiments, A and B are independently unsubstituted C₁-C₂₀ unsaturated aliphatic.

In some embodiments, A and B are independently optionally substituted C₁-C₆ saturated or unsaturated aliphatic. In some embodiments, A and B are independently substituted C₁-C₆ saturated or unsaturated aliphatic. In some embodiments, A and B are independently substituted C₁-C₆ saturated aliphatic. In some embodiments, A and B are independently substituted C₁-C₆ unsaturated aliphatic. In some embodiments, A and B are independently unsubstituted C₁-C₆ saturated or unsaturated aliphatic. In some embodiments, A and B are independently unsubstituted C₁-C₆ saturated aliphatic. In some embodiments, A and B are independently unsubstituted C₁-C₆ unsaturated aliphatic.

In some embodiments, A and B are independently optionally substituted alkyl. In some embodiments, A and B are independently substituted alkyl. In some embodiments, A and B are independently unsubstituted alkyl. In some embodiments, A and B are independently optionally substituted C₁-C₂₀ alkyl. In some embodiments, A and B are independently substituted C₁-C₂₀ alkyl. In some embodiments, A and B are independently unsubstituted C₁-C₂₀ alkyl. In some embodiments, A and B are independently optionally substituted C₁-C₆ alkyl. In some embodiments, A and B are independently substituted C₁-C₆ alkyl. In some embodiments, A and B are independently unsubstituted C₁-C₆ alkyl. In some embodiments, A and B are independently —CH₃.

In some embodiments, A and B are independently optionally substituted alkenyl. In some embodiments, A and B are independently substituted alkenyl. In some embodiments, A and B are independently unsubstituted alkenyl. In some embodiments, A and B are independently optionally substituted C₁-C₂₀ alkenyl. In some embodiments, A and B are independently substituted C₁-C₂₀ alkenyl. In some embodiments, A and B are independently unsubstituted C₁-C₂₀ alkenyl. In some embodiments, A and B are independently optionally substituted C₁-C₆ alkenyl. In some embodiments, A and B are independently substituted C₁-C₆ alkenyl. In some embodiments, A and B are independently unsubstituted C₁-C₆ alkenyl.

In some embodiments, A is optionally substituted saturated or unsaturated aliphatic. In some embodiments, A is substituted saturated or unsaturated aliphatic. In some embodiments, A is substituted saturated aliphatic. In some embodiments, A is substituted unsaturated aliphatic. In some embodiments, A is unsubstituted saturated or unsaturated aliphatic. In some embodiments, A is unsubstituted saturated aliphatic. In some embodiments, A is unsubstituted unsaturated aliphatic.

In some embodiments, A is optionally substituted C₁-C₂₀ saturated or unsaturated aliphatic. In some embodiments, A is substituted C₁-C₂₀ saturated or unsaturated aliphatic. In some embodiments, A is substituted C₁-C₂₀ saturated aliphatic. In some embodiments, A is substituted C₁-C₂₀ unsaturated aliphatic. In some embodiments, A is unsubstituted C₁-C₂₀ saturated or unsaturated aliphatic. In some embodiments, A is unsubstituted C₁-C₂₀ saturated aliphatic. In some embodiments, A is unsubstituted C₁-C₂₀ unsaturated aliphatic.

In some embodiments, A is optionally substituted C₁-C₆ saturated or unsaturated aliphatic. In some embodiments, A is substituted C₁-C₆ saturated or unsaturated aliphatic. In some embodiments, A is substituted C₁-C₆ saturated aliphatic. In some embodiments, A is substituted C₁-C₆ unsaturated aliphatic. In some embodiments, A is unsubstituted C₁-C₆ saturated or unsaturated aliphatic. In some embodiments, A is unsubstituted C₁-C₆ saturated aliphatic. In some embodiments, A is unsubstituted C₁-C₆ unsaturated aliphatic.

In some embodiments, A is optionally substituted alkyl. In some embodiments, A is substituted alkyl. In some embodiments, A is unsubstituted alkyl. In some embodiments, A is optionally substituted C₁-C₂₀ alkyl. In some embodiments, A is substituted C₁-C₂₀ alkyl. In some embodiments, A is unsubstituted C₁-C₂₀ alkyl. In some embodiments, A is optionally substituted C₁-C₆ alkyl. In some embodiments, A is substituted C₁-C₆ alkyl. In some embodiments, A is unsubstituted C₁-C₆ alkyl. In some embodiments, A is —CH₃.

In some embodiments, A is optionally substituted alkenyl. In some embodiments, A is substituted alkenyl. In some embodiments, A is unsubstituted alkenyl. In some embodiments, A is optionally substituted C₁-C₂₀ alkenyl. In some embodiments, A is substituted C₁-C₂₀ alkenyl. In some embodiments, A is unsubstituted C₁-C₂₀ alkenyl. In some embodiments, A is optionally substituted C₁-C₆ alkenyl. In some embodiments, A is substituted C₁-C₆ alkenyl. In some embodiments, A is unsubstituted C₁-C₆ alkenyl.

In some embodiments, A is optionally substituted alkynyl. In some embodiments, A is substituted alkynyl. In some embodiments, A is unsubstituted alkynyl. In some embodiments, A is optionally substituted C₁-C₂₀ alkynyl. In some embodiments, A is substituted C₁-C₂₀ alkynyl. In some embodiments, A is unsubstituted C₁-C₂₀ alkynyl. In some embodiments, A is optionally substituted C₁-C₆ alkynyl. In some embodiments, A is substituted C₁-C₆ alkynyl.

In some embodiments, A is unsubstituted C₁-C₆ alkynyl.

In some embodiments, B is optionally substituted saturated or unsaturated aliphatic. In some embodiments, B is substituted saturated or unsaturated aliphatic. In some embodiments, B is substituted saturated aliphatic. In some embodiments, B is substituted unsaturated aliphatic. In some embodiments, B is unsubstituted saturated or unsaturated aliphatic. In some embodiments, B is unsubstituted saturated aliphatic. In some embodiments, B is unsubstituted unsaturated aliphatic.

In some embodiments, B is optionally substituted C₁-C₂₀ saturated or unsaturated aliphatic. In some embodiments, B is substituted C₁-C₂₀ saturated or unsaturated aliphatic. In some embodiments, B is substituted C₁-C₂₀ saturated aliphatic. In some embodiments, B is substituted C₁-C₂₀ unsaturated aliphatic. In some embodiments, B is unsubstituted C₁-C₂₀ saturated or unsaturated aliphatic. In some embodiments, B is unsubstituted C₁-C₂₀ saturatefR5d aliphatic. In some embodiments, B is unsubstituted C₁-C₂₀ unsaturated aliphatic.

In some embodiments, B is optionally substituted C₁-C₆ saturated or unsaturated aliphatic. In some embodiments, B is substituted C₁-C₆ saturated or unsaturated aliphatic. In some embodiments, B is substituted C₁-C₆ saturated aliphatic. In some embodiments, B is substituted C₁-C₆ unsaturated aliphatic. In some embodiments, B is unsubstituted C₁-C₆ saturated or unsaturated aliphatic. In some embodiments, B is unsubstituted C₁-C₆ saturated aliphatic. In some embodiments, B is unsubstituted C₁-C₆ unsaturated aliphatic.

In some embodiments, B is optionally substituted alkyl. In some embodiments, B is substituted alkyl. In some embodiments, B is unsubstituted alkyl. In some embodiments, B is optionally substituted C₁-C₂₀ alkyl. In some embodiments, B is substituted C₁-C₂₀ alkyl. In some embodiments, B is unsubstituted C₁-C₂₀ alkyl. In some embodiments, B is optionally substituted C₁-C₆ alkyl. In some embodiments, B is substituted C₁-C₆ alkyl. In some embodiments, B is unsubstituted C₁-C₆ alkyl. In some embodiments, B is —CH₃.

In some embodiments, B is optionally substituted alkenyl. In some embodiments, B is substituted alkenyl. In some embodiments, B is unsubstituted alkenyl. In some embodiments, B is optionally substituted C₁-C₂₀ alkenyl. In some embodiments, B is substituted C₁-C₂₀ alkenyl. In some embodiments, B is unsubstituted C₁-C₂₀ alkenyl. In some embodiments, B is optionally substituted C₁-C₆ alkenyl. In some embodiments, B is substituted C₁-C₆ alkenyl. In some embodiments, B is unsubstituted C₁-C₆ alkenyl.

In some embodiments, A and B are independently optionally substituted saturated or unsaturated aliphatic, and n is 1. In some embodiments, A and B are independently optionally substituted C₁-C₆ saturated or unsaturated aliphatic, and n is 1. In some embodiments, A and B are independently unsubstituted C₁-C₆ saturated or unsaturated aliphatic, and n is 1. In some embodiments, A and B are independently optionally substituted alkyl, and n is 1. In some embodiments, A and B are independently optionally substituted C₁-C₆ alkyl, and n is 1. In some embodiments, A and B are independently unsubstituted C₁-C₆ alkyl, and n is 1. In some embodiments, A and B are independently —CH₃, and n is 1.

In some embodiments, A and B are independently optionally substituted saturated or unsaturated aliphatic, and n is 2. In some embodiments, A and B are independently optionally substituted C₁-C₆ saturated or unsaturated aliphatic, and n is 2. In some embodiments, A and B are independently unsubstituted C₁-C₆ saturated or unsaturated aliphatic, and n is 2. In some embodiments, A and B are independently optionally substituted alkyl, and n is 2. In some embodiments, A and B are independently optionally substituted C₁-C₆ alkyl, and n is 2. In some embodiments, A and B are independently unsubstituted C₁-C₆ alkyl, and n is 2. In some embodiments, A and B are independently —CH₃, and n is 2.

In some embodiments, A and B are independently optionally substituted saturated or unsaturated aliphatic, and n is 3. In some embodiments, A and B are independently optionally substituted C₁-C₆ saturated or unsaturated aliphatic, and n is 3. In some embodiments, A and B are independently unsubstituted C₁-C₆ saturated or unsaturated aliphatic, and n is 3. In some embodiments, A and B are independently optionally substituted alkyl, and n is 3. In some embodiments, A and B are independently optionally substituted C₁-C₆ alkyl, and n is 3. In some embodiments, A and B are independently unsubstituted C₁-C₆ alkyl, and n is 3. In some embodiments, A and B are independently —CH₃, and n is 3.

In some embodiments, A and B combine to form optionally substituted C₂-C₆ alkylene. In some embodiments, A and B combine to form optionally substituted branched C₂-C₆ alkylene. In some embodiments, A and B combine to form optionally substituted unbranched C₂-C₆ alkylene. In some embodiments, A and B combine to form optionally substituted ethylene. In some embodiments, A and B combine to form optionally substituted n-propylene. In some embodiments, A and B combine to form optionally substituted n-butylene. In some embodiments, A and B combine to form optionally substituted n-pentylene. In some embodiments, A and B combine to form optionally substituted n-hexylene.

In some embodiments, A and B combine to form substituted C₂-C₆ alkylene. In some embodiments, A and B combine to form substituted branched C₂-C₆ alkylene. In some embodiments, A and B combine to form substituted unbranched C₂-C₆ alkylene. In some embodiments, A and B combine to form unsubstituted C₂-C₆ alkylene. In some embodiments, A and B combine to form unsubstituted branched C₂-C₆ alkylene. In some embodiments, A and B combine to form unsubstituted unbranched C₂-C₆ alkylene. In some embodiments, A and B combine to form ethylene. In some embodiments, A and B combine to form n-propylene. In some embodiments, A and B combine to form n-butylene. In some embodiments, A and B combine to form n-pentylene. In some embodiments, A and B combine to form n-hexylene.

In some embodiments, A and B combine to form optionally substituted C₂-C₆ alkylene, and n is 1. In some embodiments, A and B combine to form optionally substituted unbranched C₂-C₆ alkylene, and n is 1. In some embodiments, A and B combine to form unsubstituted C₂-C₆ alkylene, and n is 1. In some embodiments, A and B combine to form unsubstituted unbranched C₂-C₆ alkylene, and n is 1. In some embodiments, A and B combine to form ethylene, and n is 1.

In some embodiments, A and B combine to form optionally substituted C₂-C₆ alkylene, and n is 2. In some embodiments, A and B combine to form optionally substituted unbranched C₂-C₆ alkylene, and n is 2. In some embodiments, A and B combine to form unsubstituted C₂-C₆ alkylene, and n is 2. In some embodiments, A and B combine to form unsubstituted unbranched C₂-C₆ alkylene, and n is 2. In some embodiments, A and B combine to form ethylene, and n is 2.

In some embodiments, A and B combine to form optionally substituted C₂-C₆ alkylene, and n is 3. In some embodiments, A and B combine to form optionally substituted unbranched C₂-C₆ alkylene, and n is 3. In some embodiments, A and B combine to form unsubstituted C₂-C₆ alkylene, and n is 3. In some embodiments, A and B combine to form unsubstituted unbranched C₂-C₆ alkylene, and n is 3. In some embodiments, A and B combine to form ethylene, and n is 3.

As defined herein, each R is independently hydrogen, a nitrogen protecting group, or a group of the formula (i), (ii), (iii), (iv) or (v):
(i)

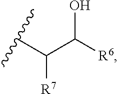

wherein one of R⁶ and R⁷ is hydrogen, and the other is

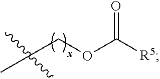

(ii)

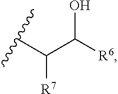

wherein one of R⁶ and R⁷ is hydrogen, and the other is

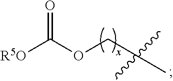

(iii)

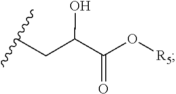

(iv)

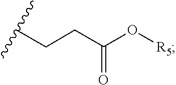

and
(v)

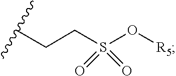

wherein each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is of Formula (i), (ii), (iii), (iv), or (v).

In some embodiments, each R is independently hydrogen, a nitrogen protecting group, or a group of the Formula (i), provided that at least one R is of Formula (i). In some embodiments, each R is independently hydrogen, a nitrogen protecting group, or a group of the Formula (ii), provided that at least one R is of Formula (ii). In some embodiments, each R is independently hydrogen, a nitrogen protecting group, or a group of the Formula (iii), provided that at least one R is of Formula (iii). In some embodiments, each R is independently hydrogen, a nitrogen protecting group, or a group of the Formula (iv), provided that at least one R is of Formula (iv). In some embodiments, each R is independently hydrogen, a nitrogen protecting group, or a group of the Formula (v), provided that at least one R is of Formula (v).

In some embodiments, each R is independently hydrogen, a nitrogen protecting group, or

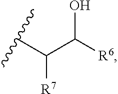

wherein one of R⁶ and R⁷ is hydrogen, and the other is

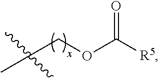

provided that at least one R is

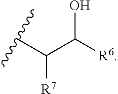

In some embodiments, at least one R is

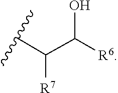

In some embodiments, at least two R are independently.

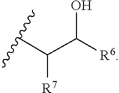

In some embodiments, at least three R are independently

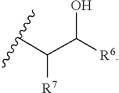

In some embodiments, at least four R are independently

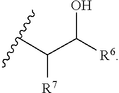

In some embodiments, at least five R are independently

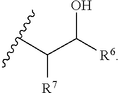

In some embodiments, at least six R are independently

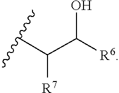

In some embodiments, at least seven R are independently

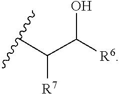

In some embodiments, at least eight R are independently

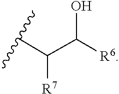

In some embodiments, each R is

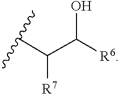

In some embodiments, one R is

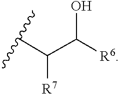

In some embodiments, two R are independently

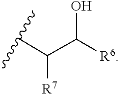

In some embodiments, three R are independently

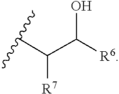

In some embodiments, four R are independently

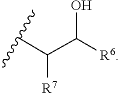

In some embodiments, five R are independently

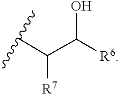

In some embodiments, six R are independently

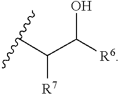

In some embodiments, seven R are independently

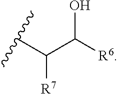

In some embodiments, eight R are independently

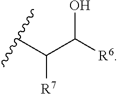

In some embodiments, at least one R is hydrogen or a nitrogen protecting group. In some embodiments, at least one R is hydrogen or

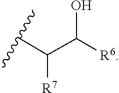

In some embodiments, at least one R is a nitrogen protecting group or

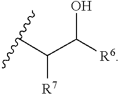

In some embodiments, at least one R is hydrogen. In some embodiments, at least one R is a nitrogen protecting group. In some embodiments, at least one R is Bn, Boc, Cbz, Fmoc, trifluoroacetyl, triphenylmethyl, acetyl, or Ts.
As defined herein, for each moiety

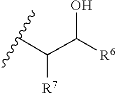

individually, one of R⁶ and R⁷ is hydrogen, and the other is

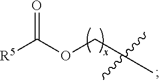

or for each moiety

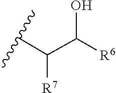

individually, one of R⁶ and R⁷ is hydrogen, and the other is

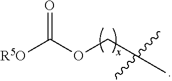

In some embodiments, for each moiety

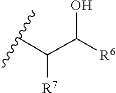

individually, one of R⁶ and R⁷ is hydrogen, and the other is

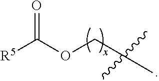

In some embodiments, R⁶ is hydrogen. In some embodiments, R⁷ is hydrogen. In some embodiments, R⁶ is

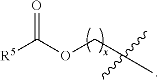

In some embodiments, R⁷ is

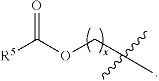

In some embodiments, at least one moiety

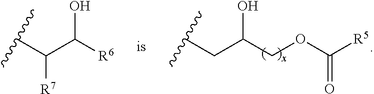

In some embodiments, each R⁶ is hydrogen. In some embodiments, each R⁷ is hydrogen. In some embodiments, each R⁶ is

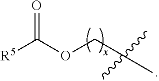

In some embodiments, each R⁷ is

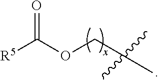

In some embodiments, each moiety

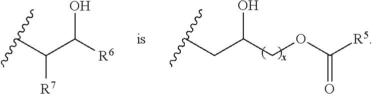

In some embodiments, each R is

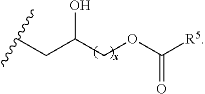

In some embodiments, one R is

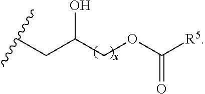

In some embodiments, two R are independently

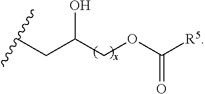

In some embodiments, three R are independently

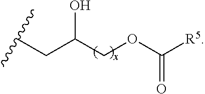

In some embodiments, four R are independently

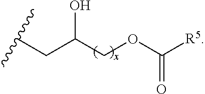

In some embodiments, five R are independently

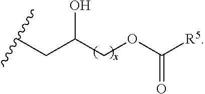

In some embodiments, six R are independently

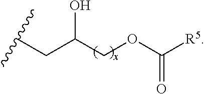

In some embodiments, seven R are independently

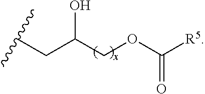

In some embodiments, eight R are independently

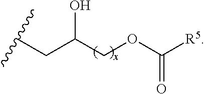

As defined herein, each instance of R⁵ is independently optionally substituted C₄-C₃₅ saturated or unsaturated aliphatic or optionally substituted C₄-C₃₅ saturated or unsaturated heteroaliphatic.

In some embodiments, each instance of R⁵ is different. In some embodiments, two instances of R⁵ are the same. In some embodiments, at least two instances of R⁵ are the same. In some embodiments, three instances of R⁵ are the same. In some embodiments, at least three instances of R⁵ are the same. In some embodiments, four instances of R⁵ are the same. In some embodiments, at least four instances of R⁵ are the same. In some embodiments, five instances of R⁵ are the same. In some embodiments, at least five instances of R⁵ are the same. In some embodiments, six instances of R⁵ are the same. In some embodiments, at least six instances of R⁵ are the same. In some embodiments, seven instances of R⁵ are the same. In some embodiments, at least seven instances of R⁵ are the same. In some embodiments, eight instances of R⁵ are the same. In some embodiments, at least eight instances of R⁵ are the same. In some embodiments, each instance of R⁵ is the same.

In some embodiments, each instance of R⁵ is independently optionally substituted C₄-C₃₅ saturated aliphatic or optionally substituted C₄-C₃₅ saturated heteroaliphatic. In some embodiments, each instance of R⁵ is independently optionally substituted C₄-C₃₅ unsaturated aliphatic or optionally substituted C₄-C₃₅ unsaturated heteroaliphatic.

In some embodiments, each instance of R⁵ is independently optionally substituted C₄-C₃₅ saturated or unsaturated aliphatic. In some embodiments, each instance of R⁵ is independently optionally substituted C₄-C₁₂ saturated or unsaturated aliphatic. In some embodiments, each instance of R⁵ is independently optionally substituted C₁₀-C₂₀ saturated or unsaturated aliphatic. In some embodiments, each instance of R⁵ is independently optionally substituted C₁₂-C₂₅ saturated or unsaturated aliphatic.

In some embodiments, each R⁵ is independently optionally substituted C₄-C₃₅ alkyl or optionally substituted C₄-C₃₅ alkenyl. In some embodiments, each R⁵ is independently optionally substituted C₄-C₁₀ alkyl or optionally substituted C₄-C₁₀ alkenyl. In some embodiments, each R⁵ is independently optionally substituted C₁₀-C₂₀ alkyl or optionally substituted C₁₀-C₂₀ alkenyl. In some embodiments, each R⁵ is independently optionally substituted C₁₂-C₂₅ alkyl or optionally substituted C₁₂-C₂₅ alkenyl.

In some embodiments, each R⁵ is independently optionally substituted C₄-C₃₅ alkyl or optionally substituted C₄-C₃₅ alkynyl. In some embodiments, each R⁵ is independently optionally substituted C₄-C₁₀ alkyl or optionally substituted C₄-C₁₀ alkynyl. In some embodiments, each R⁵ is independently optionally substituted C₁₀-C₂₀ alkyl or optionally substituted C₁₀-C₂₀ alkynyl. In some embodiments, each R⁵ is independently optionally substituted C₁₂-C₂₅ alkyl or optionally substituted C₁₂-C₂₅ alkynyl.

In some embodiments, each R⁵ is independently optionally substituted C₄-C₃₅ alkynyl or optionally substituted C₄-C₃₅ alkenyl. In some embodiments, each R⁵ is independently optionally substituted C₄-C₁₀ alkynyl or optionally substituted C₄-C₁₀ alkenyl. In some embodiments, each R⁵ is independently optionally substituted C₁₀-C₂₀ alkynyl or optionally substituted C₁₀-C₂₀ alkenyl. In some embodiments, each R⁵ is independently optionally substituted C₁₂-C₂₅ alkynyl or optionally substituted C₁₂-C₂₅ alkenyl.

In some embodiments, each R⁵ is independently branched C₄-C₃₅ alkyl. In some embodiments, each R⁵ is independently branched C₄-C₁₂ alkyl. In some embodiments, each R⁵ is independently branched C₁₀-C₂₀ alkyl. In some embodiments, each R⁵ is independently branched C₁₂-C₂₅ alkyl. In some embodiments, each R⁵ is independently unbranched C₄-C₃₅ alkyl. In some embodiments, each R⁵ is independently unbranched C₄-C₁₂ alkyl. In some embodiments, each R⁵ is independently unbranched C₁₀-C₂₀ alkyl. In some embodiments, each R⁵ is independently unbranched C₁₂-C₂₅ alkyl.

In some embodiments, each R⁵ is independently branched C₄-C₃₅ alkenyl. In some embodiments, each R⁵ is independently branched C₄-C₃₅ alkenyl comprising a single double bond. In some embodiments, each R⁵ is independently branched C₄-C₃₅ alkenyl comprising a single (Z) double bond. In some embodiments, each R⁵ is independently branched C₄-C₁₂ alkenyl. In some embodiments, each R⁵ is independently branched C₁₀-C₂₀ alkenyl. In some embodiments, each R⁵ is independently branched C₁₂-C₂₅ alkenyl.

In some embodiments, each R⁵ is independently unbranched C₄-C₃₅ alkenyl. In some embodiments, each R⁵ is independently unbranched C₄-C₃₅ alkenyl comprising a single double bond. In some embodiments, each R⁵ is independently unbranched C₄-C₃₅ alkenyl comprising a single (Z) double bond. In some embodiments, each R⁵ is independently unbranched C₄-C₃₅ alkenyl comprising two double bonds. In some embodiments, each R⁵ is independently unbranched C₄-C₃₅ alkenyl comprising two (Z) double bonds. In some embodiments, each R⁵ is independently unbranched C₄-C₃₅ alkenyl comprising

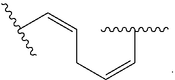 [SMILES: */C=C\C/C=C\*]

In some embodiments, each R⁵ is independently unbranched C₄-C₃₅ alkenyl comprising three double bonds. In some embodiments, each R⁵ is independently unbranched C₄-C₃₅ alkenyl comprising three (Z) double bonds. In some embodiments, each R⁵ is independently unbranched C₄-C₃₅ alkenyl comprising only (Z) double bonds.

In some embodiments, each R⁵ is independently unbranched C₄-C₁₂ alkenyl. In some embodiments, each R⁵ is independently unbranched C₄-C₁₂ alkenyl comprising a single double bond. In some embodiments, each R⁵ is independently unbranched C₄-C₁₂ alkenyl comprising a single (Z) double bond. In some embodiments, each R⁵ is independently unbranched C₄-C₁₂ alkenyl comprising two double bonds. In some embodiments, each R⁵ is independently unbranched C₄-C₁₂ alkenyl comprising two (Z) double bonds. In some embodiments, each R⁵ is independently unbranched C₄-C₁₂ alkenyl comprising

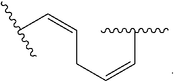 [SMILES: */C=C\C/C=C\*]

In some embodiments, each R⁵ is independently unbranched C₄-C₁₂ alkenyl comprising three double bonds. In some embodiments, each R⁵ is independently unbranched C₄-C₁₂ alkenyl comprising three (Z) double bonds. In some embodiments, each R⁵ is independently unbranched C₄-C₁₂ alkenyl comprising only (Z) double bonds.

In some embodiments, each R⁵ is independently unbranched C₁₀-C₂₀ alkenyl. In some embodiments, each R⁵ is independently unbranched C₁₀-C₂₀ alkenyl comprising a single double bond. In some embodiments, each R⁵ is independently unbranched C₁₀-C₂₀ alkenyl comprising a single (Z) double bond. In some embodiments, each R⁵ is independently unbranched C₁₀-C₂₀ alkenyl comprising two double bonds. In some embodiments, each R⁵ is independently unbranched C₁₀-C₂₀ alkenyl comprising two (Z) double bonds. In some embodiments, each R⁵ is independently unbranched C₁₀-C₂₀ alkenyl comprising

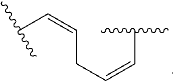 [SMILES: */C=C\C/C=C\*]

In some embodiments, each R⁵ is independently unbranched C₁₀-C₂₀ alkenyl comprising three double bonds. In some embodiments, each R⁵ is independently unbranched C₁₀-C₂₀ alkenyl comprising three (Z) double bonds. In some embodiments, each R⁵ is independently unbranched C₁₀-C₂₀ alkenyl comprising only (Z) double bonds.

In some embodiments, each R⁵ is independently unbranched C₁₂-C₂₅ alkenyl. In some embodiments, each R⁵ is independently unbranched C₁₂-C₂₅ alkenyl comprising a single double bond. In some embodiments, each R⁵ is independently unbranched C₁₂-C₂₅ alkenyl comprising a single (Z) double bond. In some embodiments, each R⁵ is independently unbranched C₁₂-C₂₅ alkenyl comprising two double bonds. In some embodiments, each R⁵ is independently unbranched C₁₂-C₂₅ alkenyl comprising two (Z) double bonds. In some embodiments each R⁵ is independently unbranched C₁₂-C₂₅ alkenyl comprising

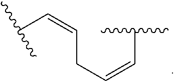 [SMILES: */C=C\C/C=C\*]

In some embodiments, each R⁵ is independently unbranched C₁₂-C₂₅ alkenyl comprising three double bonds. In some embodiments, each R⁵ is independently unbranched C₁₂-C₂₅ alkenyl comprising three (Z) double bonds. In some embodiments, each R⁵ is independently unbranched C₁₂-C₂₅ alkenyl comprising only (Z) double bonds.

In some embodiments, each R⁵ is independently unbranched C₁₁ alkenyl. In some embodiments, each R⁵ is independently unbranched C₁₁ alkenyl comprising a single double bond. In some embodiments, each R⁵ is independently unbranched C₁₁ alkenyl comprising a single (Z) double bond. In some embodiments, each R⁵ is independently unbranched C₁₁ alkenyl comprising two double bonds. In some embodiments, each R⁵ is independently unbranched C₁₁ alkenyl comprising two (Z) double bonds. In some embodiments, each R⁵ is independently unbranched C₁₁ alkenyl comprising

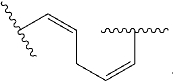 [SMILES: */C=C\C/C=C\*]

In some embodiments, each R⁵ is independently unbranched C₁₁ alkenyl comprising three double bonds. In some embodiments, each R⁵ is independently unbranched C₁₁ alkenyl comprising three (Z) double bonds. In some embodiments, each R⁵ is independently unbranched C₁₁ alkenyl comprising only (Z) double bonds.

In some embodiments, each R⁵ is independently branched C₄-C₃₅ alkynyl. In some embodiments, each R⁵ is independently branched C₄-C₁₂ alkynyl. In some embodiments, R⁵ is independently branched C₁₀-C₂₀ alkynyl. In some embodiments, each R⁵ is independently branched C₁₂-C₂₅ alkynyl. In some embodiments, each R⁵ is independently unbranched C₄-C₃₅ alkynyl. In some embodiments, each R⁵ is independently unbranched C₄-C₁₂ alkynyl. In some embodiments, each R⁵ is independently unbranched C₁₀-C₂₀ alkynyl. In some embodiments, each R⁵ is independently unbranched C₁₂-C₂₅ alkynyl.

In some embodiments, each instance of R⁵ is independently optionally substituted C₄-C₃₅ saturated or unsaturated heteroaliphatic. In some embodiments, each instance of R⁵ is independently optionally substituted C₄-C₃₅ saturated or unsaturated heteroaliphatic comprising at least one O, N, or S atom. In some embodiments, each instance of R⁵ is independently optionally substituted C₄-C₃₅ saturated or unsaturated heteroaliphatic comprising at least one O or S atom. In some embodiments, each instance of R⁵ is independently optionally substituted C₄-C₁₂ saturated or unsaturated heteroaliphatic. In some embodiments, each instance of R⁵ is independently optionally substituted C₄-C₁₂ saturated or unsaturated heteroaliphatic comprising at least one O, N, or S atom. In some embodiments, each instance of R⁵ is independently optionally substituted C₄-C₁₂ saturated or unsaturated heteroaliphatic comprising at least one O or S atom. In some embodiments, each instance of R⁵ is independently optionally substituted C₁₀-C₂₀ saturated or unsaturated heteroaliphatic. In some embodiments, each instance of R⁵ is independently optionally substituted C₁₀-C₂₀ saturated or unsaturated heteroaliphatic comprising at least one O, N, or S atom. In some embodiments, each instance of R⁵ is independently optionally substituted C₁₀-C₂₀ saturated or unsaturated heteroaliphatic comprising at least one O or S atom. In some embodiments, each instance of R⁵ is independently optionally substituted C₁₂-C₂₅ saturated or unsaturated heteroaliphatic. In some embodiments, each instance of R⁵ is independently optionally substituted C₁₂-C₂₅ saturated or unsaturated heteroaliphatic comprising at least one O, N, or S atom. In some embodiments, each instance of R⁵ is independently optionally substituted C₁₂-C₂₅ saturated or unsaturated heteroaliphatic comprising at least one O or S atom.

In some embodiments, each R⁵ is independently optionally substituted C₄-C₃₅ heteroalkyl or optionally substituted C₄-C₃₅ heteroalkenyl. In some embodiments, each R⁵ is independently optionally substituted C₄-C₁₀ heteroalkyl or optionally substituted C₄-C₁₀ heteroalkenyl. In some embodiments, each R⁵ is independently optionally substituted C₁₀-C₂₀ heteroalkyl or optionally substituted C₁₀-C₂₀ heteroalkenyl. In some embodiments, each R⁵ is independently optionally substituted C₁₂-C₂₅ heteroalkyl or optionally substituted C₁₂-C₂₅ heteroalkenyl.

In some embodiments, each R⁵ is independently optionally substituted C₄-C₃₅ heteroalkyl or optionally substituted C₄-C₃₅ heteroalkynyl. In some embodiments, each R⁵ is independently optionally substituted C₄-C₁₀ heteroalkyl or optionally substituted C₄-C₁₀ heteroalkynyl. In some embodiments, each R⁵ is independently optionally substituted C₁₀-C₂₀ heteroalkyl or optionally substituted C₁₀-C₂₀ heteroalkynyl. In some embodiments, each R⁵ is independently optionally substituted C₁₂-C₂₅ heteroalkyl or optionally substituted C₁₂-C₂₅ heteroalkynyl.

In some embodiments, each R⁵ is independently optionally substituted C₄-C₃₅ heteroalkynyl or optionally substituted C₄-C₃₅ heteroalkenyl. In some embodiments, each R⁵ is independently optionally substituted C₄-C₁₀ heteroalkynyl or optionally substituted C₄-C₁₀ heteroalkenyl. In some embodiments, each R⁵ is independently optionally substituted C₁₀-C₂₀ heteroalkynyl or optionally substituted C₁₀-C₂₀ heteroalkenyl. In some embodiments, each R⁵ is independently optionally substituted C₁₂-C₂₅ heteroalkynyl or optionally substituted C₁₂-C₂₅ heteroalkenyl.

In some embodiments, each R⁵ is independently branched C₄-C₃₅ heteroalkyl. In some embodiments, R⁵ is independently branched C₄-C₁₂ heteroalkyl. In some embodiments, each R⁵ is independently branched C₁₀-C₂₀ heteroalkyl. In some embodiments, each R⁵ is independently branched C₁₂-C₂₅ heteroalkyl. In some embodiments, each R⁵ is independently unbranched C₄-C₃₅ heteroalkyl. In some embodiments, each R⁵ is independently unbranched C₄-C₁₂ heteroalkyl. In some embodiments, each R⁵ is independently unbranched C₁₀-C₂₀ heteroalkyl. In some embodiments, each R⁵ is independently unbranched C₁₂-C₂₅ heteroalkyl.

In some embodiments, each R⁵ is independently branched C₄-C₃₅ heteroalkenyl. In some embodiments, each R⁵ is independently branched C₄-C₁₂ heteroalkenyl. In some embodiments, each R⁵ is independently branched C₁₀-C₂₀ heteroalkenyl. In some embodiments, each R⁵ is independently branched C₁₂-C₂₅ heteroalkenyl. In some embodiments, each R⁵ is independently unbranched C₄-C₃₅ heteroalkenyl. In some embodiments, each R⁵ is independently unbranched C₄-C₁₂ heteroalkenyl. In some embodiments, each R⁵ is independently unbranched C₁₀-C₂₀ heteroalkenyl. In some embodiments, each R⁵ is independently unbranched C₁₂-C₂₅ heteroalkenyl.

In some embodiments, each R⁵ is independently branched C₄-C₃₅ heteroalkynyl. In some embodiments, each R⁵ is independently branched C₄-C₁₂ heteroalkynyl. In some embodiments, each R⁵ is independently branched C₁₀-C₂₀ heteroalkynyl. In some embodiments, each R⁵ is independently branched C₁₂-C₂₅ heteroalkynyl. In some embodiments, each R⁵ is independently unbranched C₄-C₃₅ heteroalkynyl. In some embodiments, each R⁵ is independently unbranched C₄-C₁₂ heteroalkynyl. In some embodiments, each R⁵ is independently unbranched C₁₀-C₂₀ heteroalkynyl. In some embodiments, each R⁵ is independently unbranched C₁₂-C₂₅ heteroalkynyl.

In some embodiments, each R⁵ is independently selected from the group consisting of:

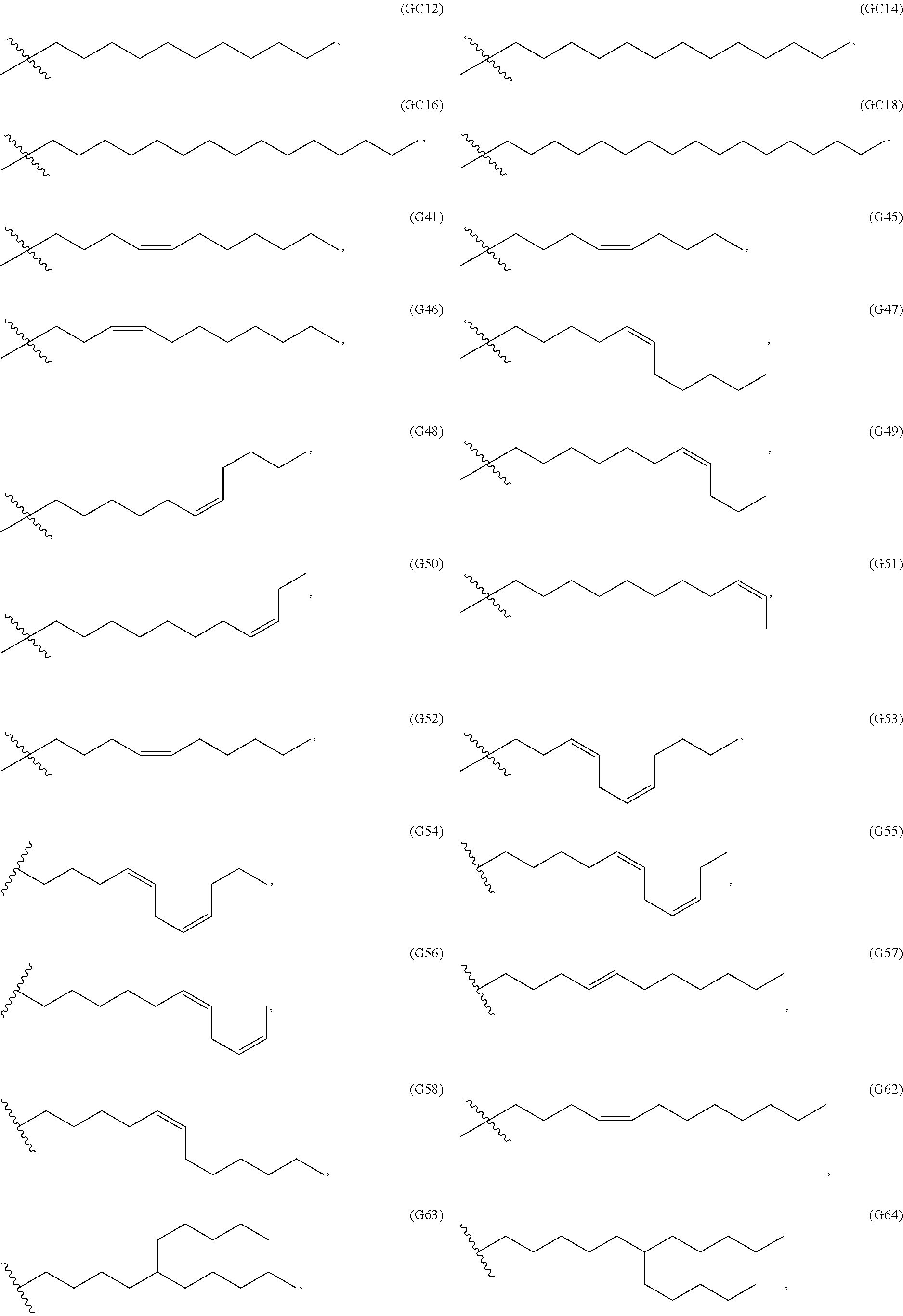

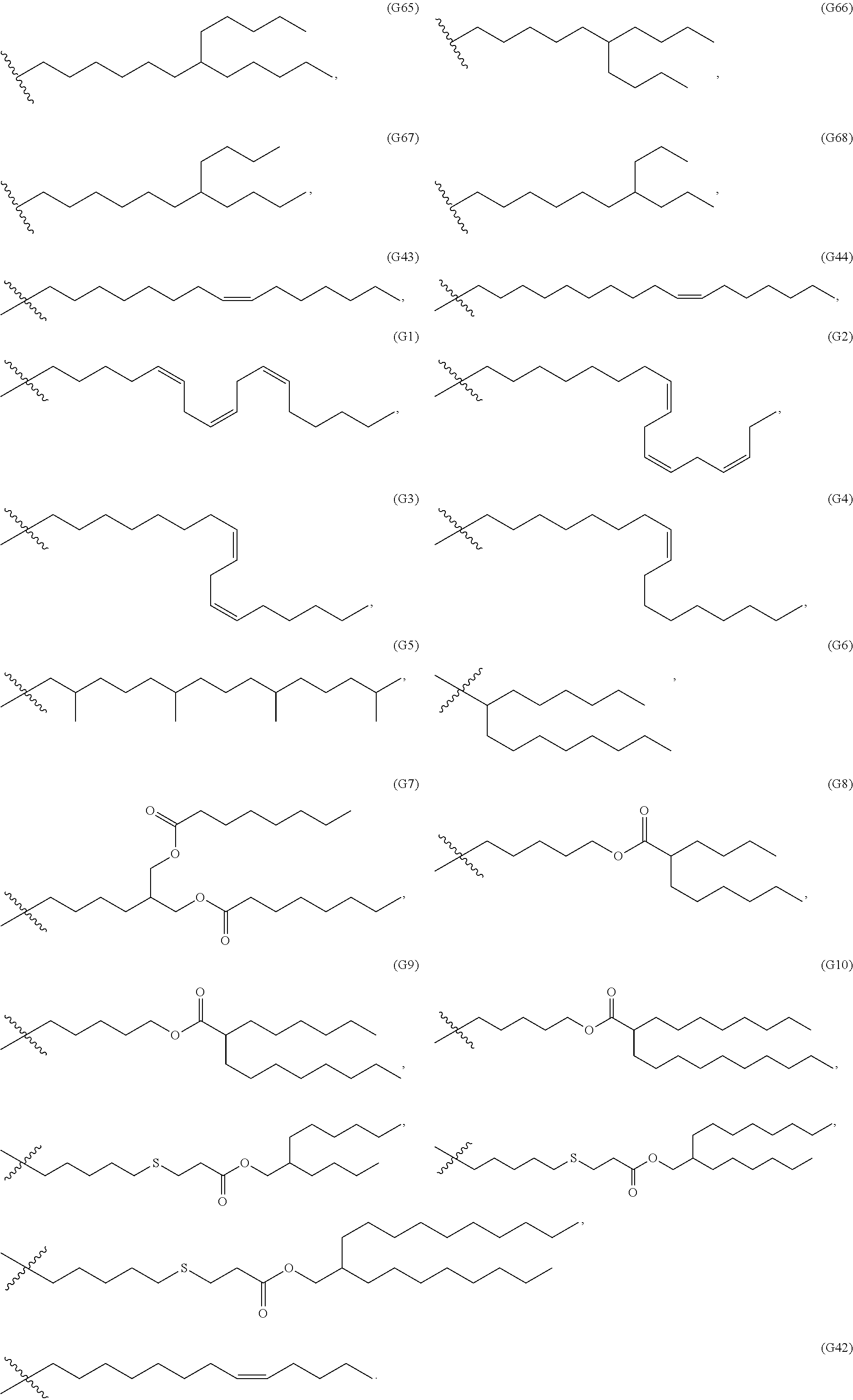

In some embodiments, each R⁵ is independently selected from the group consisting of:

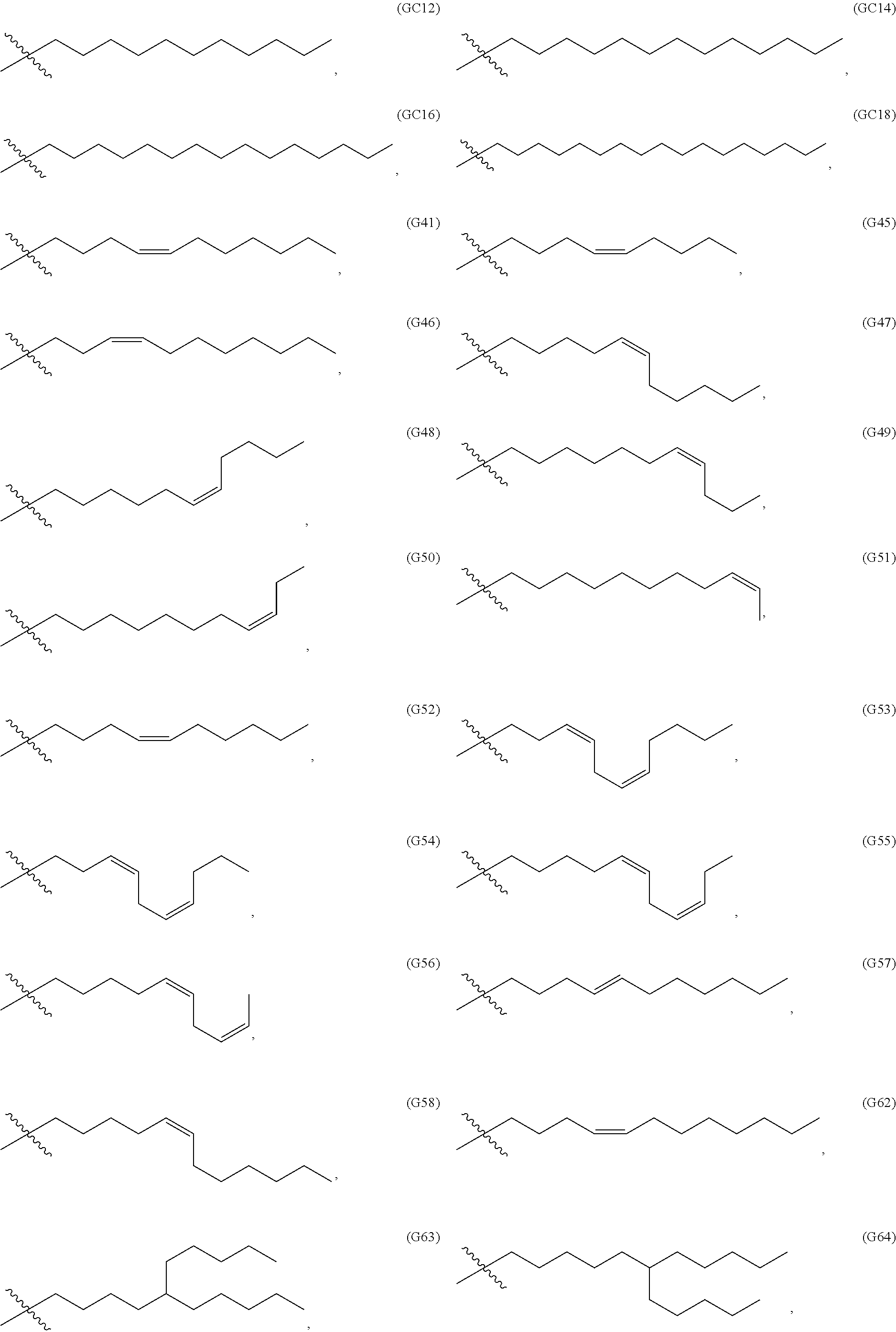

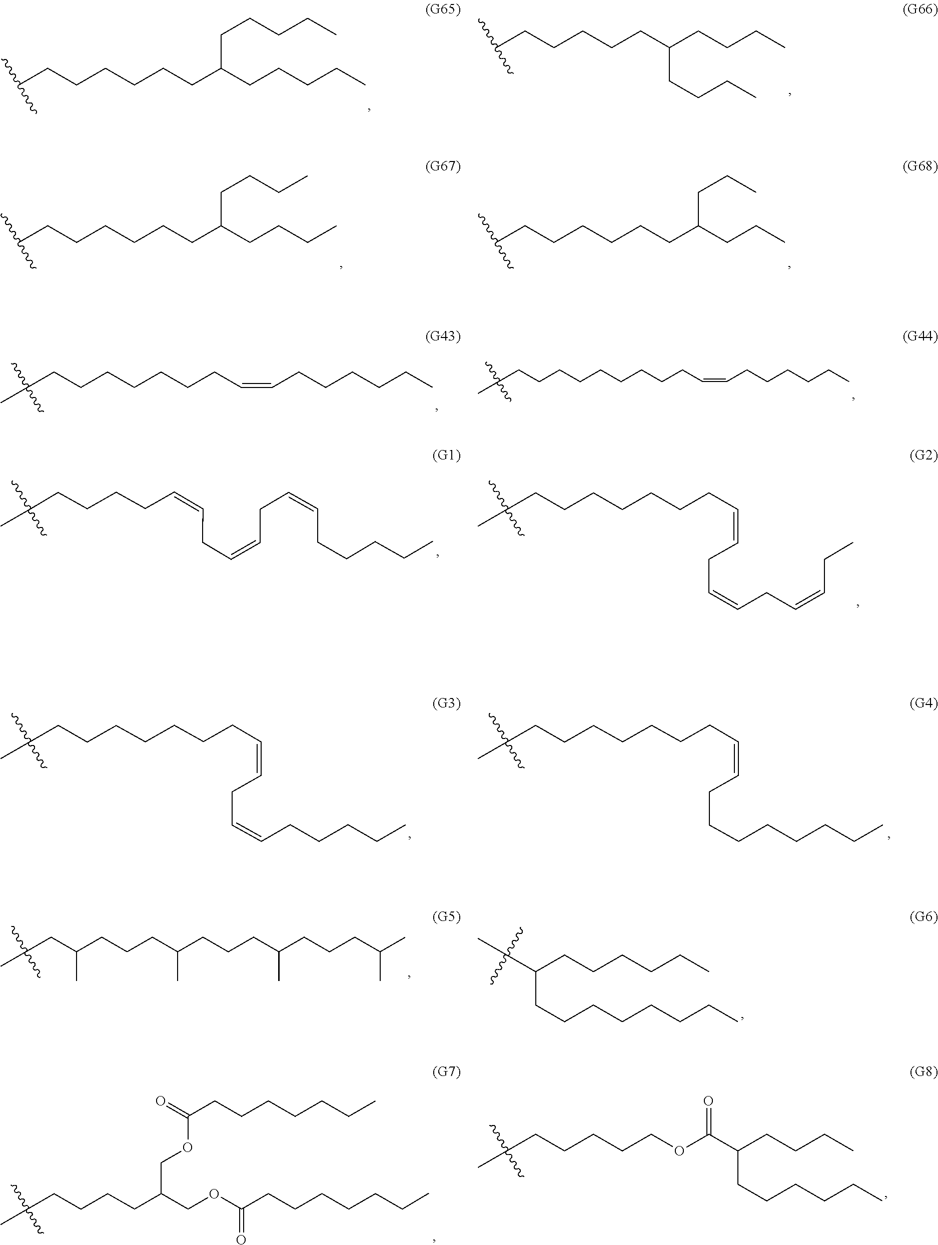

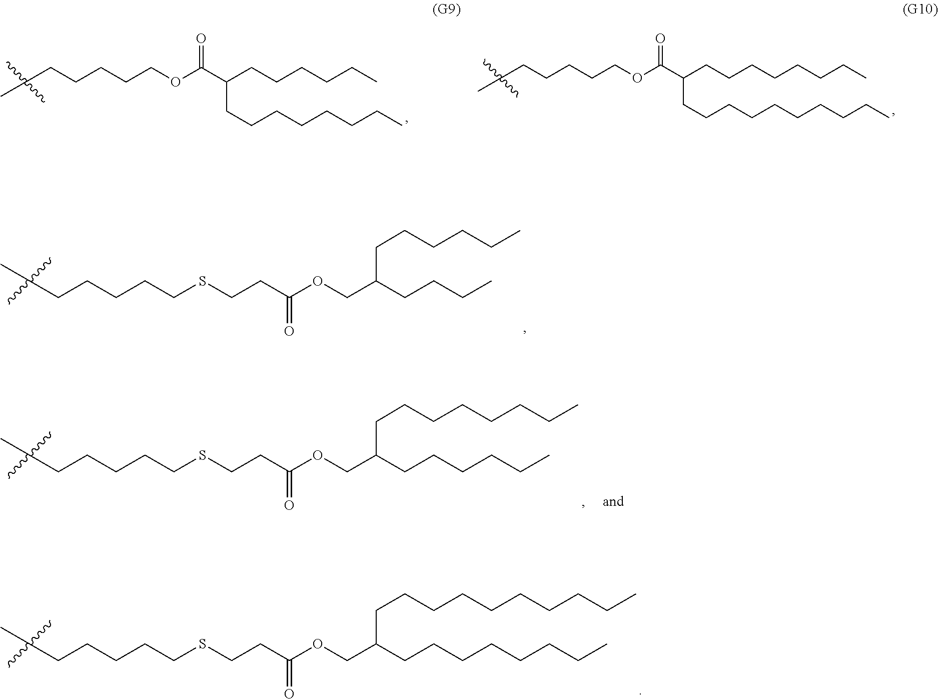 [SMILES: *CCCCCOC(=O)C(CCCCCC)CCCCCCCC ; *CCCCCOC(=O)C(CCCCCCCC)CCCCCCCCCC ; *CCCCCSCCC(=O)OCC(CCCC)CCCCCC ; *CCCCCSCCC(=O)OCC(CCCCCC)CCCCCCCC ; *CCCCCSCCC(=O)OCC(CCCCCCCC)CCCCCCCCCC]

In some embodiments, each R⁵ is independently selected from the group consisting of:

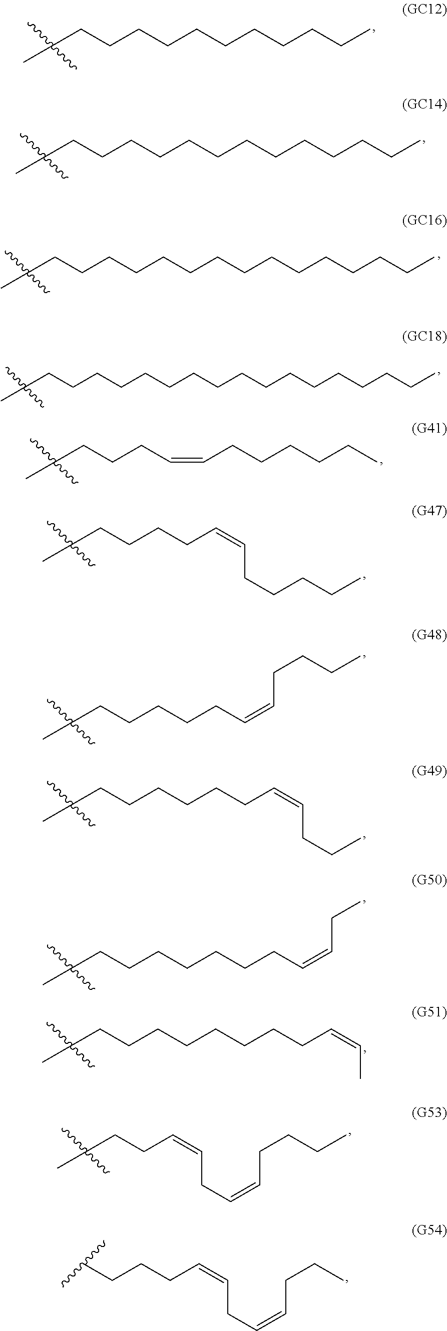

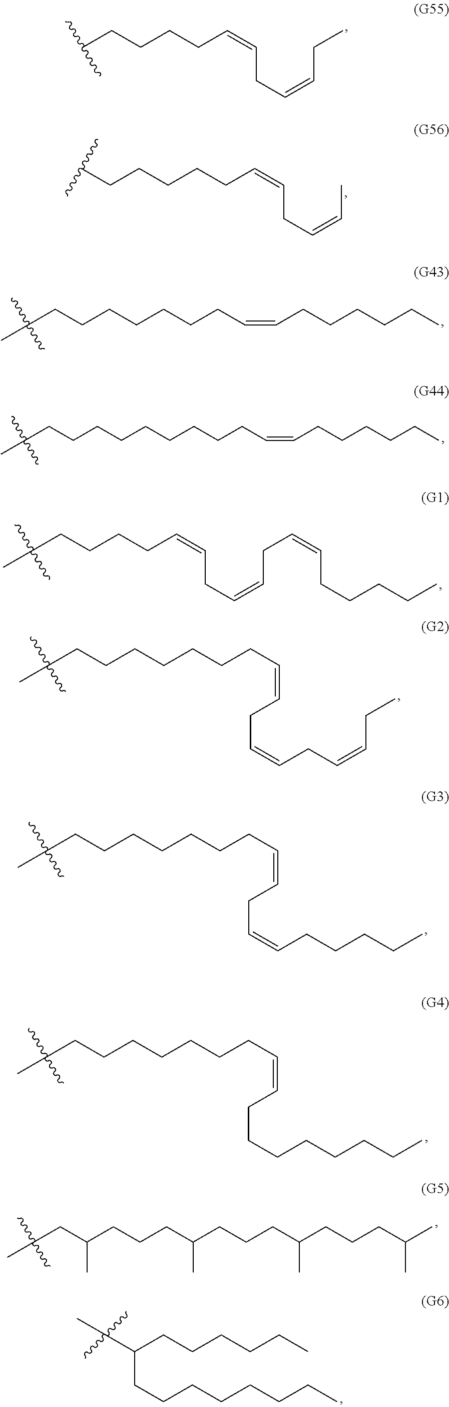

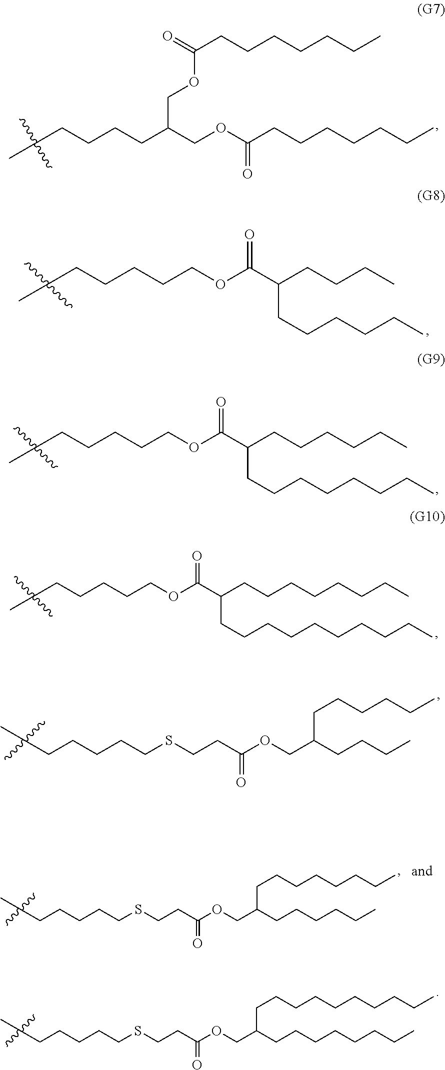

In some embodiments, each R⁵ is independently selected from the group consisting of:

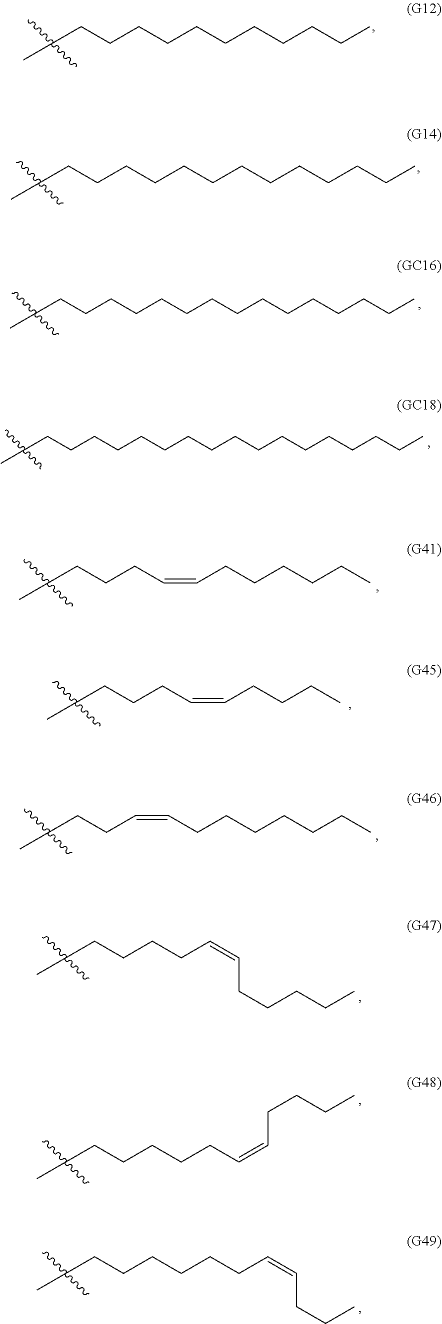

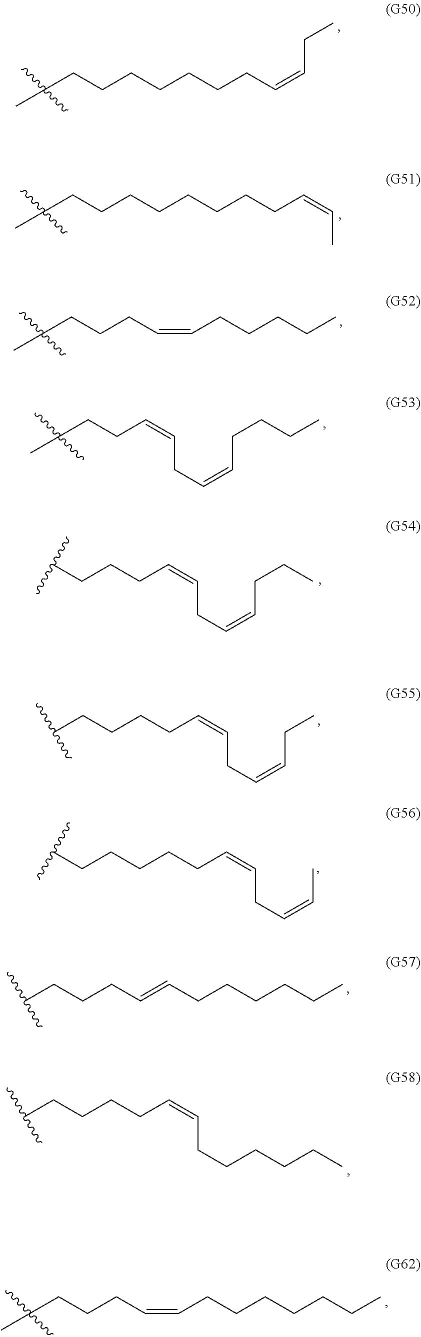

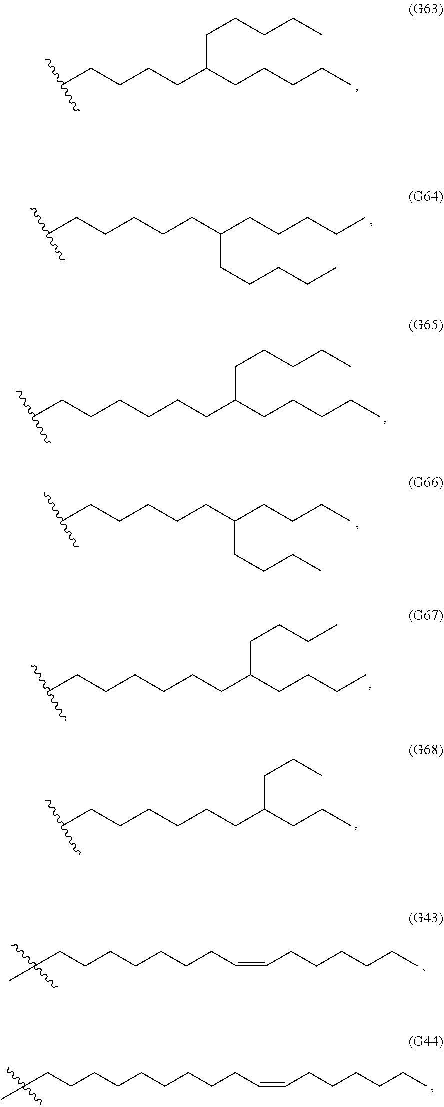

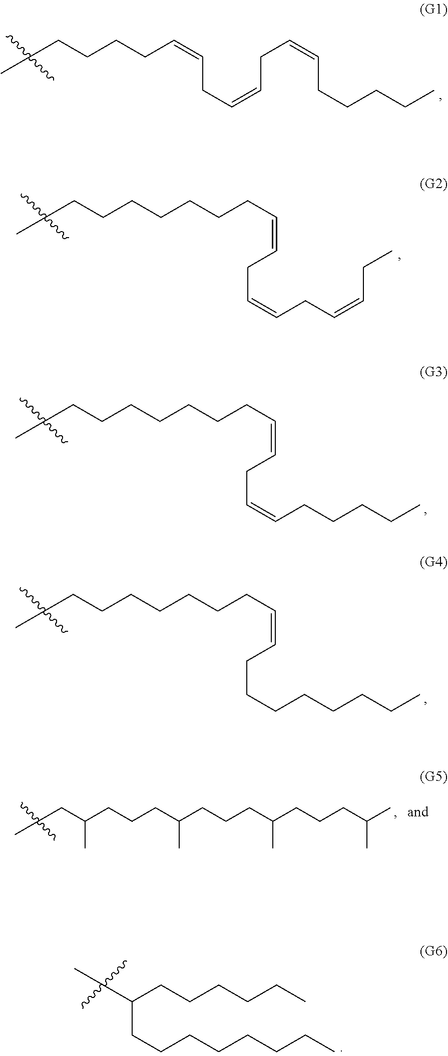 [SMILES: *CCCC/C=C\C/C=C\C/C=C\CCCCC ; *CCCCCCC/C=C\C/C=C\C/C=C\CC ; *CCCCCCC/C=C\C/C=C\CCCCC ; *CCCCCCC/C=C\CCCCCCCC ; *CC(C)CCCC(C)CCCC(C)CCCC(C)C ; *C(CCCCCC)CCCCCCCC]

In some embodiments, each R⁵ is independently selected from the group consisting of:

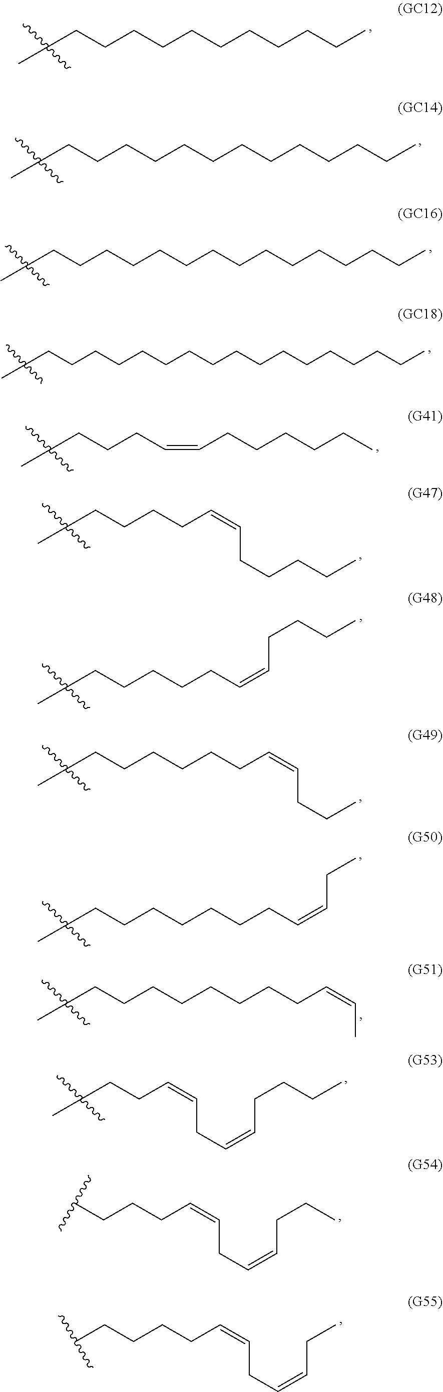

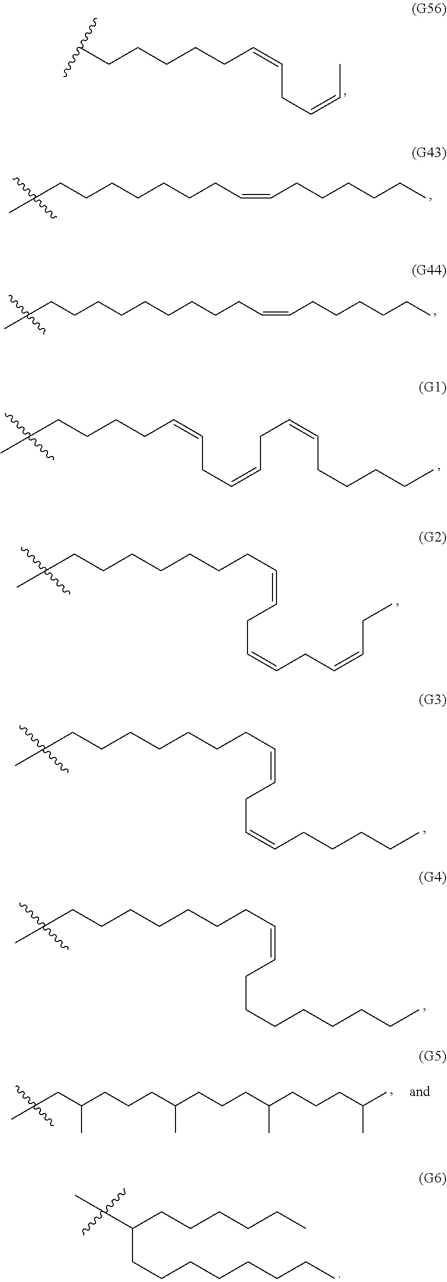

In some embodiments, each R⁵ is independently selected from the group consisting of:

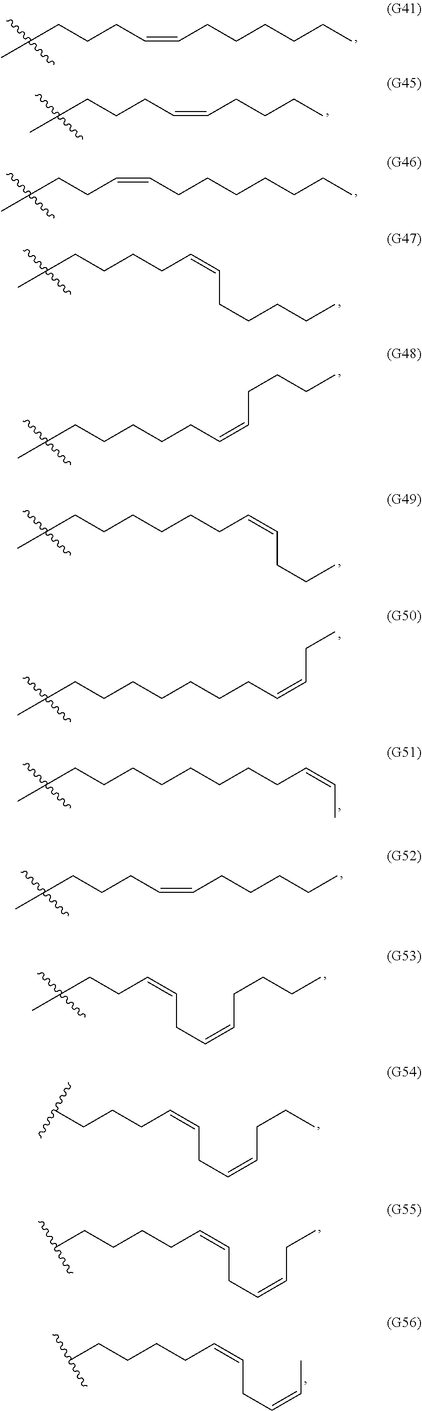

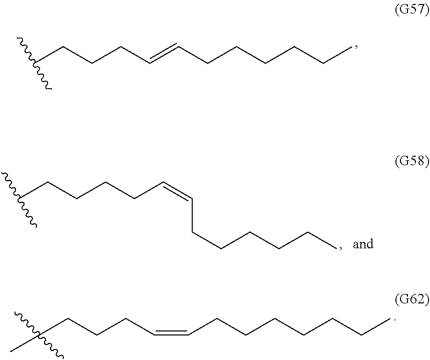 [SMILES: *CCC/C=C/CCCCCC ; *CCCC/C=C\CCCCCC ; *CCC/C=C\CCCCCCC]

In some embodiments, each R⁵ is independently selected from the group consisting of:

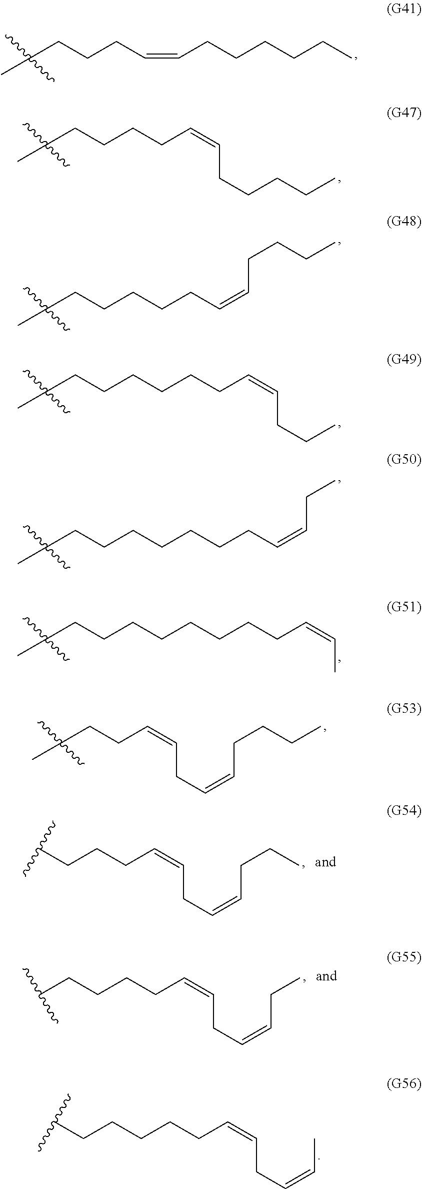

In some embodiments, R⁵ is

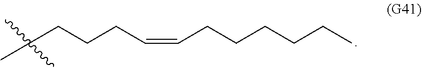 [SMILES: *CCC/C=C\CCCCCC]

In some embodiments, each R⁵ is independently selected from the group consisting of:

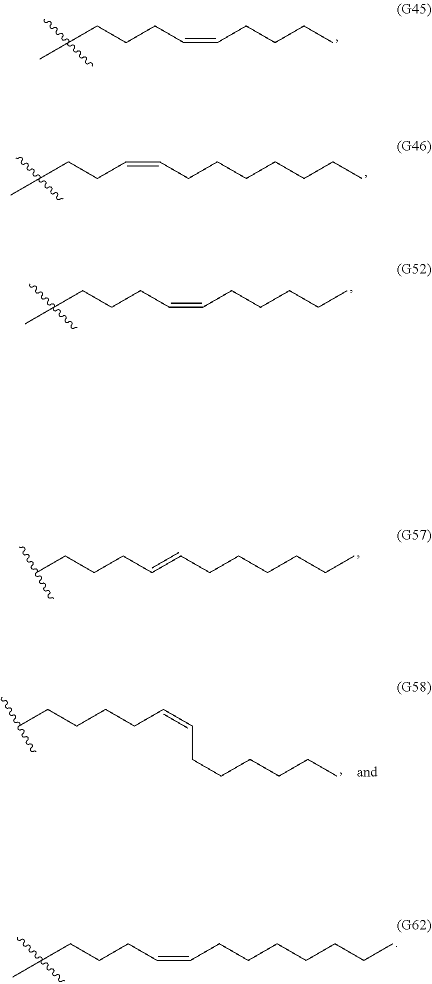

In some embodiments, each R⁵ is independently selected from the group consisting of:

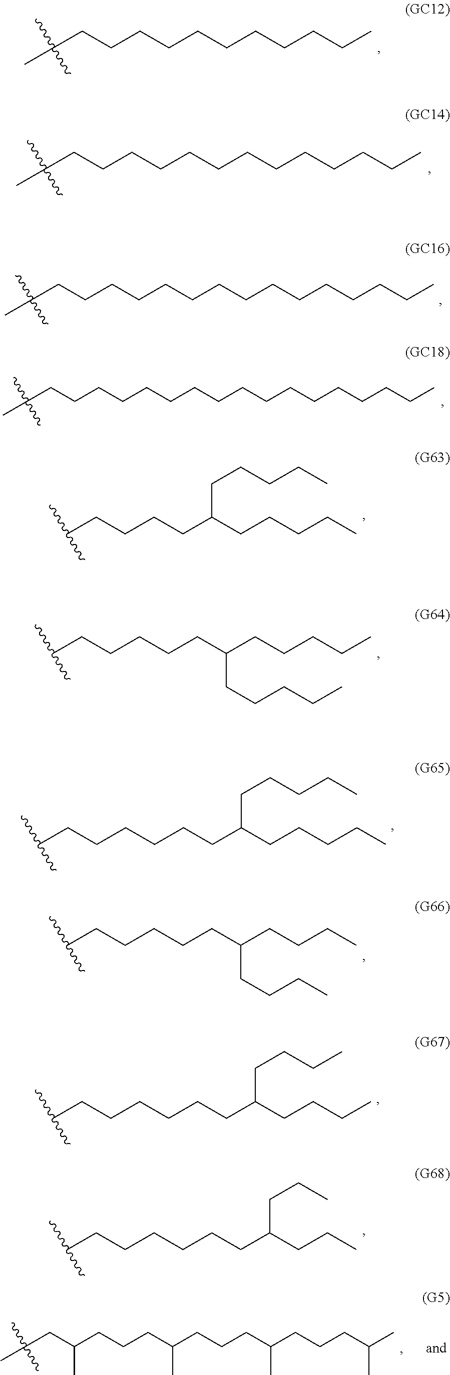

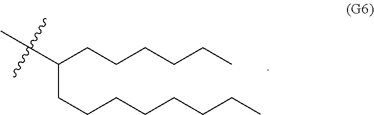 [SMILES: *C(CCCCCC)CCCCCCCC]

In some embodiments, each R⁵ is independently selected from the group consisting of:

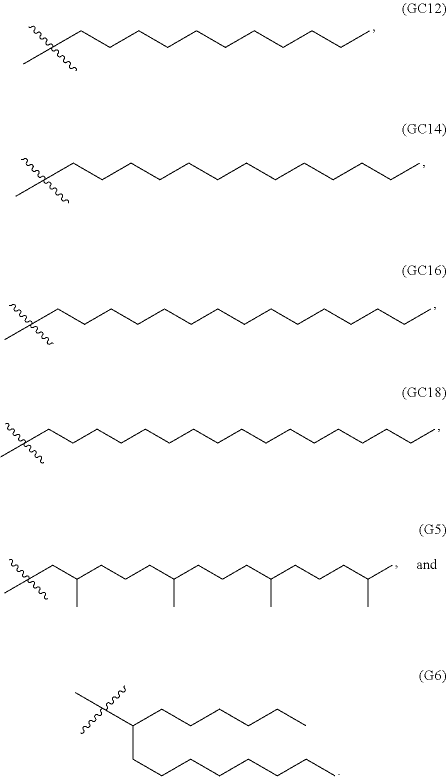 [SMILES: *CCCCCCCCCCC ; *CCCCCCCCCCCCC ; *CCCCCCCCCCCCCCC ; *CCCCCCCCCCCCCCCCC ; *CC(C)CCCC(C)CCCC(C)CCCC(C)C ; *C(CCCCCC)CCCCCCCC]

In some embodiments, each R⁵ is independently selected from the group consisting of:

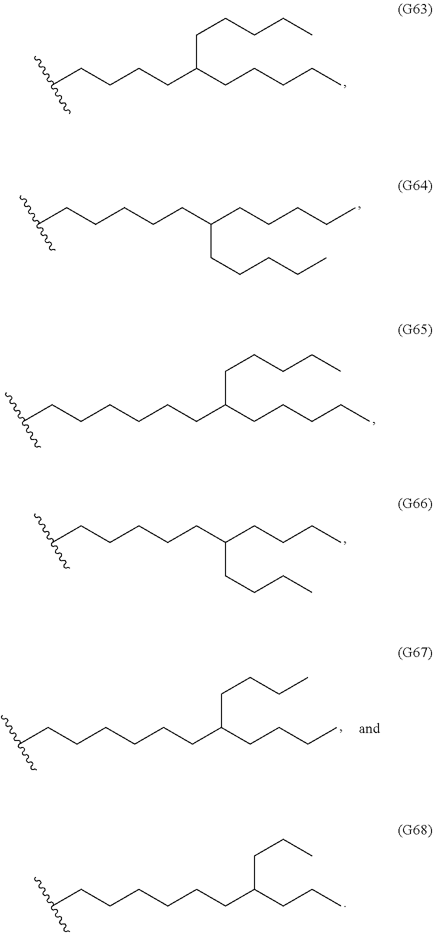 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

In some embodiments, each R⁵ is independently selected from the group consisting of:

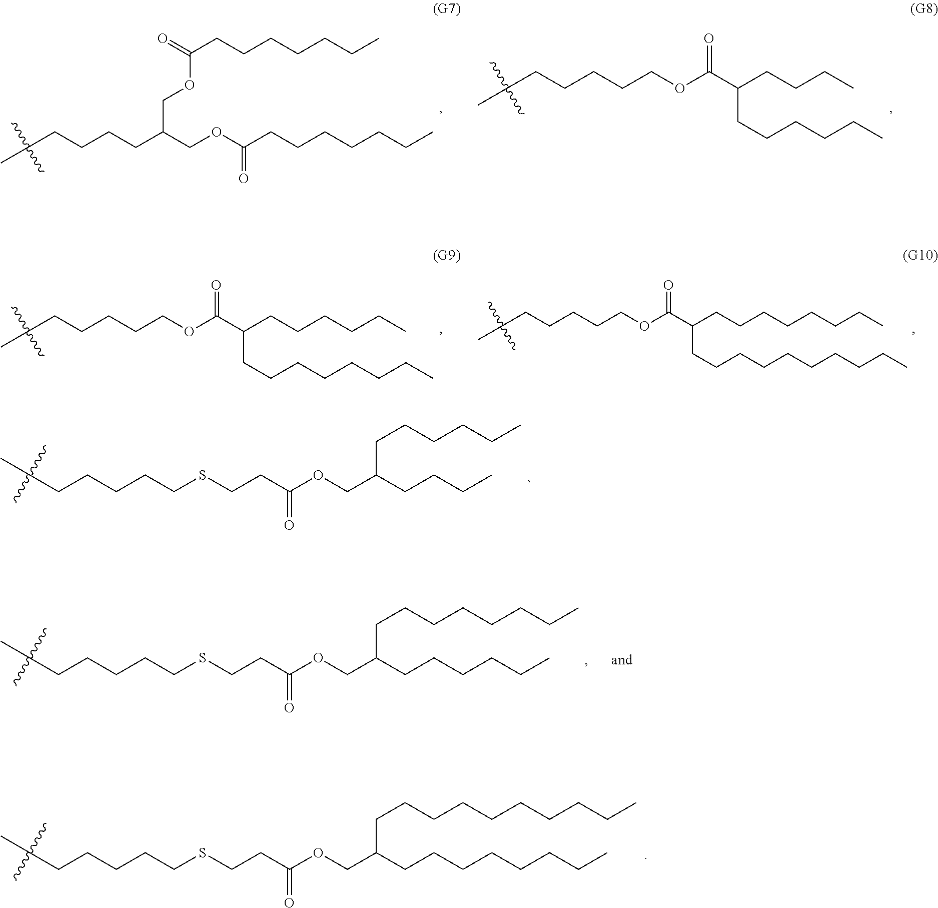

In some embodiments, each R⁵ is independently selected from the group consisting of:

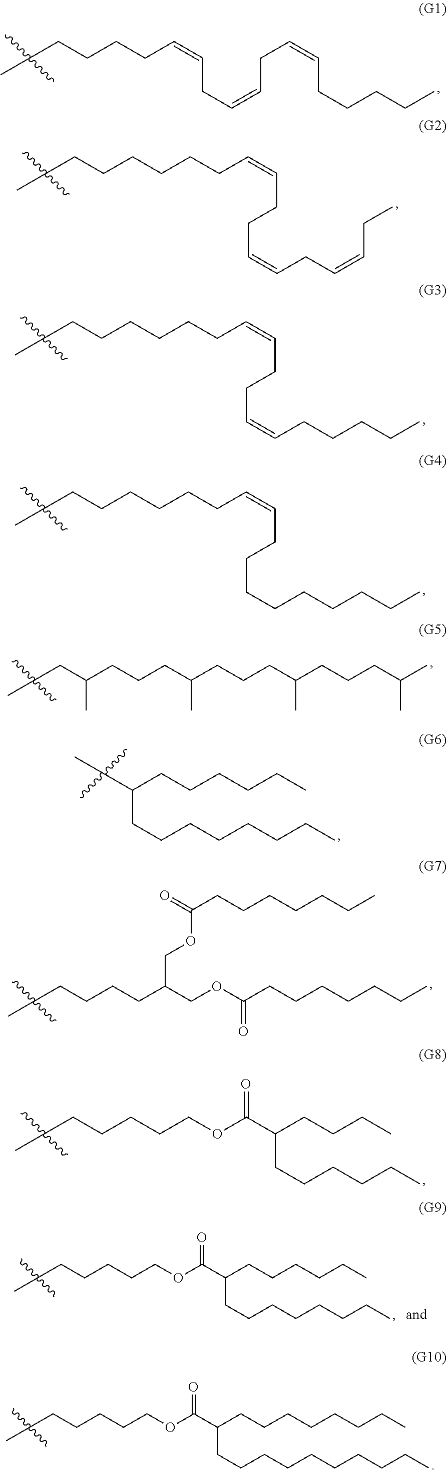

In some embodiments, at least one instance of R⁵ is optionally substituted C₄-C₃₅ saturated aliphatic or optionally substituted C₄-C₃₅ saturated heteroaliphatic. In some embodiments, at least one instance of R⁵ is optionally substituted C₄-C₃₅ unsaturated aliphatic or optionally substituted C₄-C₃₅ unsaturated heteroaliphatic.

In some embodiments, at least one instance of R⁵ is optionally substituted C₄-C₃₅ saturated or unsaturated aliphatic. In some embodiments, at least one instance of R⁵ is optionally substituted C₄-C₁₂ saturated or unsaturated aliphatic. In some embodiments, at least one instance of R⁵ is optionally substituted C₁₀-C₂₀ saturated or unsaturated aliphatic. In some embodiments, at least one instance of R⁵ is optionally substituted C₁₂-C₂₅ saturated or unsaturated aliphatic.

In some embodiments, at least one instance of R⁵ is optionally substituted C₄-C₃₅ alkyl or optionally substituted C₄-C₃₅ alkenyl. In some embodiments, at least one instance of R⁵ is optionally substituted C₄-C₁₀ alkyl or optionally substituted C₄-C₁₀ alkenyl. In some embodiments, at least one instance of R⁵ is optionally substituted C₁₀-C₂₀ alkyl or optionally substituted C₁₀-C₂₀ alkenyl. In some embodiments, at least one instance of R⁵ is optionally substituted C₁₂-C₂₅ alkyl or optionally substituted C₁₂-C₂₅ alkenyl.

In some embodiments, at least one instance of R⁵ is optionally substituted C₄-C₃₅ alkyl or optionally substituted C₄-C₃₅ alkynyl. In some embodiments, at least one instance of R⁵ is optionally substituted C₄-C₁₀ alkyl or optionally substituted C₄-C₁₀ alkynyl. In some embodiments, at least one instance of R⁵ is optionally substituted C₁₀-C₂₀ alkyl or optionally substituted C₁₀-C₂₀ alkynyl. In some embodiments, at least one instance of R⁵ is optionally substituted C₁₂-C₂₅ alkyl or optionally substituted C₁₂-C₂₅ alkynyl.

In some embodiments, at least one instance of R⁵ is optionally substituted C₄-C₃₅ alkynyl or optionally substituted C₄-C₃₅ alkenyl. In some embodiments, at least one instance of R⁵ is optionally substituted C₄-C₁₀ alkynyl or optionally substituted C₄-C₁₀ alkenyl. In some embodiments, at least one instance of R⁵ is optionally substituted C₁₀-C₂₀ alkynyl or optionally substituted C₁₀-C₂₀ alkenyl. In some embodiments, at least one instance of R⁵ is optionally substituted C₁₂-C₂₅ alkynyl or optionally substituted C₁₂-C₂₅ alkenyl.

In some embodiments, at least one instance of R⁵ is branched C₄-C₃₅ alkyl. In some embodiments, at least one instance of R⁵ is branched C₄-C₁₂ alkyl. In some embodiments, at least one instance of R⁵ is branched C₁₀-C₂₀ alkyl. In some embodiments, at least one instance of R⁵ is branched C₁₂-C₂₅ alkyl. In some embodiments, at least one instance of R⁵ is unbranched C₄-C₃₅ alkyl. In some embodiments, at least one instance of R⁵ is unbranched C₄-C₁₂ alkyl. In some embodiments, at least one instance of R⁵ is unbranched C₁₀-C₂₀ alkyl. In some embodiments, at least one instance of R⁵ is unbranched C₁₂-C₂₅ alkyl.

In some embodiments, at least one instance of R⁵ is branched C₄-C₃₅ alkenyl. In some embodiments, at least one instance of R⁵ is branched C₄-C₃₅ alkenyl comprising a single double bond. In some embodiments, at least one instance of R⁵ is branched C₄-C₃₅ alkenyl comprising a single (Z) double bond. In some embodiments, at least one instance of R⁵ is branched C₄-C₁₂ alkenyl. In some embodiments, at least one instance of R⁵ is branched C₁₀-C₂₀ alkenyl. In some embodiments, at least one instance of R⁵ is branched C₁₂-C₂₅ alkenyl.

In some embodiments, at least one instance of R⁵ is unbranched C₄-C₃₅ alkenyl. In some embodiments, at least one instance of R⁵ is unbranched C₄-C₃₅ alkenyl comprising a single double bond. In some embodiments, at least one instance of R⁵ is unbranched C₄-C₃₅ alkenyl comprising a single (Z) double bond. In some embodiments, at least one instance of R⁵ is unbranched C₄-C₃₅ alkenyl comprising two double bonds. In some embodiments, at least one instance of R⁵ is unbranched C₄-C₃₅ alkenyl comprising two (Z) double bonds. In some embodiments, at least one instance of R⁵ is unbranched C₄-C₃₅ alkenyl comprising

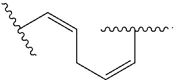 [SMILES: */C=C\C/C=C\*]

In some embodiments, at least one instance of R⁵ is unbranched C₄-C₃₅ alkenyl comprising three double bonds. In some embodiments, at least one instance of R⁵ is unbranched C₄-C₃₅ alkenyl comprising three (Z) double bonds. In some embodiments, at least one instance of R⁵ is unbranched C₄-C₃₅ alkenyl comprising only (Z) double bonds.

In some embodiments, at least one instance of R⁵ is unbranched C₄-C₁₂ alkenyl. In some embodiments, at least one instance of R⁵ is unbranched C₄-C₁₂ alkenyl comprising a single double bond. In some embodiments, at least one instance of R⁵ is unbranched C₄-C₁₂ alkenyl comprising a single (Z) double bond. In some embodiments, at least one instance of R⁵ is unbranched C₄-C₁₂ alkenyl comprising two double bonds. In some embodiments, at least one instance of R⁵ is unbranched C₄-C₁₂ alkenyl comprising two (Z) double bonds. In some embodiments, at least one instance of R⁵ is unbranched C₄-C₁₂ alkenyl comprising

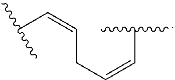 [SMILES: */C=C\C/C=C\*]

In some embodiments, at least one instance of R⁵ is unbranched C₄-C₁₂ alkenyl comprising three double bonds. In some embodiments, at least one instance of R⁵ is unbranched C₄-C₁₂ alkenyl comprising three (Z) double bonds. In some embodiments, at least one instance of R⁵ is unbranched C₄-C₁₂ alkenyl comprising only (Z) double bonds.

In some embodiments, at least one instance of R⁵ is unbranched C₁₀-C₂₀ alkenyl. In some embodiments, at least one instance of R⁵ is unbranched C₁₀-C₂₀ alkenyl comprising a single double bond. In some embodiments, at least one instance of R⁵ is unbranched C₁₀-C₂₀ alkenyl comprising a single (Z) double bond. In some embodiments, at least one instance of R⁵ is unbranched C₁₀-C₂₀ alkenyl comprising two double bonds. In some embodiments, at least one instance of R⁵ is unbranched C₁₀-C₂₀ alkenyl comprising two (Z) double bonds. In some embodiments, at least one instance of R⁵ is unbranched C₁₀-C₂₀ alkenyl comprising

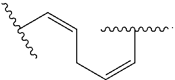 [SMILES: */C=C\C/C=C\*]

In some embodiments, at least one instance of R⁵ is unbranched C₁₀-C₂₀ alkenyl comprising three double bonds. In some embodiments, at least one instance of R⁵ is unbranched C₁₀-C₂₀ alkenyl comprising three (Z) double bonds. In some embodiments, at least one instance of R⁵ is unbranched C₁₀-C₂₀ alkenyl comprising only (Z) double bonds.

In some embodiments, at least one instance of R⁵ is unbranched C₁₂-C₂₅ alkenyl. In some embodiments, at least one instance of R⁵ is unbranched C₁₂-C₂₅ alkenyl comprising a single double bond. In some embodiments, at least one instance of R⁵ is unbranched C₁₂-C₂₅ alkenyl comprising a single (Z) double bond. In some embodiments, at least one instance of R⁵ is unbranched C₁₂-C₂₅ alkenyl comprising two double bonds. In some embodiments, at least one instance of R⁵ is unbranched C₁₂-C₂₅ alkenyl comprising two (Z) double bonds. In some embodiments, at least one instance of R⁵ is unbranched C₁₂-C₂₅ alkenyl comprising

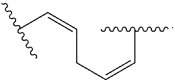 [SMILES: */C=C\C/C=C\*]

In some embodiments, at least one instance of R⁵ is unbranched C₁₂-C₂₅ alkenyl comprising three double bonds. In some embodiments, at least one instance of R⁵ is unbranched C₁₂-C₂₅ alkenyl comprising three (Z) double bonds. In some embodiments, at least one instance of R⁵ is unbranched C₁₂-C₂₅ alkenyl comprising only (Z) double bonds.

In some embodiments, at least one instance of R⁵ is unbranched C₁₁ alkenyl. In some embodiments, at least one instance of R⁵ is unbranched C₁₁ alkenyl comprising a single double bond. In some embodiments, at least one instance of R⁵ is unbranched C₁₁ alkenyl comprising a single (Z) double bond. In some embodiments, at least one instance of R⁵ is unbranched C₁₁ alkenyl comprising two double bonds. In some embodiments, at least one instance of R⁵ is unbranched C₁₁ alkenyl comprising two (Z) double bonds. In some embodiments, at least one instance of R⁵ is unbranched C₁₁ alkenyl comprising

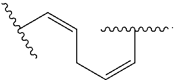 [SMILES: */C=C\C/C=C\*]

In some embodiments, at least one instance of R⁵ is unbranched C₁₁ alkenyl comprising three double bonds. In some embodiments, at least one instance of R⁵ is unbranched C₁₁ alkenyl comprising three (Z) double bonds. In some embodiments, at least one instance of R⁵ is unbranched C₁₁ alkenyl comprising only (Z) double bonds.

In some embodiments, at least one instance of R⁵ is branched C₄-C₃₅ alkynyl. In some embodiments, at least one instance of R⁵ is branched C₄-C₁₂ alkynyl. In some embodiments, R⁵ is branched C₁₀-C₂₀ alkynyl. In some embodiments, at least one instance of R⁵ is branched C₁₂-C₂₅ alkynyl. In some embodiments, at least one instance of R⁵ is unbranched C₄-C₃₅ alkynyl. In some embodiments, at least one instance of R⁵ is unbranched C₄-C₁₂ alkynyl. In some embodiments, at least one instance of R⁵ is unbranched C₁₀-C₂₀ alkynyl. In some embodiments, at least one instance of R⁵ is unbranched C₁₂-C₂₅ alkynyl.

In some embodiments, at least one instance of R⁵ is optionally substituted C₄-C₃₅ saturated or unsaturated heteroaliphatic. In some embodiments, at least one instance of R⁵ is optionally substituted C₄-C₃₅ saturated or unsaturated heteroaliphatic comprising at least one O, N, or S atom. In some embodiments, at least one instance of R⁵ is optionally substituted C₄-C₃₅ saturated or unsaturated heteroaliphatic comprising at least one O or S atom. In some embodiments, at least one instance of R⁵ is optionally substituted C₄-C₁₂ saturated or unsaturated heteroaliphatic. In some embodiments, at least one instance of R⁵ is optionally substituted C₄-C₁₂ saturated or unsaturated heteroaliphatic comprising at least one O, N, or S atom. In some embodiments, at least one instance of R⁵ is optionally substituted C₄-C₁₂ saturated or unsaturated heteroaliphatic comprising at least one O or S atom. In some embodiments, at least one instance of R⁵ is optionally substituted C₁₀-C₂₀ saturated or unsaturated heteroaliphatic. In some embodiments, at least one instance of R⁵ is optionally substituted C₁₀-C₂₀ saturated or unsaturated heteroaliphatic comprising at least one O, N, or S atom. In some embodiments, at least one instance of R⁵ is optionally substituted C₁₀-C₂₀ saturated or unsaturated heteroaliphatic comprising at least one O or S atom. In some embodiments, at least one instance of R⁵ is optionally substituted C₁₂-C₂₅ saturated or unsaturated heteroaliphatic. In some embodiments, at least one instance of R⁵ is optionally substituted C₁₂-C₂₅ saturated or unsaturated heteroaliphatic comprising at least one O, N, or S atom. In some embodiments, at least one instance of R⁵ is optionally substituted C₁₂-C₂₅ saturated or unsaturated heteroaliphatic comprising at least one O or S atom.

In some embodiments, at least one instance of R⁵ is optionally substituted C₄-C₃₅ heteroalkyl or optionally substituted C₄-C₃₅ heteroalkenyl. In some embodiments, at least one instance of R⁵ is optionally substituted C₄-C₁₀ heteroalkyl or optionally substituted C₄-C₁₀ heteroalkenyl. In some embodiments, at least one instance of R⁵ is optionally substituted C₁₀-C₂₀ heteroalkyl or optionally substituted C₁₀-C₂₀ heteroalkenyl. In some embodiments, at least one instance of R⁵ is optionally substituted C₁₂-C₂₅ heteroalkyl or optionally substituted C₁₂-C₂₅ heteroalkenyl.

In some embodiments, at least one instance of R⁵ is optionally substituted C₄-C₃₅ heteroalkyl or optionally substituted C₄-C₃₅ heteroalkynyl. In some embodiments, at least one instance of R⁵ is optionally substituted C₄-C₁₀ heteroalkyl or optionally substituted C₄-C₁₀ heteroalkynyl. In some embodiments, at least one instance of R⁵ is optionally substituted C₁₀-C₂₀ heteroalkyl or optionally substituted C₁₀-C₂₀ heteroalkynyl. In some embodiments, at least one instance of R⁵ is optionally substituted C₁₂-C₂₅ heteroalkyl or optionally substituted C₁₂-C₂₅ heteroalkynyl.

In some embodiments, at least one instance of R⁵ is optionally substituted C₄-C₃₅ heteroalkynyl or optionally substituted C₄-C₃₅ heteroalkenyl. In some embodiments, at least one instance of R⁵ is optionally substituted C₄-C₁₀ heteroalkynyl or optionally substituted C₄-C₁₀ heteroalkenyl. In some embodiments, at least one instance of R⁵ is optionally substituted C₁₀-C₂₀ heteroalkynyl or optionally substituted C₁₀-C₂₀ heteroalkenyl. In some embodiments, at least one instance of R⁵ is optionally substituted C₁₂-C₂₅ heteroalkynyl or optionally substituted C₁₂-C₂₅ heteroalkenyl.

In some embodiments, at least one instance of R⁵ is branched C₄-C₃₅ heteroalkyl. In some embodiments, R⁵ is branched C₄-C₁₂ heteroalkyl. In some embodiments, at least one instance of R⁵ is branched C₁₀-C₂₀ heteroalkyl. In some embodiments, at least one instance of R⁵ is branched C₁₂-C₂₅ heteroalkyl. In some embodiments, at least one instance of R⁵ is unbranched C₄-C₃₅ heteroalkyl. In some embodiments, at least one instance of R⁵ is unbranched C₄-C₁₂ heteroalkyl. In some embodiments, at least one instance of R⁵ is unbranched C₁₀-C₂₀ heteroalkyl. In some embodiments, at least one instance of R⁵ is unbranched C₁₂-C₂₅ heteroalkyl.

In some embodiments, at least one instance of R⁵ is branched C₄-C₃₅ heteroalkenyl. In some embodiments, at least one instance of R⁵ is branched C₄-C₁₂ heteroalkenyl. In some embodiments, at least one instance of R⁵ is branched C₁₀-C₂₀ heteroalkenyl. In some embodiments, at least one instance of R⁵ is branched C₁₂-C₂₅ heteroalkenyl. In some embodiments, at least one instance of R⁵ is unbranched C₄-C₃₅ heteroalkenyl. In some embodiments, at least one instance of R⁵ is unbranched C₄-C₁₂ heteroalkenyl. In some embodiments, at least one instance of R⁵ is unbranched C₁₀-C₂₀ heteroalkenyl. In some embodiments, at least one instance of R⁵ is unbranched C₁₂-C₂₅ heteroalkenyl.

In some embodiments, at least one instance of R⁵ is branched C₄-C₃₅ heteroalkynyl. In some embodiments, at least one instance of R⁵ is branched C₄-C₁₂ heteroalkynyl. In some embodiments, at least one instance of R⁵ is branched C₁₀-C₂₀ heteroalkynyl. In some embodiments, at least one instance of R⁵ is branched C₁₂-C₂₅ heteroalkynyl. In some embodiments, at least one instance of R⁵ is unbranched C₄-C₃₅ heteroalkynyl. In some embodiments, at least one instance of R⁵ is unbranched C₄-C₁₂ heteroalkynyl. In some embodiments, at least one instance of R⁵ is unbranched C₁₀-C₂₀ heteroalkynyl. In some embodiments, at least one instance of R⁵ is unbranched C₁₂-C₂₅ heteroalkynyl.

In some embodiments, at least one instance of R⁵ is selected from the group consisting of:

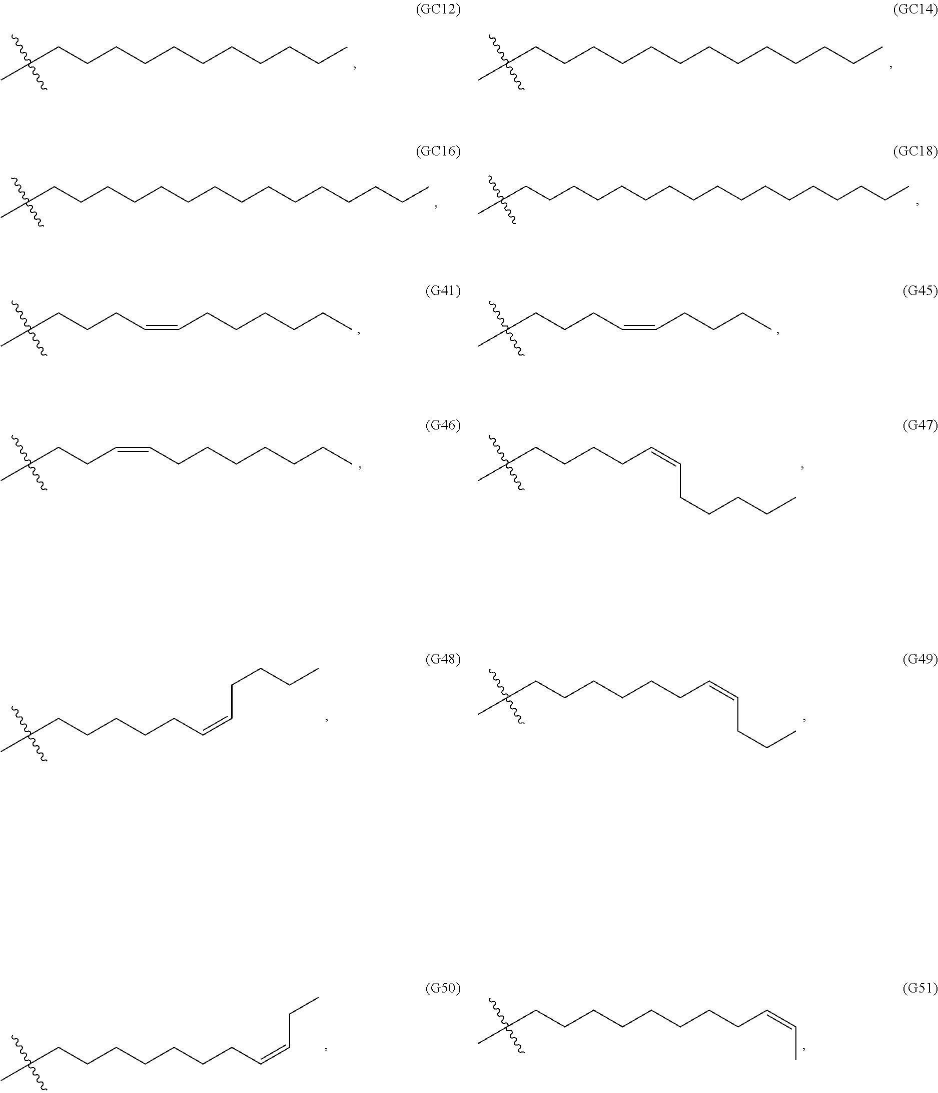

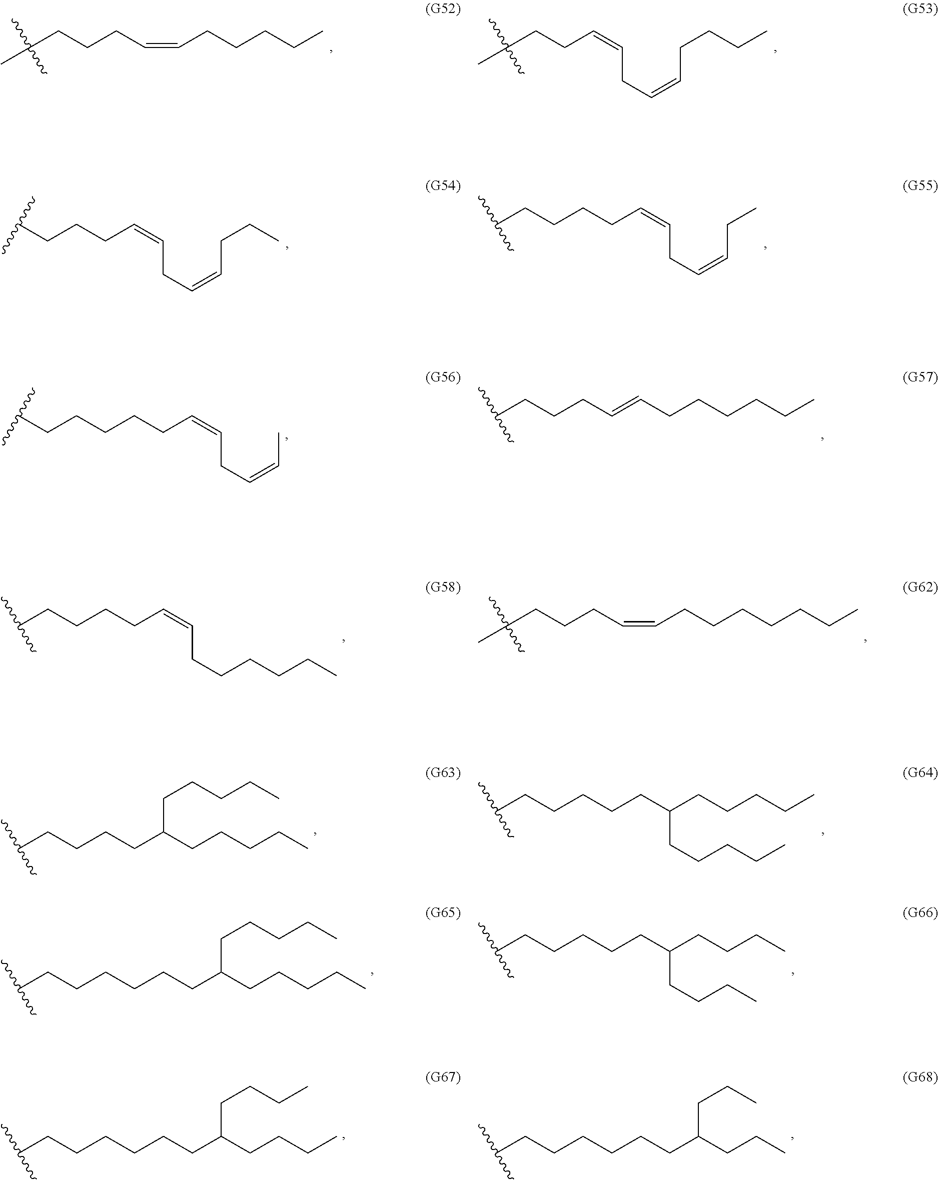

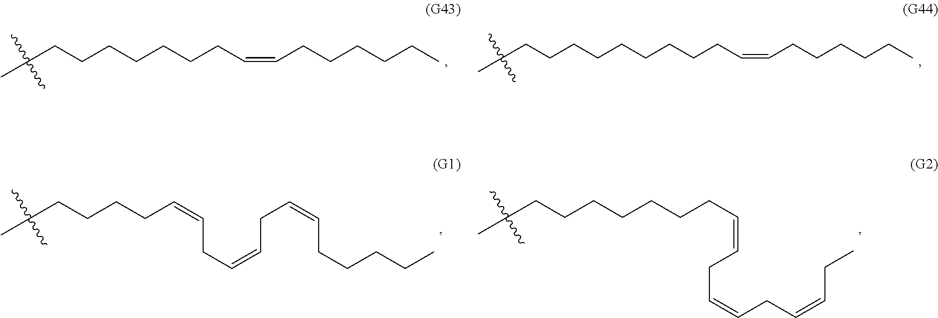 [SMILES: *CCCCCCC/C=C\CCCCCC ; *CCCCCCCCC/C=C\CCCCCC ; *CCCC/C=C\C/C=C\C/C=C\CCCCC ; *CCCCCCC/C=C\C/C=C\C/C=C\CC]

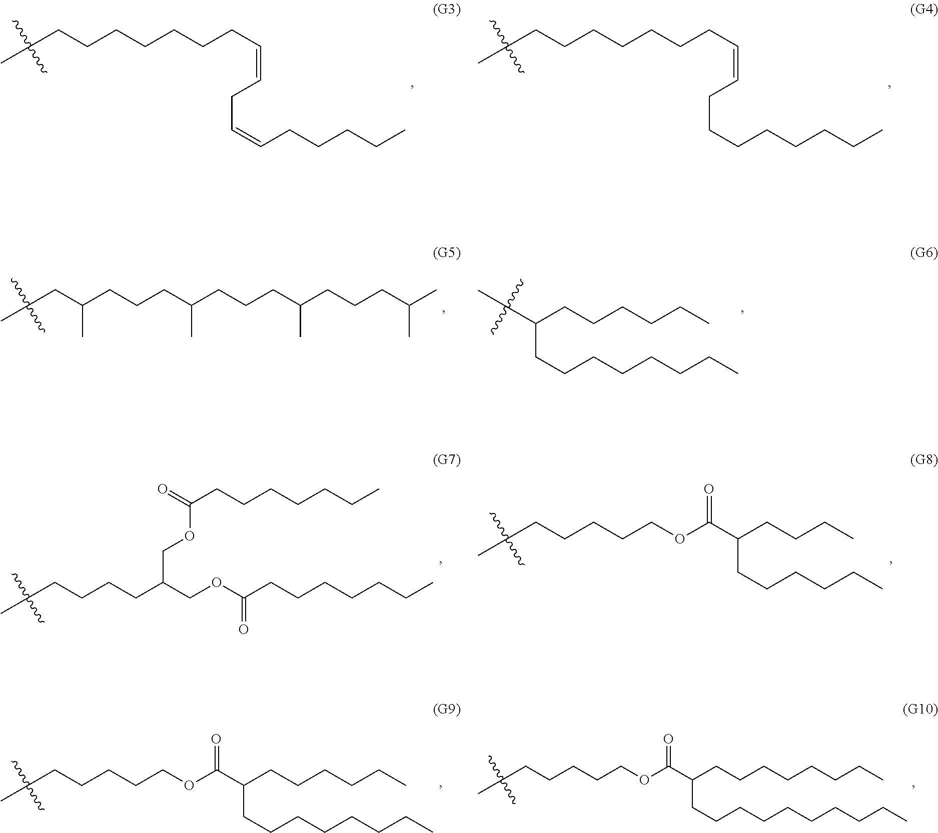

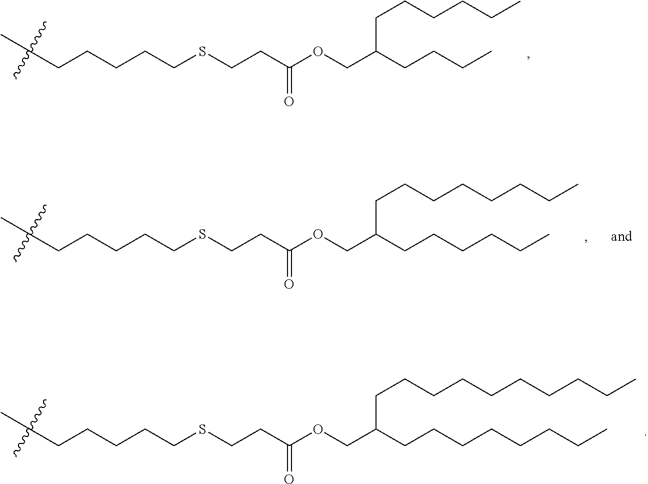 [SMILES: *CCCCCSCCC(=O)OCC(CCCC)CCCCCC ; *CCCCCSCCC(=O)OCC(CCCCCC)CCCCCCCC ; *CCCCCSCCC(=O)OCC(CCCCCCCC)CCCCCCCCCC]

In some embodiments, at least one instance of R⁵ is selected from the group consisting of:

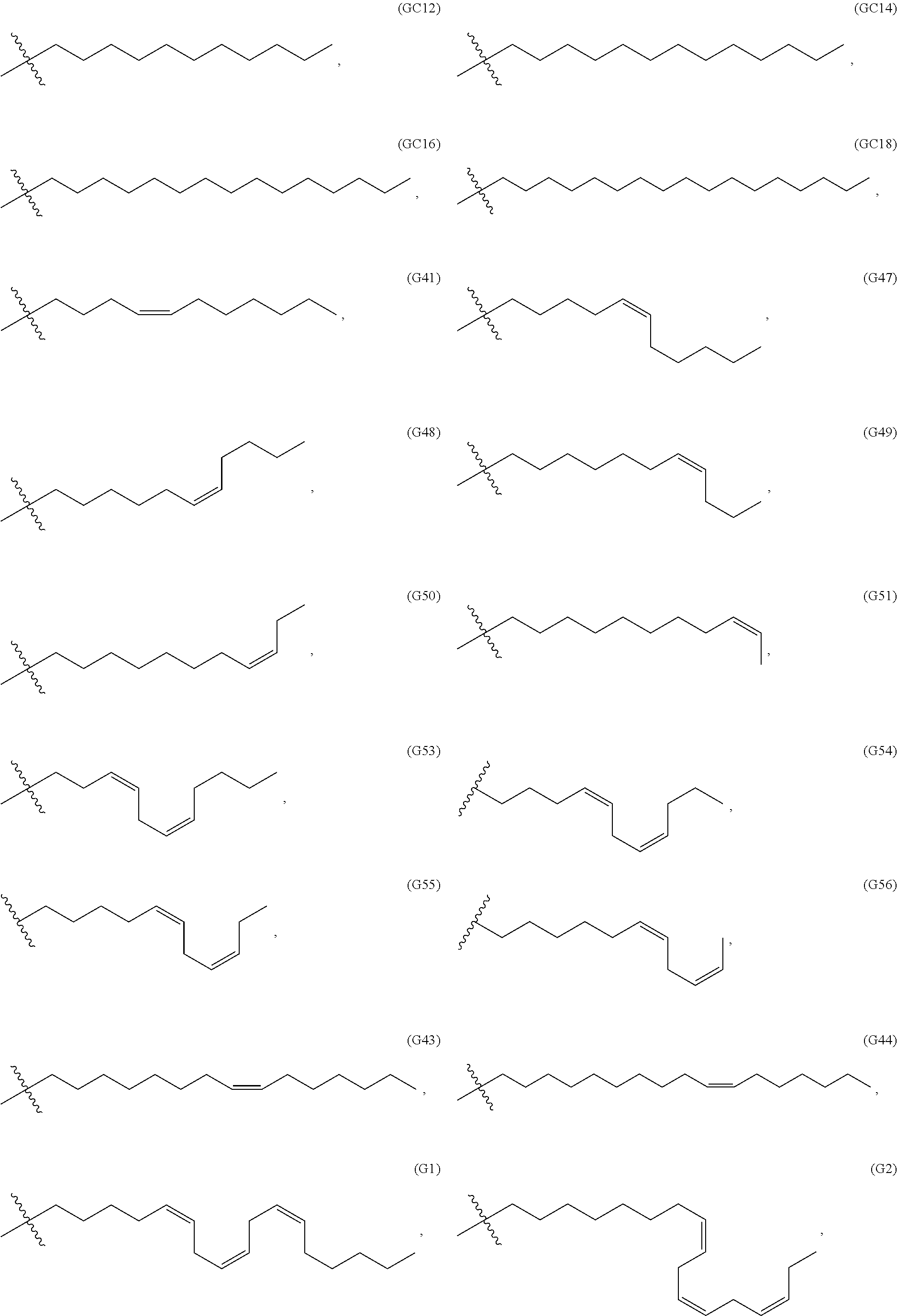

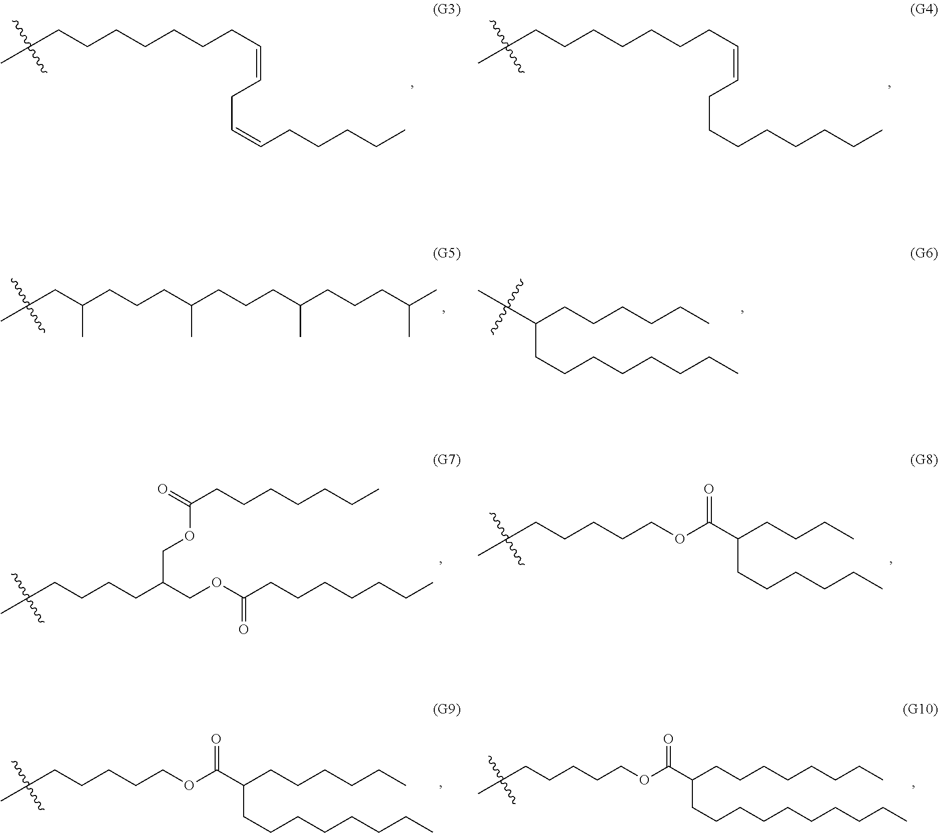

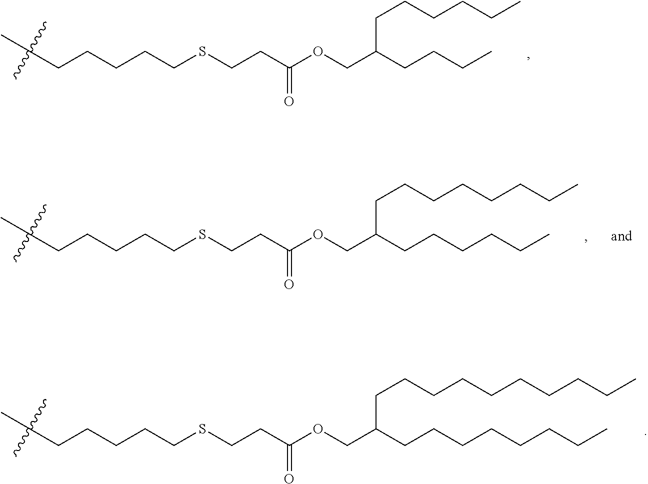 [SMILES: *CCCCCSCCC(=O)OCC(CCCC)CCCCCC ; *CCCCCSCCC(=O)OCC(CCCCCC)CCCCCCCC ; *CCCCCSCCC(=O)OCC(CCCCCCCC)CCCCCCCCCC]

In some embodiments, at least one instance of R⁵ is selected from the group consisting of:

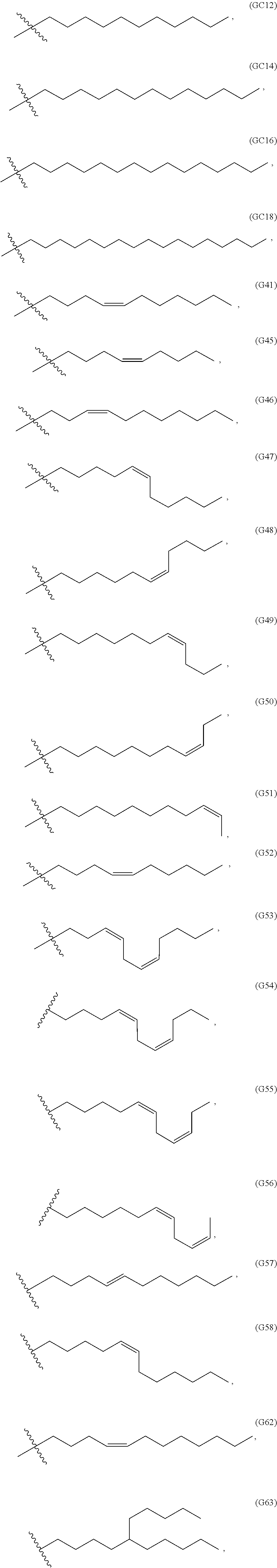

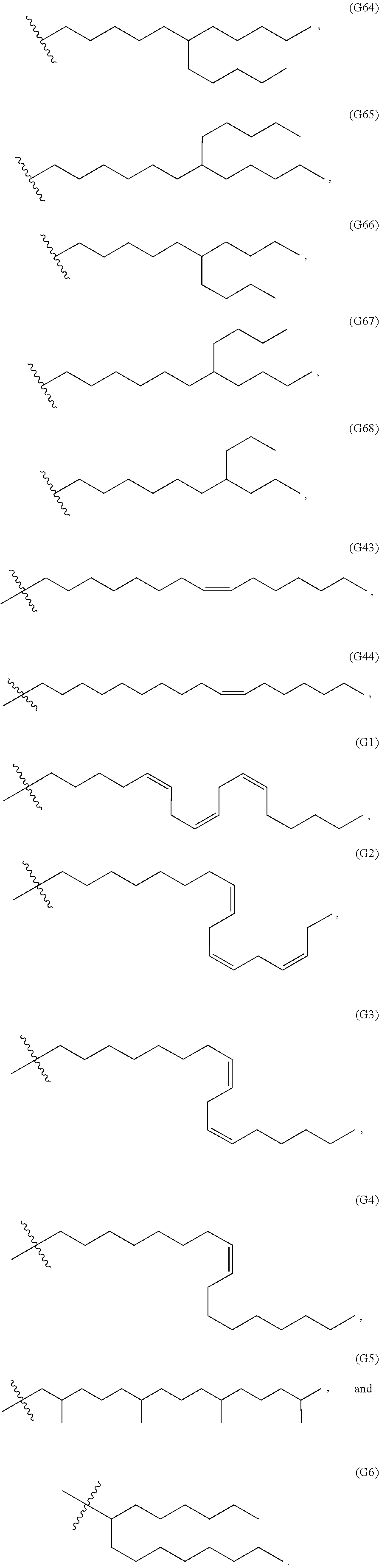

In some embodiments, at least one instance of R⁵ is selected from the group consisting of:

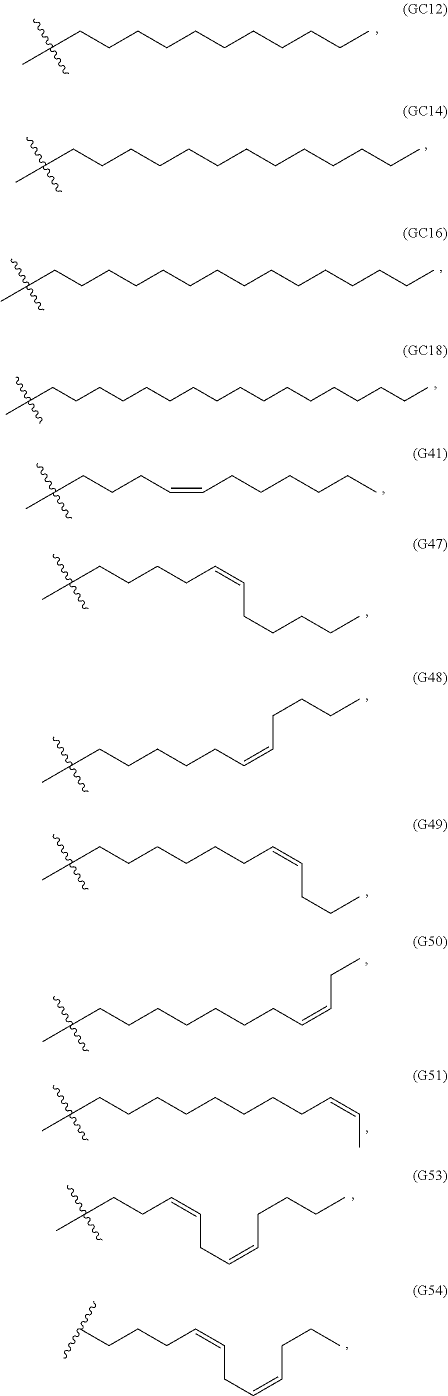

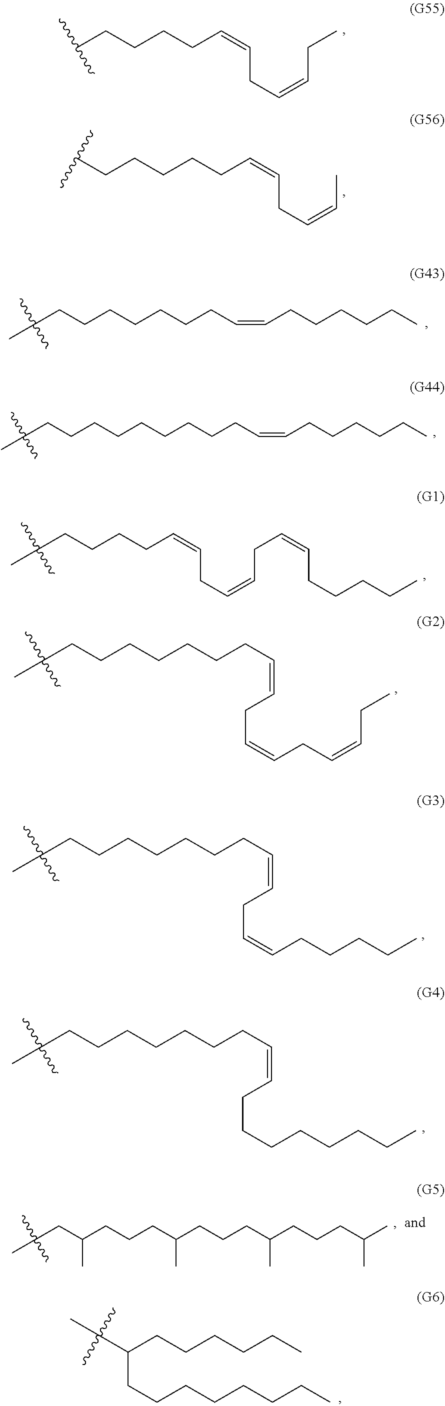

In some embodiments, at least one instance of R⁵ is selected from the group consisting of:

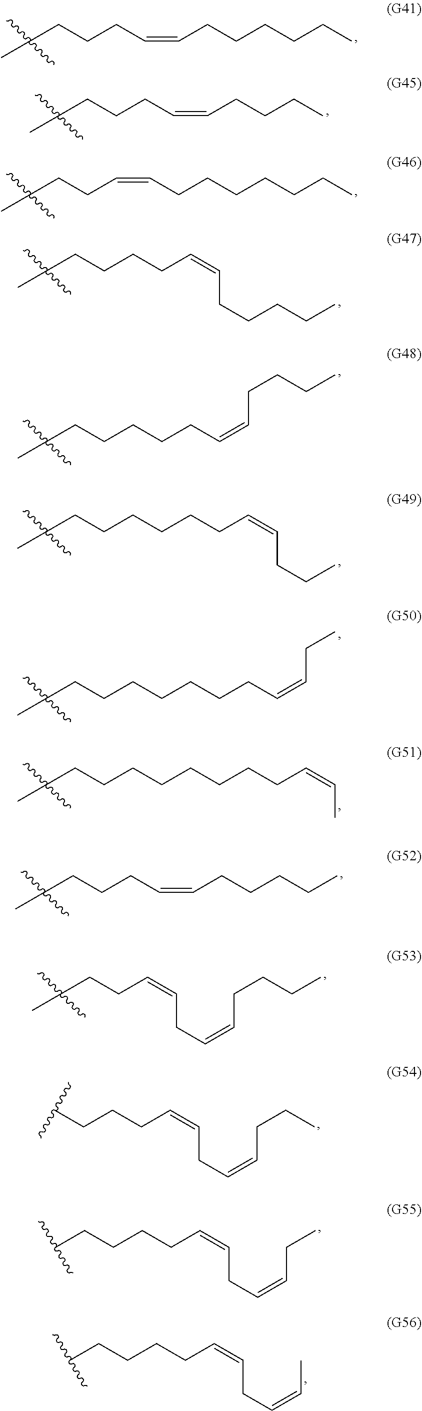

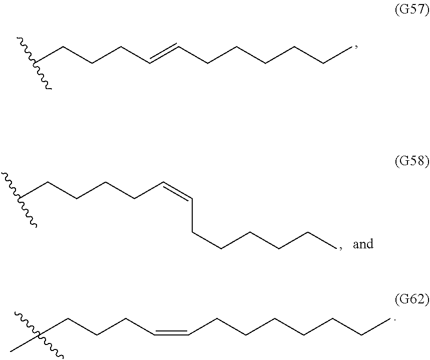 [SMILES: *CCC/C=C/CCCCCC ; *CCCC/C=C\CCCCCC ; *CCC/C=C\CCCCCCC]

In some embodiments, at least one instance of R⁵ is selected from the group consisting of:

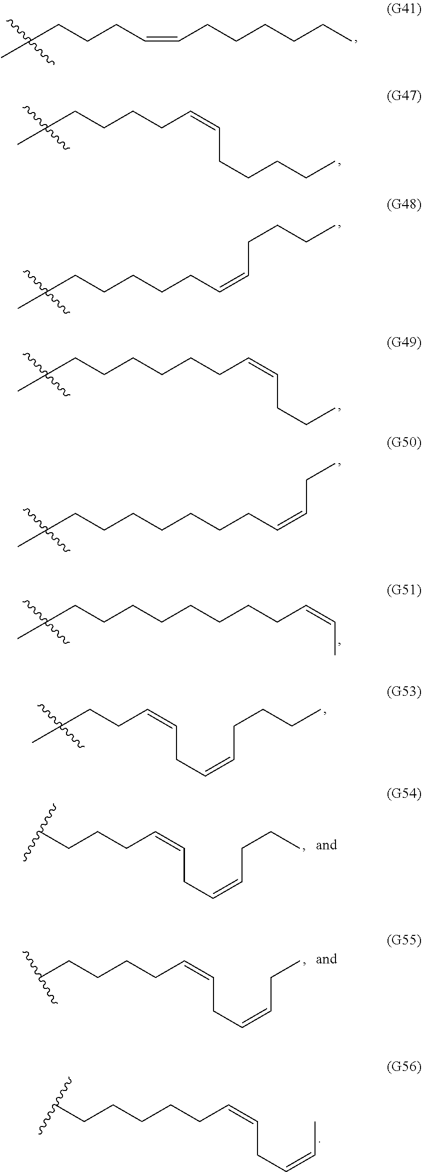

In some embodiments, R⁵ is

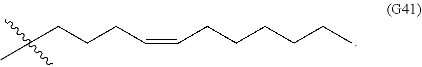 [SMILES: *CCC/C=C\CCCCCC]

In some embodiments, at least one instance of R⁵ is selected from the group consisting of:

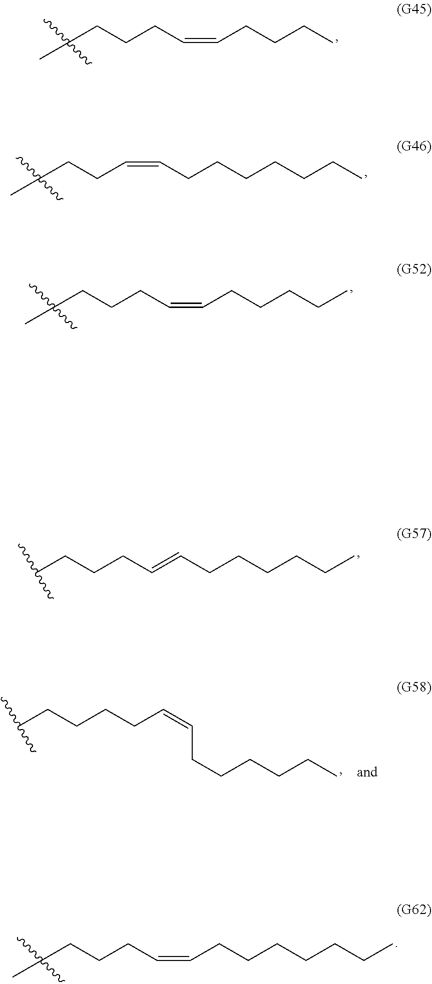

In some embodiments, at least one instance of R⁵ is selected from the group consisting of:

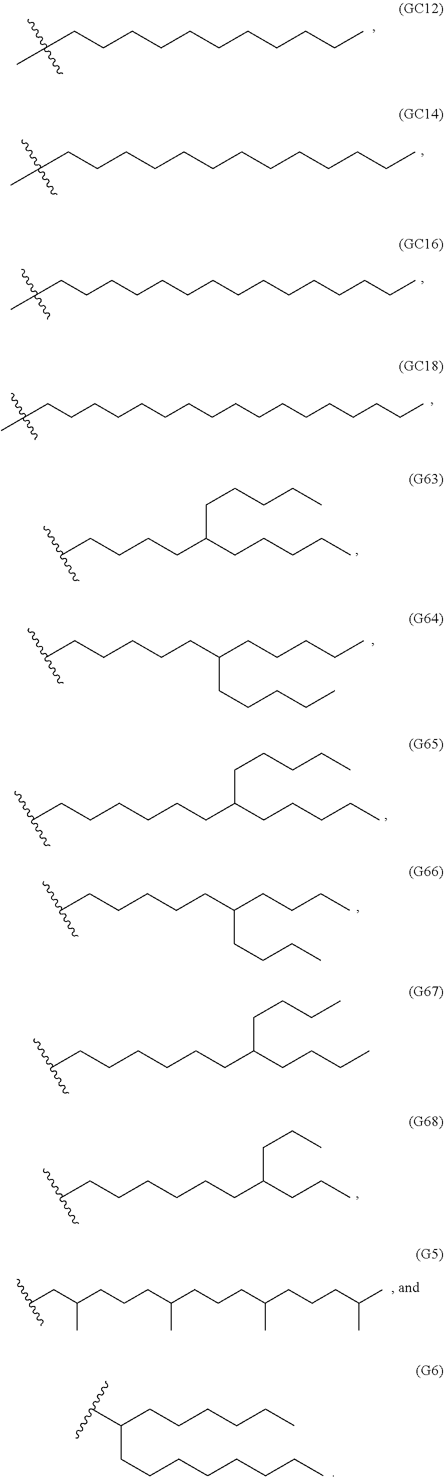

In some embodiments, at least one instance of R⁵ is selected from the group consisting of:

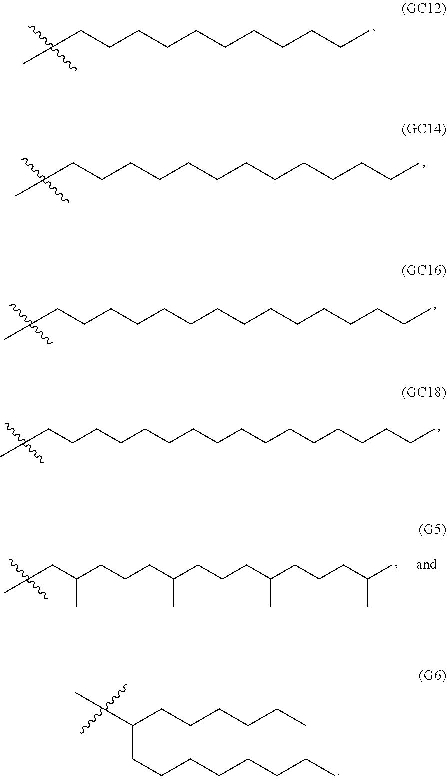 [SMILES: *CCCCCCCCCCC ; *CCCCCCCCCCCCC ; *CCCCCCCCCCCCCCC ; *CCCCCCCCCCCCCCCCC ; *CC(C)CCCC(C)CCCC(C)CCCC(C)C ; *C(CCCCCC)CCCCCCCC]

In some embodiments, at least one instance of R⁵ is selected from the group consisting of:

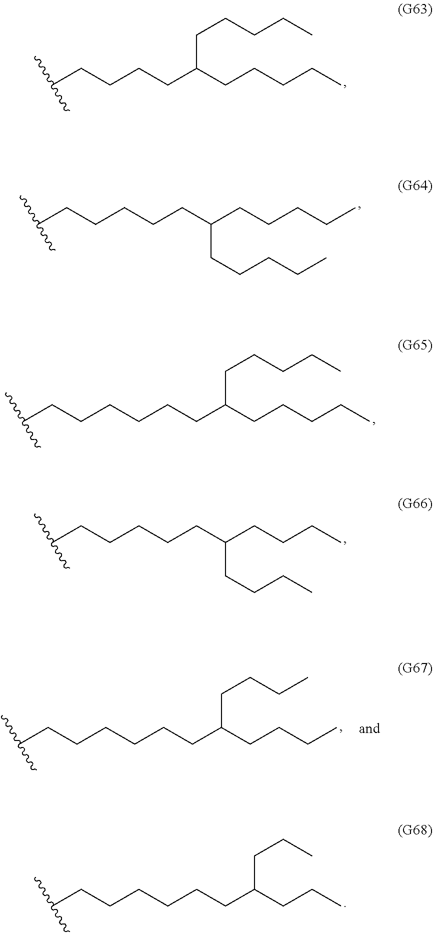 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

In some embodiments, at least one instance of R⁵ is selected from the group consisting

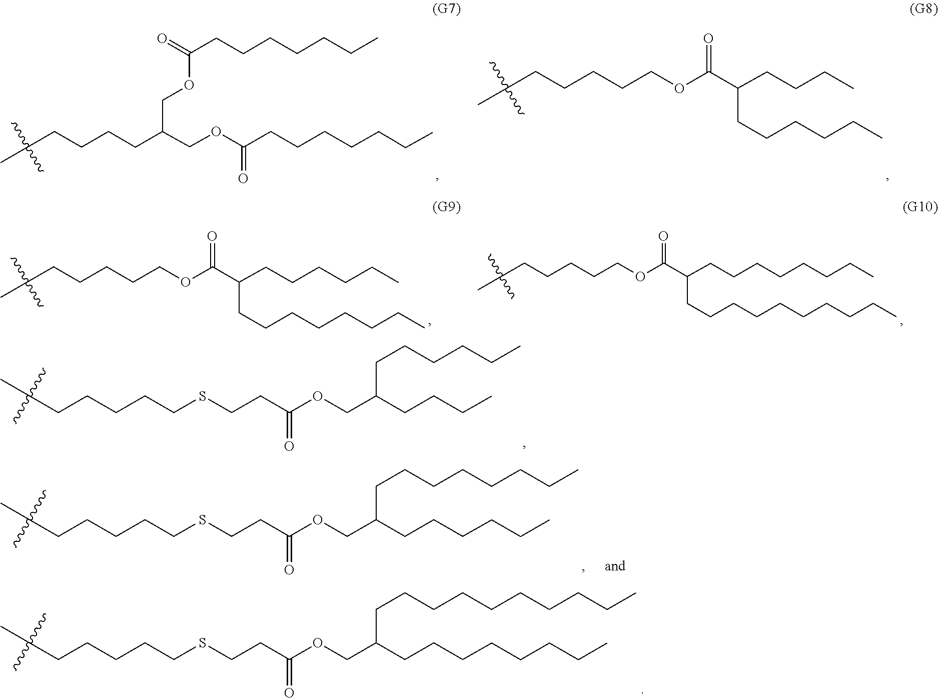

In some embodiments, at least one instance of R⁵ is selected from the group consisting

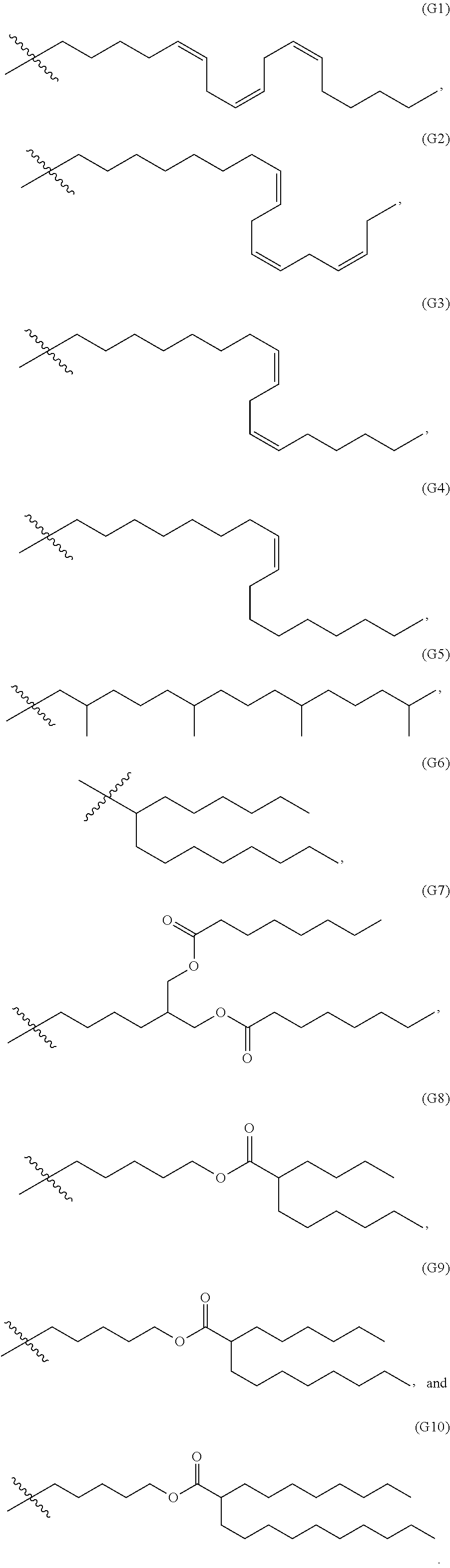

In a particular embodiment, R⁵ is G41. In a particular embodiment, R⁵ is G45. In a particular embodiment, R⁵ is G46. In a particular embodiment, R⁵ is G47. In a particular embodiment, R⁵ is G48. In a particular embodiment, R⁵ is G49. In a particular embodiment, R⁵ is G50. In a particular embodiment, R⁵ is G51. In a particular embodiment, R⁵ is G52. In a particular embodiment, R⁵ is G53. In a particular embodiment, R⁵ is G54. In a particular embodiment, R⁵ is G55. In a particular embodiment, R⁵ is G56. In a particular embodiment, R⁵ is G57. In a particular embodiment, R⁵ is G58. In a particular embodiment, R⁵ is G62. In a particular embodiment, R⁵ is G63. In a particular embodiment, R⁵ is G64. In a particular embodiment, R⁵ is G65. In a particular embodiment, R⁵ is G66. In a particular embodiment, R⁵ is G67. In a particular embodiment, R⁵ is G68. In a particular embodiment, R⁵ is GC12. In a particular embodiment, R⁵ is GC14. In a particular embodiment, R⁵ is GC16. In a particular embodiment, R⁵ is GC18. In a particular embodiment, R⁵ is G42. In a particular embodiment, R⁵ is G43. In a particular embodiment, R⁵ is G44. In a particular embodiment, R⁵ is G1. In a particular embodiment, R⁵ is G2. In a particular embodiment, R⁵ is G3. In a particular embodiment, R⁵ is G4. In a particular embodiment, R⁵ is G5. In a particular embodiment, R⁵ is G6. In a particular embodiment, R⁵ is G7. In a particular embodiment, R⁵ is G8. In a particular embodiment, R⁵ is G9. In a particular embodiment, R⁵ is G10. In a particular embodiment, R⁵ is

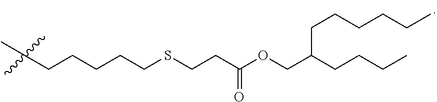 [SMILES: *CCCCCSCCC(=O)OCC(CCCC)CCCCCC]

In a particular embodiment, R⁵ is

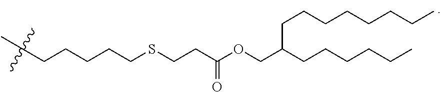 [SMILES: *CCCCCSCCC(=O)OCC(CCCCCC)CCCCCCCC]

In a particular embodiment, R⁵ is

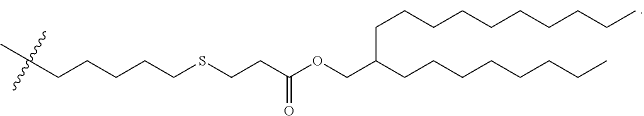 [SMILES: *CCCCCSCCC(=O)OCC(CCCCCCCC)CCCCCCCCCC]

In any of the embodiments of R described herein, one or more carbon atoms may be replaced with a heteroatom, e.g., to form a chemical functional group such as an ester. In some embodiments, R⁵ comprises a functional group that is biodegradable. In some embodiments, the biodegradable functional group is an ester.

As defined herein, each x independently is an integer from 1 to 10, inclusive. In some embodiments, each x independently is an integer from 1 to 9, inclusive. In some embodiments, each x independently is an integer from 1 to 8, inclusive. In some embodiments, each x independently is an integer from 1 to 7, inclusive. In some embodiments, each x independently is an integer from 1 to 6, inclusive. In some embodiments, each x independently is an integer from 1 to 5, inclusive. In some embodiments, each x independently is an integer from 1 to 4, inclusive. In some embodiments, each x independently is an integer from 1 to 3, inclusive. In some embodiments, each x independently is an integer from 1 to 2, inclusive.

In some embodiments, each x is the same. In some embodiments, at least two instances of x are the same. In some embodiments, at least three instances of x are the same. In some embodiments, at least four instances of x are the same. In some embodiments, at least five instances of x are the same. In some embodiments, at least six instances of x are the same. In some embodiments, at least seven instances of x are the same. In some embodiments, at least eight instances of x are the same. In some embodiments, six instances of x are the same. In some embodiments, seven instances of x are the same. In some embodiments, eight instances of x are the same.

In some embodiments, at least one x is 1. In some embodiments, at least one x is 2. In some embodiments, at least one x is 3. In some embodiments, at least one x is 4. In some embodiments, at least one x is 5. In some embodiments, at least one x is 6. In some embodiments, at least one x is 7. In some embodiments, at least one x is 8. In some embodiments, at least one x is 9. In some embodiments, at least one x is 10.

In some embodiments, each x is 1. In some embodiments, each x is 2. In some embodiments, each x is 3. In some embodiments, each x is 4. In some embodiments, each x is 5. In some embodiments, each x is 6. In some embodiments, each x is 7. In some embodiments, each x is 8. In some embodiments, each x is 9. In some embodiments, each x is 10.

In some embodiments, one x is 1. In some embodiments, two x are 1. In some embodiments, three x are 1. In some embodiments, four x are 1. In some embodiments, five x are 1. In some embodiments, six x are 1. In some embodiments, seven x are 1. In some embodiments, eight x are 1.

In some embodiments, the compound is of Formula (XI), or a pharmaceutically acceptable salt thereof, wherein each R is

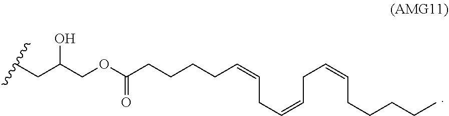 [SMILES: *CC(O)COC(=O)CCCC/C=C\C/C=C\C/C=C\CCCCC]

In some embodiments, the compound is of Formula (XI), or a pharmaceutically acceptable salt thereof, wherein each R is

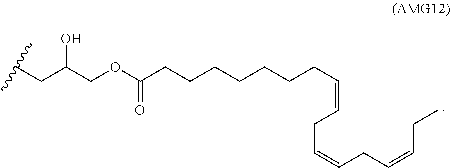 [SMILES: *CC(O)COC(=O)CCCCCCC/C=C\C/C=C\C/C=C\CC]

In some embodiments, the compound is of Formula (XI), or a pharmaceutically acceptable salt thereof, wherein each R

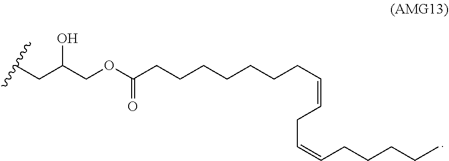 [SMILES: *CC(O)COC(=O)CCCCCCC/C=C\C/C=C\CCCCC]

In some embodiments, the compound is of Formula (XI), or a pharmaceutically acceptable salt thereof, wherein each R

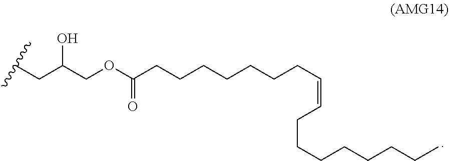 [SMILES: *CC(O)COC(=O)CCCCCCC/C=C\CCCCCCCC]

In some embodiments, the compound is of Formula (XI), or a pharmaceutically acceptable salt thereof, wherein each R

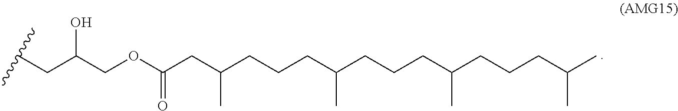 [SMILES: *CC(O)COC(=O)CC(C)CCCC(C)CCCC(C)CCCC(C)C]

In some embodiments, the compound is of Formula (XI), or a pharmaceutically acceptable salt thereof, wherein each R is

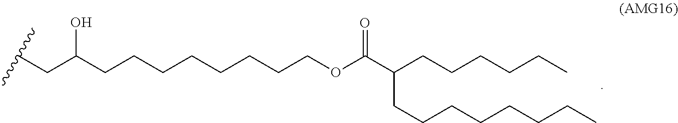 [SMILES: *CC(O)CCCCCCCCOC(=O)C(CCCCCC)CCCCCCCC]

In some embodiments, the compound is of Formula (XI), or a pharmaceutically acceptable salt thereof, wherein each R is

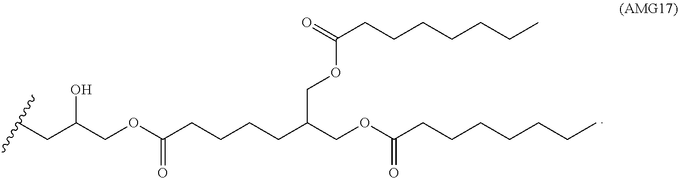 [SMILES: *CC(O)COC(=O)CCCCC(COC(=O)CCCCCCC)COC(=O)CCCCCCC]

In some embodiments, the compound is of Formula (XI), or a pharmaceutically acceptable salt thereof, wherein each

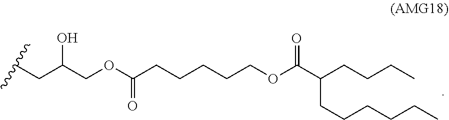 [SMILES: *CC(O)COC(=O)CCCCCOC(=O)C(CCCC)CCCCCC]

In some embodiments, the compound is of Formula (XI), or a pharmaceutically acceptable salt thereof, wherein each R

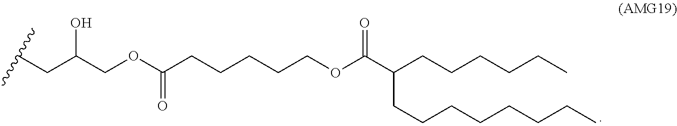 [SMILES: *CC(O)COC(=O)CCCCCOC(=O)C(CCCCCC)CCCCCCCC]

In some embodiments, the compound is of Formula (XI), or a pharmaceutically acceptable salt thereof, wherein each R is

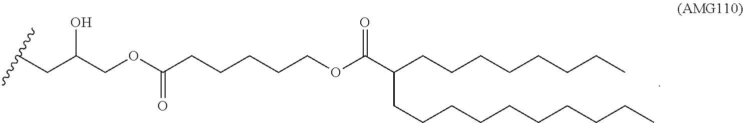 [SMILES: *CC(O)COC(=O)CCCCCOC(=O)C(CCCCCCCC)CCCCCCCCCC]

In some embodiments, the compound is of Formula (XII), or a pharmaceutically acceptable salt thereof, wherein each R is

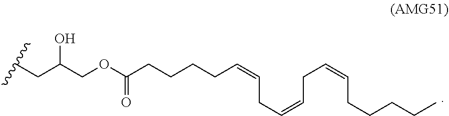 [SMILES: *CC(O)COC(=O)CCCC/C=C\C/C=C\C/C=C\CCCCC]

In some embodiments, the compound is of Formula (XII), or a pharmaceutically acceptable salt thereof, wherein each R is

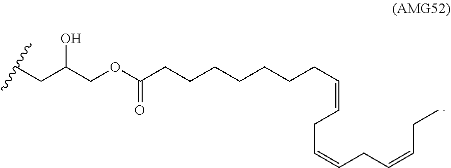 [SMILES: *CC(O)COC(=O)CCCCCCC/C=C\C/C=C\C/C=C\CC]

In some embodiments, the compound is of Formula (XII), or a pharmaceutically acceptable salt thereof, wherein each R is

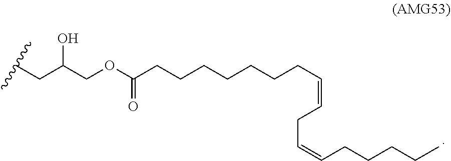 [SMILES: *CC(O)COC(=O)CCCCCCC/C=C\C/C=C\CCCCC]

In some embodiments, the compound is of Formula (XII), or a pharmaceutically acceptable salt thereof, wherein each R is

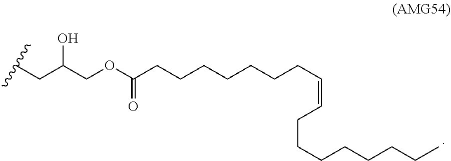 [SMILES: *CC(O)COC(=O)CCCCCCC/C=C\CCCCCCCC]

In some embodiments, the compound is of Formula (XII), or a pharmaceutically acceptable salt thereof, wherein each R is

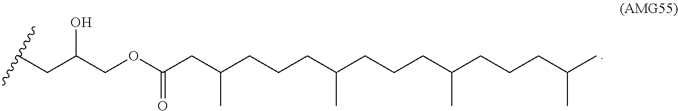 [SMILES: *CC(O)COC(=O)CC(C)CCCC(C)CCCC(C)CCCC(C)C]

In some embodiments, the compound is of Formula (XII), or a pharmaceutically acceptable salt thereof, wherein each R is

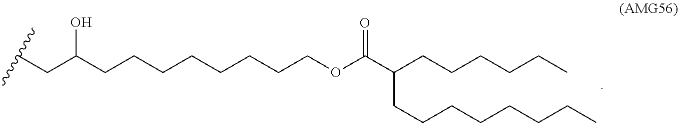 [SMILES: *CC(O)CCCCCCCCOC(=O)C(CCCCCC)CCCCCCCC]

In some embodiments, the compound is of Formula (XII), or a pharmaceutically acceptable salt thereof, wherein each R is

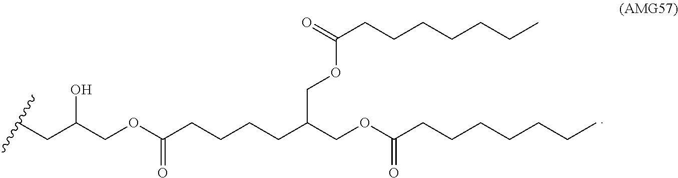 [SMILES: *CC(O)COC(=O)CCCCC(COC(=O)CCCCCCC)COC(=O)CCCCCCC]

In some embodiments, the compound is of Formula (XII), or a pharmaceutically acceptable salt thereof, wherein each R is

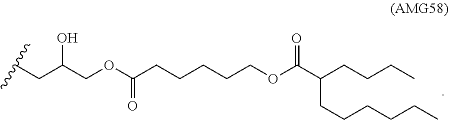 [SMILES: *CC(O)COC(=O)CCCCCOC(=O)C(CCCC)CCCCCC]

In some embodiments, the compound is of Formula (XII), or a pharmaceutically acceptable salt thereof, wherein each R is

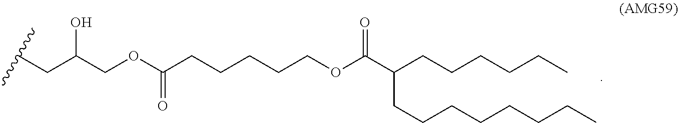 [SMILES: *CC(O)COC(=O)CCCCCOC(=O)C(CCCCCC)CCCCCCCC]

In some embodiments, the compound is of Formula (XII), or a pharmaceutically acceptable salt thereof, wherein each R is

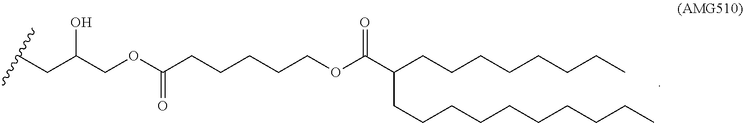 [SMILES: *CC(O)COC(=O)CCCCCOC(=O)C(CCCCCCCC)CCCCCCCCCC]

In some embodiments, the compound of Formula (XIII) is of Formula (XIII-A):

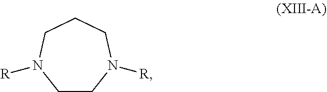

or a pharmaceutically acceptable salt, solvate, tautomer, stereoisomer, or isotopically labeled derivative thereof.

In some embodiments, the compound is of Formula (XIII), or a pharmaceutically acceptable salt thereof, wherein R¹ is hydrogen and each R is

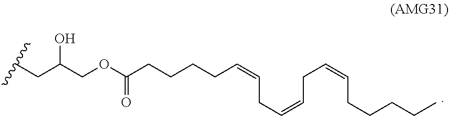 [SMILES: *CC(O)COC(=O)CCCC/C=C\C/C=C\C/C=C\CCCCC]

In some embodiments, the compound is of Formula (XIII), or a pharmaceutically acceptable salt thereof, wherein R¹ is hydrogen and each R is

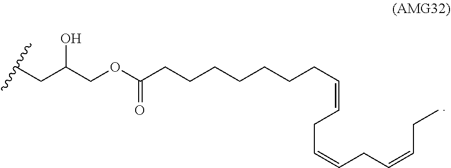 [SMILES: *CC(O)COC(=O)CCCCCCC/C=C\C/C=C\C/C=C\CC]

In some embodiments, the compound is of Formula (XIII), or a pharmaceutically acceptable salt thereof, wherein R¹ is hydrogen and each R is

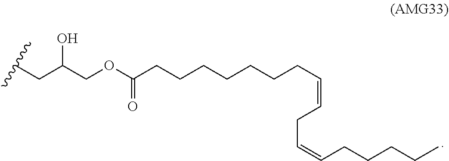 [SMILES: *CC(O)COC(=O)CCCCCCC/C=C\C/C=C\CCCCC]

In some embodiments, the compound is of Formula (XIII), or a pharmaceutically acceptable salt thereof, wherein R¹ is hydrogen and each R is

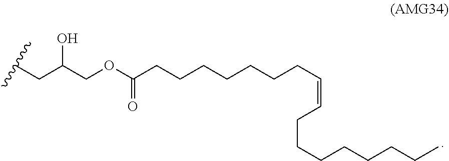 [SMILES: *CC(O)COC(=O)CCCCCCC/C=C\CCCCCCCC]

In some embodiments, the compound is of Formula (XIII), or a pharmaceutically acceptable salt thereof, wherein R¹ is hydrogen and each R is

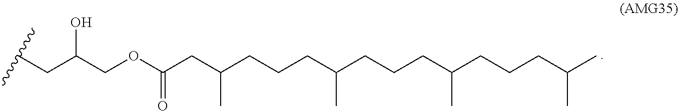 [SMILES: *CC(O)COC(=O)CC(C)CCCC(C)CCCC(C)CCCC(C)C]

In some embodiments, the compound is of Formula (XIII), or a pharmaceutically acceptable salt thereof, wherein R¹ is hydrogen and each R is

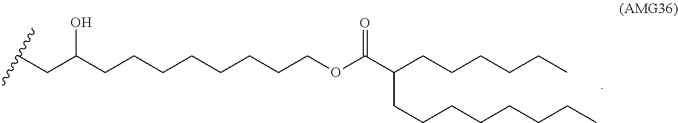 [SMILES: *CC(O)CCCCCCCCOC(=O)C(CCCCCC)CCCCCCCC]

In some embodiments, the compound is of Formula (XIII), or a pharmaceutically acceptable salt thereof, wherein R¹ is hydrogen and each R is

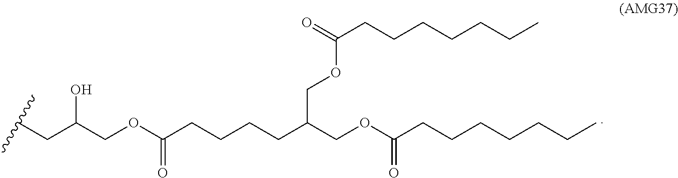 [SMILES: *CC(O)COC(=O)CCCCC(COC(=O)CCCCCCC)COC(=O)CCCCCCC]

In some embodiments, the compound is of Formula (XIII), or a pharmaceutically acceptable salt thereof, wherein R¹ is hydrogen and each R is

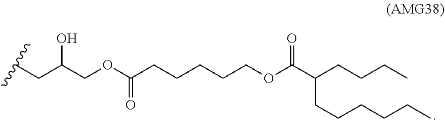 [SMILES: *CC(O)COC(=O)CCCCCOC(=O)C(CCCC)CCCCCC]

In some embodiments, the compound is of Formula (XIII), or a pharmaceutically acceptable salt thereof, wherein R¹ is hydrogen and each R is

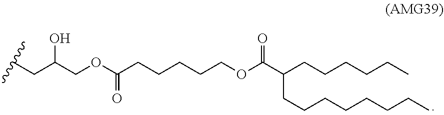 [SMILES: *CC(O)COC(=O)CCCCCOC(=O)C(CCCCCC)CCCCCCCC]

In some embodiments, the compound is of Formula (XIII), or a pharmaceutically acceptable salt thereof, wherein R¹ is hydrogen and each R is

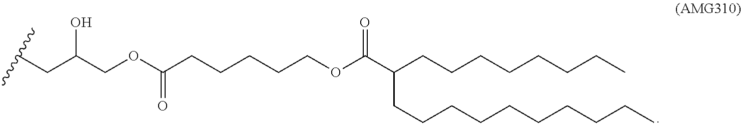 [SMILES: *CC(O)COC(=O)CCCCCOC(=O)C(CCCCCCCC)CCCCCCCCCC]

In some embodiments, the compound of Formula (XIII) is of Formula (XIII-A):

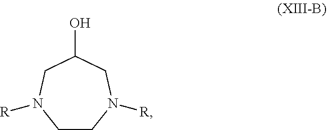

or a pharmaceutically acceptable salt, solvate, tautomer, stereoisomer, or isotopically labeled derivative thereof.

In some embodiments, the compound is of Formula (XIII), or a pharmaceutically acceptable salt thereof, wherein R¹ is —OH and each R is

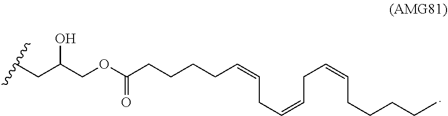 [SMILES: *CC(O)COC(=O)CCCC/C=C\C/C=C\C/C=C\CCCCC]

In some embodiments, the compound is of Formula (XIII), or a pharmaceutically acceptable salt thereof, wherein R¹ is —OH and each R is

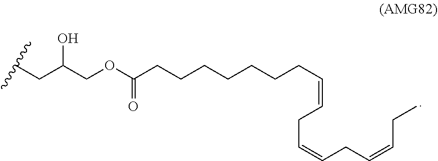 [SMILES: *CC(O)COC(=O)CCCCCCC/C=C\C/C=C\C/C=C\CC]

In some embodiments, the compound is of Formula (XIII), or a pharmaceutically acceptable salt thereof, wherein R¹ is —OH and each R is

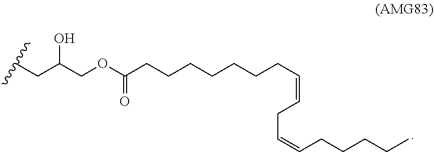 [SMILES: *CC(O)COC(=O)CCCCCCC/C=C\C/C=C\CCCCC]

In some embodiments, the compound is of Formula (XIII) or a pharmaceutically acceptable salt thereof, wherein R¹ is —OH and each R is

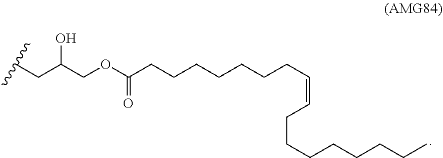 [SMILES: *CC(O)COC(=O)CCCCCCC/C=C\CCCCCCCC]

In some embodiments, the compound is of Formula (XIII), or a pharmaceutically acceptable salt thereof, wherein R¹ is —OH and each R is

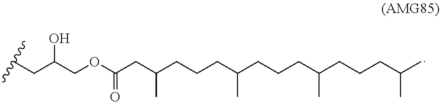 [SMILES: *CC(O)COC(=O)CC(C)CCCC(C)CCCC(C)CCCC(C)C]

In some embodiments, the compound is of Formula (XIII), or a pharmaceutically acceptable salt thereof, wherein R¹ is —OH and each R is

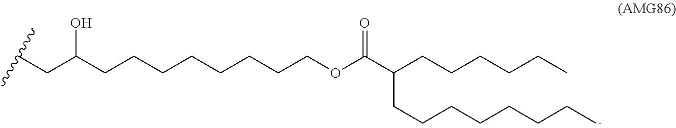 [SMILES: *CC(O)CCCCCCCCOC(=O)C(CCCCCC)CCCCCCCC]

In some embodiments, the compound is of Formula (XIII), or a pharmaceutically acceptable salt thereof, wherein R¹ is —OH and each R is

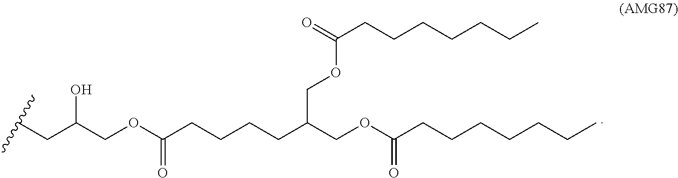 [SMILES: *CC(O)COC(=O)CCCCC(COC(=O)CCCCCCC)COC(=O)CCCCCCC]

In some embodiments, the compound is of Formula (XIII), or a pharmaceutically acceptable salt thereof, wherein R¹ is —OH and each R is

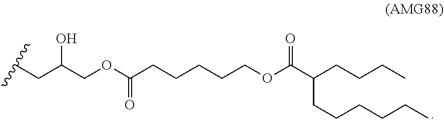 [SMILES: *CC(O)COC(=O)CCCCCOC(=O)C(CCCC)CCCCCC]

In some embodiments, the compound is of Formula (XIII), or a pharmaceutically acceptable salt thereof, wherein R¹ is —OH and each R is

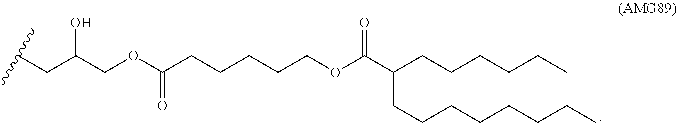 [SMILES: *CC(O)COC(=O)CCCCCOC(=O)C(CCCCCC)CCCCCCCC]

In some embodiments, the compound is of Formula (XIII), or a pharmaceutically acceptable salt thereof, wherein R¹ is —OH and each R is

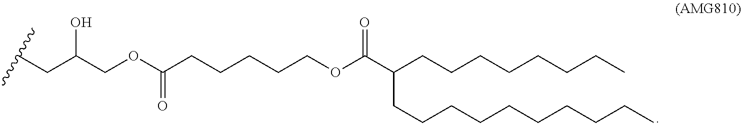 [SMILES: *CC(O)COC(=O)CCCCCOC(=O)C(CCCCCCCC)CCCCCCCCCC]

In some embodiments, the compound of Formula (I) is of Formula (II):

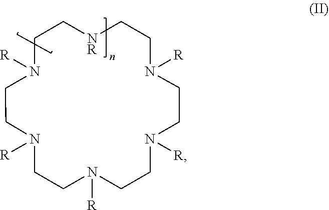

or a pharmaceutically acceptable salt, solvate, tautomer, stereoisomer, or isotopically labeled derivative thereof. In some embodiments, the compound of Formula (I) is of Formula (II), or a pharmaceutically acceptable salt thereof.

In some embodiments, the compound is of the formula:

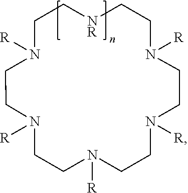

or a pharmaceutically acceptable salt thereof.

In some embodiments, the compound of Formula (I) is of Formula (III):

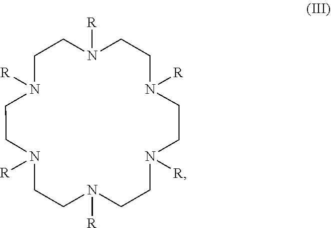

or a pharmaceutically acceptable salt, solvate, tautomer, stereoisomer, or isotopically labeled derivative thereof. In some embodiments, the compound of Formula (I) is of Formula (III), or a pharmaceutically acceptable salt thereof.

In some embodiments, the compound of Formula (I) is of Formula (III), or a pharmaceutically acceptable salt thereof, wherein each R is

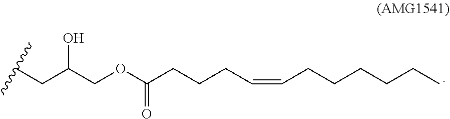 [SMILES: *CC(O)COC(=O)CCC/C=C\CCCCCC]

In some embodiments, the compound of Formula (I) is of Formula (III), or a pharmaceutically acceptable salt thereof, wherein each R is

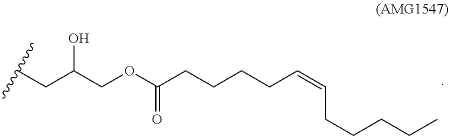 [SMILES: *CC(O)COC(=O)CCCC/C=C\CCCCC]

In some embodiments, the compound of Formula (I) is of Formula (III), or a pharmaceutically acceptable salt thereof, wherein each R is

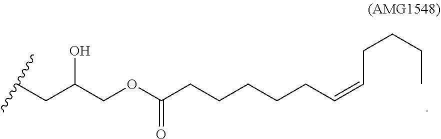 [SMILES: *CC(O)COC(=O)CCCCC/C=C\CCCC]

In some embodiments, the compound of Formula (I) is of Formula (III), or a pharmaceutically acceptable salt thereof, wherein each R is

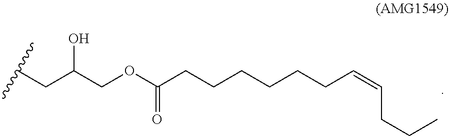 [SMILES: *CC(O)COC(=O)CCCCCC/C=C\CCC]

In some embodiments, the compound of Formula (I) is of Formula (III), or a pharmaceutically acceptable salt thereof, wherein each R is

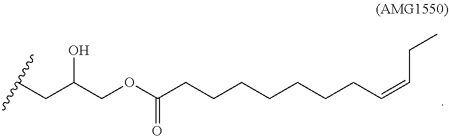 [SMILES: *CC(O)COC(=O)CCCCCCC/C=C\CC]

In some embodiments, the compound of Formula (I) is of Formula (III), or a pharmaceutically acceptable salt thereof, wherein each R is

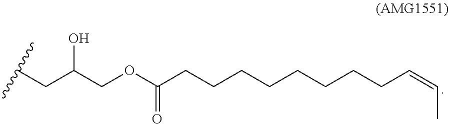 [SMILES: *CC(O)COC(=O)CCCCCCCC/C=C\C]

In some embodiments, the compound of Formula (I) is of Formula (III), or a pharmaceutically acceptable salt thereof, wherein each R is

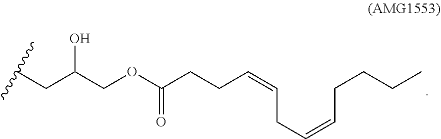 [SMILES: *CC(O)COC(=O)CC/C=C\C/C=C\CCCC]

In some embodiments, the compound of Formula (I) is of Formula (III), or a pharmaceutically acceptable salt thereof, wherein each R is

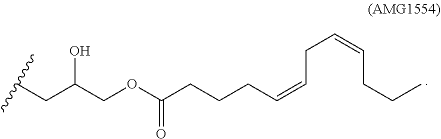 [SMILES: *CC(O)COC(=O)CCC/C=C\C/C=C\CCC]

In some embodiments, the compound of Formula (I) is of Formula (III), or a pharmaceutically acceptable salt thereof, wherein each R is

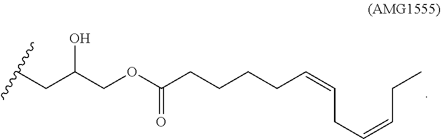 [SMILES: *CC(O)COC(=O)CCCC/C=C\C/C=C\CC]

In some embodiments, the compound of Formula (I) is of Formula (III), or a pharmaceutically acceptable salt thereof, wherein each R is

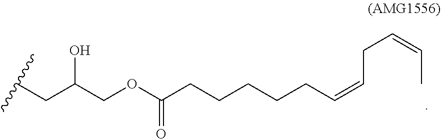 [SMILES: *CC(O)COC(=O)CCCCC/C=C\C/C=C\C]

In some embodiments, the compound of Formula (I) is of Formula (III), or a pharmaceutically acceptable salt thereof, wherein each R is

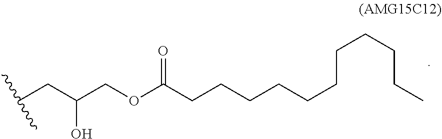 [SMILES: *CC(O)COC(=O)CCCCCCCCCCC]

In some embodiments, the compound of Formula (I) is of Formula (III), or a pharmaceutically acceptable salt thereof, wherein each R is

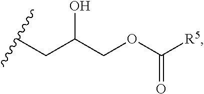

and wherein each R⁵ is

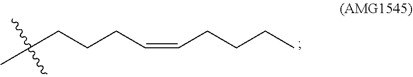 [SMILES: *CCC/C=C\CCCC]

each R⁵ is

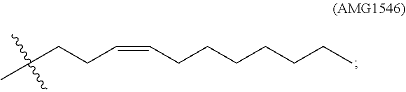 [SMILES: *CC/C=C\CCCCCCC]

each R⁵ is

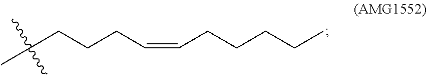 [SMILES: *CCC/C=C\CCCCC]

each R⁵ is

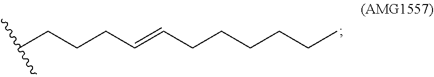 [SMILES: *CCC/C=C/CCCCCC]

each R⁵ is

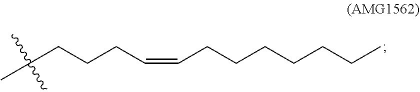 [SMILES: *CCC/C=C\CCCCCCC]

each R⁵ is

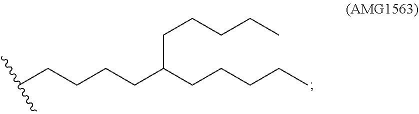 [SMILES: *CCCCC(CCCCC)CCCCC]

each R⁵ is

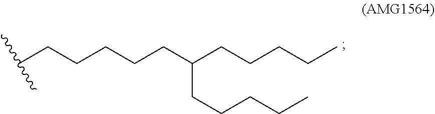 [SMILES: *CCCCCC(CCCCC)CCCCC]

each R⁵ is

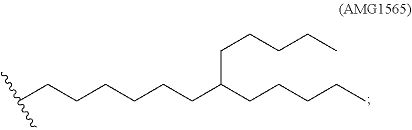 [SMILES: *CCCCCCC(CCCCC)CCCCC]

each R⁵ is

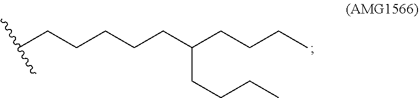 [SMILES: *CCCCCC(CCCC)CCCC]

each R⁵ is

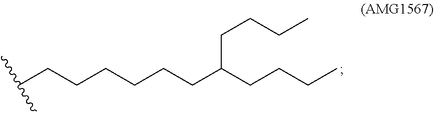 [SMILES: *CCCCCCC(CCCC)CCCC]

or each R⁵ is

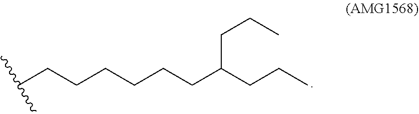 [SMILES: *CCCCCCC(CCC)CCC]

In some embodiments, the compound of Formula (I) is of Formula (III), or a pharmaceutically acceptable salt thereof, wherein each R is

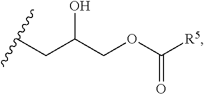

and wherein each R⁵ is

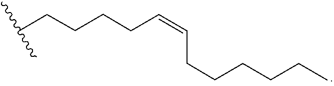 [SMILES: *CCCC/C=C\CCCCCC]

In some embodiments, the compound of Formula (I) is of Formula (III), or a pharmaceutically acceptable salt thereof, wherein each R is

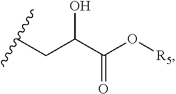

and wherein each R⁵ is

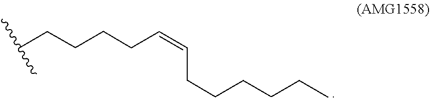 [SMILES: *CCCC/C=C\CCCCCC]

In some embodiments, the compound of Formula (I) is of Formula (III), or a pharmaceutically acceptable salt thereof, wherein each R is

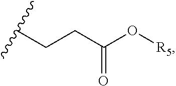

and wherein each R⁵ is

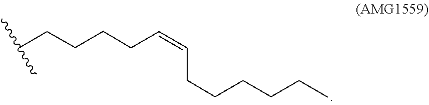 [SMILES: *CCCC/C=C\CCCCCC]

In some embodiments, the compound of Formula (I) is of Formula (III), or a pharmaceutically acceptable salt thereof, wherein each R is

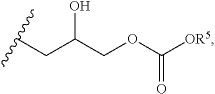

and wherein each R⁵ is

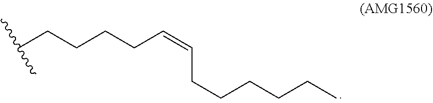 [SMILES: *CCCC/C=C\CCCCCC]

In some embodiments, the compound of Formula (I) is of Formula (III), or a pharmaceutically acceptable salt thereof, wherein each R is

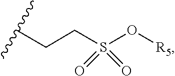

and wherein each R⁵ is

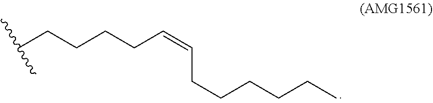 [SMILES: *CCCC/C=C\CCCCCC]

In some embodiments, the compound of Formula (I) is AMG1541, wherein each chiral center has (R) configuration. In some embodiments, the compound of Formula (I) is AMG1541, wherein each chiral center has (S) configuration. In some embodiments, the compound of Formula (I) is AMG1545, wherein each chiral center has (R) configuration. In some embodiments, the compound of Formula (I) is AMG1545, wherein each chiral center has (S) configuration. In some embodiments, the compound of Formula (I) is AMG1546, wherein each chiral center has (R) configuration. In some embodiments, the compound of Formula (I) is AMG1546, wherein each chiral center has (S) configuration. In some embodiments, the compound of Formula (I) is AMG1547, wherein each chiral center has (R) configuration. In some embodiments, the compound of Formula (I) is AMG1547, wherein each chiral center has (S) configuration. In some embodiments, the compound of Formula (I) is AMG1548, wherein each chiral center has (R) configuration. In some embodiments, the compound of Formula (I) is AMG1548, wherein each chiral center has (S) configuration. In some embodiments, the compound of Formula (I) is AMG1549, wherein each chiral center has (R) configuration. In some embodiments, the compound of Formula (I) is AMG1549, wherein each chiral center has (S) configuration. In some embodiments, the compound of Formula (I) is AMG1550, wherein each chiral center has (R) configuration. In some embodiments, the compound of Formula (I) is AMG1550, wherein each chiral center has (S) configuration. In some embodiments, the compound of Formula (I) is AMG1551, wherein each chiral center has (R) configuration. In some embodiments, the compound of Formula (I) is AMG1551, wherein each chiral center has (S) configuration. In some embodiments, the compound of Formula (I) is AMG1552, wherein each chiral center has (R) configuration. In some embodiments, the compound of Formula (I) is AMG1552, wherein each chiral center has (S) configuration. In some embodiments, the compound of Formula (I) is AMG1553, wherein each chiral center has (R) configuration. In some embodiments, the compound of Formula (I) is AMG1553, wherein each chiral center has (S) configuration. In some embodiments, the compound of Formula (I) is AMG1554, wherein each chiral center has (R) configuration. In some embodiments, the compound of Formula (I) is AMG1554, wherein each chiral center has (S) configuration. In some embodiments, the compound of Formula (I) is AMG1555, wherein each chiral center has (R) configuration. In some embodiments, the compound of Formula (I) is AMG1555, wherein each chiral center has (S) configuration. In some embodiments, the compound of Formula (I) is AMG1556, wherein each chiral center has (R) configuration. In some embodiments, the compound of Formula (I) is AMG1556, wherein each chiral center has (S) configuration. In some embodiments, the compound of Formula (I) is AMG1557, wherein each chiral center has (R) configuration. In some embodiments, the compound of Formula (I) is AMG1557, wherein each chiral center has (S) configuration. In some embodiments, the compound of Formula (I) is AMG1558, wherein each chiral center has (R) configuration. In some embodiments, the compound of Formula (I) is AMG1558, wherein each chiral center has (S) configuration. In some embodiments, the compound of Formula (I) is AMG1559, wherein each chiral center has (R) configuration. In some embodiments, the compound of Formula (I) is AMG1559, wherein each chiral center has (S) configuration. In some embodiments, the compound of Formula (I) is AMG1560, wherein each chiral center has (R) configuration. In some embodiments, the compound of Formula (I) is AMG1560, wherein each chiral center has (S) configuration. In some embodiments, the compound of Formula (I) is AMG1561, wherein each chiral center has (R) configuration. In some embodiments, the compound of Formula (I) is AMG1561, wherein each chiral center has (S) configuration. In some embodiments, the compound of Formula (I) is AMG1562, wherein each chiral center has (R) configuration. In some embodiments, the compound of Formula (I) is AMG1562, wherein each chiral center has (S) configuration. In some embodiments, the compound of Formula (I) is AMG1563, wherein each chiral center has (R) configuration. In some embodiments, the compound of Formula (I) is AMG1563, wherein each chiral center has (S) configuration. In some embodiments, the compound of Formula (I) is AMG1564, wherein each chiral center has (R) configuration. In some embodiments, the compound of Formula (I) is AMG1564, wherein each chiral center has (S) configuration. In some embodiments, the compound of Formula (I) is AMG1565, wherein each chiral center has (R) configuration. In some embodiments, the compound of Formula (I) is AMG1565, wherein each chiral center has (S) configuration. In some embodiments, the compound of Formula (I) is AMG1566, wherein each chiral center has (R) configuration. In some embodiments, the compound of Formula (I) is AMG1566, wherein each chiral center has (S) configuration. In some embodiments, the compound of Formula (I) is AMG1567, wherein each chiral center has (R) configuration. In some embodiments, the compound of Formula (I) is AMG1567, wherein each chiral center has (S) configuration. In some embodiments, the compound of Formula (I) is AMG1568, wherein each chiral center has (R) configuration. In some embodiments, the compound of Formula (I) is AMG1568, wherein each chiral center has (S) configuration. In some embodiments, the compound of Formula (I) is AMG15C12, wherein each chiral center has (R) configuration. In some embodiments, the compound of Formula (I) is AMG15C12, wherein each chiral center has (S) configuration.

In some embodiments, the compound of Formula (I) is of Formula (IV):

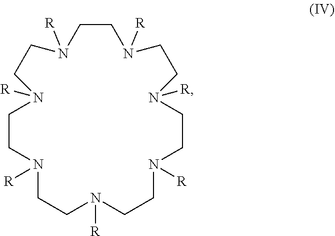

or a pharmaceutically acceptable salt, solvate, tautomer, stereoisomer, or isotopically labeled derivative thereof. In some embodiments, the compound of Formula (I) is of Formula (IV), or a pharmaceutically acceptable salt thereof.

In some embodiments, the compound of Formula (I) is of Formula (IV), or a pharmaceutically acceptable salt thereof, wherein each R is

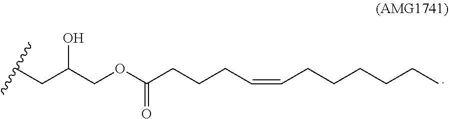 [SMILES: *CC(O)COC(=O)CCC/C=C\CCCCCC]

In some embodiments, the compound of Formula (I) is of Formula (IV), or a pharmaceutically acceptable salt thereof, wherein each R is

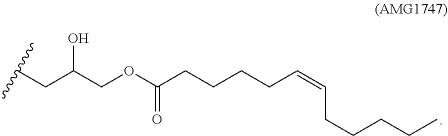 [SMILES: *CC(O)COC(=O)CCCC/C=C\CCCCC]

In some embodiments, the compound of Formula (I) is of Formula (IV), or a pharmaceutically acceptable salt thereof, wherein each R is

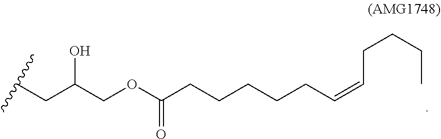 [SMILES: *CC(O)COC(=O)CCCCC/C=C\CCCC]

In some embodiments, the compound of Formula (I) is of Formula (IV), or a pharmaceutically acceptable salt thereof, wherein each R is

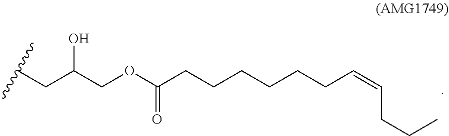 [SMILES: *CC(O)COC(=O)CCCCCC/C=C\CCC]

In some embodiments, the compound of Formula (I) is of Formula (IV), or a pharmaceutically acceptable salt thereof, wherein each R is

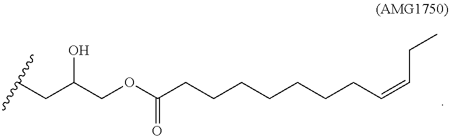 [SMILES: *CC(O)COC(=O)CCCCCCC/C=C\CC]

In some embodiments, the compound of Formula (I) is of Formula (IV), or a pharmaceutically acceptable salt thereof, where each R is

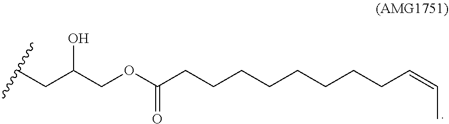 [SMILES: *CC(O)COC(=O)CCCCCCCC/C=C\C]

In some embodiments, the compound of Formula (I) is of Formula (IV), or a pharmaceutically acceptable salt thereof, wherein each R is

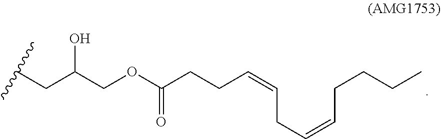 [SMILES: *CC(O)COC(=O)CC/C=C\C/C=C\CCCC]

In some embodiments, the compound of Formula (I) is of Formula (IV), or a pharmaceutically acceptable salt thereof, wherein each R is

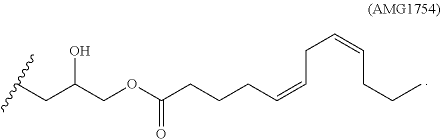 [SMILES: *CC(O)COC(=O)CCC/C=C\C/C=C\CCC]

In some embodiments, the compound of Formula (I) is of Formula (IV), or a pharmaceutically acceptable salt thereof, wherein each R

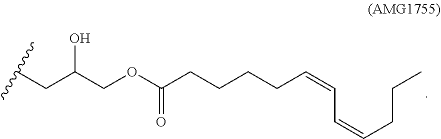 [SMILES: *CC(O)COC(=O)CCCC/C=C\C=C/CCC]

In some embodiments, the compound of Formula (I) is of Formula (IV), or a pharmaceutically acceptable salt thereof, wherein each

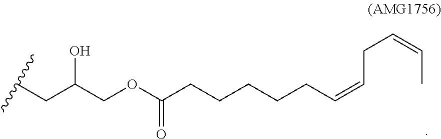 [SMILES: *CC(O)COC(=O)CCCCC/C=C\C/C=C\C]

In some embodiments, the compound of Formula (I) is of Formula (IV), or a pharmaceutically acceptable salt thereof, wherein each R is

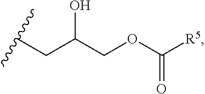

and wherein each R⁵ is

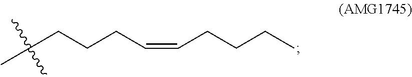 [SMILES: *CCC/C=C\CCCC]

each R⁵ is

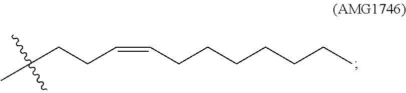 [SMILES: *CC/C=C\CCCCCCC]

each R⁵ is

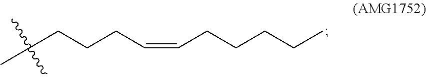 [SMILES: *CCC/C=C\CCCCC]

each R⁵ is

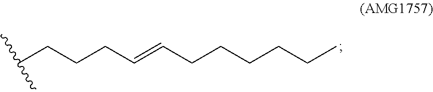 [SMILES: *CCC/C=C/CCCCCC]

each R⁵ is

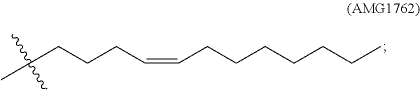 [SMILES: *CCC/C=C\CCCCCCC]

each R⁵ is

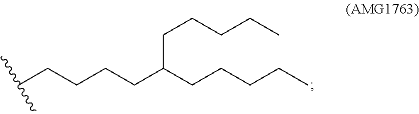 [SMILES: *CCCCC(CCCCC)CCCCC]

each R⁵ is

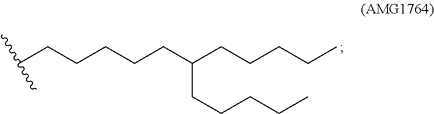 [SMILES: *CCCCCC(CCCCC)CCCCC]

each R⁵ is

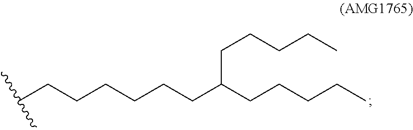 [SMILES: *CCCCCCC(CCCCC)CCCCC]

each R⁵ is

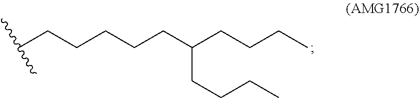 [SMILES: *CCCCCC(CCCC)CCCC]

each R⁵ is

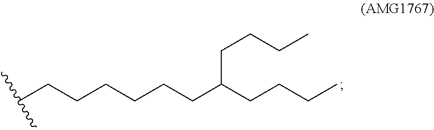 [SMILES: *CCCCCCC(CCCC)CCCC]

or each R⁵ is

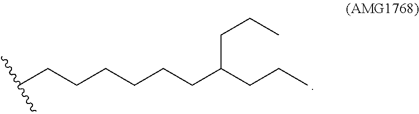 [SMILES: *CCCCCCC(CCC)CCC]

In some embodiments, the compound of Formula (I) is of Formula (IV), or a pharmaceutically acceptable salt thereof, wherein each R is

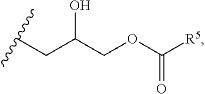

and wherein each R⁵ is

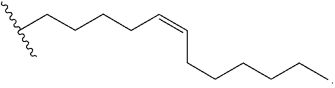 [SMILES: *CCCC/C=C\CCCCCC]

In some embodiments, the compound of Formula (I) is of Formula (IV), or a pharmaceutically acceptable salt thereof, wherein each R is

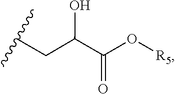

and wherein each R⁵ is

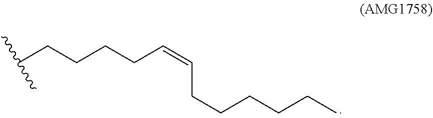 [SMILES: *CCCC/C=C\CCCCCC]

In some embodiments, the compound of Formula (I) is of Formula (IV), or a pharmaceutically acceptable salt thereof, wherein each R is

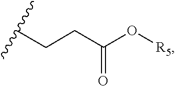

wherein each R⁵ is

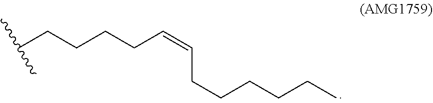 [SMILES: *CCCC/C=C\CCCCCC]

In some embodiments, the compound of Formula (I) is of Formula (IV), or a pharmaceutically acceptable salt thereof, wherein each R is

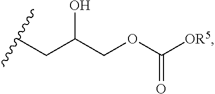

and wherein each R⁵ is

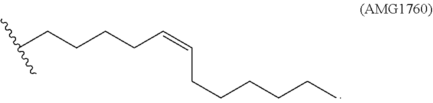 [SMILES: *CCCC/C=C\CCCCCC]

In some embodiments, the compound of Formula (I) is of Formula (IV), or a pharmaceutically acceptable salt thereof, wherein each R is

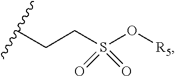

and wherein each R⁵ is

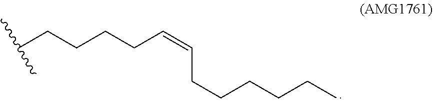 [SMILES: *CCCC/C=C\CCCCCC]

In some embodiments, the compound of Formula (I) is AMG1741, wherein each chiral center has (R) configuration. In some embodiments, the compound of Formula (I) is AMG1741, wherein each chiral center has (S) configuration. In some embodiments, the compound of Formula (I) is AMG1745, wherein each chiral center has (R) configuration. In some embodiments, the compound of Formula (I) is AMG1745, wherein each chiral center has (S) configuration. In some embodiments, the compound of Formula (I) is AMG1746, wherein each chiral center has (R) configuration. In some embodiments, the compound of Formula (I) is AMG1746, wherein each chiral center has (S) configuration. In some embodiments, the compound of Formula (I) is AMG1747, wherein each chiral center has (R) configuration. In some embodiments, the compound of Formula (I) is AMG1747, wherein each chiral center has (S) configuration. In some embodiments, the compound of Formula (I) is AMG1748, wherein each chiral center has (R) configuration. In some embodiments, the compound of Formula (I) is AMG1748, wherein each chiral center has (S) configuration. In some embodiments, the compound of Formula (I) is AMG1749, wherein each chiral center has (R) configuration. In some embodiments, the compound of Formula (I) is AMG1749, wherein each chiral center has (S) configuration. In some embodiments, the compound of Formula (I) is AMG1750, wherein each chiral center has (R) configuration. In some embodiments, the compound of Formula (I) is AMG1750, wherein each chiral center has (S) configuration. In some embodiments, the compound of Formula (I) is AMG1751, wherein each chiral center has (R) configuration. In some embodiments, the compound of Formula (I) is AMG1751, wherein each chiral center has (S) configuration. In some embodiments, the compound of Formula (I) is AMG1752, wherein each chiral center has (R) configuration. In some embodiments, the compound of Formula (I) is AMG1752, wherein each chiral center has (S) configuration. In some embodiments, the compound of Formula (I) is AMG1753, wherein each chiral center has (R) configuration. In some embodiments, the compound of Formula (I) is AMG1753, wherein each chiral center has (S) configuration. In some embodiments, the compound of Formula (I) is AMG1754, wherein each chiral center has (R) configuration. In some embodiments, the compound of Formula (I) is AMG1754, wherein each chiral center has (S) configuration. In some embodiments, the compound of Formula (I) is AMG1755, wherein each chiral center has (R) configuration. In some embodiments, the compound of Formula (I) is AMG1755, wherein each chiral center has (S) configuration. In some embodiments, the compound of Formula (I) is AMG1756, wherein each chiral center has (R) configuration. In some embodiments, the compound of Formula (I) is AMG1756, wherein each chiral center has (S) configuration. In some embodiments, the compound of Formula (I) is AMG1757, wherein each chiral center has (R) configuration. In some embodiments, the compound of Formula (I) is AMG1757, wherein each chiral center has (S) configuration. In some embodiments, the compound of Formula (I) is AMG1758, wherein each chiral center has (R) configuration. In some embodiments, the compound of Formula (I) is AMG1758, wherein each chiral center has (S) configuration. In some embodiments, the compound of Formula (I) is AMG1759, wherein each chiral center has (R) configuration. In some embodiments, the compound of Formula (I) is AMG1759, wherein each chiral center has (S) configuration. In some embodiments, the compound of Formula (I) is AMG1760, wherein each chiral center has (R) configuration. In some embodiments, the compound of Formula (I) is AMG1760, wherein each chiral center has (S) configuration. In some embodiments, the compound of Formula (I) is AMG1761, wherein each chiral center has (R) configuration. In some embodiments, the compound of Formula (I) is AMG1761, wherein each chiral center has (S) configuration. In some embodiments, the compound of Formula (I) is AMG1762, wherein each chiral center has (R) configuration. In some embodiments, the compound of Formula (I) is AMG1762, wherein each chiral center has (S) configuration. In some embodiments, the compound of Formula (I) is AMG1763, wherein each chiral center has (R) configuration. In some embodiments, the compound of Formula (I) is AMG1763, wherein each chiral center has (S) configuration. In some embodiments, the compound of Formula (I) is AMG1764, wherein each chiral center has (R) configuration. In some embodiments, the compound of Formula (I) is AMG1764, wherein each chiral center has (S) configuration. In some embodiments, the compound of Formula (I) is AMG1765, wherein each chiral center has (R) configuration. In some embodiments, the compound of Formula (I) is AMG1765, wherein each chiral center has (S) configuration. In some embodiments, the compound of Formula (I) is AMG1766, wherein each chiral center has (R) configuration. In some embodiments, the compound of Formula (I) is AMG1766, wherein each chiral center has (S) configuration. In some embodiments, the compound of Formula (I) is AMG1767, wherein each chiral center has (R) configuration. In some embodiments, the compound of Formula (I) is AMG1767, wherein each chiral center has (S) configuration. In some embodiments, the compound of Formula (I) is AMG1768, wherein each chiral center has (R) configuration. In some embodiments, the compound of Formula (I) is AMG1768, wherein each chiral center has (S) configuration.

In some embodiments, the compound of Formula (I) is of Formula (V):

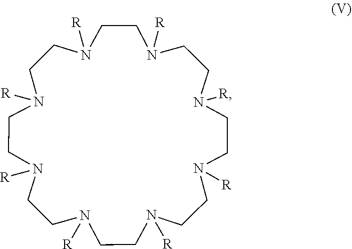

or a pharmaceutically acceptable salt, solvate, tautomer, stereoisomer, or isotopically labeled derivative thereof. In some embodiments, the compound of Formula (I) is of Formula (V), or a pharmaceutically acceptable salt thereof.

In some embodiments, the compound of Formula (I) is of Formula (V), or a pharmaceutically acceptable salt thereof, wherein each R is

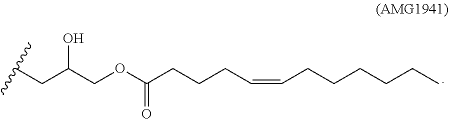 [SMILES: *CC(O)COC(=O)CCC/C=C\CCCCCC]

In some embodiments, the compound of Formula (I) is of Formula (V), or a pharmaceutically acceptable salt thereof, wherein each R is

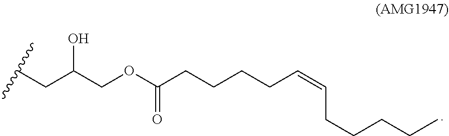 [SMILES: *CC(O)COC(=O)CCCC/C=C\CCCCC]

In some embodiments, the compound of Formula (I) is of Formula (V), or a pharmaceutically acceptable salt thereof, wherein each R is

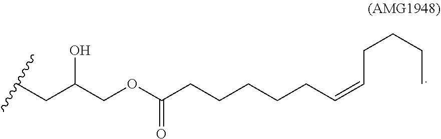 [SMILES: *CC(O)COC(=O)CCCCC/C=C\CCCC]

In some embodiments, the compound of Formula (I) is of Formula (V), or a pharmaceutically acceptable salt thereof, wherein each R is

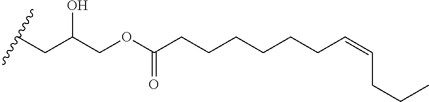 [SMILES: *CC(O)COC(=O)CCCCCC/C=C\CCC]

In some embodiments, the compound of Formula (I) is of Formula (V), or a pharmaceutically acceptable salt thereof, wherein each R is

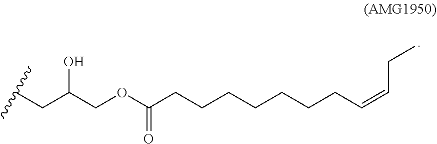 [SMILES: *CC(O)COC(=O)CCCCCCC/C=C\CC]

In some embodiments, the compound of Formula (I) is of Formula (V), or a pharmaceutically acceptable salt thereof, wherein each R is

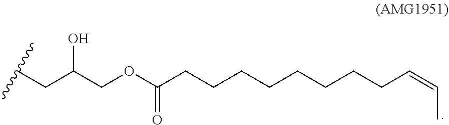 [SMILES: *CC(O)COC(=O)CCCCCCCC/C=C\C]

In some embodiments, the compound of Formula (I) is of Formula (V), or a pharmaceutically acceptable salt thereof, wherein each R is

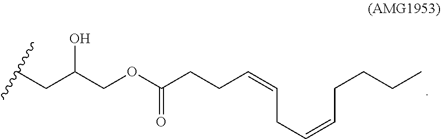 [SMILES: *CC(O)COC(=O)CC/C=C\C/C=C\CCCC]

In some embodiments, the compound of Formula (I) is of Formula (V), or a pharmaceutically acceptable salt thereof, wherein each R is

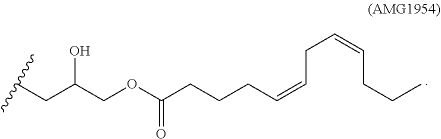 [SMILES: *CC(O)COC(=O)CCC/C=C\C/C=C\CCC]

In some embodiments, the compound of Formula (I) is of Formula (V), or a pharmaceutically acceptable salt thereof, wherein each R is

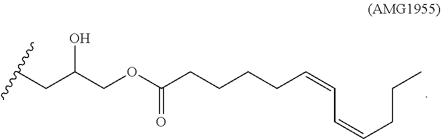 [SMILES: *CC(O)COC(=O)CCCC/C=C\C=C/CCC]

In some embodiments, the compound of Formula (I) is of Formula (V), or a pharmaceutically acceptable salt thereof, wherein each R is

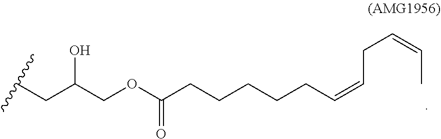 [SMILES: *CC(O)COC(=O)CCCCC/C=C\C/C=C\C]

In some embodiments, the compound of Formula (I) is of Formula (V), or a pharmaceutically acceptable salt thereof, wherein each R is

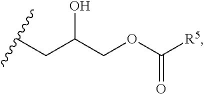

and wherein each R⁵ is

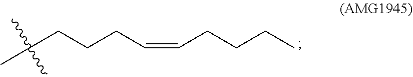 [SMILES: *CCC/C=C\CCCC]

each R⁵ is

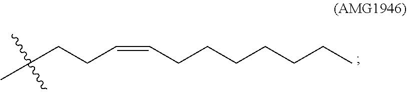 [SMILES: *CC/C=C\CCCCCCC]

each R⁵ is

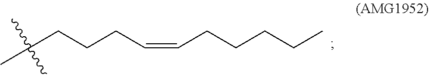 [SMILES: *CCC/C=C\CCCCC]

each R⁵ is

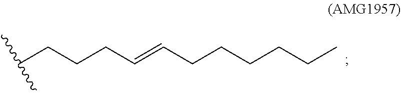 [SMILES: *CCC/C=C/CCCCCC]

each R⁵ is

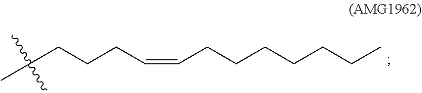 [SMILES: *CCC/C=C\CCCCCCC]

each R⁵ is

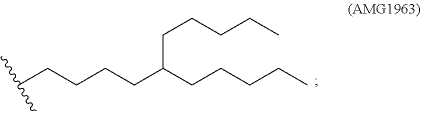 [SMILES: *CCCCC(CCCCC)CCCCC]

each R⁵ is

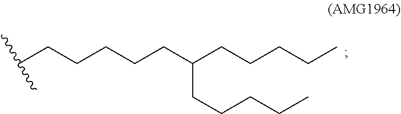 [SMILES: *CCCCCC(CCCCC)CCCCC]

each R⁵ is

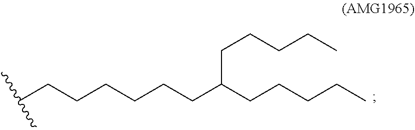 [SMILES: *CCCCCCC(CCCCC)CCCCC]

each R⁵ is

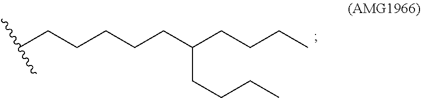 [SMILES: *CCCCCC(CCCC)CCCC]

each R⁵ is

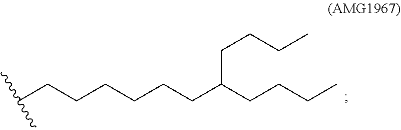 [SMILES: *CCCCCCC(CCCC)CCCC]

or each R⁵ is

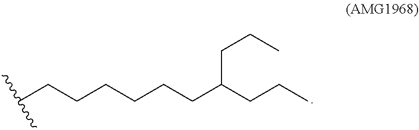 [SMILES: *CCCCCCC(CCC)CCC]

In some embodiments, the compound of Formula (I) is of Formula (V), or a pharmaceutically acceptable salt thereof, wherein each R is

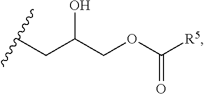

and wherein each R⁵ is

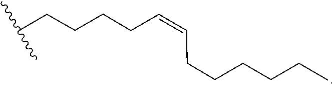 [SMILES: *CCCC/C=C\CCCCCC]

In some embodiments, the compound of Formula (I) is of Formula (V), or a pharmaceutically acceptable salt thereof, wherein each R is

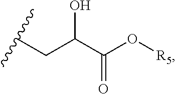

and wherein each R⁵ is

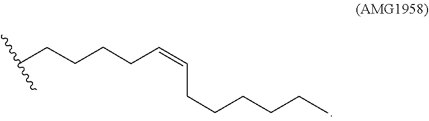 [SMILES: *CCCC/C=C\CCCCCC]

In some embodiments, the compound of Formula (I) is of Formula (V), or a pharmaceutically acceptable salt thereof, wherein each R is

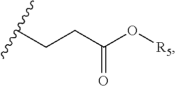

and wherein each R⁵ is

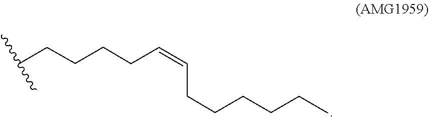 [SMILES: *CCCC/C=C\CCCCCC]

In some embodiments, the compound of Formula (I) is of Formula (V), or a pharmaceutically acceptable salt thereof, wherein each R is

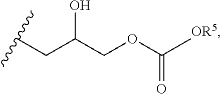 [SMILES: *CC(O)COC(C)=O]

and wherein each R⁵ is

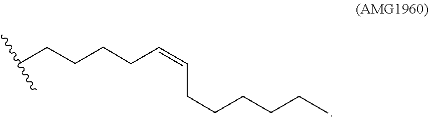 [SMILES: *CCCC/C=C\CCCCCC]

In some embodiments, the compound of Formula (I) is of Formula (V), or a pharmaceutically acceptable salt thereof, wherein each R is

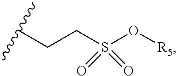

and wherein each R⁵ is

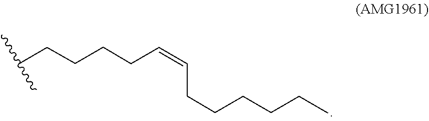 [SMILES: *CCCC/C=C\CCCCCC]

In some embodiments, the compound of Formula (I) is AMG1941, wherein each chiral center has (R) configuration. In some embodiments, the compound of Formula (I) is AMG1941, wherein each chiral center has (S) configuration. In some embodiments, the compound of Formula (I) is AMG1945, wherein each chiral center has (R) configuration. In some embodiments, the compound of Formula (I) is AMG1945, wherein each chiral center has (S) configuration. In some embodiments, the compound of Formula (I) is AMG1946, wherein each chiral center has (R) configuration. In some embodiments, the compound of Formula (I) is AMG1946, wherein each chiral center has (S) configuration. In some embodiments, the compound of Formula (I) is AMG1947, wherein each chiral center has (R) configuration. In some embodiments, the compound of Formula (I) is AMG1947, wherein each chiral center has (S) configuration. In some embodiments, the compound of Formula (I) is AMG1948, wherein each chiral center has (R) configuration. In some embodiments, the compound of Formula (I) is AMG1948, wherein each chiral center has (S) configuration. In some embodiments, the compound of Formula (I) is AMG1949, wherein each chiral center has (R) configuration. In some embodiments, the compound of Formula (I) is AMG1949, wherein each chiral center has (S) configuration. In some embodiments, the compound of Formula (I) is AMG1950, wherein each chiral center has (R) configuration. In some embodiments, the compound of Formula (I) is AMG1950, wherein each chiral center has (S) configuration. In some embodiments, the compound of Formula (I) is AMG1951, wherein each chiral center has (R) configuration. In some embodiments, the compound of Formula (I) is AMG1951, wherein each chiral center has (S) configuration. In some embodiments, the compound of Formula (I) is AMG1952, wherein each chiral center has (R) configuration. In some embodiments, the compound of Formula (I) is AMG1952, wherein each chiral center has (S) configuration. In some embodiments, the compound of Formula (I) is AMG1953, wherein each chiral center has (R) configuration. In some embodiments, the compound of Formula (I) is AMG1953, wherein each chiral center has (S) configuration. In some embodiments, the compound of Formula (I) is AMG1954, wherein each chiral center has (R) configuration. In some embodiments, the compound of Formula (I) is AMG1954, wherein each chiral center has (S) configuration. In some embodiments, the compound of Formula (I) is AMG1955, wherein each chiral center has (R) configuration. In some embodiments, the compound of Formula (I) is AMG1955, wherein each chiral center has (S) configuration. In some embodiments, the compound of Formula (I) is AMG1956, wherein each chiral center has (R) configuration. In some embodiments, the compound of Formula (I) is AMG1956, wherein each chiral center has (S) configuration. In some embodiments, the compound of Formula (I) is AMG1957, wherein each chiral center has (R) configuration. In some embodiments, the compound of Formula (I) is AMG1957, wherein each chiral center has (S) configuration. In some embodiments, the compound of Formula (I) is AMG1958, wherein each chiral center has (R) configuration. In some embodiments, the compound of Formula (I) is AMG1958, wherein each chiral center has (S) configuration. In some embodiments, the compound of Formula (I) is AMG1959, wherein each chiral center has (R) configuration. In some embodiments, the compound of Formula (I) is AMG1959, wherein each chiral center has (S) configuration. In some embodiments, the compound of Formula (I) is AMG1960, wherein each chiral center has (R) configuration. In some embodiments, the compound of Formula (I) is AMG1960, wherein each chiral center has (S) configuration. In some embodiments, the compound of Formula (I) is AMG1961, wherein each chiral center has (R) configuration. In some embodiments, the compound of Formula (I) is AMG1961, wherein each chiral center has (S) configuration. In some embodiments, the compound of Formula (I) is AMG1962, wherein each chiral center has (R) configuration. In some embodiments, the compound of Formula (I) is AMG1962, wherein each chiral center has (S) configuration. In some embodiments, the compound of Formula (I) is AMG1963, wherein each chiral center has (R) configuration. In some embodiments, the compound of Formula (I) is AMG1963, wherein each chiral center has (S) configuration. In some embodiments, the compound of Formula (I) is AMG1964, wherein each chiral center has (R) configuration. In some embodiments, the compound of Formula (I) is AMG1964, wherein each chiral center has (S) configuration. In some embodiments, the compound of Formula (I) is AMG1965, wherein each chiral center has (R) configuration. In some embodiments, the compound of Formula (I) is AMG1965, wherein each chiral center has (S) configuration. In some embodiments, the compound of Formula (I) is AMG1966, wherein each chiral center has (R) configuration. In some embodiments, the compound of Formula (I) is AMG1966, wherein each chiral center has (S) configuration. In some embodiments, the compound of Formula (I) is AMG1967, wherein each chiral center has (R) configuration. In some embodiments, the compound of Formula (I) is AMG1967, wherein each chiral center has (S) configuration. In some embodiments, the compound of Formula (I) is AMG1968, wherein each chiral center has (R) configuration. In some embodiments, the compound of Formula (I) is AMG1968, wherein each chiral center has (S) configuration.

In some embodiments, the compound of Formula (I) is of Formula (VI):

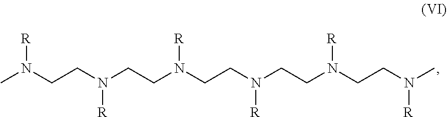

or a pharmaceutically acceptable salt, solvate, tautomer, stereoisomer, or isotopically labeled derivative thereof. In some embodiments, the compound of Formula (I) is of Formula (VI), or a pharmaceutically acceptable salt thereof.

In some embodiments, the compound of Formula (I) is of Formula (VI), or a pharmaceutically acceptable salt thereof, wherein each R is

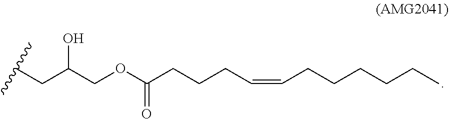 [SMILES: *CC(O)COC(=O)CCC/C=C\CCCCCC]

In some embodiments, the compound of Formula (I) is of Formula (VI), or a pharmaceutically acceptable salt thereof, wherein each R is

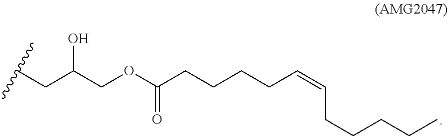 [SMILES: *CC(O)COC(=O)CCCC/C=C\CCCCC]

In some embodiments, the compound of Formula (I) is of Formula (VI), or a pharmaceutically acceptable salt thereof, wherein each R is

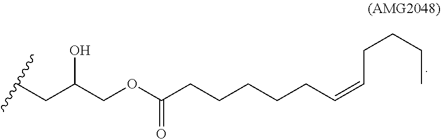 [SMILES: *CC(O)COC(=O)CCCCC/C=C\CCCC]

In some embodiments, the compound of Formula (I) is of Formula (VI), or a pharmaceutically acceptable salt thereof, wherein each R is

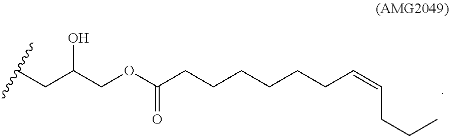 [SMILES: *CC(O)COC(=O)CCCCCC/C=C\CCC]

In some embodiments, the compound of Formula (I) is of Formula (VI), or a pharmaceutically acceptable salt thereof, wherein each R is

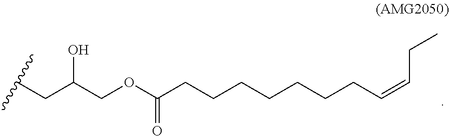 [SMILES: *CC(O)COC(=O)CCCCCCC/C=C\CC]

In some embodiments, the compound of Formula (I) is of Formula (VI), or a pharmaceutically acceptable salt thereof, wherein each R is

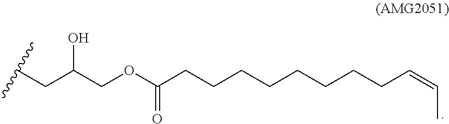 [SMILES: *CC(O)COC(=O)CCCCCCCC/C=C\C]

In some embodiments, the compound of Formula (I) is of Formula (VI), or a pharmaceutically acceptable salt thereof, wherein each R is

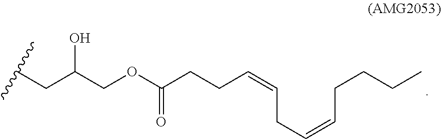 [SMILES: *CC(O)COC(=O)CC/C=C\C/C=C\CCCC]

In some embodiments, the compound of Formula (I) is of Formula (VI), or a pharmaceutically acceptable salt thereof, wherein each R is

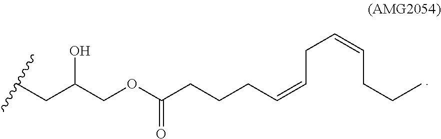 [SMILES: *CC(O)COC(=O)CCC/C=C\C/C=C\CCC]

In some embodiments, the compound of Formula (I) is of Formula (VI), or a pharmaceutically acceptable salt thereof, wherein each R is

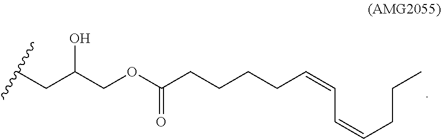 [SMILES: *CC(O)COC(=O)CCCC/C=C\C=C/CCC]

In some embodiments, the compound of Formula (I) is of Formula (VI), or a pharmaceutically acceptable salt thereof, wherein each R is

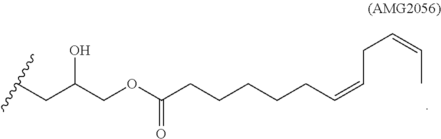 [SMILES: *CC(O)COC(=O)CCCCC/C=C\C/C=C\C]

In some embodiments, the compound of Formula (I) is of Formula (VI), or a pharmaceutically acceptable salt thereof, wherein each R is

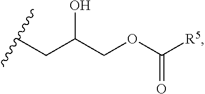

and wherein each R⁵ is

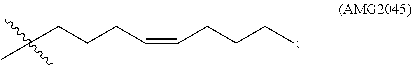 [SMILES: *CCC/C=C\CCCC]

each R⁵ is

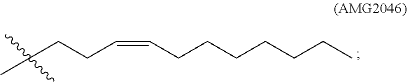 [SMILES: *CC/C=C\CCCCCCC]

each R⁵ is

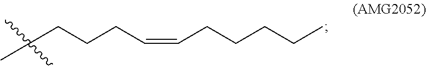 [SMILES: *CCC/C=C\CCCCC]

each R⁵ is

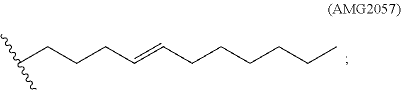 [SMILES: *CCC/C=C/CCCCCC]

each R⁵ is

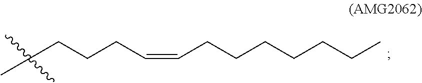 [SMILES: *CCC/C=C\CCCCCCC]

each R⁵ is

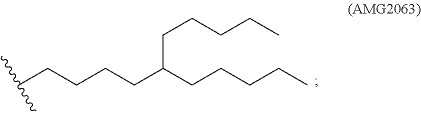 [SMILES: *CCCCC(CCCCC)CCCCC]

each R⁵ is

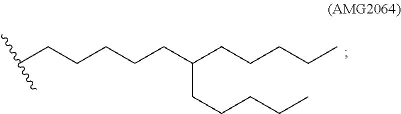 [SMILES: *CCCCCC(CCCCC)CCCCC]

each R⁵ is

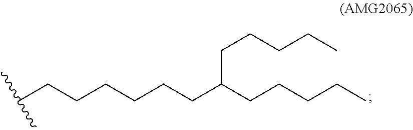 [SMILES: *CCCCCCC(CCCCC)CCCCC]

each R⁵ is

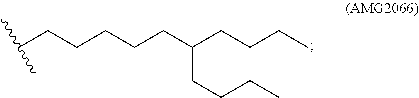 [SMILES: *CCCCCC(CCCC)CCCC]

each R⁵ is

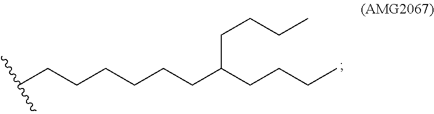 [SMILES: *CCCCCCC(CCCC)CCCC]

or each R⁵ is

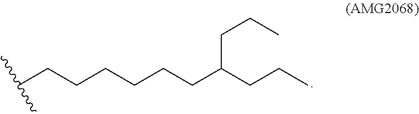 [SMILES: *CCCCCCC(CCC)CCC]

In some embodiments, the compound of Formula (I) is of Formula (VI), or a pharmaceutically acceptable salt thereof, wherein each R is

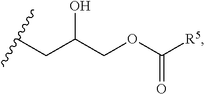

and wherein each R⁵ is

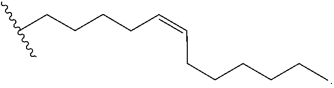 [SMILES: *CCCC/C=C\CCCCCC]

In some embodiments, the compound of Formula (I) is of Formula (VI), or a pharmaceutically acceptable salt thereof, wherein each R is

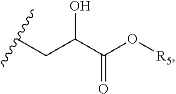

and wherein each R⁵ is

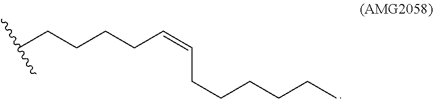 [SMILES: *CCCC/C=C\CCCCCC]

In some embodiments, the compound of Formula (I) is of Formula (VI), or a pharmaceutically acceptable salt thereof, wherein each R is

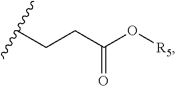

and wherein each R⁵ is

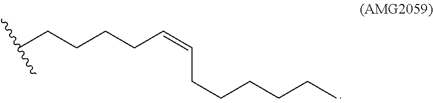 [SMILES: *CCCC/C=C\CCCCCC]

In some embodiments, the compound of Formula (I) is of Formula (VI), or a pharmaceutically acceptable salt thereof, wherein each R is

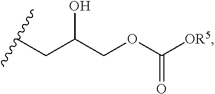

and wherein each R⁵ is

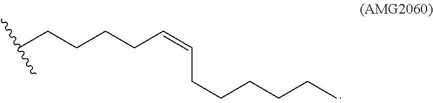 [SMILES: *CCCC/C=C\CCCCCC]

In some embodiments, the compound of Formula (I) is of Formula (VI), or a pharmaceutically acceptable salt thereof, wherein each R is

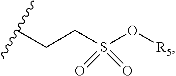

and wherein each R⁵ is

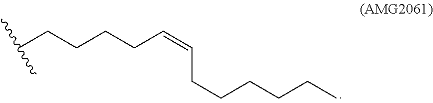 [SMILES: *CCCC/C=C\CCCCCC]

In some embodiments, the compound of Formula (I) is AMG2041, wherein each chiral center has (R) configuration. In some embodiments, the compound of Formula (I) is AMG2041, wherein each chiral center has (S) configuration. In some embodiments, the compound of Formula (I) is AMG2045, wherein each chiral center has (R) configuration. In some embodiments, the compound of Formula (I) is AMG2045, wherein each chiral center has (S) configuration. In some embodiments, the compound of Formula (I) is AMG2046, wherein each chiral center has (R) configuration. In some embodiments, the compound of Formula (I) is AMG2046, wherein each chiral center has (S) configuration. In some embodiments, the compound of Formula (I) is AMG2047, wherein each chiral center has (R) configuration. In some embodiments, the compound of Formula (I) is AMG2047, wherein each chiral center has (S) configuration. In some embodiments, the compound of Formula (I) is AMG2048, wherein each chiral center has (R) configuration. In some embodiments, the compound of Formula (I) is AMG2048, wherein each chiral center has (S) configuration. In some embodiments, the compound of Formula (I) is AMG2049, wherein each chiral center has (R) configuration. In some embodiments, the compound of Formula (I) is AMG2049, wherein each chiral center has (S) configuration. In some embodiments, the compound of Formula (I) is AMG2050, wherein each chiral center has (R) configuration. In some embodiments, the compound of Formula (I) is AMG2050, wherein each chiral center has (S) configuration. In some embodiments, the compound of Formula (I) is AMG2051, wherein each chiral center has (R) configuration. In some embodiments, the compound of Formula (I) is AMG2051, wherein each chiral center has (S) configuration. In some embodiments, the compound of Formula (I) is AMG2052, wherein each chiral center has (R) configuration. In some embodiments, the compound of Formula (I) is AMG2052, wherein each chiral center has (S) configuration. In some embodiments, the compound of Formula (I) is AMG2053, wherein each chiral center has (R) configuration. In some embodiments, the compound of Formula (I) is AMG2053, wherein each chiral center has (S) configuration. In some embodiments, the compound of Formula (I) is AMG2054, wherein each chiral center has (R) configuration. In some embodiments, the compound of Formula (I) is AMG2054, wherein each chiral center has (S) configuration. In some embodiments, the compound of Formula (I) is AMG2055, wherein each chiral center has (R) configuration. In some embodiments, the compound of Formula (I) is AMG2055, wherein each chiral center has (S) configuration. In some embodiments, the compound of Formula (I) is AMG2056, wherein each chiral center has (R) configuration. In some embodiments, the compound of Formula (I) is AMG2056, wherein each chiral center has (S) configuration. In some embodiments, the compound of Formula (I) is AMG2057, wherein each chiral center has (R) configuration. In some embodiments, the compound of Formula (I) is AMG2057, wherein each chiral center has (S) configuration. In some embodiments, the compound of Formula (I) is AMG2058, wherein each chiral center has (R) configuration. In some embodiments, the compound of Formula (I) is AMG2058, wherein each chiral center has (S) configuration. In some embodiments, the compound of Formula (I) is AMG2059, wherein each chiral center has (R) configuration. In some embodiments, the compound of Formula (I) is AMG2059, wherein each chiral center has (S) configuration. In some embodiments, the compound of Formula (I) is AMG2060, wherein each chiral center has (R) configuration. In some embodiments, the compound of Formula (I) is AMG2060, wherein each chiral center has (S) configuration. In some embodiments, the compound of Formula (I) is AMG2061, wherein each chiral center has (R) configuration. In some embodiments, the compound of Formula (I) is AMG2061, wherein each chiral center has (S) configuration. In some embodiments, the compound of Formula (I) is AMG2062, wherein each chiral center has (R) configuration. In some embodiments, the compound of Formula (I) is AMG2062, wherein each chiral center has (S) configuration. In some embodiments, the compound of Formula (I) is AMG2063, wherein each chiral center has (R) configuration. In some embodiments, the compound of Formula (I) is AMG2063, wherein each chiral center has (S) configuration. In some embodiments, the compound of Formula (I) is AMG2064, wherein each chiral center has (R) configuration. In some embodiments, the compound of Formula (I) is AMG2064, wherein each chiral center has (S) configuration. In some embodiments, the compound of Formula (I) is AMG2065, wherein each chiral center has (R) configuration. In some embodiments, the compound of Formula (I) is AMG2065, wherein each chiral center has (S) configuration. In some embodiments, the compound of Formula (I) is AMG2066, wherein each chiral center has (R) configuration. In some embodiments, the compound of Formula (I) is AMG2066, wherein each chiral center has (S) configuration. In some embodiments, the compound of Formula (I) is AMG2067, wherein each chiral center has (R) configuration. In some embodiments, the compound of Formula (I) is AMG2067, wherein each chiral center has (S) configuration. In some embodiments, the compound of Formula (I) is AMG2068, wherein each chiral center has (R) configuration. In some embodiments, the compound of Formula (I) is AMG2068, wherein each chiral center has (S) configuration.

In some embodiments, the compound of Formula (I) is:

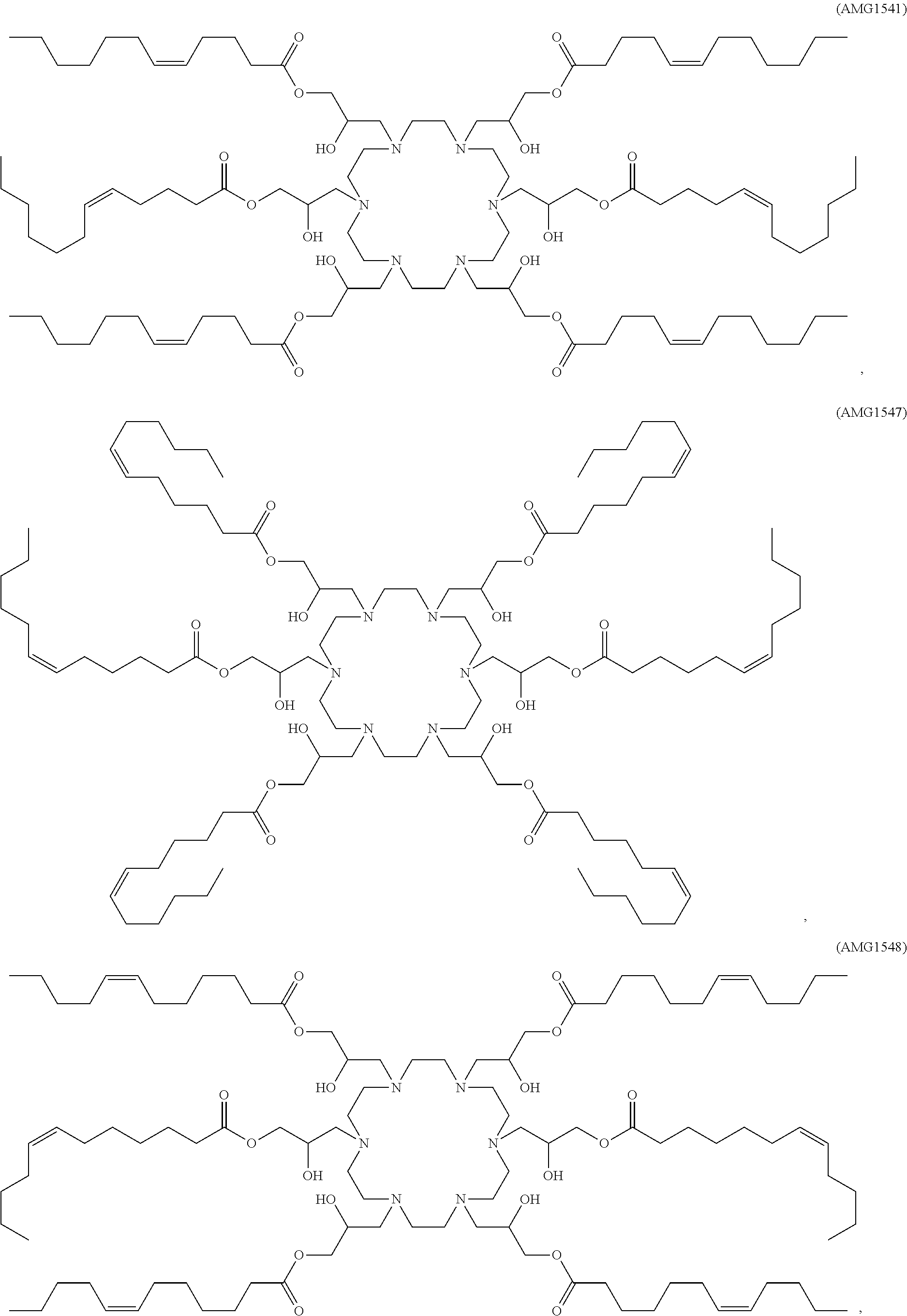

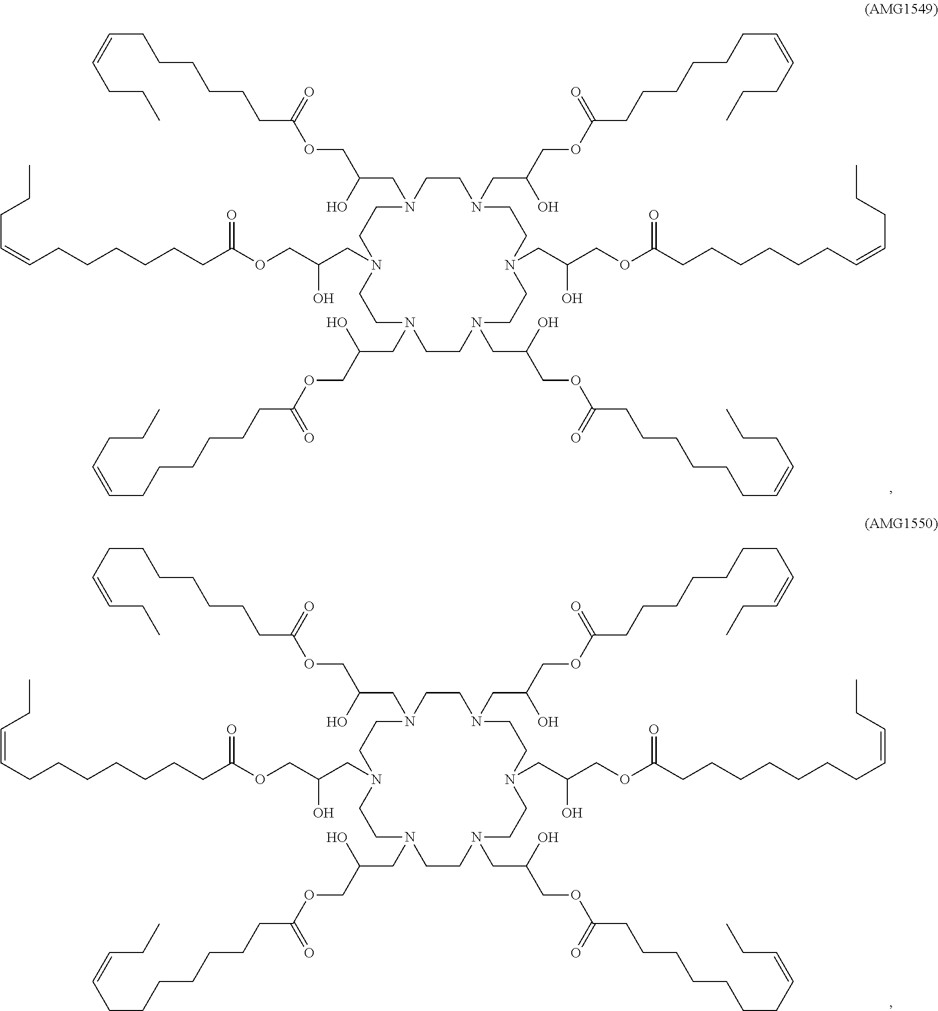 [SMILES: CCC/C=C\CCCCCCC(=O)OCC(O)CN1CCN(CC(O)COC(=O)CCCCCC/C=C\CCC)CCN(CC(O)COC(=O)CCCCCC/C=C\CCC)CCN(CC(O)COC(=O)CCCCCC/C=C\CCC)CCN(CC(O)COC(=O)CCCCCC/C=C\CCC)CCN(CC(O)COC(=O)CCCCCC/C=C\CCC)CC1 ; CC/C=C\CCCCCCCC(=O)OCC(O)CN1CCN(CC(O)COC(=O)CCCCCCC/C=C\CC)CCN(CC(O)COC(=O)CCCCCCC/C=C\CC)CCN(CC(O)COC(=O)CCCCCCC/C=C\CC)CCN(CC(O)COC(=O)CCCCCCC/C=C\CC)CCN(CC(O)COC(=O)CCCCCCC/C=C\CC)CC1]

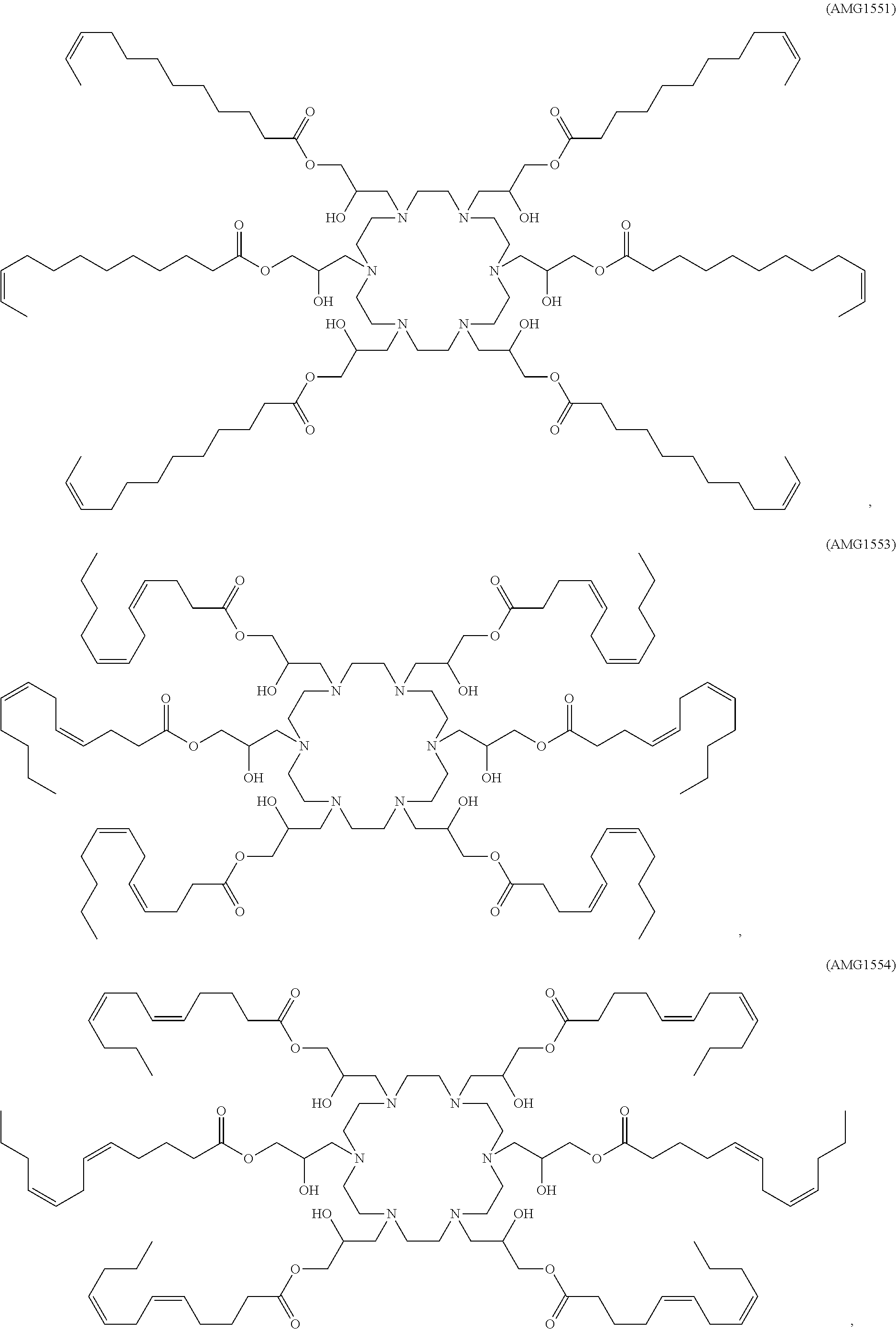

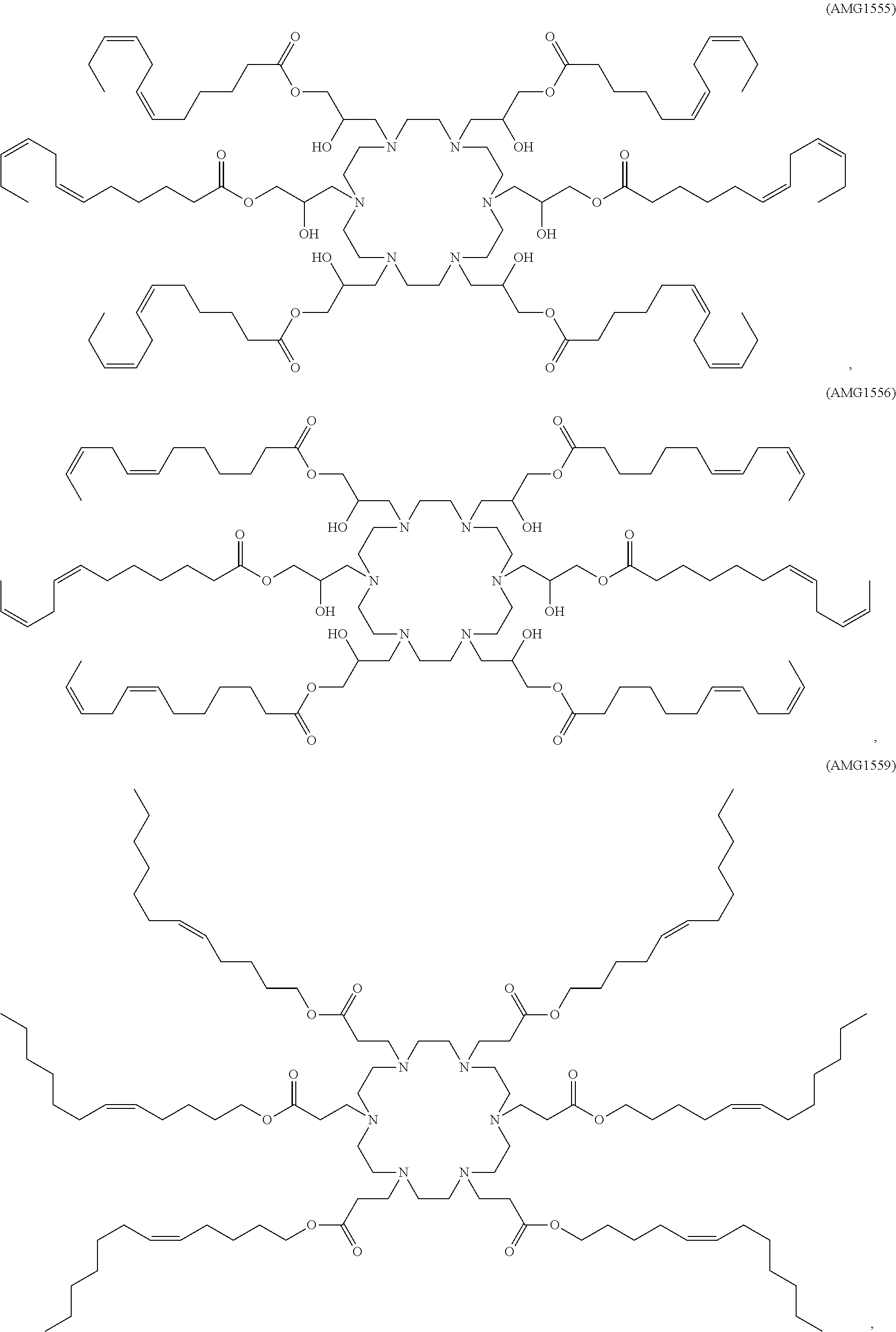

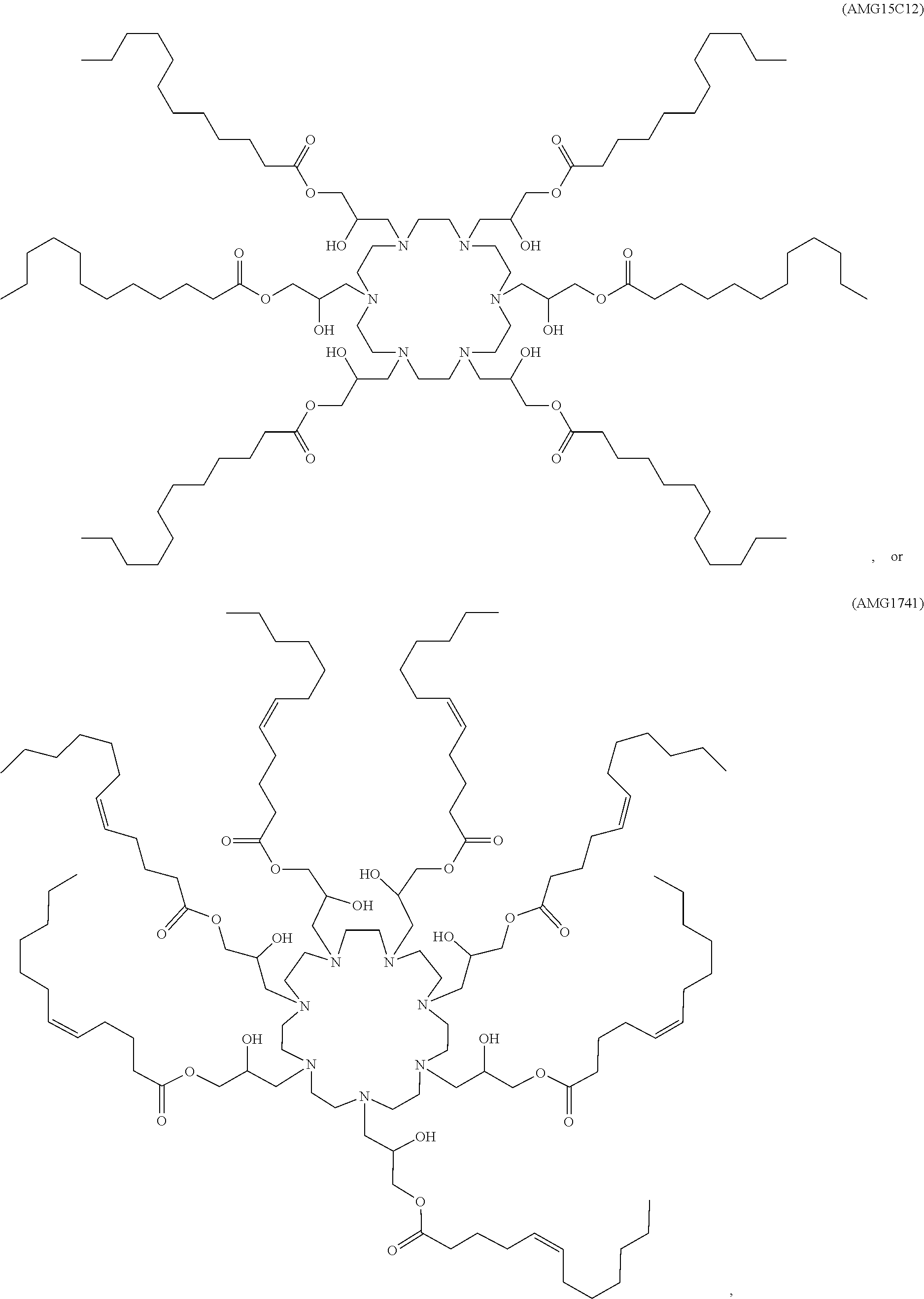

or a pharmaceutically acceptable salt, solvate, tautomer, stereoisomer, or isotopically labeled derivative thereof.

In some embodiments, the compound of Formula (I) is:

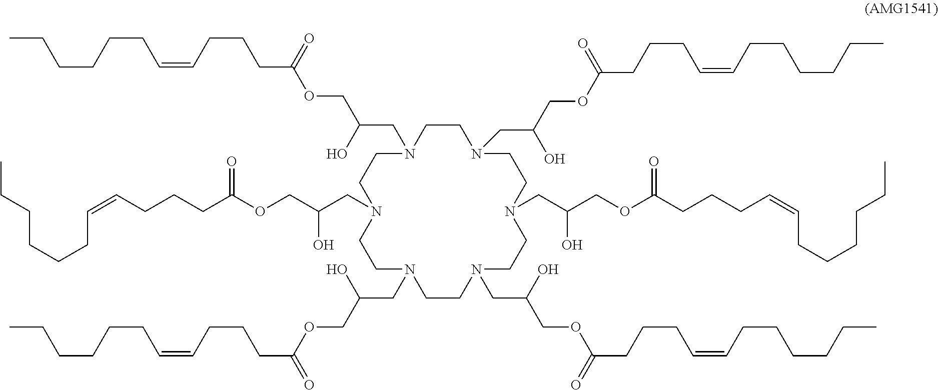 [SMILES: CCCCCC/C=C\CCCC(=O)OCC(O)CN1CCN(CC(O)COC(=O)CCC/C=C\CCCCCC)CCN(CC(O)COC(=O)CCC/C=C\CCCCCC)CCN(CC(O)COC(=O)CCC/C=C\CCCCCC)CCN(CC(O)COC(=O)CCC/C=C\CCCCCC)CCN(CC(O)COC(=O)CCC/C=C\CCCCCC)CC1]

or a pharmaceutically acceptable salt, solvate, tautomer, stereoisomer, or isotopically labeled derivative thereof.

### Pharmaceutical Compositions and Administration

The present disclosure provides compositions comprising a compound provided herein (e.g., a compound of Formula (I), (XI), (XII), (XIII)), or a pharmaceutically acceptable salt, solvate, tautomer, stereoisomer, or isotopically labeled derivative thereof, and an agent. The present disclosure also provides compositions comprising a compound provided herein (e.g., a compound of Formula (I), (XI), (XII), (XIII)), or a pharmaceutically acceptable salt thereof, and an agent.

The present disclosure also provides compositions (e.g., pharmaceutical compositions) comprising a compound provided herein (e.g., a compound of Formula (I), (XI), (XII), (XIII)), or a pharmaceutically acceptable salt, solvate, tautomer, stereoisomer, or isotopically labeled derivative thereof, or a composition provided herein and an excipient (e.g., a pharmaceutically acceptable excipient). The present disclosure also provides compositions (e.g., pharmaceutical compositions) comprising a compound provided herein (e.g., a compound of Formula (I), (XI), (XII), (XIII)), or a pharmaceutically acceptable salt thereof, or a composition provided herein and an excipient (e.g., a pharmaceutically acceptable excipient). In one aspect, the composition is a pharmaceutical composition. In one aspect, the composition is a pharmaceutical composition, and the excipient is a pharmaceutically acceptable excipient.

In some embodiments, the composition further comprises an agent.

In certain embodiments, the compound described herein is provided in an effective amount in the pharmaceutical composition. In certain embodiments, the effective amount is an amount effective for delivering an agent to a subject or cell. In certain embodiments, the effective amount is an amount effective for delivering a polynucleotide to a subject or cell. In certain embodiments, the effective amount is an amount effective for delivering mRNA to a subject or cell. In certain embodiments, the effective amount is an amount effective for chimeric antigen receptor T cell therapy.

In certain embodiments, the composition further comprises one or more of a PEG-lipid, sterol, or a phospholipid. In certain embodiments, the composition comprises a PEG-lipid. In some embodiments, the composition comprises a sterol. In certain embodiments, the composition comprises a phospholipid. In certain embodiments, the composition further comprises a PEG-lipid and a sterol. In certain embodiments, the composition further comprises a PEG-lipid and a phospholipid. In certain embodiments, the composition further comprises a sterol and a phospholipid. In certain embodiments, the composition comprises one or more of a PEG-lipid, sterol, or a phospholipid and is formulated as a particle. In some embodiments, the composition comprises one or more of a PEG-lipid, sterol, or a phospholipid and is formulated as a nanoparticle or microparticle. In certain embodiments, the composition comprises one or more of a PEG-lipid, sterol, or a phospholipid and is formulated as a lipid nanoparticle. In certain embodiments, the composition comprises one or more of a PEG-lipid, sterol, or a phospholipid and is formulated as a micelle, liposome, or lipoplex.

In some embodiments, the composition comprises approximately 20-70 molar % of the compound, or a pharmaceutically acceptable salt thereof. In some embodiments, the composition comprises approximately 25-65 molar % of the compound, or a pharmaceutically acceptable salt thereof. In some embodiments, the composition comprises approximately 20-60 molar % of the compound, or a pharmaceutically acceptable salt thereof. In some embodiments, the composition comprises approximately 30-60 molar % of the compound, or a pharmaceutically acceptable salt thereof. In some embodiments, the composition comprises approximately 35-60 molar % of the compound, or a pharmaceutically acceptable salt thereof. In some embodiments, the composition comprises approximately 40-60 molar % of the compound, or a pharmaceutically acceptable salt thereof. In some embodiments, the composition comprises approximately 45-60 molar % of the compound, or a pharmaceutically acceptable salt thereof. In some embodiments, the composition comprises approximately 50-60 molar % of the compound, or a pharmaceutically acceptable salt thereof. In some embodiments, the composition comprises approximately 55-60 molar % of the compound, or a pharmaceutically acceptable salt thereof. In some embodiments, the composition comprises approximately 20-30 molar % of the compound, or a pharmaceutically acceptable salt thereof. In some embodiments, the composition comprises approximately 20-35 molar % of the compound, or a pharmaceutically acceptable salt thereof. In some embodiments, the composition comprises approximately 20-40 molar % of the compound, or a pharmaceutically acceptable salt thereof. In some embodiments, the composition comprises approximately 20-45 molar % of the compound, or a pharmaceutically acceptable salt thereof. In some embodiments, the composition comprises approximately 20-50 molar % of the compound, or a pharmaceutically acceptable salt thereof. In some embodiments, the composition comprises approximately 20-55 molar % of the compound, or a pharmaceutically acceptable salt thereof. In some embodiments, the composition comprises approximately 30, approximately 35, approximately 40, approximately 45, approximately 50, approximately 55, or approximately 60 molar % of the compound, or a pharmaceutically acceptable salt thereof.

In some embodiments, the composition comprises approximately 0-75 molar % of a sterol. In some embodiments, the composition comprises approximately 5-60 molar % of a sterol. In some embodiments, the composition comprises approximately 5-50 molar % of a sterol. In some embodiments, the composition comprises approximately 5-40 molar % of a sterol. In some embodiments, the composition comprises approximately 5-30 molar % of a sterol. In some embodiments, the composition comprises approximately 5-20 molar % of a sterol. In some embodiments, the composition comprises approximately 10-60 molar % of a sterol. In some embodiments, the composition comprises approximately 20-60 molar % of a sterol. In some embodiments, the composition comprises approximately 30-60 molar % of a sterol. In some embodiments, the composition comprises approximately 40-60 molar % of a sterol. In some embodiments, the composition comprises approximately 50-60 molar % of a sterol. In some embodiments, the composition comprises approximately 20-55 molar % of a sterol. In some embodiments, the composition comprises approximately 25-50 molar % of a sterol. In some embodiments, the composition comprises approximately 5, approximately 7, approximately 8.5, approximately 10, approximately 12.5, approximately 15, approximately 17, approximately 20, approximately 23.5, approximately 25, approximately 27.75, approximately 28.5, approximately 30, approximately 32.75, approximately 35, approximately 37, approximately 37.75, approximately 38.5, approximately 40, approximately 42, approximately 45, approximately 48.5, approximately 50, approximately 55, approximately 57, approximately 58.5, or approximately 60 molar % of a sterol. In some embodiments, the composition comprises approximately 7, approximately 8.5, approximately 17, approximately 23.5, approximately 27.75, approximately 28.5, approximately 32.75, approximately 37, approximately 37.75, approximately 38.5, approximately 42, approximately 48.5, approximately 57, or approximately 58.5 molar % of a sterol.

In some embodiments, the composition comprises approximately 0-75 molar % of cholesterol. In some embodiments, the composition comprises approximately 5-60 molar % of cholesterol. In some embodiments, the composition comprises approximately 5-50 molar % of cholesterol. In some embodiments, the composition comprises approximately 5-40 molar % of cholesterol. In some embodiments, the composition comprises approximately 5-30 molar % of cholesterol. In some embodiments, the composition comprises approximately 5-20 molar % of cholesterol. In some embodiments, the composition comprises approximately 10-60 molar % of cholesterol. In some embodiments, the composition comprises approximately 20-60 molar % of cholesterol. In some embodiments, the composition comprises approximately 30-60 molar % of cholesterol. In some embodiments, the composition comprises approximately 40-60 molar % of cholesterol. In some embodiments, the composition comprises approximately 50-60 molar % of cholesterol. In some embodiments, the composition comprises approximately 20-55 molar % of cholesterol. In some embodiments, the composition comprises approximately 25-50 molar % of cholesterol. In some embodiments, the composition comprises approximately 5, approximately 7, approximately 8.5, approximately 10, approximately 12.5, approximately 15, approximately 17, approximately 20, approximately 23.5, approximately 25, approximately 27.75, approximately 28.5, approximately 30, approximately 32.75, approximately 35, approximately 37, approximately 37.75, approximately 38.5, approximately 40, approximately 42, approximately 45, approximately 48.5, approximately 50, approximately 55, approximately 57, approximately 58.5, or approximately 60 molar % of cholesterol. In some embodiments, the composition comprises approximately 7, approximately 8.5, approximately 17, approximately 23.5, approximately 27.75, approximately 28.5, approximately 32.75, approximately 37, approximately 37.75, approximately 38.5, approximately 42, approximately 48.5, approximately 57, or approximately 58.5 molar % of cholesterol.

In some embodiments, the composition comprises approximately 0-20 molar % of a PEG-lipid. In some embodiments, the composition comprises approximately 0-15 molar % of a PEG-lipid. In some embodiments, the composition comprises approximately 0-10 molar % of a PEG-lipid. In some embodiments, the composition comprises approximately 0-5 molar % of a PEG-lipid. In some embodiments, the composition comprises approximately 1-20 molar % of a PEG-lipid. In some embodiments, the composition comprises approximately 1-15 molar % of a PEG-lipid. In some embodiments, the composition comprises approximately 1-10 molar % of a PEG-lipid. In some embodiments, the composition comprises approximately 1-5 molar % of a PEG-lipid. In some embodiments, the composition comprises approximately 1-3 molar % of a PEG-lipid. In some embodiments, the composition comprises approximately 0.5, approximately 0.75, approximately 1.0, approximately 1.25, approximately 1.5, approximately 1.75, approximately 2.0, approximately 2.25, approximately 2.5, approximately 2.75, approximately 3.0, approximately 3.25, or approximately 3.5 molar % of a PEG-lipid.

In some embodiments, the composition comprises approximately 0-20 molar % of DMPE-PEG2k. In some embodiments, the composition comprises approximately 0-15 molar % of DMPE-PEG2k. In some embodiments, the composition comprises approximately 0-10 molar % of DMPE-PEG2k. In some embodiments, the composition comprises approximately 0-5 molar % of DMPE-PEG2k. In some embodiments, the composition comprises approximately 1-20 molar % of DMPE-PEG2k. In some embodiments, the composition comprises approximately 1-15 molar % of DMPE-PEG2k. In some embodiments, the composition comprises approximately 1-10 molar % of DMPE-PEG2k. In some embodiments, the composition comprises approximately 1-5 molar % of DMPE-PEG2k. In some embodiments, the composition comprises approximately 1-3 molar % of DMPE-PEG2k. In some embodiments, the composition comprises approximately 0.5, approximately 0.75, approximately 1.0, approximately 1.25, approximately 1.5, approximately 1.75, approximately 2.0, approximately 2.25, approximately 2.5, approximately 2.75, approximately 3.0, approximately 3.25, or approximately 3.5 molar % of DMPE-PEG2k.

In some embodiments, the composition comprises approximately 0-50 molar % of a phospholipid. In some embodiments, the composition comprises approximately 0-40 molar % of a phospholipid. In some embodiments, the composition comprises approximately 10-15 molar % of a phospholipid. In some embodiments, the composition comprises approximately 10-20 molar % of a phospholipid. In some embodiments, the composition comprises approximately 10-25 molar % of a phospholipid. In some embodiments, the composition comprises approximately 10-30 molar % of a phospholipid. In some embodiments, the composition comprises approximately 10-35 molar % of a phospholipid. In some embodiments, the composition comprises approximately 10-40 molar % of a phospholipid. In some embodiments, the composition comprises approximately 10-45 molar % of a phospholipid. In some embodiments, the composition comprises approximately 10-50 molar % of a phospholipid. In some embodiments, the composition comprises approximately 5-45 molar % of a phospholipid. In some embodiments, the composition comprises approximately 15-45 molar % of a phospholipid. In some embodiments, the composition comprises approximately 20-45 molar % of a phospholipid. In some embodiments, the composition comprises approximately 25-45 molar % of a phospholipid. In some embodiments, the composition comprises approximately 5, approximately 10, approximately 15, approximately 20, approximately 30, approximately 35, approximately 40, approximately 45, or approximately 50 molar % of a phospholipid.

In some embodiments, the composition comprises approximately 0-50 molar % of DOPE. In some embodiments, the composition comprises approximately 0-40 molar % of DOPE. In some embodiments, the composition comprises approximately 10-15 molar % of DOPE. In some embodiments, the composition comprises approximately 10-20 molar % of DOPE. In some embodiments, the composition comprises approximately 10-25 molar % of DOPE. In some embodiments, the composition comprises approximately 10-30 molar % of DOPE. In some embodiments, the composition comprises approximately 10-35 molar % of DOPE. In some embodiments, the composition comprises approximately 10-40 molar % of DOPE. In some embodiments, the composition comprises approximately 10-45 molar % of DOPE. In some embodiments, the composition comprises approximately 10-50 molar % of DOPE. In some embodiments, the composition comprises approximately 5-45 molar % of DOPE. In some embodiments, the composition comprises approximately 15-45 molar % of DOPE. In some embodiments, the composition comprises approximately 20-45 molar % of DOPE. In some embodiments, the composition comprises approximately 25-45 molar % of DOPE. In some embodiments, the composition comprises approximately 5, approximately 10, approximately 15, approximately 20, approximately 30, approximately 35, approximately 40, approximately 45, or approximately 50 molar % of DOPE.

In some embodiments, the composition comprises approximately 0-50 molar % of DSPC. In some embodiments, the composition comprises approximately 0-40 molar % of DSPC. In some embodiments, the composition comprises approximately 10-15 molar % of DSPC. In some embodiments, the composition comprises approximately 10-20 molar % of DSPC. In some embodiments, the composition comprises approximately 10-25 molar % of DSPC. In some embodiments, the composition comprises approximately 10-30 molar % of DSPC. In some embodiments, the composition comprises approximately 10-35 molar % of DSPC. In some embodiments, the composition comprises approximately 10-40 molar % of DSPC. In some embodiments, the composition comprises approximately 10-45 molar % of DSPC. In some embodiments, the composition comprises approximately 10-50 molar % of DSPC. In some embodiments, the composition comprises approximately 5-45 molar % of DSPC. In some embodiments, the composition comprises approximately 15-45 molar % of DSPC. In some embodiments, the composition comprises approximately 20-45 molar % of DSPC. In some embodiments, the composition comprises approximately 25-45 molar % of DSPC. In some embodiments, the composition comprises approximately 5, approximately 10, approximately 15, approximately 20, approximately 30, approximately 35, approximately 40, approximately 45, or approximately 50 molar % of DSPC.

In some embodiments, the composition comprises about a 20:1 ratio of the compound, or a pharmaceutically acceptable salt thereof:agent by mass. In some embodiments, the composition comprises about a 17.5:1 ratio of the compound, or a pharmaceutically acceptable salt thereof:agent by mass. In some embodiments, the composition comprises about a 15:1 ratio of the compound, or a pharmaceutically acceptable salt thereof:agent by mass. In some embodiments, the composition comprises about a 12.5:1 ratio of the compound, or a pharmaceutically acceptable salt thereof:agent by mass. In some embodiments, the composition comprises about a 10:1 ratio of the compound, or a pharmaceutically acceptable salt thereof:agent by mass. In some embodiments, the composition comprises about a 7.5:1 ratio of the compound, or a pharmaceutically acceptable salt thereof:agent by mass. In some embodiments, the composition comprises about a 5:1 ratio of the compound, or a pharmaceutically acceptable salt thereof:agent by mass. In some embodiments, the composition comprises about a 2.5:1 ratio of the compound, or a pharmaceutically acceptable salt thereof:agent by mass.

In some embodiments, the composition comprises about a 20:1 ratio of the compound, or a pharmaceutically acceptable salt thereof:polynucleotide by mass. In some embodiments, the composition comprises about a 17.5:1 ratio of the compound, or a pharmaceutically acceptable salt thereof:polynucleotide by mass. In some embodiments, the composition comprises about a 15:1 ratio of the compound, or a pharmaceutically acceptable salt thereof:polynucleotide by mass. In some embodiments, the composition comprises about a 12.5:1 ratio of the compound, or a pharmaceutically acceptable salt thereof:polynucleotide by mass. In some embodiments, the composition comprises about a 10:1 ratio of the compound, or a pharmaceutically acceptable salt thereof:polynucleotide by mass. In some embodiments, the composition comprises about a 7.5:1 ratio of the compound, or a pharmaceutically acceptable salt thereof:polynucleotide by mass. In some embodiments, the composition comprises about a 5:1 ratio of the compound, or a pharmaceutically acceptable salt thereof:polynucleotide by mass. In some embodiments, the composition comprises about a 2.5:1 ratio of the compound, or a pharmaceutically acceptable salt thereof:polynucleotide by mass.

In some embodiments, the composition comprises about a 20:1 ratio of the compound, or a pharmaceutically acceptable salt thereof:mRNA by mass. In some embodiments, the composition comprises about a 17.5:1 ratio of the compound, or a pharmaceutically acceptable salt thereof:mRNA by mass. In some embodiments, the composition comprises about a 15:1 ratio of the compound, or a pharmaceutically acceptable salt thereof:mRNA by mass. In some embodiments, the composition comprises about a 12.5:1 ratio of the compound, or a pharmaceutically acceptable salt thereof:mRNA by mass. In some embodiments, the composition comprises about a 10:1 ratio of the compound, or a pharmaceutically acceptable salt thereof:mRNA by mass. In some embodiments, the composition comprises about a 7.5:1 ratio of the compound, or a pharmaceutically acceptable salt thereof:mRNA by mass. In some embodiments, the composition comprises about a 5:1 ratio of the compound, or a pharmaceutically acceptable salt thereof:mRNA by mass. In some embodiments, the composition comprises about a 2.5:1 ratio of the compound, or a pharmaceutically acceptable salt thereof:mRNA by mass.

In some embodiments, the composition comprises the compound, or a pharmaceutically acceptable salt thereof, a phospholipid, a PEG lipid, and a sterol. In some embodiments, the composition comprises the compound, or a pharmaceutically acceptable salt thereof, DOPE, DMPE-PEG2k, and cholesterol. In some embodiments, the composition comprises the compound, or a pharmaceutically acceptable salt thereof, DSPC, DMPE-PEG2k, and cholesterol.

In some embodiments, the composition comprises approximately 30-60% the compound, or a pharmaceutically acceptable salt thereof, approximately 10-30% of a phospholipid, approximately 1.5-3% of a PEG lipid, and approximately 7-58.5% a sterol. In some embodiments, the composition comprises approximately 30-60% the compound, or a pharmaceutically acceptable salt thereof, approximately 10-30% of DOPE, approximately 1.5-3% of DMPE-PEG2k, and approximately 7-58.5% cholesterol. In some embodiments, the composition comprises approximately 30-60% the compound, or a pharmaceutically acceptable salt thereof, approximately 10-30% of DSPC, approximately 1.5-3% of DMPE-PEG2k, and approximately 7-58.5% cholesterol.

In some embodiments, the composition comprises approximately 20-30% of a phospholipid, approximately 1.5-3% of a PEG-lipid, approximately 30% of the compound, or a pharmaceutically acceptable salt thereof, and approximately 37-48.5% of a sterol. In some embodiments, the composition comprises approximately 30% of a phospholipid, approximately 3% of a PEG-lipid, approximately 30% of the compound, or a pharmaceutically acceptable salt thereof, and approximately 37% of a sterol. In some embodiments, the composition comprises approximately 20% of a phospholipid, approximately 1.5% of a PEG-lipid, approximately 30% of the compound, or a pharmaceutically acceptable salt thereof, and approximately 48.5% of a sterol. In some embodiments, the composition comprises approximately 30% of a phospholipid, approximately 2.25% of a PEG-lipid, approximately 30% of the compound, or a pharmaceutically acceptable salt thereof, and approximately 37.75% of a sterol.

In some embodiments, the composition comprises approximately 20-30% of DOPE, approximately 1.5-3% of DMPE-PEG-2k, approximately 30% of the compound, or a pharmaceutically acceptable salt thereof, and approximately 37-48.5% of cholesterol. In some embodiments, the composition comprises approximately 30% of DOPE, approximately 3% of DMPE-PEG-2k, approximately 30% of the compound, or a pharmaceutically acceptable salt thereof, and approximately 37% of cholesterol. In some embodiments, the composition comprises approximately 20% of DOPE, approximately 1.5% of DMPE-PEG-2k, approximately 30% of the compound, or a pharmaceutically acceptable salt thereof, and approximately 48.5% of cholesterol. In some embodiments, the composition comprises approximately 30% of DOPE, approximately 2.25% of DMPE-PEG-2k, approximately 30% of the compound, or a pharmaceutically acceptable salt thereof, and approximately 37.75% of cholesterol.

In some embodiments, the composition comprises approximately 10-30% of a phospholipid, approximately 2.25-3% of a PEG-lipid, approximately 30-45% of the compound, or a pharmaceutically acceptable salt thereof, and approximately 32.75-57% of a sterol. In some embodiments, the composition comprises approximately 20% of a phospholipid, approximately 2.25% of a PEG-lipid, approximately 45% of the compound, or a pharmaceutically acceptable salt thereof, and approximately 32.75% of a sterol. In some embodiments, the composition comprises approximately 30% of a phospholipid, approximately 2.25% of a PEG-lipid, approximately 30% of the compound, or a pharmaceutically acceptable salt thereof, and approximately 37.75% of a sterol. In some embodiments, the composition comprises approximately 10% of a phospholipid, approximately 3% of a PEG-lipid, approximately 30% of the compound, or a pharmaceutically acceptable salt thereof, and approximately 57% of a sterol.

In some embodiments, the composition comprises approximately 10-30% of DSPC, approximately 2.25-3% of DMPE-PEG-2k, approximately 30-45% of the compound, or a pharmaceutically acceptable salt thereof, and approximately 32.75-57% of cholesterol. In some embodiments, the composition comprises approximately 20% of DSPC, approximately 2.25% of DMPE-PEG-2k, approximately 45% of the compound, or a pharmaceutically acceptable salt thereof, and approximately 32.75% of cholesterol. In some embodiments, the composition comprises approximately 30% of DSPC, approximately 2.25% of DMPE-PEG-2k, approximately 30% of the compound, or a pharmaceutically acceptable salt thereof, and approximately 37.75% of cholesterol. In some embodiments, the composition comprises approximately 10% of DSPC, approximately 3% of DMPE-PEG-2k, approximately 30% of the compound, or a pharmaceutically acceptable salt thereof, and approximately 57% of cholesterol.

In some embodiments, the composition comprises approximately 20-30% the compound, or a pharmaceutically acceptable salt thereof, approximately 30-40% of a phospholipid, approximately 2.5% of a PEG lipid, and approximately 27.5-47.5% a sterol.

In some embodiments, the composition comprises approximately 30-40% of a phospholipid, approximately 25-30% of the compound, or a pharmaceutically acceptable salt thereof, approximately 2.5% of a PEG lipid, and approximately 27.5-37.5% of a sterol. In some embodiments, the composition comprises approximately 35% of a phospholipid, approximately 30% of the compound, or a pharmaceutically acceptable salt thereof, approximately 2.5% of a PEG lipid, and approximately 32.5% of a sterol. In some embodiments, the composition comprises approximately 30% of a phospholipid, approximately 30% of the compound, or a pharmaceutically acceptable salt thereof, approximately 2.5% of a PEG lipid, and approximately 37.5% of a sterol. In some embodiments, the composition comprises approximately 40% of a phospholipid, approximately 30% of the compound, or a pharmaceutically acceptable salt thereof, approximately 2.5% of a PEG lipid, and approximately 27.5% of a sterol. In some embodiments, the composition comprises approximately 40% of a phospholipid, approximately 25% of the compound, or a pharmaceutically acceptable salt thereof, approximately 2.5% of a PEG lipid, and approximately 32.5% of a sterol.

In some embodiments, the composition comprises approximately 30-40% of a phospholipid, approximately 25-30% of the compound, or a pharmaceutically acceptable salt thereof, approximately 2.5% of a DMPE-PEG2k, and approximately 27.5-37.5% of cholesterol. In some embodiments, the composition comprises approximately 35% of a phospholipid, approximately 30% of the compound, or a pharmaceutically acceptable salt thereof, approximately 2.5% of a DMPE-PEG2k, and approximately 32.5% of cholesterol. In some embodiments, the composition comprises approximately 30% of a phospholipid, approximately 30% of the compound, or a pharmaceutically acceptable salt thereof, approximately 2.5% of a DMPE-PEG2k, and approximately 37.5% of cholesterol. In some embodiments, the composition comprises approximately 40% of a phospholipid, approximately 30% of the compound, or a pharmaceutically acceptable salt thereof, approximately 2.5% of a DMPE-PEG2k, and approximately 27.5% of cholesterol. In some embodiments, the composition comprises approximately 40% of a phospholipid, approximately 25% of the compound, or a pharmaceutically acceptable salt thereof, approximately 2.5% of a DMPE-PEG2k, and approximately 32.5% of cholesterol.

In some embodiments, the composition comprises approximately 30-40% of DSPC, approximately 25-30% of the compound, or a pharmaceutically acceptable salt thereof, approximately 2.5% of a DMPE-PEG2k, and approximately 27.5-37.5% of cholesterol. In some embodiments, the composition comprises approximately 35% of DSPC, approximately 30% of the compound, or a pharmaceutically acceptable salt thereof, approximately 2.5% of a DMPE-PEG2k, and approximately 32.5% of cholesterol. In some embodiments, the composition comprises approximately 30% of DSPC, approximately 30% of the compound, or a pharmaceutically acceptable salt thereof, approximately 2.5% of a DMPE-PEG2k, and approximately 37.5% of cholesterol. In some embodiments, the composition comprises approximately 40% of DSPC, approximately 30% of the compound, or a pharmaceutically acceptable salt thereof, approximately 2.5% of a DMPE-PEG2k, and approximately 27.5% of cholesterol. In some embodiments, the composition comprises approximately 40% of DSPC, approximately 25% of the compound, or a pharmaceutically acceptable salt thereof, approximately 2.5% of a DMPE-PEG2k, and approximately 32.5% of cholesterol.

In some embodiments, the composition comprises approximately 30-40% of DOPE, approximately 25-30% of the compound, or a pharmaceutically acceptable salt thereof, approximately 2.5% of a DMPE-PEG2k, and approximately 27.5-37.5% of cholesterol. In some embodiments, the composition comprises approximately 35% of DOPE, approximately 30% of the compound, or a pharmaceutically acceptable salt thereof, approximately 2.5% of a DMPE-PEG2k, and approximately 32.5% of cholesterol. In some embodiments, the composition comprises approximately 30% of DOPE, approximately 30% of the compound, or a pharmaceutically acceptable salt thereof, approximately 2.5% of a DMPE-PEG2k, and approximately 37.5% of cholesterol. In some embodiments, the composition comprises approximately 40% of DOPE, approximately 30% of the compound, or a pharmaceutically acceptable salt thereof, approximately 2.5% of a DMPE-PEG2k, and approximately 27.5% of cholesterol. In some embodiments, the composition comprises approximately 40% of DOPE, approximately 25% of the compound, or a pharmaceutically acceptable salt thereof, approximately 2.5% of a DMPE-PEG2k, and approximately 32.5% of cholesterol.

In some embodiments, the composition comprises approximately 35% of the compound, or a pharmaceutically acceptable salt thereof, approximately 16% of a phospholipid, approximately 46.5% of a sterol, and approximately 2.5% of a PEG-lipid. In some embodiments, the composition comprises approximately 35% of the compound, or a pharmaceutically acceptable salt thereof, approximately 16% of DOPE, approximately 46.5% of cholesterol, and approximately 2.5% of DMPE-PEG.

Pharmaceutical compositions described herein can be prepared by any method known in the art of pharmaceutics. In general, such preparatory methods include bringing a compound, agent, or particle described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents such as calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor*, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (II) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound, agent, or particle described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound, agent, or particle in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds, agents, or particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound, agent, or particle required to achieve an effective amount will vary from subject to subject, depending, for example, on species, age, and general condition of a subject, severity of the side effects or disorder, identity of the particular compound, agent or particle, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound, agent, or particle described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound, agent, or particle described herein. In certain embodiments, a dose described herein includes independently between 1 mg and 3 mg, inclusive, of a compound, agent, or particle described herein. In certain embodiments, a dose described herein includes independently between 3 mg and 10 mg, inclusive, of a compound, agent, or particle described herein. In certain embodiments, a dose described herein includes independently between 10 mg and 30 mg, inclusive, of a compound, agent, or particle described herein. In certain embodiments, a dose described herein includes independently between 30 mg and 100 mg, inclusive, of a compound, agent, or particle described herein.

In certain embodiments, a dose (e.g., a single dose, or any dose of multiple doses) described herein includes independently between 0.001 mg/kg and 25 mg/kg, between 0.01 mg/kg and 10 mg/kg, between 0.05 mg/kg and 5 mg/kg, between 0.1 mg/kg and 2.5 mg/kg, between 0.1 mg/kg and 1 mg/kg, between 0.2 mg/kg and 0.8 mg/kg, or between 0.25 mg/kg and 0.75 mg/kg, inclusive, of a compound, agent, or particle described herein. In certain embodiments, a dose described herein includes independently between 0.1 mg/kg and 0.1 mg/kg, inclusive, of a compound, agent, or particle described herein. In certain embodiments, a dose described herein includes independently between 0.1 mg/kg and 1 mg/kg, inclusive, of a compound, agent, or particle described herein. In certain embodiments, a dose described herein includes independently between 0.1 mg/kg and 2.5 mg/kg, inclusive, of a compound, agent, or particle described herein. In certain embodiments, a dose described herein includes independently between 2.5 mg/kg and 5 mg/kg, inclusive, of a compound, agent, or particle described herein. In certain embodiments, a dose described herein includes independently between 5 mg/kg and 25 mg/kg, inclusive, of a compound, agent, or particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that treat a disease in a subject in need thereof, prevent a disease in a subject in need thereof, or reduce the risk to develop a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, polynucleotides, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., lung disease or liver disease). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

### PEG-Lipid

In some embodiments, the PEG-lipid is a PEG-phospholipid or PEG-glyceride lipid.

In certain embodiments, the PEG-lipid is a PEG-phospholipid. In certain embodiments, the PEG-phospholipid is a PEG-phosphoethanolamine. In some embodiments, the PEG-phospholipid is a PEG-phosphatidylcholine.

In certain embodiments, the PEG component has a molecular weight of about 350, about 550, about 750, about 1000, about 2000, about 3000, or about 5000. In some embodiments, the PEG component has a molecular weight of about 750, about 1000, about 2000, about 3000, or about 5000. In certain embodiments, the PEG component has a molecular weight of 500-1000, 1000-2000, 2000-3000, or 3000-4000. In certain embodiments, the PEG component has a molecular weight of about 1000, about 2000, or about 3000. In some embodiments, the PEG component has a molecular weight of about 2000.

In certain embodiments, the PEG-lipid is stearoyl-substituted (Cis). In some embodiments, the PEG-phospholipid is palmitoyl-substituted (C₁₆). In certain embodiments, the PEG-phospholipid is myristoyl-substituted (C₁₄).

In certain embodiments, the PEG-lipid is selected from the group consisting of 1,2-distearoyl-sn-glycero-3-phosphoethanolamine-N-[methoxy(polyethylene glycol)-5000](C₁₈PEG5000), 1,2-dipalmitoyl-sn-glycero-3-phosphoethanolamine-N-[methoxy(polyethylene glycol)-5000](C₁₆PEG5000), 1,2-dimyristoyl-sn-glycero-3-phosphoethanolamine-N-[methoxy(polyethylene glycol)-5000](C₁₄PEG5000), 1,2-distearoyl-sn-glycero-3-phosphoethanolamine-N-[methoxy(polyethylene glycol)-3000](C₁₈PEG3000), 1,2-dipalmitoyl-sn-glycero-3-phosphoethanolamine-N-[methoxy(polyethylene glycol)-3000](C₁₆PEG3000), 1,2-dimyristoyl-sn-glycero-3-phosphoethanolamine-N-[methoxy(polyethylene glycol)-3000](C₁₄PEG3000), 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N-[methoxy(polyethylene glycol)-2000](C₁₆PEG2000), 1,2-dimyristoyl-sn-glycero-3-phosphoethanolamine-N-[methoxy(polyethylene glycol)-2000](C₁₄PEG2000), 1,2-distearoyl-sn-glycero-3-phosphoethanolamine-N-[methoxy(polyethylene glycol)-1000](C₁₈PEG1000), 1,2-dipalmitoyl-sn-glycero-3-phosphoethanolamine-N-[methoxy(polyethylene glycol)-1000](C₁₆PEG1000), 1,2-dimyristoyl-sn-glycero-3-phosphoethanolamine-N-[methoxy(polyethylene glycol)-1000](C₁₄PEG1000), 1,2-distearoyl-sn-glycero-3-phosphoethanolamine-N-[methoxy(polyethylene glycol)-750](C₁₈PEG750), 1,2-dipalmitoyl-sn-glycero-3-phosphoethanolamine-N-[methoxy(polyethylene glycol)-750](C₁₆PEG750), and 1,2-dimyristoyl-sn-glycero-3-phosphoethanolamine-N-[methoxy(polyethylene glycol)-750](C₁₄PEG750). In some embodiments, the PEG-lipid is selected from the group consisting of 1,2-dimyristoyl-sn-glycero-3-phosphoethanolamine-polyethylene glycol (DMPE-PEG), 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N-[methoxy(polyethylene glycol)-2000](C₁₆PEG2000), and 1,2-dimyristoyl-sn-glycero-3-phosphoethanolamine-N-[methoxy(polyethylene glycol)-2000](C₁₄PEG2000). In certain embodiments, the PEG-lipid is selected from the group consisting of 1,2-dimyristoyl-sn-glycero-3-phosphoethanolamine-polyethylene glycol (DMPE-PEG), 1,2-dimyristoyl-sn-glycero-3-phosphoethanolamine-N-[methoxy(polyethylene glycol)-5000](C₁₄PEG5000), 1,2-dimyristoyl-sn-glycero-3-phosphoethanolamine-N-[methoxy(polyethylene glycol)-3000](C₁₄PEG3000), 1,2-dimyristoyl-sn-glycero-3-phosphoethanolamine-N-[methoxy(polyethylene glycol)-2000](C₁₄PEG2000), 1,2-dimyristoyl-sn-glycero-3-phosphoethanolamine-N-[methoxy(polyethylene glycol)-1000](C₁₄PEG1000), and 1,2-dimyristoyl-sn-glycero-3-phosphoethanolamine-N-[methoxy(polyethylene glycol)-750](C₁₄PEG750). In certain embodiments, the PEG-phospholipid is 1,2-dimyristoyl-sn-glycero-3-phosphoethanolamine-N-[methoxy(polyethylene glycol)-2000](C₁₄PEG2000). In some embodiments, the PEG-lipid is 1,2-dimyristoyl-sn-glycero-3-phosphoethanolamine-polyethylene glycol (DMPE-PEG).

In some embodiments, the PEG-lipid is a PEG-glyceride lipid. In certain embodiments, the PEG-lipid is 1,2-distearoyl-rac-glycero-3-methoxypolyethylene glycol-2000 (DSG-PEG2000) or 1,2-dimyristoyl-rac-glycero-3-methoxypolyethylene glycol-2000 (DMG-PEG2000). In some embodiments, the PEG-lipid is 1,2-distearoyl-rac-glycero-3-methoxypolyethylene glycol-2000 (DSG-PEG2000). In certain embodiments, the PEG-lipid is 1,2-dimyristoyl-rac-glycero-3-methoxypolyethylene glycol-2000 (DMG-PEG2000).

### Sterol

In certain embodiments, the sterol is cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, or a derivative thereof. In some embodiments, the sterol is cholesterol, or a derivative thereof. In certain embodiments, the sterol is cholesterol.

### Phospholipid

In certain embodiments, the phospholipid is a phosphoethanolamine or phosphatidylcholine. In some embodiments, the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE). In some embodiments, the phospholipid is a phosphoethanolamine. In certain embodiments, the phospholipid is 1,2-distearoyl-sn-glycero-3-phosphorylethanolamine (DSPE) or 1,2-dioleoyl-sn-glycero-3-phosphoethanolamine (DOPE). In certain embodiments, the phospholipid is 1,2-dioleoyl-sn-glycero-3-phosphoethanolamine (DOPE) or distearoylphosphatidylcholine (DSPC). In certain embodiments, the phospholipid is 1,2-distearoyl-sn-glycero-3-phosphorylethanolamine (DSPE). In certain embodiments, the phospholipid is 1,2-dioleoyl-sn-glycero-3-phosphoethanolamine (DOPE). In some embodiments, the phospholipid is a phosphatidylcholine. In some embodiments, the phospholipid is 1,2-distearoyl-sn-glycero-3-phosphocholine (DSPC).

### Agents

In certain embodiments, the composition further comprises an agent. In some embodiments, the agent is an organic molecule, inorganic molecule, polynucleotide, protein, peptide, targeting agent, an isotopically labeled chemical compound, vaccine, an immunological agent, or an agent useful in bioprocessing.

In some embodiments, the agent and the compound, or the pharmaceutically acceptable salt thereof, are not covalently attached.

Agents that are delivered by the systems (e.g., pharmaceutical compositions) described herein may be (e.g., therapeutic or prophylactic), diagnostic, cosmetic, or nutraceutical agents. Any chemical compound to be administered to a subject may be delivered using the complexes, picoparticles, nanoparticles (e.g., lipid nanoparticles), microparticles, micelles, or liposomes, described herein. In some embodiments, the agent is an organic molecule, inorganic molecule, protein, peptide, polynucleotide, targeting agent, an isotopically labeled chemical compound, vaccine, an immunological agent, or an agent useful in bioprocessing (e.g., intracellular manufacturing of proteins, such as a cell's bioprocessing of a commercially useful chemical or fuel). For example, intracellular delivery of an agent may be useful in bioprocessing by maintaining the cell's health and/or growth, e.g., in the manufacturing of proteins. Any chemical compound to be administered to a subject or contacted with a cell may be delivered to the subject or cell using the compositions.

Exemplary agents that may be included in a composition described herein include, but are not limited to, small molecules, organometallic compounds, polynucleotides, proteins, peptides, carbohydrates, monosaccharides, oligosaccharides, polysaccharides, nucleoproteins, mucoproteins, lipoproteins, small molecules linked to proteins, glycoproteins, steroids, nucleotides, oligonucleotides, polynucleotides, nucleosides, antisense oligonucleotides, lipids, hormones, vitamins, cells, metals, targeting agents, isotopically labeled chemical compounds, drugs (e.g., compounds approved for human or veterinary use by the U.S. Food and Drug Administration as provided in the Code of Federal Regulations), vaccines, immunological agents, agents useful in bioprocessing, and mixtures thereof. The targeting agents are described in more detail herein. In certain embodiments, the agents are nutraceutical agents. In certain embodiments, the agents are pharmaceutical agents (e.g., a therapeutic or prophylactic agent). In certain embodiments, the agent is an antibiotic agent (e.g., an anti-bacterial, anti-viral, or anti-fungal agent), anesthetic, steroidal agent, anti-proliferative agent, anti-inflammatory agent, anti-angiogenesis agent, anti-neoplastic agent, anti-cancer agent, anti-diabetic agent, antigen, vaccine, antibody, decongestant, antihypertensive, sedative, birth control agent, progestational agent, anti-cholinergic, analgesic, immunosuppressant, anti-depressant, anti-psychotic, P-adrenergic blocking agent, diuretic, cardiovascular active agent, vasoactive agent, non-steroidal, nutritional agent, anti-allergic agent, or pain-relieving agent. Vaccines may comprise isolated proteins or peptides, inactivated organisms and viruses, dead organisms and viruses, genetically altered organisms or viruses, polynucleotide (e.g., mRNA), and cell extracts. Therapeutic and prophylactic agents may be combined with interleukins, interferon, cytokines, and adjuvants such as cholera toxin, alum, and Freund's adjuvant, etc.

In certain embodiments, an agent to be delivered or used in a composition described herein is a polynucleotide. In certain embodiments, the agent is plasmid DNA (pDNA). In certain embodiments, the agent is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the agent is RNA. In certain embodiments, the agent is small interfering RNA (siRNA). In certain embodiments, the agent is messenger RNA (mRNA). In certain embodiments, the agent is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In some embodiments, the RNA is coding RNA or non-coding RNA. In some embodiments, the coding RNA is messenger RNA (mRNA). In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the RNA is single-stranded RNA, heterogeneous nuclear RNA, satellite RNA, viral RNA, or viral satellite RNA. In some embodiments, the agent is ribozyme or flexizyme. In some embodiments, the polynucleotide is a DNA. In some embodiments, the DNA is a plasmid DNA (pDNA).

In certain embodiments, the polynucleotide may be provided as an antisense agent or RNAi. See, e.g., Fire et al., Nature 391:806-811, 1998. Antisense therapy is meant to include, e.g., administration or in situ provision of single- or double-stranded polynucleotides, or derivatives thereof, which specifically hybridize, e.g., bind, under cellular conditions, with cellular mRNA and/or genomic DNA, or mutants thereof, so as to inhibit the expression of the encoded protein, e.g., by inhibiting transcription and/or translation. See, e.g., Crooke, “Molecular mechanisms of action of antisense drugs,” Biochim. Biophys. Acta 1489(1):31-44, 1999; Crooke, “Evaluating the mechanism of action of anti-proliferative antisense drugs,” Antisense Nucleic Acid Drug Dev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, pDNA, siRNA, dsRNA, shRNA, miRNA, mRNA, tRNA, asRNA, and/or RNAi can be designed and/or predicted using one or more of a large number of available algorithms. To give but a few examples, the following resources can be utilized to design and/or predict polynucleotides: algorithms found at Alnylum Online; Dharmacon Online; OligoEngine Online; Molecula Online; Ambion Online; BioPredsi Online; RNAi Web Online; Chang Bioscience Online; Invitrogen Online; LentiWeb Online GenScript Online; Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271.

The polynucleotide included in a composition may be of any size or sequence, and they may be single- or double-stranded. In certain embodiments, the polynucleotide includes at least about 30, at least about 100, at least about 300, at least about 1,000, at least about 3,000, or at least about 10,000 base pairs. In certain embodiments, the polynucleotide includes less than about 10,000, less than about 3,000, less than about 1,000, less than about 300, less than about 100, or less than about 30 base pairs. Combinations of the above ranges (e.g., at least about 100 and less than about 1,000) are also within the scope of the invention. The polynucleotide may be provided by any means known in the art. In certain embodiments, the polynucleotide is engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry. The polynucleotide may be isolated and/or purified. In certain embodiments, the polynucleotide is substantially free of impurities. In certain embodiments, the polynucleotide is at least about 50%, at least about 60%, at least about 70%, at least about 80%, at least about 90%, at least about 95%, or at least about 99% free of impurities.

The polynucleotide may be modified by physical, chemical, and/or biological means. The modifications include methylation, phosphorylation, and end-capping, etc. In certain embodiments, the modifications lead to increased stability of the polynucleotide.

Wherever a polynucleotide is employed in the composition, a derivative of the polynucleotide may also be used. These derivatives include products resulted from modifications of the polynucleotide in the base moieties, sugar moieties, and/or phosphate moieties of the polynucleotide. Modified base moieties include, but are not limited to, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, 0(6)-methylguanine, and 2-thiocytidine. Modified sugar moieties include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single polynucleotide. These modified polynucleotides may be provided by any means known in the art; however, as will be appreciated by those of skill in the art, the modified polynucleotides may be prepared using synthetic chemistry in vitro.

The polynucleotide described herein may be in any form, such as a circular plasmid, a linearized plasmid, a cosmid, a viral genome, a modified viral genome, and an artificial chromosome.

The polynucleotide described herein may be of any sequence. In certain embodiments, the polynucleotide encodes a protein or peptide. The encoded protein may be an enzyme, structural protein, receptor, soluble receptor, ion channel, active (e.g., pharmaceutically active) protein, cytokine, interleukin, antibody, antibody fragment, antigen, coagulation factor, albumin, growth factor, hormone, and insulin, etc. The polynucleotide may also comprise regulatory regions to control the expression of a gene. These regulatory regions may include, but are not limited to, promoters, enhancer elements, repressor elements, TATA boxes, ribosomal binding sites, and stop sites for transcription, etc. In certain embodiments, the polynucleotide is not intended to encode a protein. For example, the polynucleotide may be used to fix an error in the genome of the cell being transfected.

In certain embodiments, the polynucleotide is immunomodulatory, e.g., immunostimulatory, or immunosuppressive.

In certain embodiments, the polynucleotide described herein comprises a sequence encoding an antigenic peptide or protein. A composition containing the polynucleotide can be delivered to a subject to induce an immunologic response sufficient to decrease the chance of a subsequent infection and/or lessen the symptoms associated with such an infection. The polynucleotide of these vaccines may be combined with interleukins, interferon, cytokines, and/or adjuvants described herein.

The antigenic protein or peptides encoded by the polynucleotide may be derived from bacterial organisms, such as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, and Camphylobacter jejuni; from viruses, such as coronavirus (e.g., SARS-CoV-2), smallpox virus, influenza A virus, influenza B virus, respiratory syncytial virus, parainfluenza virus, measles virus, HIV virus, varicella-zoster virus, herpes simplex 1 virus, herpes simplex 2 virus, cytomegalovirus, Epstein-Barr virus, rotavirus, rhinovirus, adenovirus, papillomavirus, poliovirus, mumps virus, rabies virus, rubella virus, coxsackieviruses, equine encephalitis virus, Japanese encephalitis virus, yellow fever virus, Rift Valley fever virus, hepatitis A virus, hepatitis B virus, hepatitis C virus, hepatitis D virus, and hepatitis E virus; and from fungal, protozoan, or parasitic organisms, such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, and Schistosoma mansoni.

In some embodiments, the antigenic protein or peptides encoded by the polynucleotide may be derived from coronavirus (e.g., SARS-CoV-2), influenza A virus, or influenza B virus. In some embodiments, the antigenic protein or peptides encoded by the polynucleotide may be derived from SARS-CoV-2 (e.g., SARS-CoV-2 B.1.617.2). In some embodiments, the antigenic protein or peptides encoded by the polynucleotide may be derived from SARS-CoV-2 B.1.617.2. In some embodiments, the antigenic protein or peptides encoded by the polynucleotide may be derived from influenza A virus (e.g., hemagglutinin). In some embodiments, the antigenic protein or peptides encoded by the polynucleotide may be derived from influenza B virus.

In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus.

In some embodiments, the RNA is an mRNA for inducing functional protein expression. In some embodiments, the RNA is an mRNA that encodes a luciferase. In some embodiments, the RNA is an mRNA that encodes one or more antigens. In some embodiments, the RNA is an mRNA that encodes a chimeric antigen receptor (CAR). In some embodiments, the antigen selected from a viral antigen, a bacterial antigen, a fungal antigen, a parasitic antigen, an influenza antigen, a tumor-associated antigen, a tumor-specific antigen, or any combination thereof. In some embodiments, the antigen comprises a tumor-specific antigen or tumor-associated antigen. In some embodiments, the antigen induces an adaptive immune response. In some embodiments, the RNA is an mRNA that induces expression of a CAR. In some embodiments, the RNA is an mRNA that induces transient expression of a CAR.

In some embodiments, the agent comprises a sequence shown in Table 1. In some embodiments, the agent comprises the sequence SARS-CoV-2 B.1.617.2 S2P (SEQ ID NO 1).

TABLE 1
Non-limiting examples of sequences

| | SEQ | |
|---|---|---|
| | ID | |
| Name | NO: | Sequence |
| SARS-CoV- | 1 | AGACAGATCGCCTGGAGACGCCATCCACGCTGTTTTGACCTCC |
| 2 B.1.617.2 | | ATAGAAGACACCGGGACCGATCCAGCCTCCGCGGCCGGGAAC |
| S2P | | GGTGCATTGGAACGCGGATTCCCCGTGCCAAGAGTGACTCACC |
| | | GTCCTTGACACGATGTTCGTATTCCTCGTATTGCTGCCACTGGT |
| | | GAGCAGCCAGTGCGTGAACCTGAGGACCAGGACCCAGCTGCC |
| | | CCCCGCCTACACCAACAGCTTCACCAGGGGCGTGTACTACCCC |
| | | GACAAGGTGTTCAGGAGCAGCGTGCTGCACAGCACCCAGGAC |
| | | CTGTTCCTGCCCTTCTTCAGCAACGTGACCTGGTTCCACGCCA |
| | | TCCACGTGAGCGGCACCAACGGCACCAAGAGGTTCGACAACC |
| | | CCGTGCTGCCCTTCAACGACGGCGTGTACTTCGCCAGCATCGA |
| | | GAAGAGCAACATCATCAGGGGCTGGATCTTCGGCACCACCCTG |
| | | GACAGCAAGACCCAGAGCCTGCTGATCGTGAACAACGCCACC |
| | | AACGTGGTGATCAAGGTGTGCGAGTTCCAGTTCTGCAACGAC |
| | | CCCTTCCTGGACGTGTACTACCACAAGAACAACAAGAGCTGG |
| | | ATGGAGAGCGGCGTGTACAGCAGCGCCAACAACTGCACCTTC |
| | | GAGTACGTGAGCCAGCCCTTCCTGATGGACCTGGAGGGCAAG |
| | | CAGGGCAACTTCAAGAACCTGAGGGAGTTCGTGTTCAAGAAC |
| | | ATCGACGGCTACTTCAAGATCTACAGCAAGCACACCCCCATCA |
| | | ACCTGGTGAGGGACCTGCCCCAGGGCTTCAGCGCCCTGGAGC |
| | | CCCTGGTGGACCTGCCCATCGGCATCAACATCACCAGGTTCCA |
| | | GACCCTGCTGGCCCTGCACAGAAGCTACCTGACCCCCGGCGA |
| | | CAGCAGCAGCGGCTGGACCGCCGGCGCCGCCGCCTACTACGT |
| | | GGGCTACCTGCAGCCAAGAACCTTCCTGCTGAAGTACAACGA |
| | | GAACGGCACCATCACCGACGCCGTGGACTGCGCCCTGGACCC |
| | | CCTGAGCGAGACAAAGTGCACCCTGAAGAGCTTCACCGTGGA |
| | | GAAGGGCATCTACCAGACCAGCAACTTCAGGGTGCAGCCCAC |
| | | CGAGAGCATCGTGAGGTTCCCCAACATCACCAACCTGTGCCCC |
| | | TTCGGCGAGGTGTTCAACGCCACCAGGTTCGCCAGCGTGTACG |
| | | CCTGGAACAGGAAGAGAATCAGCAACTGCGTGGCCGACTACA |
| | | GCGTGCTGTACAACAGCGCCAGCTTCAGCACCTTCAAGTGCTA |
| | | CGGCGTGAGCCCCACCAAGCTGAACGACCTGTGCTTCACCAA |
| | | CGTGTACGCCGACAGCTTCGTGATCAGGGGCGACGAGGTGAG |
| | | GCAGATCGCCCCCGGCCAGACCGGCAAGATCGCCGACTACAA |
| | | CTACAAGCTGCCCGACGACTTCACCGGCTGCGTGATCGCCTGG |
| | | AACAGCAACAACCTGGACAGCAAGGTGGGCGGCAACTACAAC |
| | | TACAGGTACAGGCTGTTCAGGAAGAGCAACCTGAAGCCCTTC |
| | | GAGAGGGACATCAGCACCGAGATCTACCAGGCCGGCAGCAAG |
| | | CCCTGCAACGGCGTGGAGGGCTTCAACTGCTACTTCCCCCTGC |
| | | AGAGCTACGGCTTCCAGCCCACCAACGGCGTGGGCTACCAGC |
| | | CCTACAGGGTGGTGGTGCTGAGCTTCGAGCTGCTGCACGCCCC |
| | | CGCCACCGTGTGCGGCCCCAAGAAGAGCACCAACCTGGTGAA |
| | | GAACAAGTGCGTGAACTTCAACTTCAACGGCCTGACCGGCAC |
| | | CGGCGTGCTGACCGAGAGCAACAAGAAGTTCCTGCCCTTCCA |
| | | GCAGTTCGGCAGGGACATCGCCGACACCACCGACGCCGTGAG |
| | | GGACCCCCAGACCCTGGAGATCCTGGACATCACCCCCTGCAGC |
| | | TTCGGCGGCGTGAGCGTGATCACCCCCGGCACCAACACCAGC |
| | | AACCAGGTGGCCGTGCTGTACCAGGGCGTGAACTGCACCGAG |
| | | GTGCCCGTGGCCATCCACGCCGACCAGCTGACCCCCACCTGG |
| | | AGGGTGTACAGCACCGGCAGCAACGTGTTCCAGACCAGGGCC |
| | | GGCTGCCTGATCGGCGCCGAGCACGTGAACAACAGCTACGAG |
| | | TGCGACATCCCCATCGGCGCCGGCATCTGCGCCAGCTACCAGA |
| | | CCCAGACCAACAGCAGGAGAAGGGCCAGGAGCGTGGCCAGC |
| | | CAGAGCATCATCGCCTACACCATGAGCCTGGGCGCCGAGAACA |
| | | GCGTGGCCTACAGCAACAACAGCATCGCCATCCCCACCAACTT |
| | | CACCATCAGCGTGACCACCGAGATCCTGCCCGTGAGCATGACC |
| | | AAGACCAGCGTGGACTGCACCATGTACATCTGCGGCGACAGC |
| | | ACCGAGTGCAGCAACCTGCTGCTGCAGTACGGCAGCTTCTGC |
| | | ACCCAGCTGAACAGGGCCCTGACCGGCATCGCCGTGGAGCAG |
| | | GACAAGAACACCCAGGAGGTGTTCGCCCAGGTGAAGCAGATC |
| | | TACAAGACCCCCCCCATCAAGGACTTCGGCGGCTTCAACTTCA |
| | | GCCAGATCCTGCCCGACCCCAGCAAGCCCAGCAAGAGAAGCT |
| | | TCATCGAGGACCTGCTGTTCAACAAGGTGACCCTGGCCGACG |
| | | CCGGCTTCATCAAGCAGTACGGCGACTGCCTGGGCGACATCGC |
| | | CGCCAGGGACCTGATCTGCGCCCAGAAGTTCAACGGCCTGAC |
| | | CGTGCTGCCCCCCCTGCTGACCGACGAGATGATCGCCCAGTAC |
| | | ACCAGCGCCCTGCTGGCCGGCACCATCACCAGCGGCTGGACA |
| | | TTCGGCGCCGGCGCCGCCCTGCAGATCCCATTCGCCATGCAGA |
| | | TGGCCTACAGGTTCAACGGCATCGGCGTGACCCAGAACGTGCT |
| | | GTACGAGAACCAGAAGCTGATCGCCAACCAGTTCAACAGCGC |
| | | CATCGGCAAGATCCAGGACAGCCTGAGCAGCACCGCCAGCGC |
| | | CCTGGGCAAGCTGCAGAACGTGGTGAACCAGAACGCCCAGGC |
| | | CCTGAACACCCTGGTGAAGCAGCTGAGCAGCAACTTCGGCGC |
| | | CATCAGCAGCGTGCTGAACGACATCCTGAGCAGGCTGGACCC |
| | | CCCCGAGGCCGAGGTGCAGATCGACAGACTGATCACCGGCAG |
| | | GCTGCAGAGCCTGCAGACCTACGTGACCCAGCAGCTGATCAG |
| | | GGCCGCCGAGATCAGGGCCAGCGCCAACCTGGCCGCCACCAA |
| | | GATGAGCGAGTGCGTGCTGGGCCAGAGCAAGAGGGTGGACTT |
| | | CTGCGGCAAGGGCTACCACCTGATGAGCTTCCCCCAGAGCGCC |
| | | CCCCACGGCGTGGTGTTCCTGCACGTGACCTACGTGCCCGCCC |
| | | AGGAGAAGAACTTCACCACCGCCCCCGCCATCTGCCACGACG |
| | | GCAAGGCCCACTTCCCCAGGGAGGGCGTGTTCGTGAGCAACG |
| | | GCACCCACTGGTTCGTGACCCAGAGGAACTTCTACGAGCCCC |
| | | AGATCATCACCACCGACAACACCTTCGTGAGCGGCAACTGCG |
| | | ACGTGGTGATCGGCATCGTGAACAACACCGTGTACGACCCCCT |
| | | GCAGCCCGAGCTGGACAGCTTCAAGGAGGAGCTGGACAAGTA |
| | | CTTCAAGAACCACACCAGCCCCGACGTGGACCTGGGCGACAT |
| | | CAGCGGCATCAACGCCAGCGTGGTGAACATCCAGAAGGAGAT |
| | | CGACAGGCTGAACGAGGTGGCCAAGAACCTGAACGAGAGCC |
| | | TGATCGACCTGCAGGAGCTGGGCAAGTACGAGCAGTACATCA |
| | | AGTGGCCCTGGTACATCTGGCTGGGCTTCATCGCCGGCCTGAT |
| | | CGCCATCGTGATGGTGACCATCATGCTGTGCTGCATGACCAGC |
| | | TGCTGCAGCTGCCTGAAGGGCTGCTGCAGCTGCGGCAGCTGC |
| | | TGCAAGTTCGACGAGGACGACAGCGAGCCCGTGCTGAAGGGC |
| | | GTGAAGCTGCACTACATCtgatagTAACGGGTGGCATCCCTGTGA |
| | | CCCCTCCCCAGTGCCTCTCCTGGCCCTGGAAGTTGCCACTCCA |
| | | GTGCCCACCAGCCTTGTCCTAATAAAATTAAGTTGCATCAAGCT |
| | | CTCTAG |

Two or more sequences may be assessed for the identity between the sequences. The terms “identical” or percent “identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using a sequence comparison algorithm or by manual alignment and visual inspection.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using a sequence comparison algorithm or by manual alignment and visual inspection.

In certain embodiments, the agent is erythropoietin (EPO), e.g., recombinant human erythropoietin (rhEPO). Erythropoietin is an essential hormone for red blood cell production, and may be used in treating hematological diseases, e.g., anemia., such as anemia resulting from chronic kidney disease, chemotherapy induced anemia in patients with cancer, inflammatory bowel disease (Crohn's disease and ulcerative colitis) and myelodysplasia from the treatment of cancer (chemotherapy and radiation). Recombinant human erythropoietins available for use include EPOGEN/PROCRIT (Epoetin alfa, rINN) and ARANESP (Darbepoetin alfa, rINN).

An agent described herein may be non-covalently (e.g., complexed or encapsulated) attached to a compound as described herein, or included in a composition described herein. In certain embodiments, upon delivery of the agent into a cell, the agent is able to interfere with the expression of a specific gene in the cell.

In certain embodiments, the agent in a composition that is delivered to a subject in need thereof may be a mixture of two or more agents that may be useful as, e.g., combination therapies. The compositions including the two or more agents can be administered to achieve a synergistic effect. In certain embodiments, the compositions including the two or more agents can be administered to improve the activity and/or bioavailability, reduce and/or modify the metabolism, inhibit the excretion, and/or modify the distribution within the body of a subject, of each one of the two or more agents. It will also be appreciated that the therapy employed may achieve a desired effect for the same disorder, and/or it may achieve different effects.

The compositions (e.g., pharmaceutical compositions) can be administered concurrently with, prior to, or subsequent to the one or more agents (e.g., pharmaceutical agents). The two or more agents may be useful for treating and/or preventing a same disease or different diseases described herein. Each one of the agents may be administered at a dose and/or on a time schedule determined for that agent. The agents may also be administered together with each other and/or with the composition described herein in a single dose or administered separately in different doses. The particular combination to employ in a regimen will take into account compatibility of the agents and/or the desired therapeutic and/or prophylactic effect to be achieved. In general, it is expected that the agents utilized in combination be utilized at levels that do not exceed the levels at which they are utilized individually. In some embodiments, the levels utilized in combination will be lower than those utilized individually.

### Targeting Agents

Since it is often desirable to target a particular cell, collection of cells, or tissue, compounds provided herein, and the complexes, liposomes, micelles, and particles (e.g., microparticles and nanoparticles) thereof, may be modified to include targeting moieties.

For example, a compound provided herein may include a targeting moiety. A variety of agents or regions that target particular cells are known in the art. See, e.g., Cotten et al., Methods Enzym. 217:618, 1993. The targeting agent may be included throughout a particle of a compound provided herein or may be only on the surface of the particle. The targeting agent may be a protein, peptide, carbohydrate, glycoprotein, lipid, small molecule, or polynucleotide, etc. The targeting agent may be used to target specific cells or tissues or may be used to promote endocytosis or phagocytosis of the particle. Examples of targeting agents include, but are not limited to, antibodies, fragments of antibodies, proteins, peptides, carbohydrates, receptor ligands, sialic acid, and aptamers, etc. If the targeting agent is included throughout a particle, the targeting agent may be included in the mixture that is used to form the particle. If the targeting agent is only on the surface of a particle, the targeting agent may be associated with (e.g., by covalent or non-covalent (e.g., electrostatic, hydrophobic, hydrogen bonding, van der Waals, iT-n stacking) interactions) the formed particle using standard chemical techniques.

### Particles

In some embodiments, a composition including a compound provided herein and an agent is in the form of a particle. In certain embodiments, the compound provided herein and agent form a complex, and the complex is in the form of a particle. In certain embodiments, the compound provided herein encapsulates the agent and is in the form of a particle. In certain embodiments, the compound provided herein is mixed with the agent, and the mixture is in the form of a particle. In some embodiments, the particle encapsulates the agent.

In certain embodiments, a complex of a compound provided herein and an agent in a composition of is in the form of a particle. In some embodiments, the particle is a nanoparticle or a microparticle. In certain embodiments, the particle is a microparticle (i.e., particle having a characteristic dimension of less than about 1 millimeter and at least about 1 micrometer, where the characteristic dimension of the particle is the smallest cross-sectional dimension of the particle). In certain embodiments, the particle is a nanoparticle (i.e., a particle having a characteristic dimension of less than about 1 micrometer and at least about 1 nanometer, where the characteristic dimension of the particle is the smallest cross-sectional dimension of the particle). In certain embodiments, the average diameter of the particle is at least about 10 nm, at least about 30 nm, at least about 100 nm, at least about 300 nm, at least about 1 μm, at least about 3 μm, at least about 10 μm, at least about 30 μm, at least about 100 μm, at least about 300 μm, or at least about 1 mm. In certain embodiments, the average diameter of the particle is less than about 1 mm, less than about 300 μm, less than about 100 μm, less than about 30 μm less than about 10 μm, less than about 3 μm, less than about 1 μm, less than about 300 nm, less than about 100 nm, less than about 30 nm, or less than about 10 nm. Combinations of the above ranges (e.g., at least about 100 nm and less than about 1 μm) are also within the scope of the present invention.

The particles described herein may include additional materials such as polymers (e.g., synthetic polymers (e.g., PEG, PLGA) and natural polymers (e.g., phospholipids)). In certain embodiments, the additional materials are approved by a regulatory agency, such as the U.S. FDA, for human and veterinary use.

The particles may be prepared using any method known in the art, such as precipitation, milling, spray drying, single and double emulsion solvent evaporation, solvent extraction, phase separation, and simple and complex coacervation. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, polydispersity, etc.). The method of preparing the particle and the conditions (e.g., solvent, temperature, concentration, and air flow rate, etc.) used may also depend on the agent being complexed, encapsulated, or mixed, and/or the composition of the matrix.

Methods developed for making particles for delivery of agents that are included in the particles are described in the literature. See, e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988.

If the particles prepared by any of the above methods have a size range outside of the desired range, the particles can be sized, for example, using a sieve. The particles may also be coated. In certain embodiments, the particles are coated with a targeting agent. In certain embodiments, the particles are coated with a surface-altering agent. In some embodiments, the particles are coated to achieve desirable surface properties (e.g., a particular charge).

In certain embodiments, the polydispersity index (PDI, determined by dynamic light scattering) of the particles described herein (e.g., particles included in a composition described herein) is between 0.01 and 0.9, between 0.1 and 0.9, between 0.1 and 0.7, between 0.1 and 0.5, between 0.01 and 0.4, between 0.03 and 0.4, between 0.1 and 0.4, between 0.01 and 0.3, between 0.03 and 0.3, or between 0.1 and 0.3.

### Micelles and Liposomes

A composition including one or more compounds provided herein and an agent may be in the form of a micelle, liposome, or lipoplex. In certain embodiments, the compound provided herein is in the form of a micelle or liposome. In certain embodiments, the agent is in the form of a micelle or liposome. In certain embodiments, the compound provided herein and agent form a complex, and the complex is in the form of a micelle or liposome. In certain embodiments, the compound provided herein encapsulates the agent and is in the form of a micelle or liposome. In certain embodiments, the compound provided herein is mixed with the agent, and the mixture is in the form of a micelle or liposome. Micelles and liposomes are particularly useful in delivering an agent, such as a hydrophobic agent. When the micelle or liposome is complexed with (e.g., encapsulates or covers) a polynucleotide, the resulting complex may be referred to as a “lipoplex.” Many techniques for preparing micelles and liposomes are known in the art, and any such method may be used herein to make micelles and liposomes.

In certain embodiments, liposomes are formed through spontaneous assembly. In some embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these liposomes have formed, reducing the size of the liposomes can be modified through input of sonic energy (sonication) or mechanical energy (extrusion). See, e.g., Walde, P. “Preparation of Vesicles (Liposomes)” In Encylopedia of Nanoscience and Nanotechnology; Nalwa, H. S. Ed. American Scientific Publishers: Los Angeles, 2004; Vol. 9, pp. 43-79; Szoka et al., “Comparative Properties and Methods of Preparation of Lipid Vesicles (Liposomes)” Ann. Rev. Biophys. Bioeng. 9:467-508, 1980; each of which is incorporated herein by reference. The preparation of lipsomes may involve preparing a compound provided herein for hydration, hydrating the compound with agitation, and sizing the vesicles to achieve a homogenous distribution of liposomes. A compound provided herein may be first dissolved in an organic solvent in a container to result in a homogeneous mixture. The organic solvent is then removed to form a polymer-derived film. This polymer-derived film is thoroughly dried to remove residual organic solvent by placing the container on a vacuum pump for a period of time. Hydration of the polymer-derived film is accomplished by adding an aqueous medium and agitating the mixture. Disruption of LMV suspensions using sonic energy typically produces small unilamellar vesicles (SUV) with diameters in the range of 15-50 nm. Lipid extrusion is a technique in which a lipid/polymer suspension is forced through a polycarbonate filter with a defined pore size to yield particles having a diameter near the pore size of the filter used. Extrusion through filters with 100 nm pores typically yields large, unilamellar polymer-derived vesicles (LUV) with a mean diameter of 120-140 nm. In certain embodiments, the amount of a compound provided herein in the liposome ranges from about 30 mol % to about 80 mol %, from about 40 mol % to about 70 mol %, or from about 60 mol % to about 70 mol %. In certain embodiments, the compound provided herein employed further complexes an agent, such as a polynucleotide. In such embodiments, the application of the liposome is the delivery of the polynucleotide.

The following scientific papers described other methods for preparing liposomes and micelles: Narang et al., “Cationic Lipids with Increased DNA Binding Affinity for Nonviral Gene Transfer in Dividing and Nondividing Cells,” Bioconjugate Chem. 16:156-68, 2005; Hofland et al., “Formation of stable cationic lipid/DNA complexes for gene transfer,” Proc. Nati. Acad. Sci. USA 93:7305-7309, July 1996; Byk et al., “Synthesis, Activity, and Structure—Activity Relationship Studies of Novel Cationic Lipids for DNA Transfer,” J. Med. Chem. 41(2):224-235, 1998; Wu et al., “Cationic Lipid Polymerization as a Novel Approach for Constructing New DNA Delivery Agents,” Bioconjugate Chem. 12:251-57, 2001; Lukyanov et al., “Micelles from lipid derivatives of water-soluble polymers as delivery systems for poorly soluble drugs,” Advanced Drug Delivery Reviews 56:1273-1289, 2004; Tranchant et al., “Physicochemical optimisation of plasmid delivery by cationic lipids,” J. Gene Med. 6:S24—S35, 2004; van Balen et al., “Liposome/Water Lipophilicity: Methods, Information Content, and Pharmaceutical Applications,” Medicinal Research Rev. 24(3):299-324, 2004.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for delivering an agent to a subject or cell. In certain embodiments, the kits are useful for delivering a polynucleotide to a subject or cell. In certain embodiments, the kits are useful for delivering mRNA to a subject or cell. In certain embodiments, the kits are useful for delivering mRNA that encodes a CAR to a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for delivering an agent to a subject or cell. In certain embodiments, the kits and instructions provide for delivering a polynucleotide to a subject or cell. In certain embodiments, the kits and instructions provide for delivering mRNA to a subject or cell. A kit described herein may include one or more additional pharmaceutical agents described herein as a separate composition.

### Methods of Treatment and Uses

Also provided herein are methods for treating and/or preventing a disease, disorder, or condition in a subject, comprising administering to the subject a composition comprising an agent and a compound (e.g., a compound of Formula (I), (XI), (XII), or (XIII)), or a pharmaceutically acceptable salt thereof. Also provided herein are compounds (e.g., a compound of Formula (I), (XI), (XII), or (XIII)), and a pharmaceutically acceptable salts thereof, and compositions provided herein for use in treating and/or preventing a disease, disorder, or condition in a subject. Also provided herein are uses of a compound provided herein (e.g., a compound of Formula (I), (XI), (XII), or (XIII)), and pharmaceutically acceptable salts thereof, or a composition provided herein in the manufacture of a medicament for treating and/or preventing a disease, disorder, or condition in a subject.

In certain embodiments, the disease, disorder, or condition is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease. In some embodiments, the disease, disorder, or condition is a genetic disease. In some embodiments, the disease, disorder, or condition is a proliferative disease. In some embodiments, the disease, disorder, or condition is a hematological disease. In some embodiments, the disease, disorder, or condition is a neurological disease. In some embodiments, the disease, disorder, or condition is a liver disease or a spleen disease. In some embodiments, the disease, disorder, or condition is a liver disease. In some embodiments, the disease, disorder, or condition is a spleen disease. In some embodiments, the disease, disorder, or condition is a lung disease. In some embodiments, the disease, disorder, or condition is a painful condition. In some embodiments, the disease, disorder, or condition is a psychiatric disorder. In some embodiments, the disease, disorder, or condition is a musculoskeletal disease. In some embodiments, the disease, disorder, or condition is a metabolic disorder. In some embodiments, the disease, disorder, or condition is an inflammatory disease. In some embodiments, the disease, disorder, or condition is an autoimmune disease.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In some embodiments, the agent is any agent provided herein. In certain embodiments, the agent is a polynucleotide. In some embodiments, the agent is mRNA.

In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus.

In some embodiments, the RNA is an mRNA for inducing functional protein expression. In some embodiments, the RNA is an mRNA that encodes a luciferase. In some embodiments, the RNA is an mRNA that encodes one or more antigens. In some embodiments, the RNA is an mRNA that encodes a chimeric antigen receptor (CAR). In some embodiments, the antigen selected from a viral antigen, a bacterial antigen, a fungal antigen, a parasitic antigen, an influenza antigen, a tumor-associated antigen, a tumor-specific antigen, or any combination thereof. In some embodiments, the antigen comprises a tumor-specific antigen or tumor-associated antigen. In some embodiments, the antigen induces an adaptive immune response. In some embodiments, the RNA is an mRNA that induces expression of a CAR. In some embodiments, the RNA is an mRNA that induces transient expression of a CAR.

### Additional Methods and Uses

Also provided herein are methods of delivering an agent (e.g., a polynucleotide) to a subject, tissue, or a cell, comprising administering to the subject or contacting the tissue or the cell with a composition comprising an agent and a compound provided herein (e.g., a compound of Formula (I), (XI), (XII), or (XIII)), or a pharmaceutically acceptable salt thereof. Also provided herein are compounds (e.g., a compound of Formula (I), (XI), (XII), or (XIII)), and a pharmaceutically acceptable salt thereof, and compositions provided herein for use in delivering an agent (e.g., a polynucleotide) to a subject, tissue, or a cell. Also provided herein are uses of a compound provided herein (e.g., a compound of Formula (I), (XI), (XII), or (XIII)), and pharmaceutically acceptable salts thereof, or a composition provided herein in the manufacture of a medicament for delivering an agent (e.g., a polynucleotide) to a subject, tissue, or a cell.

In some embodiments, provided herein are methods of delivering an agent (e.g., a polynucleotide) to a subject or a cell, comprising administering to the subject or contacting the cell with a composition comprising an agent and a compound provided herein (e.g., a compound of Formula (I), (XI), (XII), or (XIII)), or a pharmaceutically acceptable salt thereof.

In some embodiments, the agent is any agent provided herein. In some embodiments, the agent is an organic molecule, inorganic molecule, polynucleotide, protein, peptide, polynucleotide, targeting agent, an isotopically labeled chemical compound, vaccine, an immunological agent, or an agent useful in bioprocessing. In certain embodiments, the agent is a polynucleotide. In some embodiments, the agent is mRNA. In some embodiments, the RNA is coding RNA or non-coding RNA. In some embodiments, the coding RNA is messenger RNA (mRNA). IN some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the agent is ribozyme or flexizyme. In some embodiments, the RNA is single-stranded RNA, heterogeneous nuclear RNA, satellite RNA, viral RNA, or viral satellite RNA. In some embodiments, the polynucleotide is a DNA. In some embodiments, the DNA is a plasmid DNA (pDNA). In some embodiments, the polynucleotide is delivered to the liver or spleen of the subject. In some embodiments, the composition is administered intravenously.

In some embodiments, the agent is an mRNA that encodes for the SARS-CoV-2 B.1.617.2 spike protein. In some embodiments, the agent is an mRNA that encodes for the SARS-CoV-2 B.1.617.2 spike protein with 984P and 985P mutations. In some embodiments, the agent is an mRNA that encodes for a hemagglutinin protein of influenza A virus. In some embodiments, the agent is an mRNA that encodes for the H3 protein of influenza A virus.

In some embodiments, the RNA is an mRNA for inducing functional protein expression. In some embodiments, the RNA is an mRNA that encodes a luciferase. In some embodiments, the RNA is an mRNA that encodes one or more antigens. In some embodiments, the RNA is an mRNA that encodes a chimeric antigen receptor (CAR). In some embodiments, the antigen selected from a viral antigen, a bacterial antigen, a fungal antigen, a parasitic antigen, an influenza antigen, a tumor-associated antigen, a tumor-specific antigen, or any combination thereof. In some embodiments, the antigen comprises a tumor-specific antigen or tumor-associated antigen. In some embodiments, the antigen induces an adaptive immune response. In some embodiments, the RNA is an mRNA that induces expression of a CAR. In some embodiments, the RNA is an mRNA that induces transient expression of a CAR.

In some embodiments, the agent is delivered to a subject. In some embodiments, the agent is delivered to the liver or spleen of the subject. In some embodiments, the agent is delivered to the liver of the subject. In certain embodiments, the agent is delivered to the spleen of the subject. In some embodiments, the polynucleotide is delivered to a subject. In some embodiments, the polynucleotide is delivered to the liver or spleen of the subject. In some embodiments, the polynucleotide is delivered to the liver of the subject. In certain embodiments, the polynucleotide is delivered to the spleen of the subject. In some embodiments, the mRNA is delivered to a subject. In some embodiments, the mRNA is delivered to the liver or spleen of the subject. In some embodiments, the mRNA is delivered to the liver of the subject. In certain embodiments, the mRNA is delivered to the spleen of the subject.

In certain embodiments, the agent is delivered to a cell. In certain embodiments, the polynucleotide is delivered to a cell. In certain embodiments, the mRNA is delivered to a cell. In some embodiments, the cell is in vivo, e.g., in an organism. In certain embodiments, the cell is in vitro, e.g., in cell culture. In some embodiments, the cell is ex vivo, meaning the cell is removed from an organism prior to the delivery.

In some embodiments, the cell is an immune cell, T cell, resident T cells, B cell, natural killer (NK) cell, cancerous cell, cell associated with a disease or disorder, epithelial cell, endothelial cell, or tumor cell. In some embodiments, the cell is a cell in tissue associated with a disease or disorder, brain tissue, central nervous system tissue, pulmonary tissue, apical surface tissue, liver tissue, intestine tissue, colon tissue, small intestine tissue, large intestine tissue, feces, bone marrow, spleen tissue, muscles tissue, joint tissue, diseased tissues, lymph node tissue, lymphatic circulation, or any combination thereof. In some embodiments, the cell is a hematopoietic stem cell or an immune cell. In some embodiments, the cell is a macrophage, monocyte, or fibroblast. In some embodiments, the cell is a dendritic cell. In some embodiments, the cell is an epithelial cell. In some embodiments, the cell is a myoblast cell. In some embodiments, the cell is a macrophage. In some embodiments, the cell is a neutrophil. In some embodiments, the cell is a monocyte. In some embodiments, the cell is a fibroblast. In some embodiments, the cell is a Hela cell, a Jurkat cell, a DC2.4 cell, a 293T-ACE2 cell, a C2C12 cell, a CD45- cell. In some embodiments, the cell is a Hela cell or a Jurkat cell.

In some embodiments, the tissue is associated with a disease or disorder. In some embodiments, the tissue is brain tissue, central nervous system tissue, pulmonary tissue, apical surface tissue, liver tissue, intestine tissue, colon tissue, small intestine tissue, large intestine tissue, feces, bone marrow, spleen tissue, muscles tissue, joint tissue, diseased tissues, lymph node tissue, lymphatic circulation, or any combination thereof.

In some embodiments, the composition is administered by any method provided herein. In certain embodiments, the composition is administered intravenously or intramuscularly. In certain embodiments, the composition is administered intravenously. In certain embodiments, the composition is administered intramuscularly.

In another aspect, provided herein is a method of preparing a compound of Formula (I′):

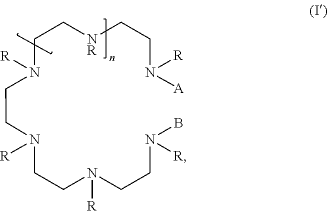

or a salt, solvate, tautomer, stereoisomer, or isotopically labeled derivative thereof, the method comprising reacting a compound of Formula (VII):

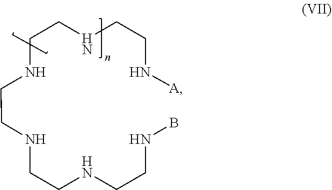

or a salt, solvate, tautomer, stereoisomer, or isotopically labeled derivative thereof, with a compound of Formula (VIII):

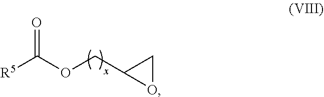

or a salt, solvate, tautomer, stereoisomer, or isotopically labeled derivative thereof, wherein:
n is 1, 2, or 3;
A and B are independently optionally substituted aliphatic; or
A and B combine to form optionally substituted C₂-C₆ alkylene;
each R is independently hydrogen, a nitrogen protecting group, or

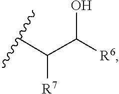

wherein one of R⁶ and R⁷ is hydrogen, and the other is

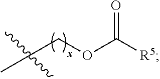

each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive; provided that at least one R is

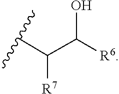

In a particular embodiment, provided herein is a method of preparing a compound of Formula (I′), or a salt thereof.

In another aspect, provided herein is a method of preparing a compound of the formula:

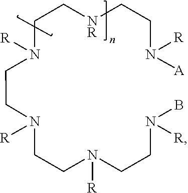

or a salt thereof, the method comprising reacting a compound of the formula:

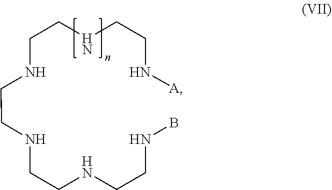

or a salt thereof, with a compound of Formula (VIII):

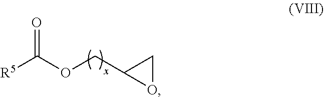

or a salt thereof, wherein:
n is 1, 2, or 3;
A and B are independently optionally substituted aliphatic; or
A and B combine to form optionally substituted C₂-C₆ alkylene;
each R is independently hydrogen, a nitrogen protecting group, or

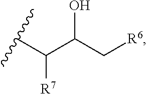

wherein one of R⁶ and R⁷ is hydrogen, and the other is

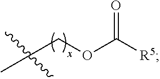

each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is

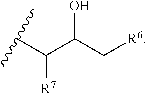

In some embodiments, the compound of Formula (VII) is of Formula (VII-a):

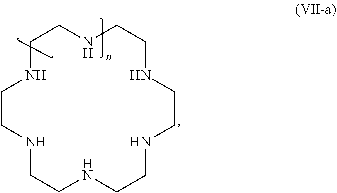

or a salt, solvate, tautomer, stereoisomer, or isotopically labeled derivative thereof. In some embodiments, the compound of Formula (VII) is of Formula (VII-a), or a salt thereof.

In some embodiments, the compound is of the formula:

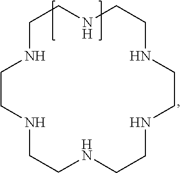

or a salt thereof.

In some embodiments, the compound of Formula (VII) is of Formula (VII-b):

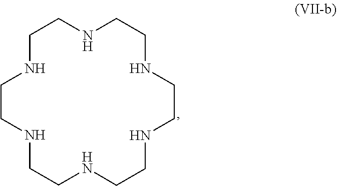 [SMILES: C1CNCCNCCNCCNCCNCCN1]

or a salt, solvate, tautomer, stereoisomer, or isotopically labeled derivative thereof. In some embodiments, the compound of Formula (VII) is of Formula (VII-b), or a salt thereof.

In some embodiments, the compound of Formula (VII) is of Formula (VII-c):

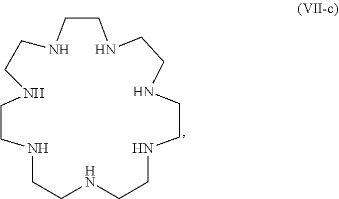 [SMILES: C1CNCCNCCNCCNCCNCCNCCN1]

or a salt, solvate, tautomer, stereoisomer, or isotopically labeled derivative thereof. In some embodiments, the compound of Formula (VII) is of Formula (VII-c), or a salt thereof.

In some embodiments, the compound of Formula (VII) is of Formula (VII-d):

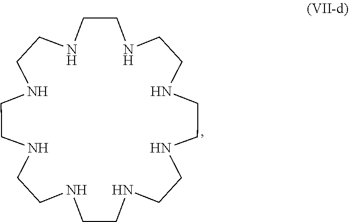 [SMILES: C1CNCCNCCNCCNCCNCCNCCNCCN1]

or a salt, solvate, tautomer, stereoisomer, or isotopically labeled derivative thereof. In some embodiments, the compound of Formula (VII) is of Formula (VII-d), or a salt thereof.

In some embodiments, the compound of Formula (VII) is of Formula (VII-e):

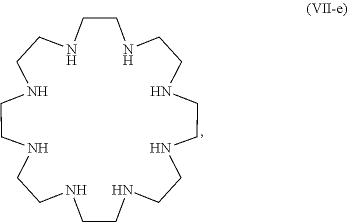 [SMILES: C1CNCCNCCNCCNCCNCCNCCNCCN1]

or a salt, solvate, tautomer, stereoisomer, or isotopically labeled derivative thereof. In some embodiments, the compound of Formula (VII) is of Formula (VII-e), or a salt thereof.

In some embodiments, the compound of Formula (VII) is of Formula (VII-f):

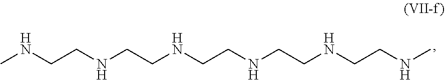

or a salt, solvate, tautomer, stereoisomer, or isotopically labeled derivative thereof. In some embodiments, the compound of Formula (VII) is of Formula (VII-f), or a salt thereof.

In another aspect, provided herein is a method of preparing a compound of Formula (XI′):

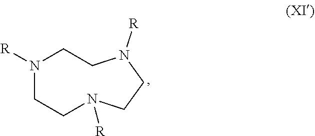

or a salt, solvate, tautomer, stereoisomer, or isotopically labeled derivative thereof, the method comprising reacting a compound of Formula (XI″):

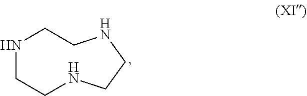

or a salt, solvate, tautomer, stereoisomer, or isotopically labeled derivative thereof, with a compound of Formula (VIII):

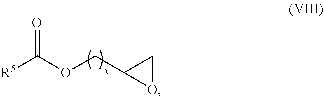

or a salt, solvate, tautomer, stereoisomer, or isotopically labeled derivative thereof, wherein:
each R is independently hydrogen, a nitrogen protecting group, or

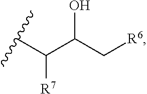

wherein one of R⁶ and R⁷ is hydrogen, and the other is

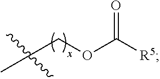

each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is

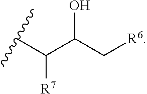

In another aspect, provided herein is a method of preparing a compound of Formula (XII′):

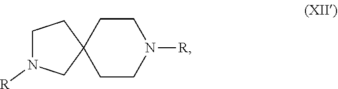

or a salt, solvate, tautomer, stereoisomer, or isotopically labeled derivative thereof, the method comprising reacting a compound of Formula (XII″):

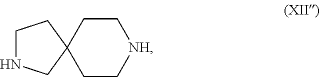

or a salt, solvate, tautomer, stereoisomer, or isotopically labeled derivative thereof, with a compound of Formula (VIII):

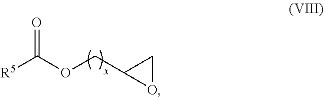

or a salt, solvate, tautomer, stereoisomer, or isotopically labeled derivative thereof, wherein:
each R is independently hydrogen, a nitrogen protecting group, or

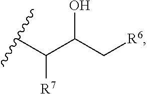

wherein one of R⁶ and R⁷ is hydrogen, and the other is

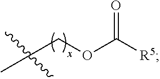

each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive;
provided that at least one R is

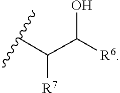

In another aspect, provided herein is a method of preparing a compound of Formula (XIII′):

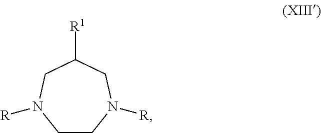

or a salt, solvate, tautomer, stereoisomer, or isotopically labeled derivative thereof, the method comprising reacting a compound of Formula (XIII″):

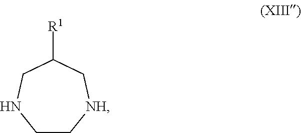

or a salt, solvate, tautomer, stereoisomer, or isotopically labeled derivative thereof, with a compound of Formula (VIII):

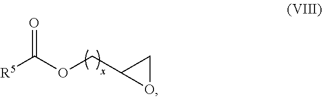

or a salt, solvate, tautomer, stereoisomer, or isotopically labeled derivative thereof, wherein:
R¹ is hydrogen, —OR^{A}, —SR^{A}, or —N(R^{A})₂,
each instance of R^{A} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, an oxygen protecting group when attached to an oxygen atom, a nitrogen protecting group when attached to a nitrogen atom, or a sulfur protecting group when attached to a sulfur atom, or wherein two instances of R^{A} attached to the same nitrogen atom are joined together with the intervening atoms to form optionally substituted heterocyclyl or optionally substituted heteroaryl;
each R is independently hydrogen, a nitrogen protecting group, or

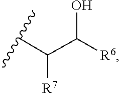

wherein one of R⁶ and R⁷ is hydrogen, and the other is

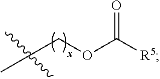

each instance of R⁵ is independently optionally substituted C₄-C₃₅ saturated or unsaturated aliphatic or optionally substituted C₄-C₃₅ saturated or unsaturated heteroaliphatic; and
each x independently is an integer from 1 to 10, inclusive; provided that at least one R is

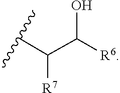

In another aspect, provided herein is a method of preparing a compound of Formula (VIII):

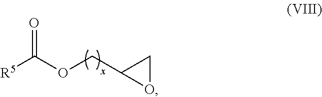

or a salt, solvate, tautomer, stereoisomer, or isotopically labeled derivative thereof, the method comprising reacting a compound of Formula (IX):

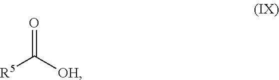

or a salt, solvate, tautomer, stereoisomer, or isotopically labeled derivative thereof, with a carbodiimide coupling agent, or a salt, solvate, tautomer, stereoisomer, or isotopically labeled derivative thereof, and a compound of Formula (X):

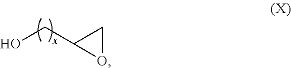

or a salt, solvate, tautomer, stereoisomer, or isotopically labeled derivative thereof, wherein:
x is an integer from 1 to 10, inclusive; and
R⁵ is optionally substituted C₄-C₃₅ unsaturated aliphatic or optionally substituted C₄-C₃₅ unsaturated heteroaliphatic.

In a particular embodiment, provided herein is a method of preparing a compound of Formula (VIII), or a salt thereof. In some embodiments, the method comprises reacting a compound of Formula (IX), or a salt thereof, with a carbodiimide coupling agent, or a salt thereof, and a compound of Formula (X), or a salt thereof.

In some embodiments, an excess of the compound of Formula (VIII) is used. In some embodiments, the excess of the compound of Formula (VIII) is one equivalent in excess of the number of reactive amines per compound of Formula (VII), or a salt thereof. In some embodiments, the excess of the compound of Formula (VIII) is five equivalents relative to the compound of Formula (VII), or a salt thereof.

In some embodiments, the reaction is performed under microwave radiation.

In some embodiments, the reaction is performed in a polar solvent. In some embodiments, the reaction is performed in a polar protic solvent. In some embodiments, the reaction is performed in an alcohol. In some embodiments, the reaction is performed in methanol, ethanol, n-propanol, or isopropanol. In some embodiments, the reaction is performed in isopropanol.

In some embodiments, the reaction is performed at about 50-130° C. In some embodiments, the reaction is performed at least 50° C. In some embodiments, the reaction is performed at least 60° C. In some embodiments, the reaction is performed at least 70° C. In some embodiments, the reaction is performed at least 80° C. In some embodiments, the reaction is performed at least 85° C. In some embodiments, the reaction is performed at about 80-100° C. In some embodiments, the reaction is performed at about 85-95° C. In some embodiments, the reaction is performed at about 90° C.

In some embodiments, the method further comprises a purification step. In some embodiments, the method further comprises chromatographic purification. In some embodiments, the chromatographic purification step comprises silica gel column chromatography.

In some embodiments, the method further comprises an isolation step. In some embodiments, the method further comprises a step of isolating the compound of Formula (I′), or a salt thereof. In some embodiments, the method further comprises a step of isolating the compound of Formula (VIII).

In some embodiments, the method further comprises a deprotection step. In some embodiments, the deprotection step is performed as an initial step. In some embodiments, the deprotection step is performed simultaneously to the reaction.

In some embodiments, the carbodiimide coupling agent is 1-ethyl-3-(3-dimethylaminopropyl)carbodiimide, dicyclohexylcarbodiimide, or diisopropylcarbodiimide, or a salt thereof. In some embodiments, the carbodiimide coupling agent is 1-ethyl-3-(3-dimethylaminopropyl)carbodiimide, dicyclohexylcarbodiimide, or diisopropylcarbodiimide, or a hydrochloride salt thereof. In some embodiments, the carbodiimide coupling agent is 1-ethyl-3-(3-dimethylaminopropyl)carbodiimide, dicyclohexylcarbodiimide, or diisopropylcarbodiimide, or a hydroiodide salt thereof. In some embodiments, the carbodiimide coupling agent is 1-ethyl-3-(3-dimethylaminopropyl)carbodiimide, or a salt thereof. In some embodiments, the carbodiimide coupling agent is 1-ethyl-3-(3-dimethylaminopropyl)carbodiimide hydrochloride. In some embodiments, the carbodiimide coupling agent is dicyclohexylcarbodiimide, or a salt thereof. In some embodiments, the carbodiimide coupling agent is diisopropylcarbodiimide, or a salt thereof.

As defined herein, R¹ is hydrogen, —OR^{A}, —SR^{A}, or —N(R^{A})₂. In some embodiments, R¹ is hydrogen or —OR^{A}. In some embodiments, R¹ is hydrogen or —OH. In some embodiments, R¹ is hydrogen or —SR^{A}. In some embodiments, R¹ is hydrogen or —SH. In some embodiments, R¹ is hydrogen or —N(R^{A})₂. In some embodiments, R¹ is hydrogen or —NH₂. In some embodiments, R¹ is hydrogen, —OR^{A}, or —SR^{A}. In some embodiments, R¹ is hydrogen, —OH, or —SH. In some embodiments, R¹ is —OR^{A}, —SR^{A}, or —N(R^{A})₂. In some embodiments, R¹ is hydrogen. In some embodiments, R¹ is —OR^{A}. In some embodiments, R¹ is —OH. In some embodiments, R¹ is —SR^{A}. In some embodiments, R¹ is —SH. In some embodiments, R¹ is —N(R^{A})₂. In some embodiments, R¹ is —NH₂.

As defined herein, each instance of R^{A} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, an oxygen protecting group when attached to an oxygen atom, a nitrogen protecting group when attached to a nitrogen atom, or a sulfur protecting group when attached to a sulfur atom, or wherein two instances of R^{A} attached to the same nitrogen atom are joined together with the intervening atoms to form optionally substituted heterocyclyl or optionally substituted heteroaryl. In some embodiments, R^{A} is —H. In some embodiments, R^{A} is optionally substituted C₁₋₁₀ aliphatic. In some embodiments, R^{A} is optionally substituted C₁₋₁₀ alkyl. In some embodiments, R^{A} is optionally substituted C₁₋₁₀ alkenyl. In some embodiments, R^{A} is optionally substituted C₁₋₁₀ alkynyl. In some embodiments, R^{A} is optionally substituted C₁₋₁₀ heteroaliphatic. In some embodiments, R^{A} is optionally substituted C₁₋₁₀ heteroalkyl. In some embodiments, R^{A} is optionally substituted C₁₋₁₀ heteroalkenyl. In some embodiments, R^{A} is optionally substituted C₁₋₁₀ heteroalkynyl. In some embodiments, R^{A} is optionally substituted acyl. In some embodiments, R^{A} is optionally substituted aryl. In some embodiments, R^{A} is optionally substituted phenyl. In some embodiments, R^{A} is optionally substituted heteroaryl. In some embodiments, R^{A} is optionally substituted 5- or 6-membered heteroaryl. In some embodiments, R^{A} is optionally substituted carbocyclyl. In some embodiments, R^{A} is optionally substituted 3- to 8-membered carbocyclyl. In some embodiments, R^{A} is optionally substituted heterocyclyl. In some embodiments, R^{A} is optionally substituted 3- to 8-membered heterocyclyl. In some embodiments, R^{A} is an oxygen protecting group. In some embodiments, R^{A} is a sulfur protecting group. In some embodiments, R^{A} is a nitrogen protecting group. In some embodiments, two instances of R^{A} attached to the same nitrogen atom are joined together with the intervening atoms to form optionally substituted heterocyclyl. In some embodiments, two instances of R^{A} attached to the same nitrogen atom are joined together with the intervening atoms to form optionally substituted heteroaryl.

As defined herein, n is 1, 2, or 3. In some embodiments, n is 1 or 2. In some embodiments, n is 1 or 3. In some embodiments, n is 2 or 3. In some embodiments, n is 1. In some embodiments, n is 2. In some embodiments, n is 3.

As defined herein, A and B are independently optionally substituted saturated or unsaturated aliphatic; or A and B combine to form optionally substituted C₂-C₆ alkylene. In some embodiments, A and B are the same. In some embodiments, A and B are independently optionally substituted saturated or unsaturated aliphatic. In some embodiments, A and B are independently substituted saturated or unsaturated aliphatic. In some embodiments, A and B are independently substituted saturated aliphatic. In some embodiments, A and B are independently substituted unsaturated aliphatic. In some embodiments, A and B are independently unsubstituted saturated or unsaturated aliphatic. In some embodiments, A and B are independently unsubstituted saturated aliphatic. In some embodiments, A and B are independently unsubstituted unsaturated aliphatic.

In some embodiments, A and B are independently optionally substituted C₁-C₆ saturated or unsaturated aliphatic. In some embodiments, A and B are independently substituted C₁-C₆ saturated or unsaturated aliphatic. In some embodiments, A and B are independently substituted C₁-C₆ saturated aliphatic. In some embodiments, A and B are independently substituted C₁-C₆ unsaturated aliphatic. In some embodiments, A and B are independently unsubstituted C₁-C₆ saturated or unsaturated aliphatic. In some embodiments, A and B are independently unsubstituted C₁-C₆ saturated aliphatic. In some embodiments, A and B are independently unsubstituted C₁-C₆ unsaturated aliphatic.

In some embodiments, A and B are independently optionally substituted alkyl. In some embodiments, A and B are independently substituted alkyl. In some embodiments, A and B are independently unsubstituted alkyl. In some embodiments, A and B are independently optionally substituted C₁-C₂₀ alkyl. In some embodiments, A and B are independently substituted C₁-C₂₀ alkyl. In some embodiments, A and B are independently unsubstituted C₁-C₂₀ alkyl. In some embodiments, A and B are independently optionally substituted C₁-C₆ alkyl. In some embodiments, A and B are independently substituted C₁-C₆ alkyl. In some embodiments, A and B are independently unsubstituted C₁-C₆ alkyl. In some embodiments, A and B are independently —CH₃.

In some embodiments, A and B combine to form optionally substituted C₂-C₆ alkylene. In some embodiments, A and B combine to form optionally substituted branched C₂-C₆ alkylene. In some embodiments, A and B combine to form optionally substituted unbranched C₂-C₆ alkylene. In some embodiments, A and B combine to form optionally substituted ethylene. In some embodiments, A and B combine to form optionally substituted n-propylene. In some embodiments, A and B combine to form optionally substituted n-butylene. In some embodiments, A and B combine to form optionally substituted n-pentylene. In some embodiments, A and B combine to form optionally substituted n-hexylene.

In some embodiments, A and B combine to form substituted C₂-C₆ alkylene. In some embodiments, A and B combine to form substituted branched C₂-C₆ alkylene. In some embodiments, A and B combine to form substituted unbranched C₂-C₆ alkylene. In some embodiments, A and B combine to form unsubstituted C₂-C₆ alkylene. In some embodiments, A and B combine to form unsubstituted branched C₂-C₆ alkylene. In some embodiments, A and B combine to form unsubstituted unbranched C₂-C₆ alkylene. In some embodiments, A and B combine to form ethylene. In some embodiments, A and B combine to form n-propylene. In some embodiments, A and B combine to form n-butylene. In some embodiments, A and B combine to form n-pentylene. In some embodiments, A and B combine to form n-hexylene.

As defined herein, each R is independently hydrogen, a nitrogen protecting group, or

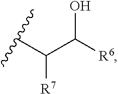

wherein one of R⁶ and R⁷ is hydrogen, and the other is

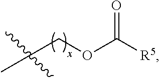

provided that at least one R is

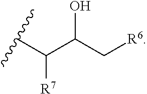

In some embodiments, each R is

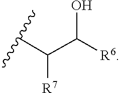

In some embodiments, one R is

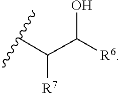

In some embodiments, two R are independently

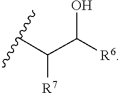

In some embodiments, three R are independently

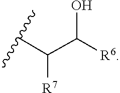

In some embodiments, four R are independently

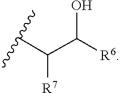

In some embodiments, five R are independently

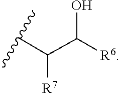

In some embodiments, six R are independently

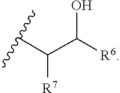

In some embodiments, seven R are independently

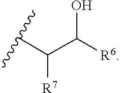

In some embodiments, eight R are independently

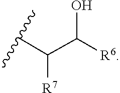

As defined herein, for each moiety

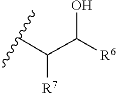

individually, one of R⁶ and R⁷ is hydrogen, and the other is

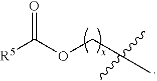

In some embodiments, each R⁶ is hydrogen. In some embodiments, each R⁷ is

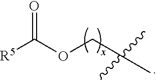

In some embodiments, each moiety

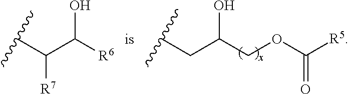

In some embodiments, each R is

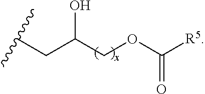

As defined herein, each instance of R⁵ is independently optionally substituted C₄-C₃₅ saturated or unsaturated aliphatic or optionally substituted C₄-C₃₅ saturated or unsaturated heteroaliphatic.

In some embodiments, each instance of R⁵ is independently optionally substituted C₄-C₃₅ saturated aliphatic or optionally substituted C₄-C₃₅ saturated heteroaliphatic. In some embodiments, each instance of R⁵ is independently optionally substituted C₄-C₃₅ unsaturated aliphatic or optionally substituted C₄-C₃₅ unsaturated heteroaliphatic.

In some embodiments, each instance of R⁵ is independently optionally substituted C₄-C₃₅ saturated or unsaturated aliphatic. In some embodiments, each instance of R⁵ is independently optionally substituted C₄-C₁₂ saturated or unsaturated aliphatic. In some embodiments, each instance of R⁵ is independently optionally substituted C₁₀-C₂₀ saturated or unsaturated aliphatic. In some embodiments, each instance of R⁵ is independently optionally substituted C₁₂-C₃₅ saturated or unsaturated aliphatic.

In some embodiments, each R⁵ is independently optionally substituted C₄-C₃₅ alkyl or optionally substituted C₄-C₃₅ alkenyl. In some embodiments, each R⁵ is independently optionally substituted C₄-C₁₀ alkyl or optionally substituted C₄-C₁₀ alkenyl. In some embodiments, each R⁵ is independently optionally substituted C₁₀-C₂₀ alkyl or optionally substituted C₁₀-C₂₀ alkenyl. In some embodiments, each R⁵ is independently optionally substituted C₁₂-C₂₅ alkyl or optionally substituted C₁₂-C₂₅ alkenyl.

In some embodiments, each R⁵ is independently branched C₄-C₃₅ alkyl. In some embodiments, each R⁵ is independently branched C₄-C₁₂ alkyl. In some embodiments, each R⁵ is independently branched C₁₀-C₂₀ alkyl. In some embodiments, each R⁵ is independently branched C₁₂-C₂₅ alkyl. In some embodiments, each R⁵ is independently unbranched C₄-C₃₅ alkyl. In some embodiments, each R⁵ is independently unbranched C₄-C₁₂ alkyl. In some embodiments, each R⁵ is independently unbranched C₁₀-C₂₀ alkyl. In some embodiments, each R⁵ is independently unbranched C₁₂-C₂₅ alkyl.

In some embodiments, each R⁵ is independently branched C₄-C₃₅ alkenyl. In some embodiments, each R⁵ is independently branched C₄-C₃₅ alkenyl comprising a single double bond. In some embodiments, each R⁵ is independently branched C₄-C₃₅ alkenyl comprising a single (Z) double bond. In some embodiments, each R⁵ is independently branched C₄-C₁₂ alkenyl. In some embodiments, each R⁵ is independently branched C₁₀-C₂₀ alkenyl. In some embodiments, each R⁵ is independently branched C₁₂-C₂₅ alkenyl.

In some embodiments, each R⁵ is independently unbranched C₄-C₃₅ alkenyl. In some embodiments, each R⁵ is independently unbranched C₄-C₃₅ alkenyl comprising a single double bond. In some embodiments, each R⁵ is independently unbranched C₄-C₃₅ alkenyl comprising a single (Z) double bond. In some embodiments, each R⁵ is independently unbranched C₄-C₃₅ alkenyl comprising two double bonds. In some embodiments, each R⁵ is independently unbranched C₄-C₃₅ alkenyl comprising two (Z) double bonds. In some embodiments, each R⁵ is independently unbranched C₄-C₃₅ alkenyl comprising

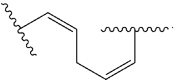 [SMILES: */C=C\C/C=C\*]

In some embodiments, each R⁵ is independently unbranched C₄-C₃₅ alkenyl comprising three double bonds. In some embodiments, each R⁵ is independently unbranched C₄-C₃₅ alkenyl comprising three (Z) double bonds. In some embodiments, each R⁵ is independently unbranched C₄-C₃₅ alkenyl comprising only (Z) double bonds.

In some embodiments, each R⁵ is independently unbranched C₁₀-C₂₀ alkenyl. In some embodiments, each R⁵ is independently unbranched C₁₀-C₂₀ alkenyl comprising a single double bond. In some embodiments, each R⁵ is independently unbranched C₁₀-C₂₀ alkenyl comprising a single (Z) double bond. In some embodiments, each R⁵ is independently unbranched C₁₀-C₂₀ alkenyl comprising two double bonds. In some embodiments, each R⁵ is independently unbranched C₁₀-C₂₀ alkenyl comprising two (Z) double bonds. In some embodiments, each R⁵ is independently unbranched C₁₀-C₂₀ alkenyl comprising

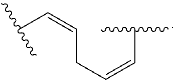 [SMILES: */C=C\C/C=C\*]

In some embodiments, each R⁵ is independently unbranched C₁₀-C₂₀ alkenyl comprising three double bonds. In some embodiments, each R⁵ is independently unbranched C₁₀-C₂₀ alkenyl comprising three (Z) double bonds. In some embodiments, each R⁵ is independently unbranched C₁₀-C₂₀ alkenyl comprising only (Z) double bonds.

In some embodiments, each R⁵ is independently unbranched C₁₁ alkenyl. In some embodiments, each R⁵ is independently unbranched C₁₁ alkenyl comprising a single double bond. In some embodiments, each R⁵ is independently unbranched C₁₁ alkenyl comprising a single (Z) double bond. In some embodiments, each R⁵ is independently unbranched C₁₁ alkenyl comprising two double bonds. In some embodiments, each R⁵ is independently unbranched C₁₁ alkenyl comprising two (Z) double bonds. In some embodiments, each R⁵ is independently unbranched C₁₁ alkenyl comprising

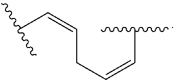 [SMILES: */C=C\C/C=C\*]

In some embodiments, each R⁵ is independently unbranched C₁₁ alkenyl comprising three double bonds. In some embodiments, each R⁵ is independently unbranched C₁₁ alkenyl comprising three (Z) double bonds. In some embodiments, each R⁵ is independently unbranched C₁₁ alkenyl comprising only (Z) double bonds.

In some embodiments, each instance of R⁵ is independently optionally substituted C₄-C₃₅ saturated or unsaturated heteroaliphatic. In some embodiments, each instance of R⁵ is independently optionally substituted C₄-C₃₅ saturated or unsaturated heteroaliphatic comprising at least one O, N, or S atom. In some embodiments, each instance of R⁵ is independently optionally substituted C₄-C₃₅ saturated or unsaturated heteroaliphatic comprising at least one O or S atom. In some embodiments, each instance of R⁵ is independently optionally substituted C₄-C₁₂ saturated or unsaturated heteroaliphatic. In some embodiments, each instance of R⁵ is independently optionally substituted C₄-C₁₂ saturated or unsaturated heteroaliphatic comprising at least one O, N, or S atom. In some embodiments, each instance of R⁵ is independently optionally substituted C₄-C₁₂ saturated or unsaturated heteroaliphatic comprising at least one O or S atom. In some embodiments, each instance of R⁵ is independently optionally substituted C₁₀-C₂₀ saturated or unsaturated heteroaliphatic. In some embodiments, each instance of R⁵ is independently optionally substituted C₁₀-C₂₀ saturated or unsaturated heteroaliphatic comprising at least one O, N, or S atom. In some embodiments, each instance of R⁵ is independently optionally substituted C₁₀-C₂₀ saturated or unsaturated heteroaliphatic comprising at least one O or S atom. In some embodiments, each instance of R⁵ is independently optionally substituted C₁₂-C₂₅ saturated or unsaturated heteroaliphatic. In some embodiments, each instance of R⁵ is independently optionally substituted C₁₂-C₂₅ saturated or unsaturated heteroaliphatic comprising at least one O, N, or S atom. In some embodiments, each instance of R⁵ is independently optionally substituted C₁₂-C₂₅ saturated or unsaturated heteroaliphatic comprising at least one O or S atom.

In some embodiments, each R⁵ is independently branched C₄-C₃₅ heteroalkyl. In some embodiments, R⁵ is independently branched C₄-C₁₂ heteroalkyl. In some embodiments, each R⁵ is independently branched C₁₀-C₂₀ heteroalkyl. In some embodiments, each R⁵ is independently branched C₁₂-C₃₅ heteroalkyl. In some embodiments, each R⁵ is independently unbranched C₄-C₃₅ heteroalkyl. In some embodiments, each R⁵ is independently unbranched C₄-C₁₂ heteroalkyl. In some embodiments, each R⁵ is independently unbranched C₁₀-C₂₀ heteroalkyl. In some embodiments, each R⁵ is independently unbranched C₁₂-C₂₅ heteroalkyl.

In some embodiments, each R⁵ is independently selected from the group consisting of: (GC12), (GC14), (GC16), (GC18), (G41), (G45), (G46), (G47), (G48), (G49), (G50), (G51), (G52), (G53), 210/300

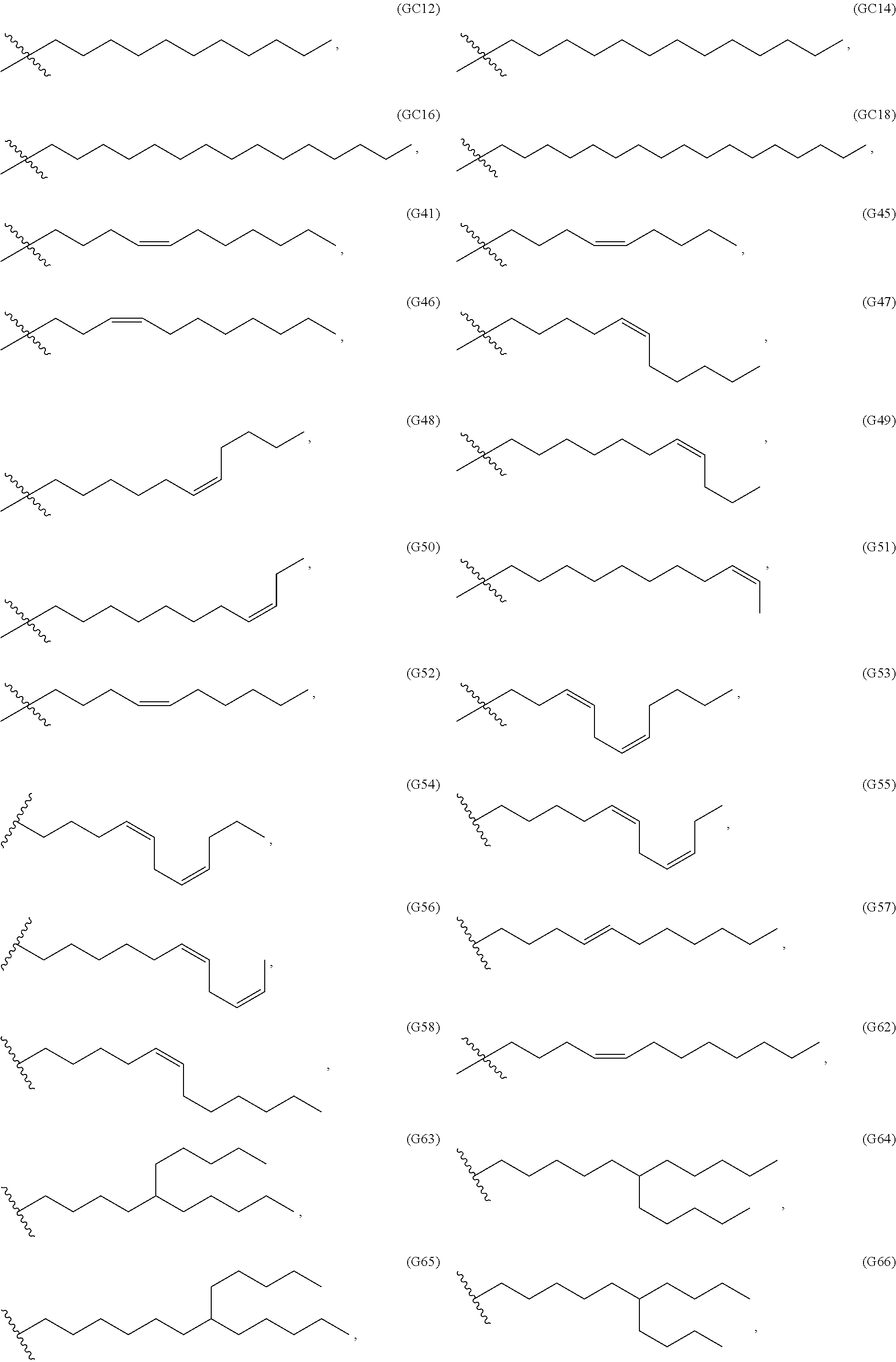

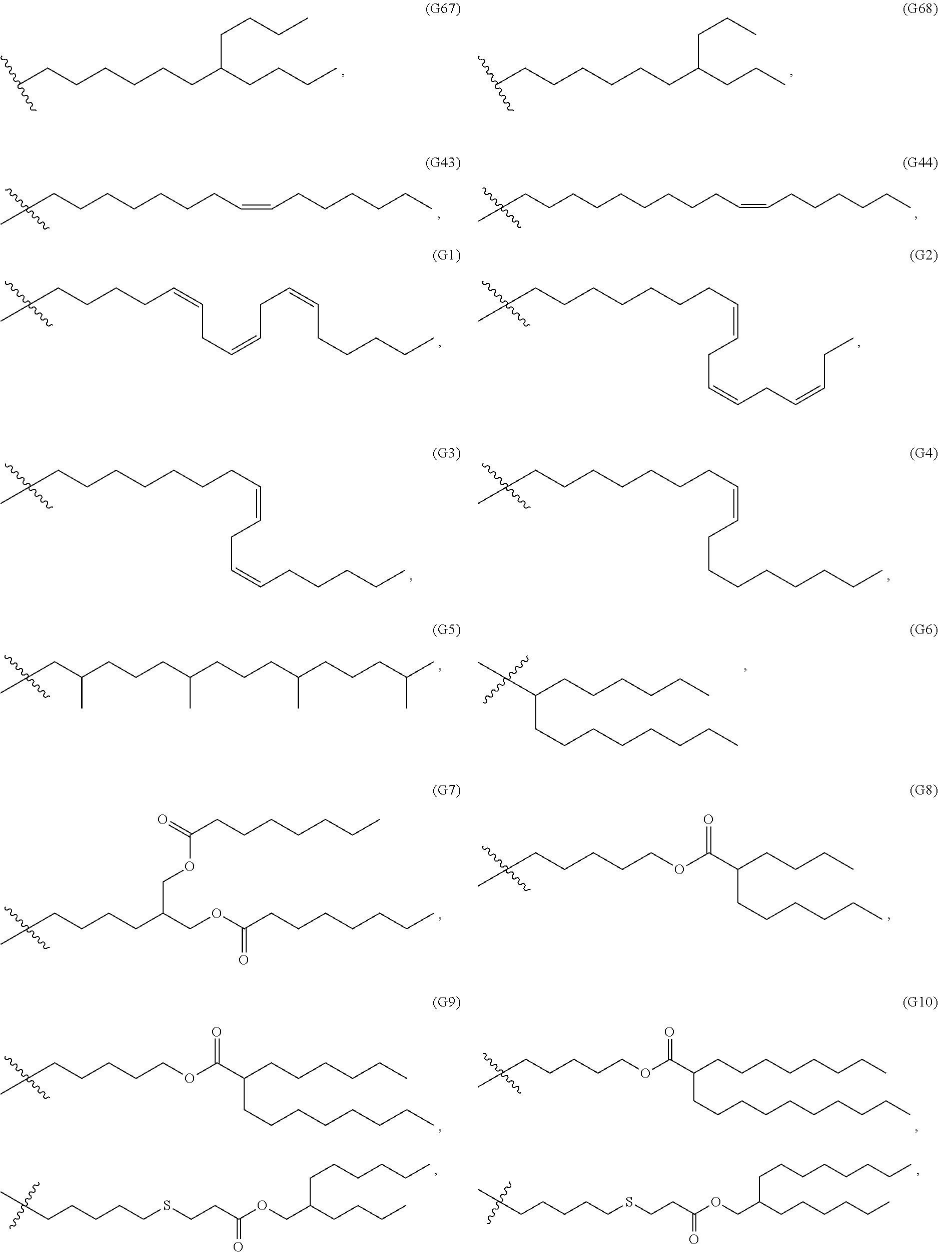

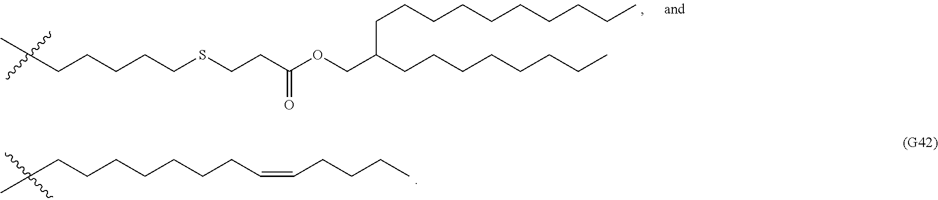 [SMILES: *CCCCCSCCC(=O)OCC(CCCCCCCC)CCCCCCCCCC ; *CCCCCCC/C=C\CCCC]

In some embodiments, each R⁵ is independently selected from the group consisting of:

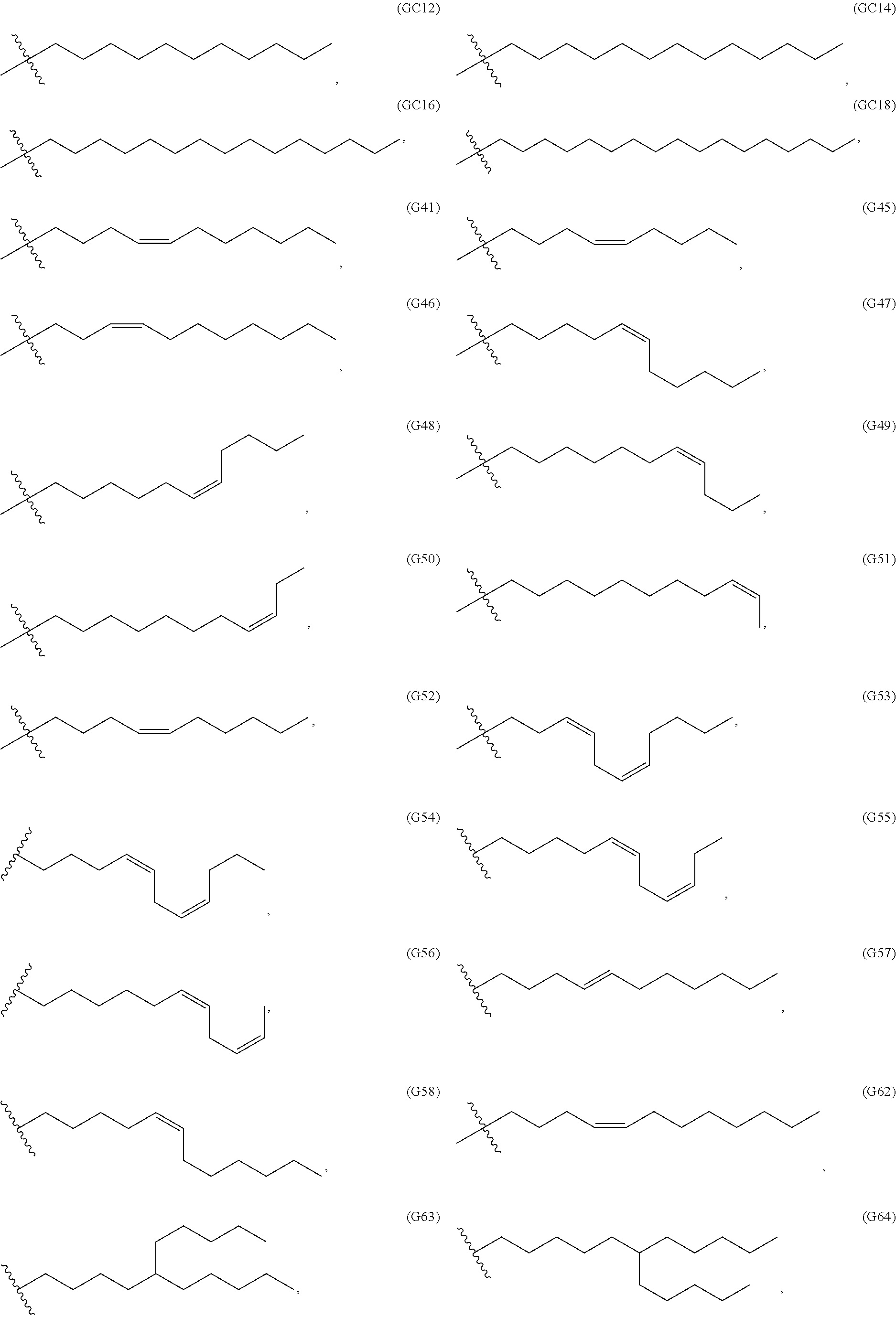

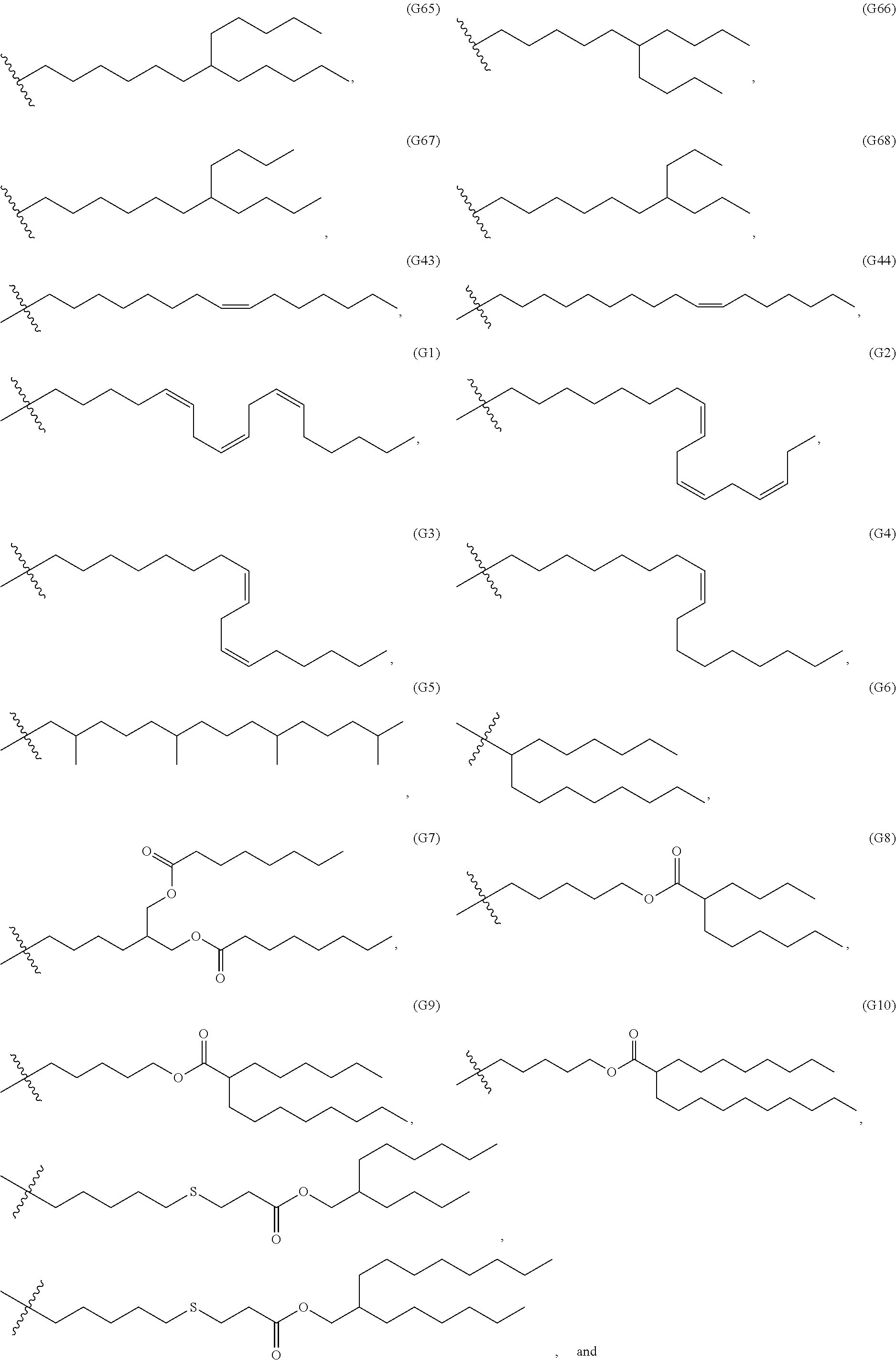

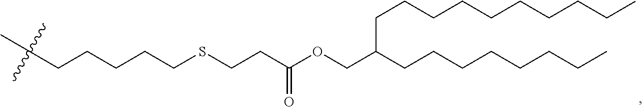 [SMILES: *CCCCCSCCC(=O)OCC(CCCCCCCC)CCCCCCCCCC]

In some embodiments, each R⁵ is independently selected from the group consisting of:

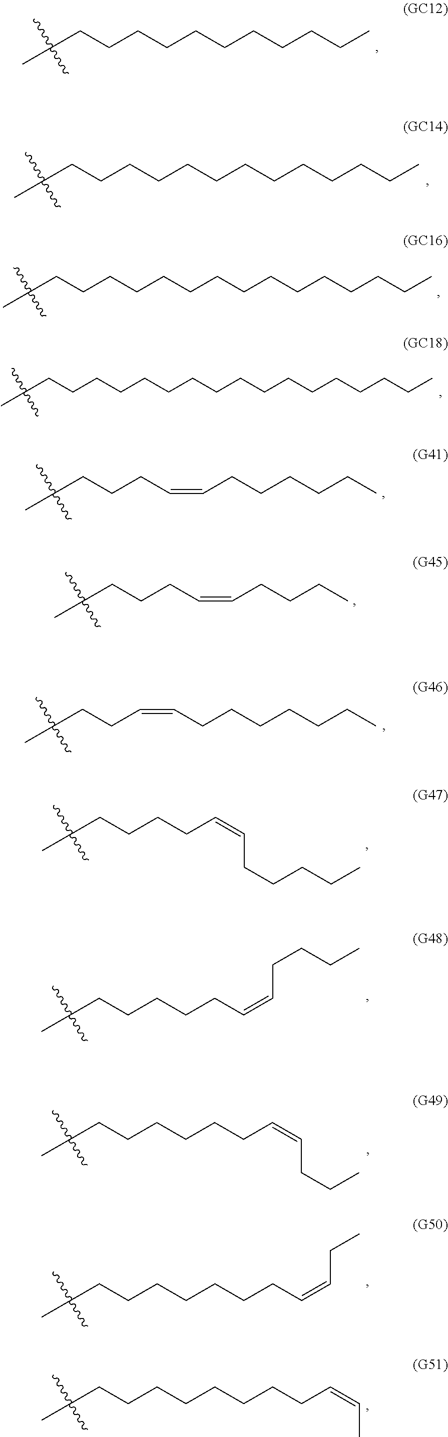

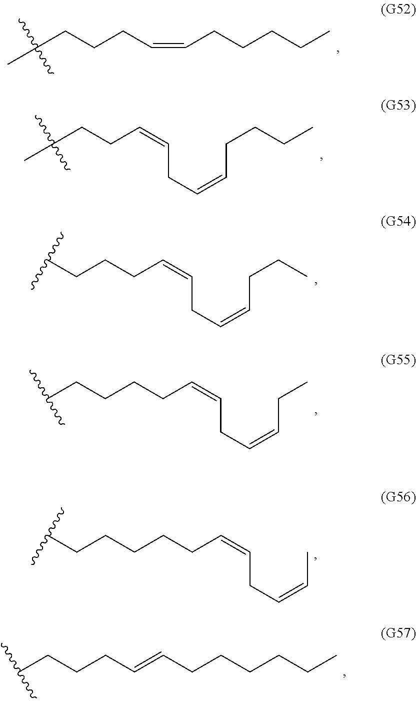 [SMILES: *CCC/C=C\CCCCC ; *CC/C=C\C/C=C\CCCC ; *CCC/C=C\C/C=C\CCC ; *CCCC/C=C\C/C=C\CC ; *CCCCC/C=C\C/C=C\C ; *CCC/C=C/CCCCCC]

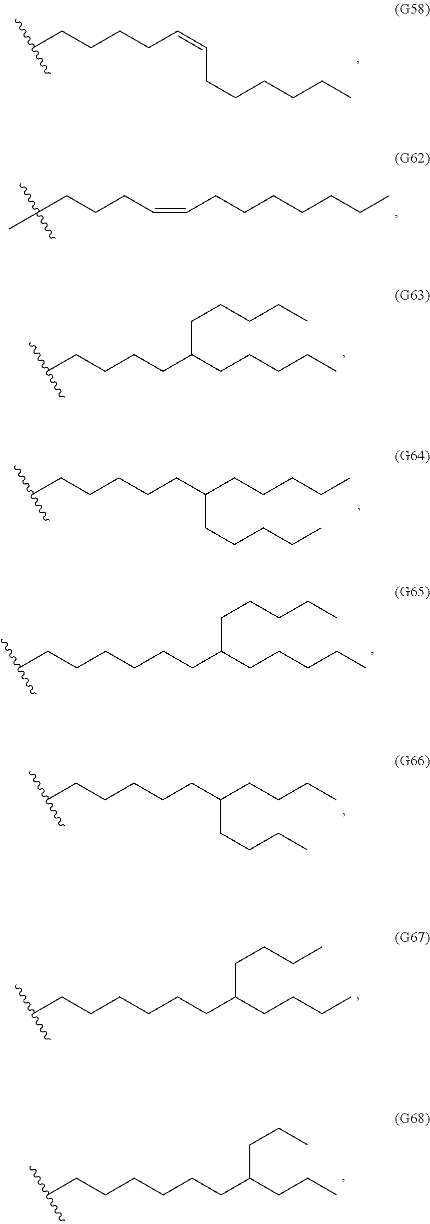

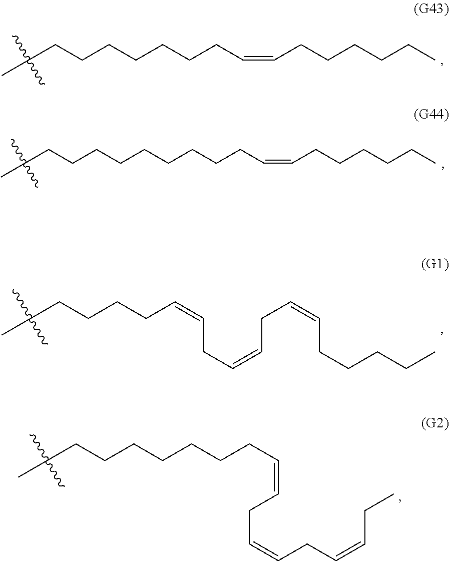 [SMILES: *CCCCCCC/C=C\CCCCCC ; *CCCCCCCCC/C=C\CCCCCC ; *CCCC/C=C\C/C=C\C/C=C\CCCCC ; *CCCCCCC/C=C\C/C=C\C/C=C\CC]

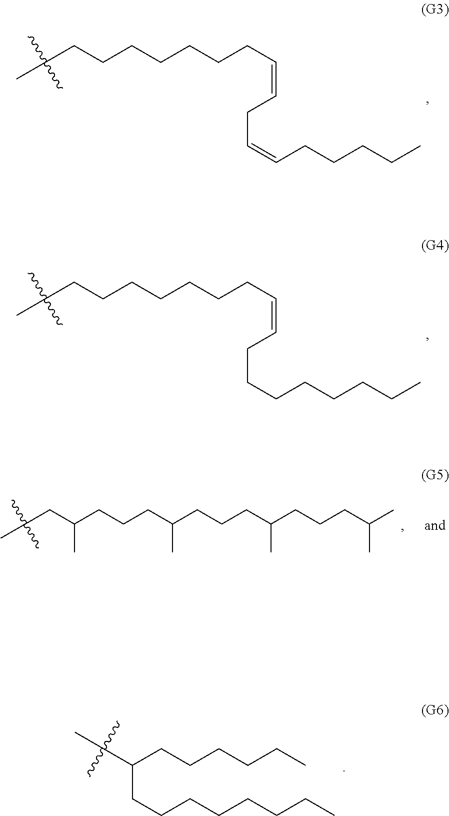 [SMILES: *CCCCCCC/C=C\C/C=C\CCCCC ; *CCCCCCC/C=C\CCCCCCCC ; *CC(C)CCCC(C)CCCC(C)CCCC(C)C ; *C(CCCCCC)CCCCCCCC]

In some embodiments, each R⁵ is independently selected from the group consisting of:

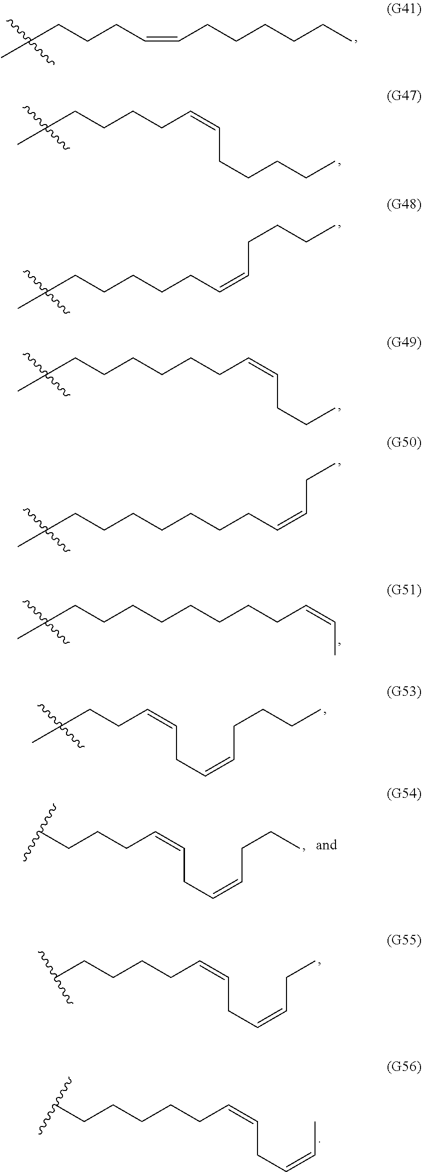

In some embodiments, R⁵ is

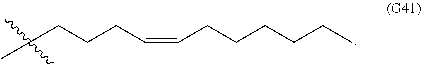 [SMILES: *CCC/C=C\CCCCCC]

In some embodiments, each R⁵ is independently selected from the group consisting of:

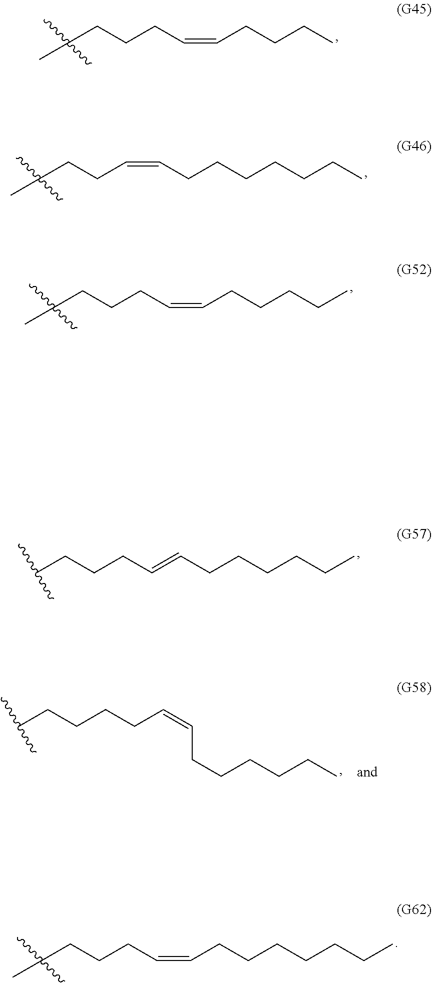

In some embodiments, each R⁵ is independently selected from the group consisting of:

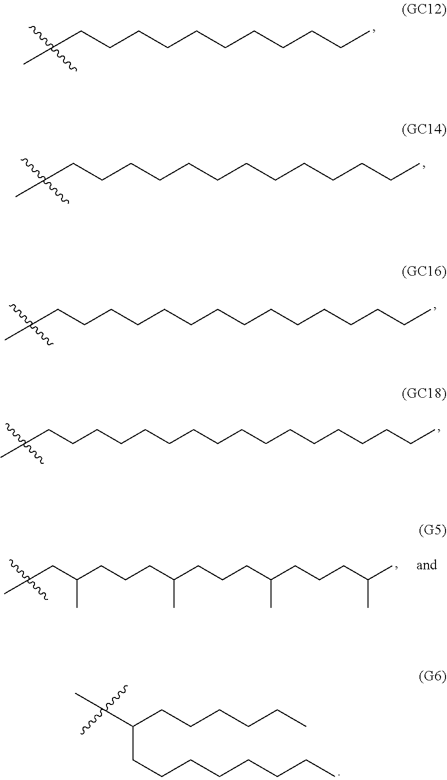 [SMILES: *CCCCCCCCCCC ; *CCCCCCCCCCCCC ; *CCCCCCCCCCCCCCC ; *CCCCCCCCCCCCCCCCC ; *CC(C)CCCC(C)CCCC(C)CCCC(C)C ; *C(CCCCCC)CCCCCCCC]

In some embodiments, each R⁵ is independently selected from the group consisting of:

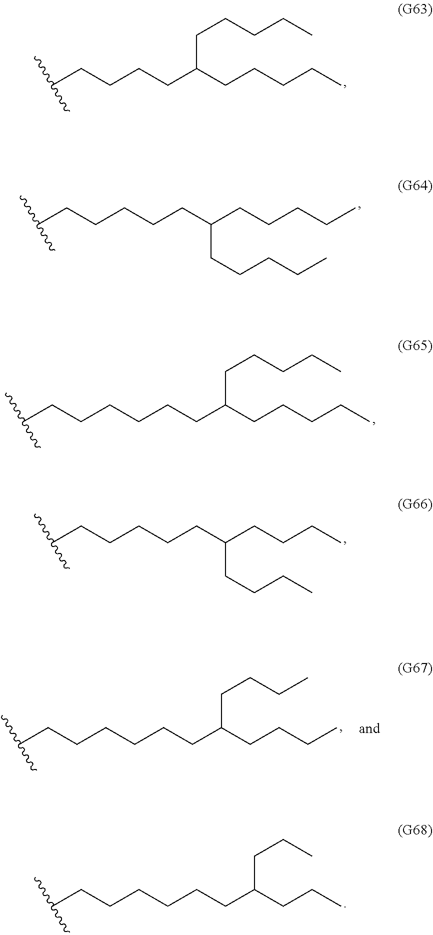 [SMILES: *CCCCC(CCCCC)CCCCC ; *CCCCCC(CCCCC)CCCCC ; *CCCCCCC(CCCCC)CCCCC ; *CCCCCC(CCCC)CCCC ; *CCCCCCC(CCCC)CCCC ; *CCCCCCC(CCC)CCC]

In some embodiments, each R⁵ is independently selected from the group consisting of:

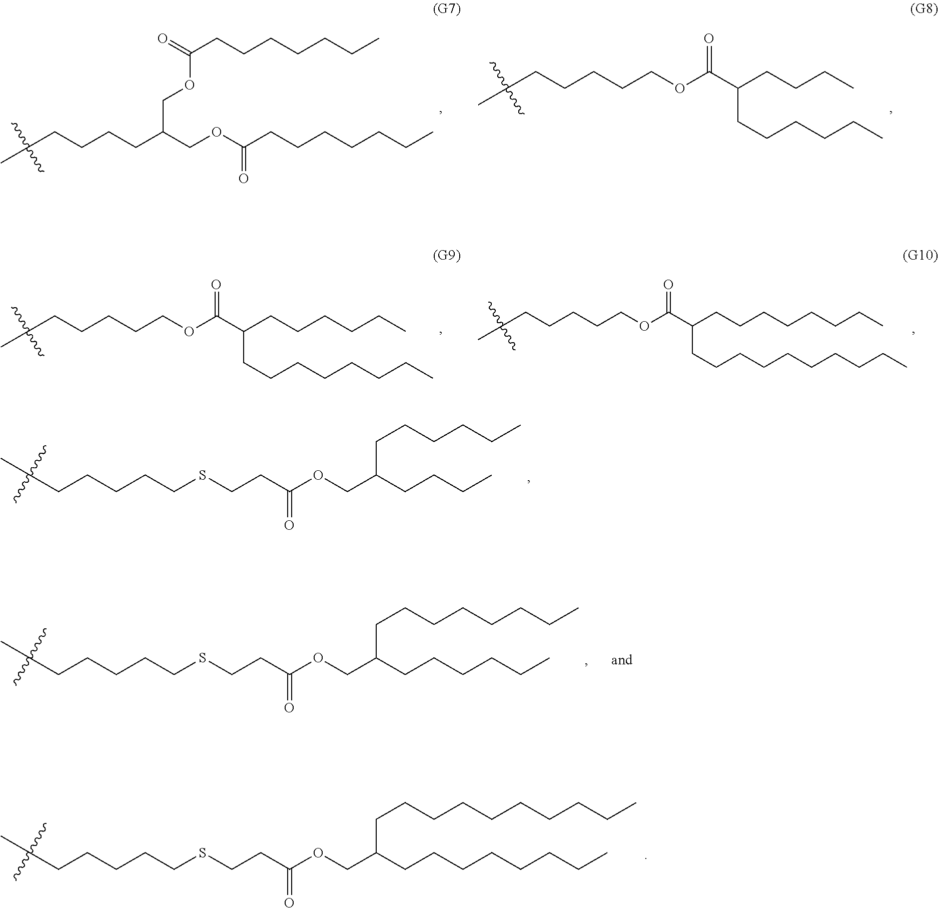

In a particular embodiment, R⁵ is G41. In a particular embodiment, R⁵ is G45. In a particular embodiment, R⁵ is G46. In a particular embodiment, R⁵ is G47. In a particular embodiment, R⁵ is G48. In a particular embodiment, R⁵ is G49. In a particular embodiment, R⁵ is G50. In a particular embodiment, R⁵ is G51. In a particular embodiment, R⁵ is G52. In a particular embodiment, R⁵ is G53. In a particular embodiment, R⁵ is G54. In a particular embodiment, R⁵ is G55. In a particular embodiment, R⁵ is G56. In a particular embodiment, R⁵ is G57. In a particular embodiment, R⁵ is G58. In a particular embodiment, R⁵ is G62. In a particular embodiment, R⁵ is G63. In a particular embodiment, R⁵ is G64. In a particular embodiment, R⁵ is G65. In a particular embodiment, R⁵ is G66. In a particular embodiment, R⁵ is G67. In a particular embodiment, R⁵ is G68. In a particular embodiment, R⁵ is GC12. In a particular embodiment, R⁵ is GC14. In a particular embodiment, R⁵ is GC16. In a particular embodiment, R⁵ is GC18. In a particular embodiment, R⁵ is G42. In a particular embodiment, R⁵ is G43. In a particular embodiment, R⁵ is G44. In a particular embodiment, R⁵ is G1. In a particular embodiment, R⁵ is G2. In a particular embodiment, R⁵ is G3. In a particular embodiment, R⁵ is G4. In a particular embodiment, R⁵ is G5. In a particular embodiment, R⁵ is G6. In a particular embodiment, R⁵ is G7. In a particular embodiment, R⁵ is G8. In a particular embodiment, R⁵ is G9. In a particular embodiment, R⁵ is G10. In a particular embodiment, R⁵ is

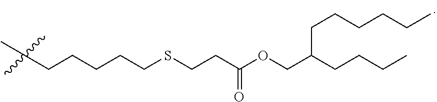 [SMILES: *CCCCCSCCC(=O)OCC(CCCC)CCCCCC]

In a particular embodiment, R⁵ is

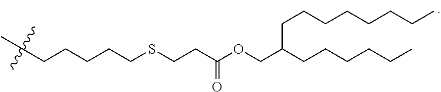 [SMILES: *CCCCCSCCC(=O)OCC(CCCCCC)CCCCCCCC]

In a particular embodiment, R⁵ is

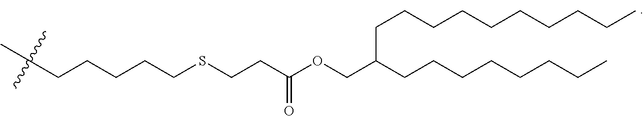 [SMILES: *CCCCCSCCC(=O)OCC(CCCCCCCC)CCCCCCCCCC]

As defined herein, each x independently is an integer from to 10, inclusive. In some embodiments, each x independently is an integer from 1 to 9, inclusive. In some embodiments, each x independently is an integer from 1 to 8, inclusive. In some embodiments, each x independently is an integer from 1 to 7, inclusive. In some embodiments, each x independently is an integer from 1 to 6, inclusive. In some embodiments, each x independently is an integer from 1 to 5, inclusive. In some embodiments, each x independently is an integer from 1 to 4, inclusive. In some embodiments, each x independently is an integer from 1 to 3, inclusive. In some embodiments, each x independently is an integer from 1 to 2, inclusive. In some embodiments, each x is the same. In some embodiments, six instances of x are the same. In some embodiments, seven instances of x are the same. In some embodiments, eight instances of x are the same. In some embodiments, x is 1. In some embodiments, each x is 1. In some embodiments, six x are 1. In some embodiments, seven x are 1. In some embodiments, eight x are 1.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Lipid Nanoparticles

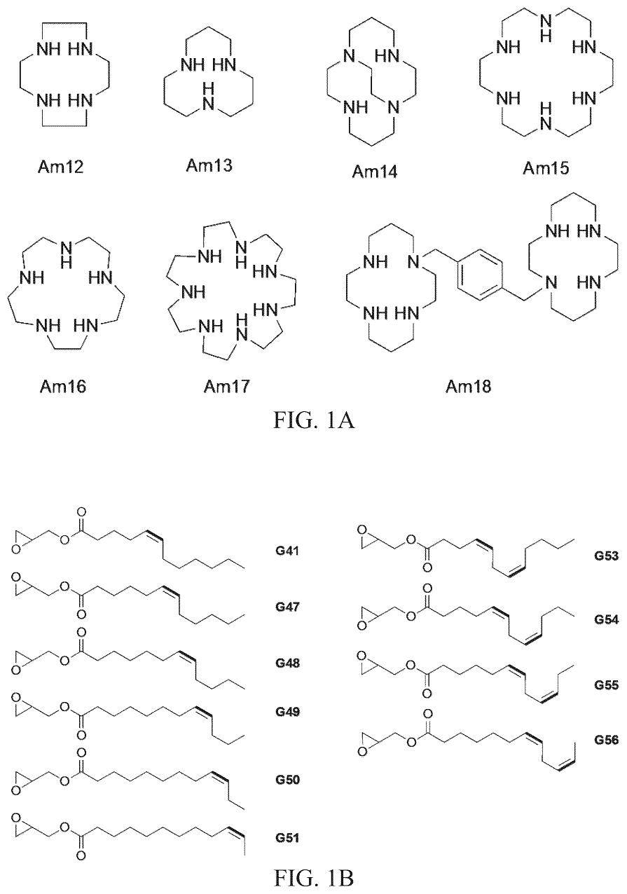

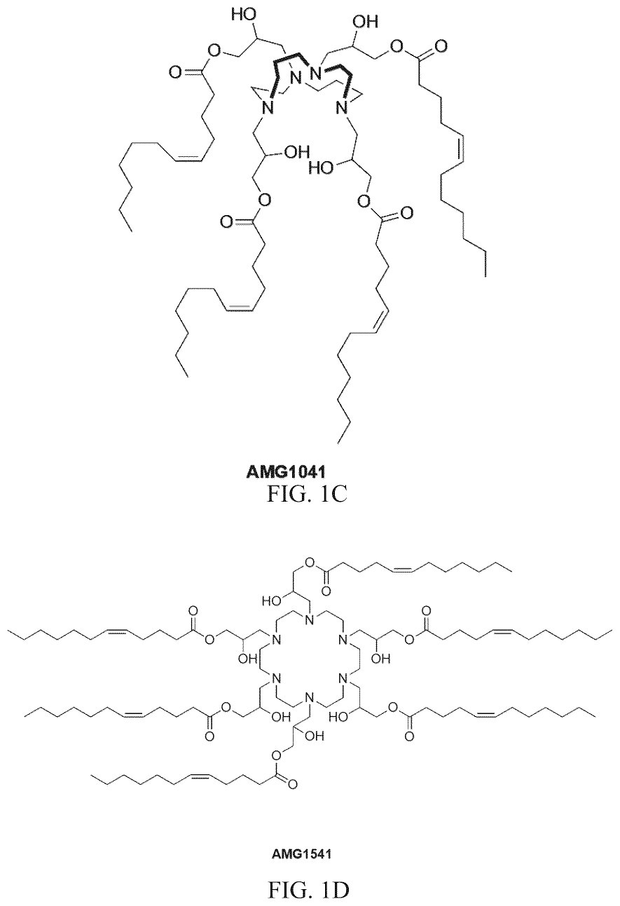

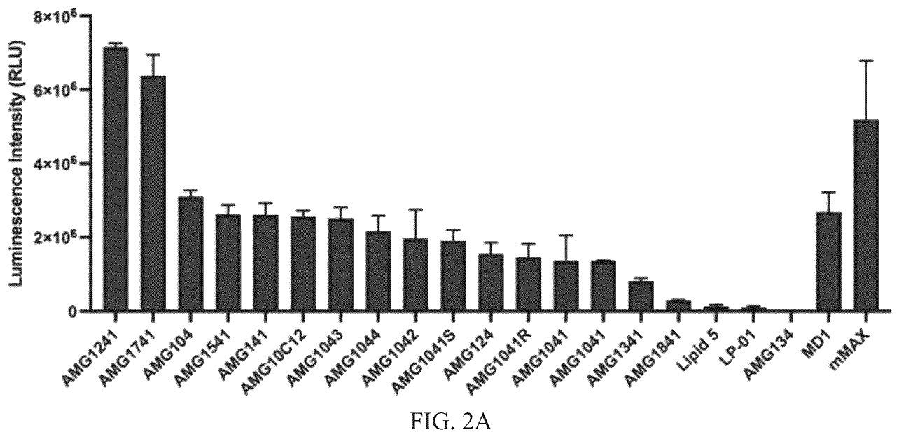

Nucleophilic ring opening of epoxides has been utilized for the synthesis of novel ionizable lipids for RNA delivery.¹¹ Previous work disclosed that lipid nanoparticles (LNPs) comprising a specific lipid, AMG1041 (FIG. 1C), showed superior protein production compared to FDA-approved lipid SM-102 when administered intramuscularly, and also showed high antibody titers in an immunogenicity experiment using mRNA expressing SARS-CoV-2 delta variant full spike protein. The chemical structure was further iteratively developed to generate a library of ionizable lipids, including ionizable lipid AMG1541 (FIGS. 1A-1D). AMG1541 showed superior protein production via intramuscular and intravenous administration. In addition, AMG1541 also produced high antibody titers against the SARS-CoV-2 delta variant, comparable to SM-102.

Synthesis of fatty acid epoxy esters. Additional lipids were generated by replacing the cyclam Am10 headgroup. The tail structure of AMG1041 was initially kept constant. Am12 and Am13 have fewer carbon and nitrogen atoms than Am10. Am14 has two nitrogen atoms bridged in cyclam Am10. Am15-Am17 have expanded ring structures from Am10. Am18 contains two Am10 headgroups linked together. The fatty acid epoxy ester G41 was synthesized by esterifying the corresponding fatty acid with glycidol.

Once AMG1541 turned out to be a potent lipid in protein expression as well as for inducing antibody titers against the full spike protein of SARS-CoV-2 or H3 protein of influenza virus, the chemical structure was further derivatized by moving the Z-alkene along the G41 tail length. This process is dubbed as the “alkene walking” strategy to improve performance. Moreover, an additional Z-alkene was added, spaced by a single methylene to prevent 7L conjugation, and then a similar “alkene walking” strategy was applied. The structures of the relevant tails are shown in FIG. 1B.

Synthesis of lipid nanoparticles (LNPs). LNPs for in vitro studies and intramuscular injections were prepared by microfluidic mixing. Briefly, the lipid components were dissolved in ethanol and, separately, mRNA encoding for firefly luciferase (fLuc) or the SARS-CoV-2 B.1.617.2 spike protein with 984P and 985P mutations was diluted into a solution of 10 mM citrate pH 3.0 at a concentration of 133 ng/μL. The two solutions were mixed in a 3:1 water:ethanol volume ratio as previously described.

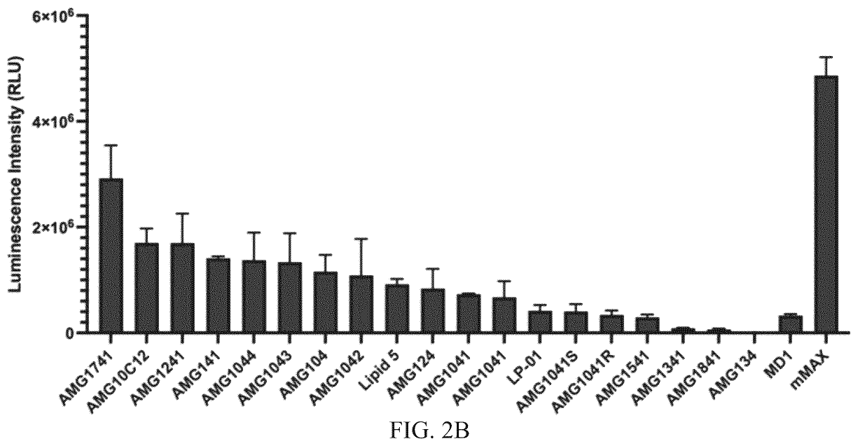

Evaluation of the LNPs in vitro. HeLa or DC2.4 cells were plated on a white, flat-bottom 96 well plate (Coming, Coming, NY) at a density of 15k cells/well and allowed to recover for 24 hrs. Each well was then dosed with 150 ng of fLuc mRNA LNPs, and, after 24 hrs., luciferase expression was determined using a Bright-Glo assay (Promega, Madison, WI). The transfection performance in each of the cell lines is shown in FIGS. 2A-2B.

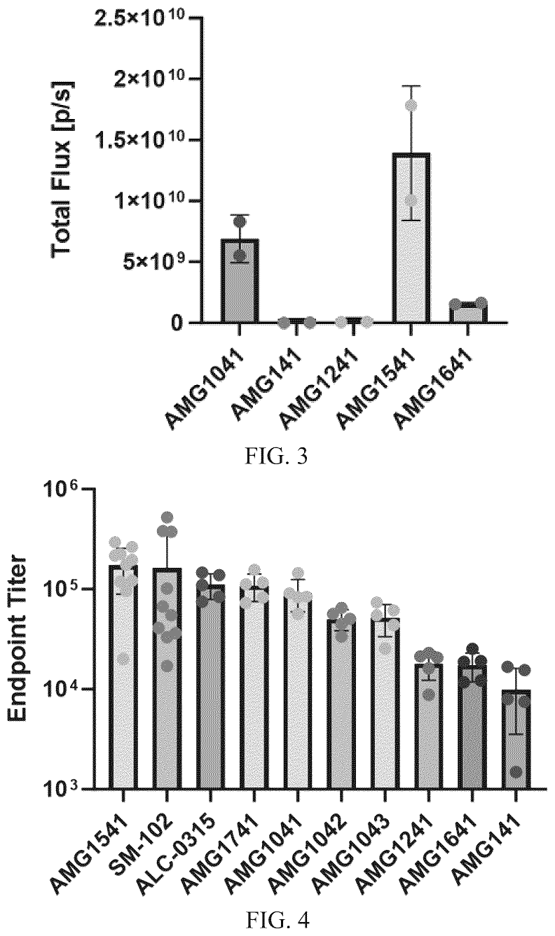

Evaluation of the LNPs via intramuscular injection. The in vivo potency of the ionizable lipids was screened through intramuscular injection of LNPs loaded with mRNA encoding firefly luciferase protein. These LNPs were formulated with a molar composition of (35:16:46.5:2.5) using one of the synthesized ionizable lipids, DOPE as a helper lipid, cholesterol, and DMPE-PEG respectively. mRNA-loaded LNPs were intramuscularly administered in mice at a dose of 1 μg, and expression of luciferase protein was monitored at 6 hours via IVIS bioluminescent imaging (FIG. 3). LNPs formulated with lipid AMG1541 showed approximately 2-fold higher fLuc expression than LNPs formulated with AMG1041, a previously-identified lipid which gives high levels of protein expression. Without wishing to be bound by theory, increased levels of protein expression may enhance the response to vaccination.

Based on the data from LNPs formulated with AMG1541, the immunogenicity elicited by these lipids was compared. Their vaccination potency was also compared against the two clinically approved mRNA LNPs used in the COVID-19 mRNA vaccines—SM-102 and ALC-0315. Briefly, 6-8 week old BALB/c mice were vaccinated with 1 g of mRNA LNPs on day 0 and on day 21, and serum was collected on day 35 to characterize the antibody response to vaccination. Antibody titers are shown in FIG. 4. AMG1541 elicited the highest antibody titers after vaccination, with titers similar to or higher than SM-102 and ALC-0315. AMG1741 and AMG1041 also elicited high levels of antibody titers.

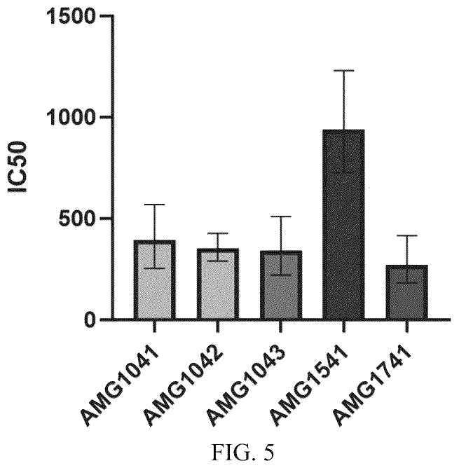

The ability of the antibody-containing serum to prevent infection was also characterized using a pseudovirus neutralization assay. Lentiviruses pseudotyped with the B.1.617.2 spike protein and containing a GFP reporter gene were incubated with serum samples prior to addition to 293T-ACE2 cells. Infection inhibition IC50 values from this assay are shown in FIG. 5. In agreement with the antibody titers, AMG1541 induced robust neutralization with an IC50 approximately 2-fold higher than lipid AMG1041.

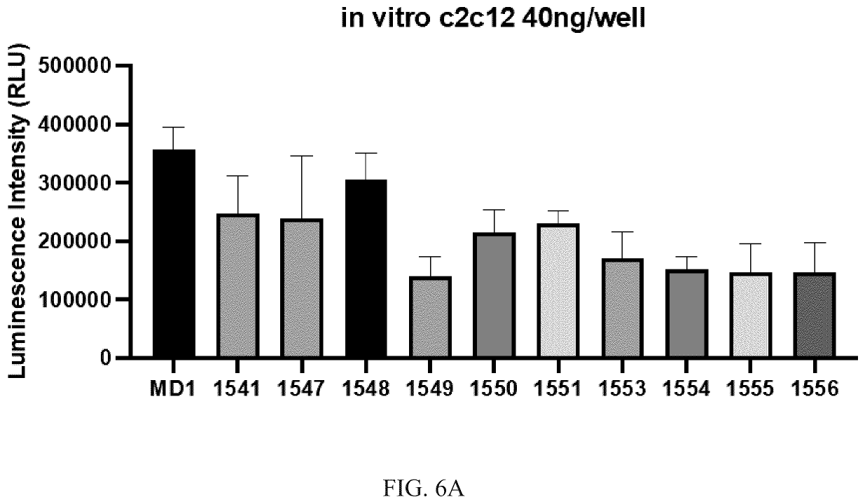

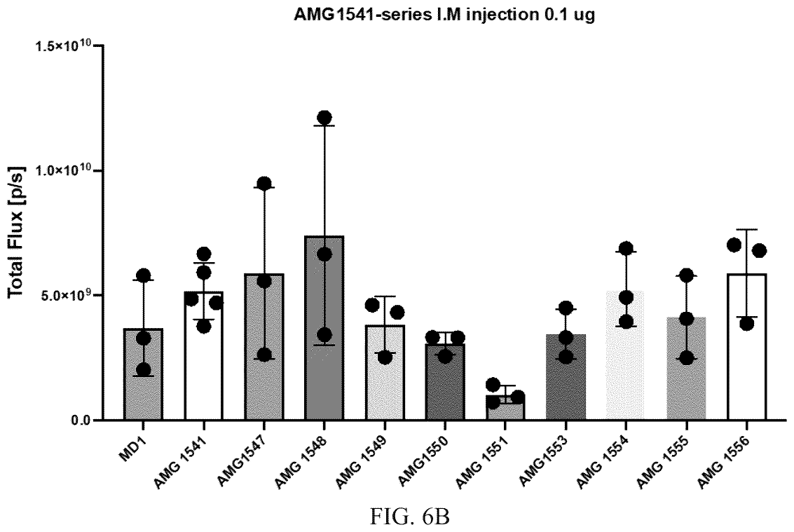

The ionizable lipids obtained from the “alkene walking” strategy were also evaluated for in vitro and in vivo protein expression via intramuscular administration. Several ionizable lipids (e.g., AMG1547, AMG1548, AMG1549, AMG1550, AMG1553, AMG1554, AMG1555, AMG1556) superseded or matched MD-1, a highly potent ionizable lipid (FIGS. 6A-6B).

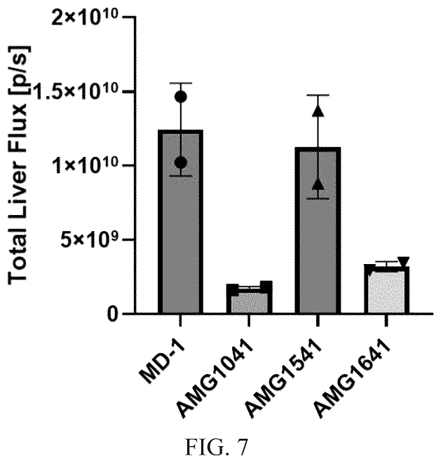

Evaluation of the LNPs via intravenous injection. The lipids were initially evaluated for intravenous protein expression by administration of a 0.25 mg/kg dose (5 g) of fLuc mRNA LNPs. At 6 hrs., AMG1541 showed comparable levels of protein expression to lipid MD-1 (FIG. 7).

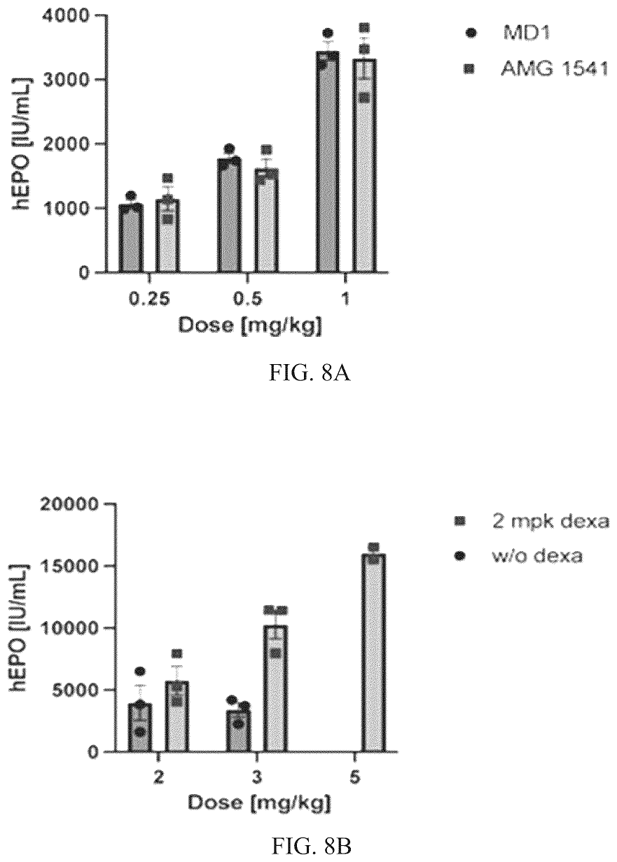

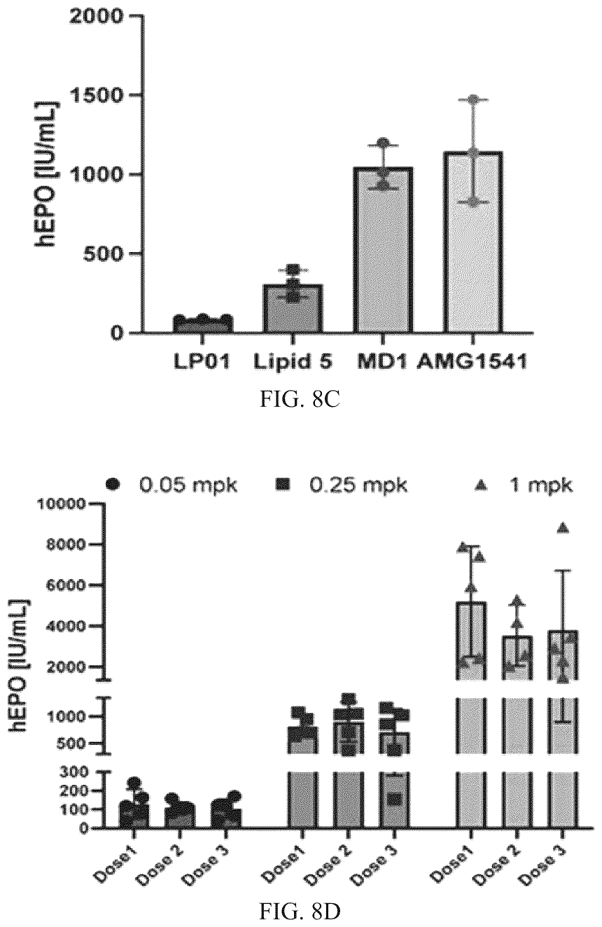

Further evaluation of AMG1541 using hEPO mRNA showed consistent levels of protein expression in liver within a dose range of 0.25-1 mg/kg. In addition, at 1 mg/kg dose, AMG1541 showed 3-4 fold higher protein expression than Lipid 5, one of the most potent lipids developed by Moderna. At higher doses beyond 2 mg/kg, AMG1541 exhibited reduced protein expression. Without wishing to be bound by theory, this reduced protein expression may be due to inflammation, which could be reversed by pretreatment of the mice with dexamethasone at 1 mg/kg dose. A repeat dosing study over 3 weeks at 0.05-1 mg/kg range showed consistent hEPO production in liver (FIG. 8).

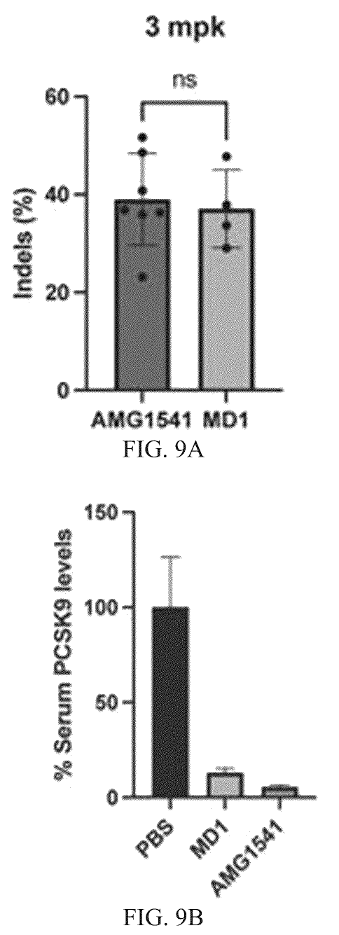

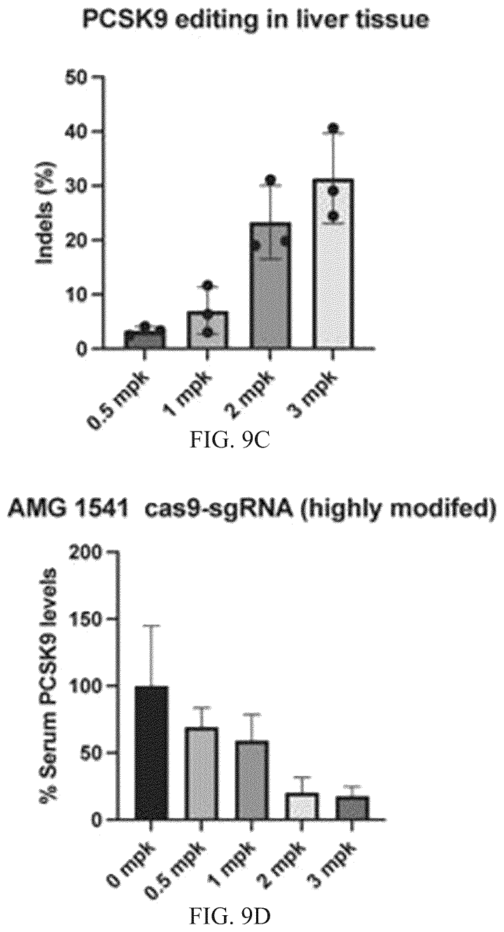

The ability of AMG1541 mRNA LNPs to induce CRISPR-Cas9 gene editing was also evaluated (FIGS. 9A-9D). At a dose of 3 mg/kg, AMG1541 generated a similar level of indels in the PCSK9 gene as MD-1, which lead to a significant reduction in serum PCSK9 levels. Dose-response behavior showed an expected increase in gene editing as the RNA dose was increased, which was accompanied by decreased serum PCSK9 protein.

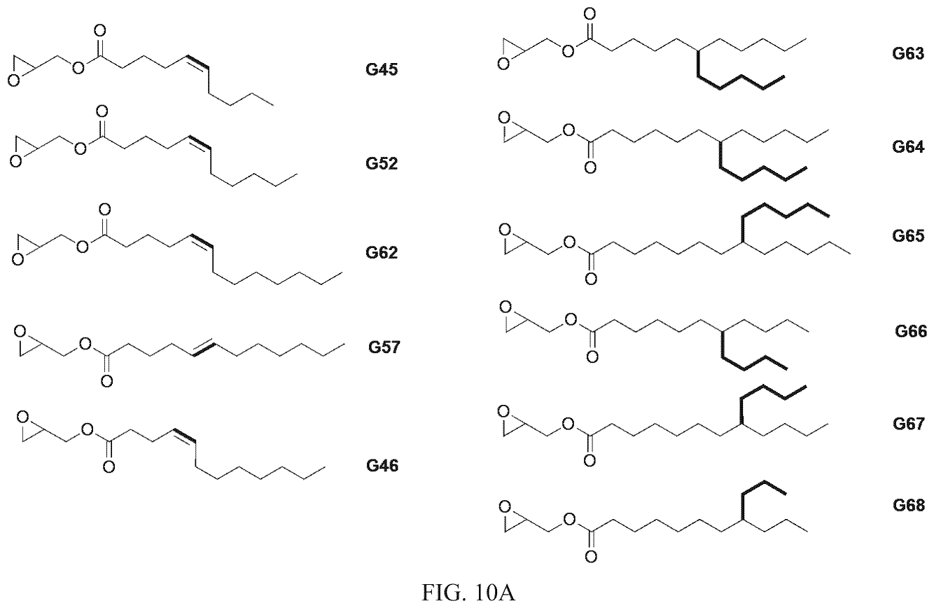

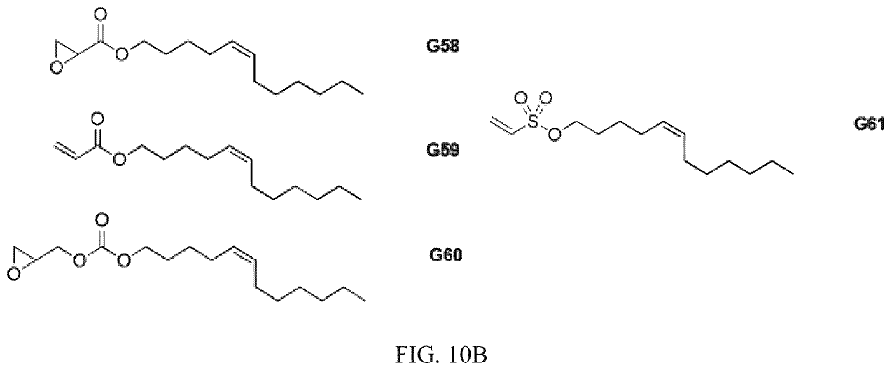

Additional lipid tails with differences in chain length, saturation, branching, etc., are used with head group Am15 to produce additional lipids (FIG. 10). When incorporated into LNPs, these lipids may further improve transfection potency, immunogenicity, and/or ability of the antibody-containing serum to prevent infection.

Based on clearance studies, AMG1541 is believed to demonstrate rapid clearance like AMG1041, which may offer the advantage of reduced toxicity compared to other ionizable lipids.

AMG1541 demonstrated equal or higher potency in intramuscular administration of mRNA compared to FDA-approved ionizable lipids such as SM-102 and ALC-0315 present in Moderna and Pfizer/BioNtech COVID vaccines. In addition, AMG1541 also demonstrated an excellent hEPO expression profile in the liver when administered intravenously, comparable to the potent ionizable lipid MD-1.Based on high transfection potency in Hela cells, some of the potent lipids are tested for mRNA delivery efficacy in Jurkat cells, an immortalized human T-cell line for potential CAR-T therapeutic applications. Jurkat cells are cultured in a RPMI-1640 medium with L-glutamine supplemented with 10% bovine serum and 1% penicillin-streptomycin. Jurkat cells are plated on a white, flat-bottom 96 well plate (Coming, Coming, NY) at a density of 100k cells/well and co-incubated dosed with 150 ng of mRNA. Luciferase expression in the cells is determined after 24 hours using a Bright-Glo assay (Promega, Madison, WI) and normalized to cell viability (Multi-tox, Promega). LNPs comprising ionizable lipids of the present disclosure may demonstrate higher mRNA transfection than positive controls (e.g., commercially used lipofectamine positive controls).

### Example 2: Synthesis and Characterization of Lipids

Various fatty acid epoxy esters are synthesized by esterifying various corresponding fatty acids with glycidol (Scheme 1, wherein R⁵ is as defined herein). The head group is reacted with the various fatty acid epoxy esters to provide ionizable lipids (Scheme 2, wherein n, A, B, R, and R⁵ are as defined herein). These lipids are named AMGXXYY, where XX is the head group number and YY is the epoxy ester number as shown in FIGS. 1A-1B and 10. For example, AMG1541 corresponds to the lipid synthesized using head group Am15 (FIG. 1A) and tail group G41 (FIG. 1B). Without wishing to be bound by any particular theory, the number of ester groups and tail length impact the transfection ability of the LNPs.

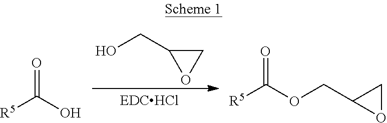

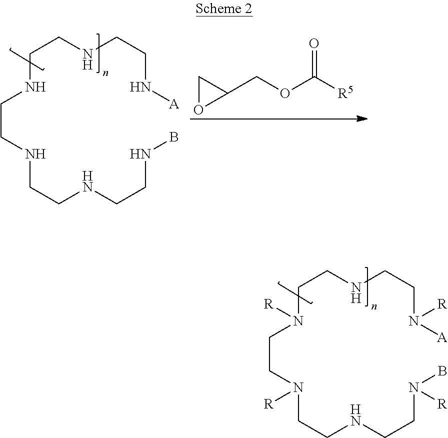

General Synthetic Methods. Anhydrous triethylamine, pyridine, EtOH, MeOH, CH₃CN, THF, DMF and CH₂Cl₂ were purchased from Sigma-Aldrich®. All other reagents were purchased from commercial sources and were used without further purification unless noted otherwise. Synthetic chemistry scale up services for selective chemical intermediates were from TCG Lifesciences. All reactions were carried out under a positive pressure of argon atmosphere and monitored by TLC on Silica Gel G-25 UV₂₅₄ (0.25 mm) unless stated otherwise. Spots were detected under UV light and/or by iodine, ethanolic solution of phosphomolybdic acid, or KMnO₄. Column flash chromatography was performed using Teledyne Isco Combi Flash® instrument equipped with UV detector, an evaporative light scattering detector (ELSD) and prepacked silica gel, alumina or reverse phase C₁₈ silica gel columns. Crude products were mixed thoroughly with celite and loaded into empty cartridges supplied by Teledyne Isco.

General synthesis of glycidyl esters. Synthesis of chemically diverse set of glycidyl esters were performed based on precedent literature and modified as needed.¹⁴ General synthetic routes for the esters G1-G10 are shown in the following scheme ((A) Synthetic route for glycidyl esters (B) Synthetic route for epoxy ester (C) Synthetic route for branched tail glycidyl esters). Next generation glycidyl esters were synthesized according to the same procedure (Steglich esterification)¹⁵ or Shiina esterification¹⁶ as shown in the following scheme (A).

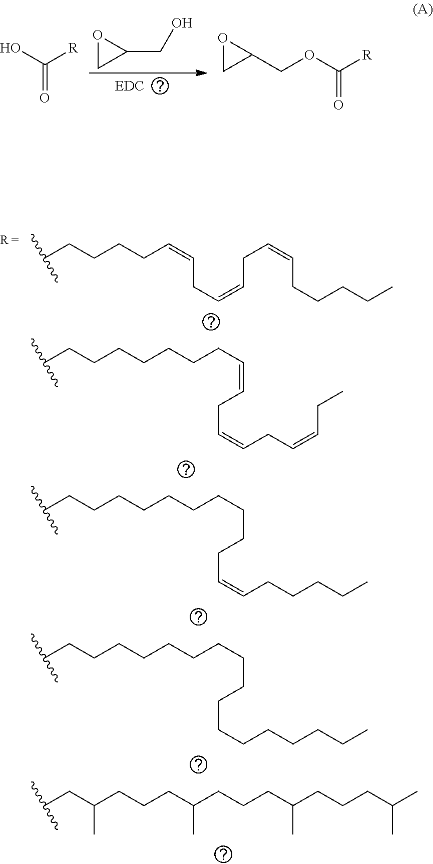

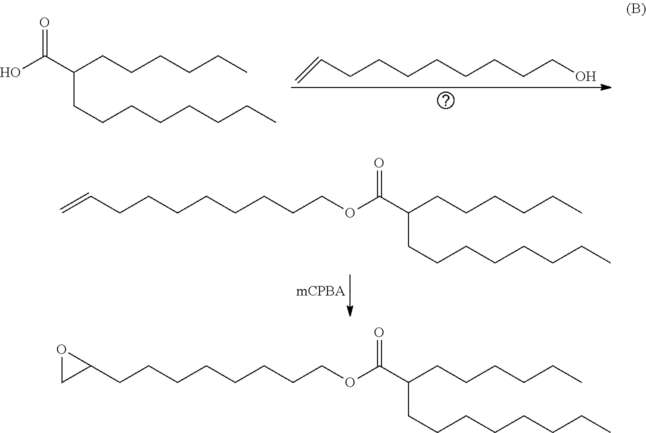

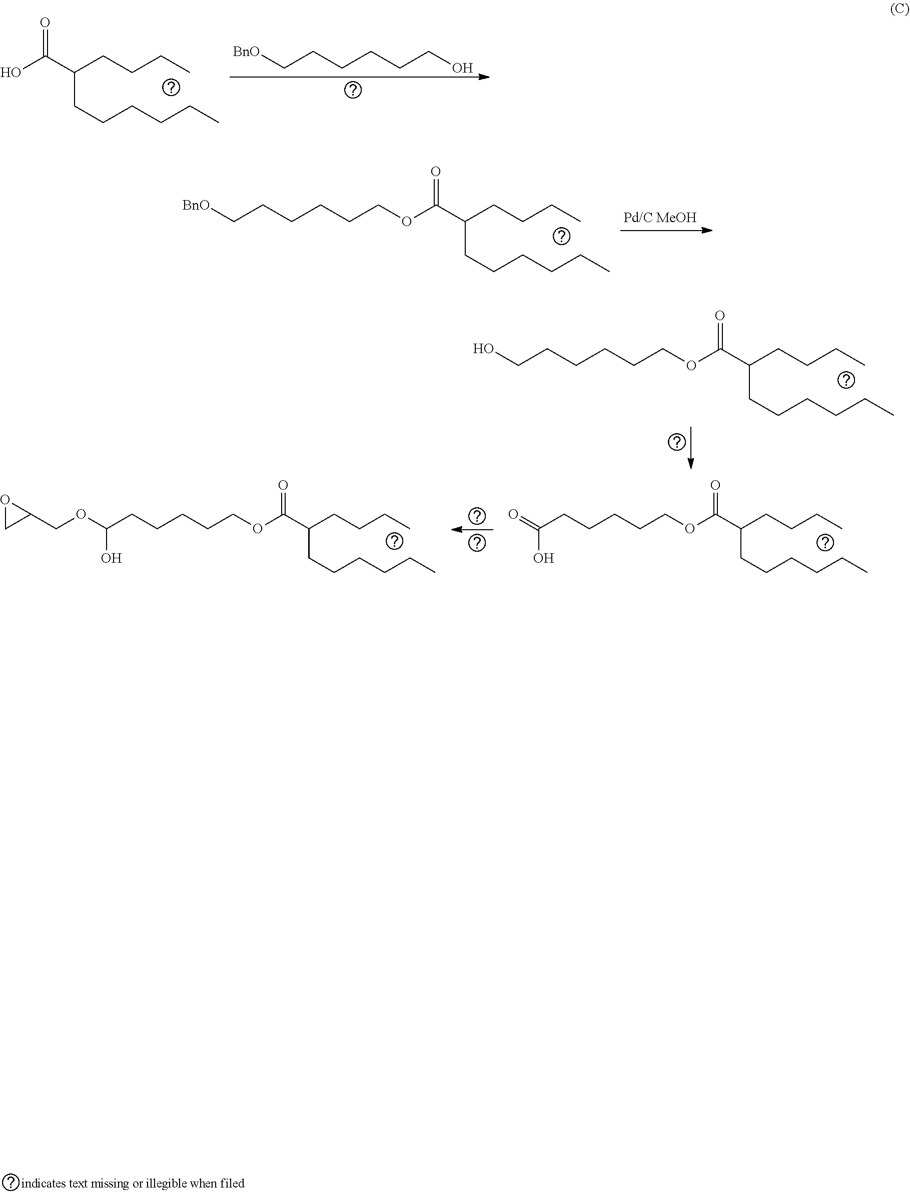 [SMILES: CCCCCCC(CCCC)C(=O)O ; OCCCCCCOCc1ccccc1 ; CCCCCCC(CCCC)C(=O)OCCCCCCOCc1ccccc1 ; CCCCCCC(CCCC)C(=O)OCCCCCCO ; CCCCCCC(CCCC)C(=O)OCCCCCC(O)OCC1CO1 ; CCCCCCC(CCCC)C(=O)OCCCCCC(=O)O]

G1. ¹H NMR (500 MHz, CDCl₃) δ 5.48-5.27 (m, 6H), 4.42 (dd, J=12.3, 3.1 Hz, 1H), 3.93 (dd, J=12.3, 6.3 Hz, 1H), 3.22 (dq, J=6.5, 3.1 Hz, 1H), 2.84 (dt, J=18.2, 5.2 Hz, 6H), 2.66 (dd, J=4.9, 2.6 Hz, 1H), 2.38 (t, J=7.5 Hz, 2H), 2.16-2.03 (m, 4H), 1.68 (p, J=7.5 Hz, 2H), 1.47-1.25 (m, 8H), 0.90 (t, J=6.8 Hz, 3H). ¹³C NMR (126 MHz, CDCl₃) δ 173.32, 130.45, 129.53, 128.43, 128.30, 128.05, 127.59, 64.80, 49.36, 44.65, 33.96, 33.94, 31.52, 29.33, 29.09, 29.07, 29.05, 27.24, 27.22, 26.85, 25.64, 24.49, 24.48, 22.58, 14.07.

G2. ¹H NMR (500 MHz, CDCl₃) δ 5.45-5.19 (m, 6H), 4.42 (dtd, J=12.2, 3.1, 1.1 Hz, 1H), 4.23-4.09 (m, 2H), 3.92 (dddd, J=12.5, 6.5, 3.2, 1.3 Hz, 2H), 2.91-2.76 (m, 5H), 2.65 (dtd, J=5.5, 2.7, 1.0 Hz, 1H), 2.43-2.27 (m, 5H), 2.14-2.02 (m, 5H), 1.72-1.54 (m, 5H), 1.33 (t, J=8.4 Hz, 19H), 0.98 (tdd, J=7.5, 3.1, 1.1 Hz, 4H). ¹³C NMR (126 MHz, CDCl₃) δ 173.56, 131.96, 130.25, 130.23, 128.29, 128.24, 127.75, 127.73, 127.11, 70.24, 65.15, 64.76, 63.35, 49.40, 44.67, 34.13, 34.08, 34.06, 34.01, 29.58, 29.56, 29.16, 29.14, 29.08, 29.02, 27.19, 25.61, 25.53, 24.88, 24.85, 24.77, 20.55, 14.28.

G3. ¹H NMR (500 MHz, CDCl₃) δ 5.44-5.28 (m, 4H), 4.42 (ddd, J=12.2, 3.1, 1.2 Hz, 1H), 3.92 (ddd, J=12.3, 6.3, 1.3 Hz, 1H), 3.25-3.18 (m, 1H), 2.88-2.82 (m, 1H), 2.81-2.75 (m, 2H), 2.68-2.63 (m, 1H), 2.40-2.31 (m, 3H), 2.06 (q, J=6.6 Hz, 4H), 1.64 (dt, J=10.9, 6.9 Hz, 3H), 1.42-1.23 (m, 20H), 0.94-0.84 (m, 4H). ¹³C NMR (126 MHz, CDCl₃) δ 173.52, 130.20, 130.02, 128.05, 127.90, 64.77, 64.75, 49.39, 44.65, 34.05, 34.03, 31.52, 29.58, 29.34, 29.16, 29.14, 29.12, 29.11, 29.08, 27.20, 27.18, 25.62, 24.85, 22.57, 14.07.

G4. ¹H NMR (500 MHz, CDCl₃) δ 5.33 (qd, J=3.9, 1.8 Hz, 2H), 4.39 (ddd, J=12.3, 3.1, 1.4 Hz, 1H), 3.90 (ddd, J=12.2, 6.3, 1.4 Hz, 1H), 3.22-3.15 (m, 1H), 2.82 (ddt, J=5.1, 4.2, 1.0 Hz, 1H), 2.63 (ddd, J=4.5, 2.7, 1.6 Hz, 1H), 2.33 (td, J=7.6, 1.3 Hz, 2H), 2.05-1.94 (m, 4H), 1.62 (p, J=6.9 Hz, 2H), 1.41-1.12 (m, 20H), 0.86 (td, J=7.0, 1.3 Hz, 3H). ¹³C NMR (126 MHz, CDCl₃) δ 173.51, 130.00, 129.72, 64.75, 49.38, 44.64, 34.05, 31.91, 29.77, 29.68, 29.52, 29.32, 29.17, 29.15, 29.09, 27.21, 27.16, 24.86, 22.68, 14.11.

G5. ¹H NMR (500 MHz, CDCl₃) δ 4.49-4.28 (m, 1H), 3.90 (ddt, J=18.6, 12.2, 5.1 Hz, 1H), 3.18 (dtq, J=21.0, 6.5, 3.2 Hz, 1H), 3.03-2.75 (m, 2H), 2.68-2.57 (m, 1H), 2.41-2.28 (m, 1H), 2.14 (tt, J=14.6, 7.9 Hz, 1H), 1.51 (ddp, J=19.9, 13.2, 6.7 Hz, 1H), 1.42-0.99 (m, 15H), 0.99-0.65 (m, 15H). ¹³C NMR (126 MHz, CDCl₃) δ 172.79, 64.69, 64.67, 64.64, 64.62, 64.60, 49.38, 49.34, 49.27, 44.67, 44.60, 44.53, 41.64, 41.61, 41.57, 41.55, 41.47, 39.36, 39.33, 37.44, 37.40, 37.35, 37.31, 37.29, 37.27, 37.24, 37.19, 37.16, 37.13, 37.09, 37.07, 37.04, 37.01, 36.98, 32.79, 32.76, 32.73, 32.71, 32.69, 32.67, 30.34, 30.29, 30.27, 27.97, 27.92, 24.80, 24.77, 24.45, 24.42, 24.34, 24.30, 22.72, 22.68, 22.63, 22.61, 22.58, 19.75, 19.71, 19.68, 19.64, 19.56.

G6. ¹H NMR (500 MHz, CDCl₃) δ 4.07 (td, J=6.7, 1.1 Hz, 1H), 2.90 (tddd, J=5.2, 4.0, 2.7, 1.2 Hz, 1H), 2.75 (ddd, J=5.2, 3.9, 1.3 Hz, 1H), 2.46 (ddd, J=5.1, 2.8, 1.3 Hz, 1H), 2.36-2.27 (m, 1H), 1.67-1.55 (m, 2H), 1.55-1.39 (m, 1H), 1.39-1.20 (m, 14H), 0.88 (tt, J=7.2, 1.2 Hz, 3H). ¹³C NMR (126 MHz, CDCl₃) δ 176.69, 64.07, 52.34, 47.08, 45.86, 32.57, 32.48, 31.87, 31.71, 29.57, 29.46, 29.37, 29.26, 29.23, 29.14, 28.70, 27.48, 27.46, 27.44, 25.97, 25.95, 22.66, 22.59, 14.10, 14.06.

G7. ¹H NMR (500 MHz, CDCl₃) δ 4.41 (dtd, J=12.3, 3.0, 1.0 Hz, 2H), 4.15-4.02 (m, 8H), 3.90 (dddd, J=12.4, 6.4, 3.0, 1.0 Hz, 2H), 3.23-3.16 (m, 2H), 2.87-2.80 (m, 2H), 2.63 (dtd, J=5.8, 2.8, 1.0 Hz, 2H), 2.42-2.33 (m, 4H), 2.32-2.25 (m, 8H), 2.06-1.93 (m, 3H), 1.62 (dtd, J=17.5, 7.3, 2.9 Hz, 11H), 1.38 (h, J=2.8 Hz, 8H), 1.33-1.20 (m, 35H), 0.92-0.83 (m, 12H). ¹³C NMR (126 MHz, CDCl₃) δ 173.78, 173.77, 173.12, 64.86, 63.84, 49.32, 44.62, 37.18, 34.24, 33.80, 31.65, 29.09, 28.90, 27.88, 26.26, 24.94, 24.89, 22.58, 14.04.

G8. ¹H NMR (500 MHz, CDCl₃) δ 4.41 (dq, J=12.3, 2.7 Hz, 1H), 4.06 (tt, J=6.8, 2.3 Hz, 2H), 3.91 (ddt, J=11.3, 6.7, 2.4 Hz, 1H), 3.20 (tq, J=7.3, 2.8 Hz, 1H), 2.84 (tt, J=5.2, 2.8 Hz, 1H), 2.64 (dq, J=5.3, 2.6 Hz, 1H), 2.36 (tt, J=7.5, 2.3 Hz, 2H), 2.34-2.25 (m, 1H), 1.66 (dddd, J=19.6, 12.1, 5.7, 3.3 Hz, 4H), 1.61-1.51 (m, 2H), 1.42 (tddt, J=13.5, 8.0, 5.8, 3.0 Hz, 4H), 1.35-1.15 (m, 11H), 0.87 (dtt, J=7.4, 5.1, 2.5 Hz, 6H). ¹³C NMR (126 MHz, CDCl₃) δ 176.60, 176.58, 173.11, 64.84, 63.72, 49.31, 45.77, 44.61, 44.60, 33.85, 32.51, 32.20, 31.68, 29.64, 29.20, 28.39, 27.41, 25.53, 24.45, 22.60, 22.57, 14.04, 13.93.

G9. ¹H NMR (500 MHz, CDCl₃) δ 4.41 (d, J=12.3 Hz, 1H), 4.07 (td, J=6.6, 2.2 Hz, 2H), 3.20 (dhept, J=6.7, 2.0 Hz, 1H), 2.84 (qt, J=3.2, 1.6 Hz, 1H), 2.64 (dt, J=4.8, 2.4 Hz, 1H), 2.37 (td, J=7.5, 2.1 Hz, 2H), 2.34-2.23 (m, 1H), 1.67 (tdd, J=15.0, 8.1, 4.3 Hz, 4H), 1.58 (t, J=7.3 Hz, 2H), 1.40 (p, J=7.7 Hz, 5H), 1.35-1.17 (m, 20H), 0.91-0.84 (m, 6H). ¹³C NMR (126 MHz, CDCl₃) δ 176.60, 173.11, 64.84, 63.72, 49.31, 45.80, 44.62, 33.86, 32.51, 31.84, 31.68, 29.55, 29.44, 29.24, 29.20, 28.40, 27.46, 27.41, 25.53, 24.46, 22.65, 22.58, 14.08, 14.05.

G10. ¹H NMR (500 MHz, CDCl₃) δ 4.42 (dd, J=12.4, 3.0 Hz, 1H), 4.07 (t, J=6.6 Hz, 2H), 3.92 (dd, J=12.3, 6.3 Hz, 1H), 3.20 (ddt, J=6.8, 4.0, 2.8 Hz, 1H), 2.85 (t, J=4.5 Hz, 1H), 2.64 (dd, J=5.0, 2.6 Hz, 1H), 2.37 (t, J=7.5 Hz, 2H), 2.31 (tt, J=8.7, 5.3 Hz, 1H), 1.67 (dp, J=14.6, 7.3 Hz, 4H), 1.58 (tt, J=12.4, 5.0 Hz, 2H), 1.48-1.36 (m, 4H), 1.26 (s, 30H), 0.88 (t, J=6.8 Hz, 6H). ¹³C NMR (126 MHz, CDCl₃) δ 176.61, 173.11, 64.84, 63.72, 49.32, 45.80, 44.62, 33.86, 32.50, 31.90, 31.85, 31.84, 29.59, 29.55, 29.52, 29.49, 29.44, 29.33, 29.25, 29.23, 28.40, 27.46, 25.53, 24.47, 22.67, 22.65, 14.09.

GC12. ¹H NMR (500 MHz, CDCl₃) δ 4.43 (dd, J=12.3, 3.1 Hz, 1H), 3.93 (dd, J=12.3, 6.3 Hz, 1H), 3.22 (ddt, J=6.0, 4.1, 2.8 Hz, 1H), 2.86 (dd, J=4.9, 4.1 Hz, 1H), 2.66 (dd, J=4.9, 2.6 Hz, 1H), 2.36 (t, J=7.6 Hz, 2H), 1.70-1.60 (m, 2H), 1.29 (d, J=19.3 Hz, 17H), 0.89 (t, J=6.9 Hz, 3H). ¹³C NMR (126 MHz, CDCl₃) δ 173.56, 64.75, 49.40, 44.67, 34.08, 31.91, 29.60, 29.45, 29.33, 29.25, 29.12, 24.88, 22.68, 14.11.

GC14. ¹H NMR (500 MHz, CDCl₃) δ 4.43 (dd, J=12.3, 3.1 Hz, 1H), 3.94 (dd, J=12.3, 6.3 Hz, 1H), 3.23 (ddd, J=6.6, 3.9, 2.1 Hz, 1H), 2.87 (t, J=4.5 Hz, 1H), 2.67 (dd, J=4.9, 2.6 Hz, 1H), 2.37 (t, J=7.6 Hz, 2H), 1.66 (p, J=7.4 Hz, 2H), 1.30 (d, J=19.7 Hz, 24H), 0.90 (t, J=6.9 Hz, 3H). ¹³C NMR (126 MHz, CDCl₃) δ 173.59, 64.76, 49.41, 44.69, 34.10, 31.93, 29.68, 29.65, 29.60, 29.46, 29.36, 29.26, 29.13, 24.89, 22.70, 14.13.

GC16. ¹H NMR (500 MHz, CDCl₃) δ 4.43 (dd, J=12.3, 3.1 Hz, 1H), 3.94 (dd, J=12.3, 6.3 Hz, 1H), 3.23 (ddt, J=6.5, 4.1, 2.1 Hz, 1H), 2.87 (t, J=4.5 Hz, 1H), 2.67 (dd, J=4.9, 2.6 Hz, 1H), 2.37 (t, J=7.6 Hz, 2H), 1.65 (p, J=7.5 Hz, 2H), 1.28 (s, 24H), 0.90 (t, J=6.9 Hz, 3H). ¹³C NMR (126 MHz, CDCl₃) δ 173.57, 64.75, 49.41, 44.70, 44.68, 34.11, 34.09, 31.93, 29.70, 29.68, 29.66, 29.65, 29.60, 29.46, 29.37, 29.28, 29.26, 29.13, 24.89, 22.70, 14.13.

GC18. ¹H NMR (500 MHz, CDCl₃) δ 4.43 (dd, J=12.3, 3.1 Hz, 1H), 3.94 (dd, J=12.3, 6.3 Hz, 1H), 3.26-3.19 (m, 1H), 2.89-2.84 (m, 1H), 2.67 (dd, J=4.9, 2.6 Hz, 1H), 2.37 (t, J=7.6 Hz, 2H), 1.66 (p, J=7.4 Hz, 2H), 1.28 (s, 32H), 0.90 (t, J=6.9 Hz, 3H). ¹³C NMR (126 MHz, CDCl₃) δ 173.58, 64.75, 49.41, 44.68, 34.09, 31.94, 29.71, 29.68, 29.67, 29.66, 29.61, 29.46, 29.37, 29.26, 29.14, 24.89, 22.71, 14.13.

G41. ¹H NMR (500 MHz, CDCl₃) δ 5.43 (dddt, J=9.8, 7.2, 6.1, 1.3 Hz, 1H), 5.38-5.28 (m, 1H), 4.42 (ddd, J=12.3, 3.1, 0.8 Hz, 1H), 3.93 (ddd, J=12.3, 6.3, 0.9 Hz, 1H), 3.25-3.18 (m, 1H), 2.88-2.83 (m, 1H), 2.66 (ddd, J=4.9, 2.6, 0.8 Hz, 1H), 2.41-2.34 (m, 3H), 2.15-2.06 (m, 3H), 2.06-1.95 (m, 3H), 1.72 (p, J=7.5 Hz, 3H), 1.41-1.22 (m, 7H), 0.94-0.85 (m, 3H). ¹³C NMR (126 MHz, CDCl₃) δ 173.37, 131.28, 128.21, 64.81, 49.37, 44.66, 33.45, 31.77, 29.66, 28.98, 27.24, 26.50, 24.82, 22.65, 14.10.

G43. ¹H NMR (500 MHz, CDCl₃) δ 5.41-5.29 (m, 2H), 4.42 (ddt, J=12.3, 3.2, 0.9 Hz, 1H), 3.92 (ddt, J=12.3, 6.3, 0.9 Hz, 1H), 3.25-3.17 (m, 1H), 2.85 (ddt, J=5.0, 4.1, 1.0 Hz, 1H), 2.65 (dt, J=4.9, 1.7 Hz, 1H), 2.40-2.32 (m, 2H), 2.02 (q, J=6.5 Hz, 4H), 1.64 (p, J=7.1 Hz, 2H), 1.40-1.22 (m, 17H), 0.93-0.86 (m, 3H). ¹³C NMR (126 MHz, CDCl₃) δ 173.53, 77.31, 77.05, 76.80, 64.77, 64.75, 49.39, 44.65, 34.05, 31.80, 31.78, 29.73, 29.71, 29.67, 29.16, 29.14, 29.08, 29.00, 28.98, 28.96, 28.95, 27.21, 27.15, 24.85, 22.65, 14.10.

G47. ¹H NMR (500 MHz, CDCl₃) δ 5.50-5.23 (m, 2H), 4.42 (ddt, J=12.3, 2.6, 1.3 Hz, 1H), 3.92 (dddd, J=12.3, 6.3, 2.6, 1.0 Hz, 1H), 3.21 (ddqd, J=6.8, 3.9, 2.7, 1.1 Hz, 1H), 2.85 (dddd, J=4.9, 4.0, 2.9, 1.5 Hz, 1H), 2.65 (dtd, J=5.3, 2.7, 1.1 Hz, 1H), 2.40-2.33 (m, 2H), 2.04 (dq, J=19.9, 7.1 Hz, 4H), 1.72-1.61 (m, 2H), 1.44-1.22 (m, 8H), 0.95-0.82 (m, 3H). ¹³C NMR (126 MHz, CDCl₃) δ 173.39, 173.38, 130.52, 129.00, 64.77, 49.37, 44.65, 33.95, 31.51, 29.38, 29.17, 27.19, 26.79, 24.49, 22.56, 14.06.

G48. ¹H NMR (500 MHz, CDCl₃) δ 5.40-5.28 (m, 2H), 4.44-4.37 (m, 1H), 3.91 (ddt, J=12.3, 6.3, 0.9 Hz, 1H), 3.23-3.16 (m, 1H), 2.83 (ddt, J=5.2, 4.3, 1.0 Hz, 1H), 2.64 (ddt, J=4.6, 2.6, 0.9 Hz, 1H), 2.35 (td, J=7.6, 1.3 Hz, 2H), 2.07-1.93 (m, 4H), 1.69-1.57 (m, 2H), 1.41-1.23 (m, 7H), 0.95-0.85 (m, 3H). ¹³C NMR (126 MHz, CDCl₃) δ 173.43, 130.15, 129.40, 64.74, 64.73, 49.35, 44.62, 34.00, 31.91, 29.33, 28.73, 26.95, 26.91, 26.90, 24.76, 22.31, 13.97, 13.96.

G49. ¹H NMR (500 MHz, CDCl₃) δ 5.41-5.29 (m, 2H), 4.41 (ddt, J=13.4, 6.2, 2.8 Hz, 1H), 3.91 (dtd, J=12.7, 6.3, 2.6 Hz, 1H), 3.24-3.15 (m, 1H), 2.87-2.80 (m, 1H), 2.64 (dtd, J=6.8, 3.5, 1.7 Hz, 1H), 2.39-2.30 (m, 2H), 2.06-1.91 (m, 4H), 1.67-1.55 (m, 2H), 1.42-1.23 (m, 8H), 0.94-0.83 (m, 3H). ¹³C NMR (126 MHz, CDCl₃) δ 173.48, 173.45, 129.82, 129.80, 64.75, 49.37, 49.35, 44.64, 44.61, 34.03, 34.01, 29.51, 29.28, 29.00, 28.85, 27.10, 24.82, 22.87, 13.79, 13.78.

G50. ¹H NMR (500 MHz, CDCl₃) δ 5.53-5.21 (m, 2H), 4.41 (dp, J=11.2, 2.7 Hz, 1H), 3.92 (ddt, J=12.2, 6.0, 2.9 Hz, 1H), 3.25-3.16 (m, 1H), 2.85 (dtq, J=6.4, 4.0, 1.4 Hz, 1H), 2.68-2.61 (m, 1H), 2.35 (tdd, J=7.6, 5.3, 2.3 Hz, 2H), 2.09-1.93 (m, 4H), 1.68-1.59 (m, 2H), 1.39-1.23 (m, 9H), 0.96 (tdd, J=8.6, 5.0, 2.3 Hz, 3H). ¹³C NMR (126 MHz, CDCl₃) δ 173.52, 173.49, 131.60, 131.58, 129.19, 129.18, 64.75, 49.39, 49.37, 44.65, 44.63, 34.06, 34.04, 29.66, 29.12, 29.07, 29.04, 27.03, 24.85, 20.50, 14.38.

G51. ¹H NMR (500 MHz, CDCl₃) δ 5.46-5.30 (m, 2H), 4.44-4.33 (m, 1H), 3.95-3.83 (m, 1H), 3.23-3.13 (m, 1H), 2.82 (dtd, J=10.2, 5.2, 2.5 Hz, 1H), 2.62 (dddt, J=11.0, 7.3, 4.7, 2.4 Hz, 1H), 2.38-2.27 (m, 3H), 2.05-1.90 (m, 2H), 1.68-1.53 (m, 6H), 1.39-1.15 (m, 13H). ¹³C NMR (126 MHz, CDCl₃) δ 173.50, 130.75, 123.58, 64.72, 64.70, 49.36, 49.33, 44.62, 44.59, 44.57, 44.55, 34.03, 33.99, 29.52, 29.49, 29.47, 29.30, 29.28, 29.25, 29.19, 29.17, 29.14, 29.07, 29.04, 26.78, 26.75, 24.84, 24.81, 12.71, 12.68.

G53. ¹H NMR (500 MHz, CDCl₃) δ 5.54-5.24 (m, 3H), 4.41 (dddt, J=10.9, 5.3, 2.8, 1.4 Hz, 1H), 3.92 (dddt, J=10.6, 5.5, 2.8, 1.5 Hz, 1H), 3.25-3.14 (m, 1H), 2.88-2.77 (m, 3H), 2.64 (dtd, J=4.8, 2.4, 1.0 Hz, 1H), 2.47-2.37 (m, 3H), 2.12-1.95 (m, 2H), 1.43-1.24 (m, 4H), 0.89 (ddddt, J=7.2, 6.1, 3.7, 2.6, 1.2 Hz, 3H). ¹³C NMR (126 MHz, CDCl₃) δ 172.80, 130.51, 129.85, 127.39, 127.37, 64.91, 64.81, 49.35, 49.33, 44.64, 33.98, 31.79, 30.31, 27.76, 26.93, 25.56, 22.68, 22.32, 22.29, 13.97, 13.95.

G54. ¹H NMR (500 MHz, CDCl₃) δ 5.46-5.23 (m, 2H), 4.50-4.30 (m, 1H), 3.89 (dddt, J=11.2, 6.0, 3.4, 1.5 Hz, 1H), 3.22-3.14 (m, 1H), 2.82 (dddd, J=5.0, 3.4, 2.4, 1.0 Hz, 1H), 2.78-2.69 (m, 1H), 2.62 (ddt, J=5.2, 2.7, 1.3 Hz, 1H), 2.47-2.27 (m, 2H), 2.16-2.06 (m, 1H), 2.01 (qdt, J=7.2, 2.3, 1.1 Hz, 2H), 1.79-1.62 (m, 2H), 1.44-1.29 (m, 2H), 0.98-0.82 (m, 3H). ¹³C NMR (126 MHz, CDCl₃) δ 173.29, 130.10, 129.30, 128.50, 127.78, 64.79, 49.33, 44.61, 33.37, 29.25, 26.46, 25.58, 24.67, 22.73, 13.75.

G55. ¹H NMR (500 MHz, CDCl₃) δ 5.46-5.22 (m, 2H), 4.39 (ddqd, J=11.5, 4.7, 3.0, 1.9 Hz, 1H), 3.89 (dddd, J=12.7, 6.5, 4.7, 1.8 Hz, 1H), 3.19 (dddt, J=6.0, 4.4, 3.0, 1.5 Hz, 1H), 2.82 (ddt, J=7.0, 4.8, 2.3 Hz, 1H), 2.78-2.67 (m, 1H), 2.63 (dtt, J=6.3, 4.6, 1.8 Hz, 1H), 2.41-2.26 (m, 3H), 2.13-1.92 (m, 3H), 1.78-1.52 (m, 3H), 1.52-1.21 (m, 2H), 1.06-0.73 (m, 3H). ¹³C NMR (126 MHz, CDCl₃) δ 173.37, 131.84, 129.30, 128.55, 127.18, 64.83, 64.80, 64.76, 49.39, 49.36, 49.33, 44.62, 33.90, 33.87, 33.82, 33.80, 29.02, 26.77, 25.49, 24.45, 24.31, 20.51, 14.25, 9.67.

G56. ¹H NMR (500 MHz, CDCl₃) δ 5.43 (ddddt, J=13.2, 6.6, 4.9, 3.3, 1.7 Hz, 1H), 5.38-5.27 (m, 4H), 4.44-4.34 (m, 3H), 3.89 (dddd, J=12.4, 9.2, 5.6, 3.0 Hz, 3H), 3.17 (dddd, J=8.2, 4.8, 3.4, 1.6 Hz, 2H), 2.85-2.79 (m, 3H), 2.78-2.68 (m, 4H), 2.65-2.59 (m, 3H), 2.33 (tdd, J=7.7, 6.4, 2.8 Hz, 6H), 2.11-1.98 (m, 4H), 1.69-1.55 (m, 10H), 1.43-1.25 (m, 10H). ¹³C NMR (126 MHz, CDCl₃) δ 173.41, 173.39, 129.75, 128.83, 128.07, 123.94, 64.78, 64.74, 49.34, 44.60, 44.58, 33.96, 33.95, 29.20, 28.69, 26.95, 25.25, 24.72, 24.69, 12.72.

### Synthesis and Characterization of Tails G59, G60, and G61

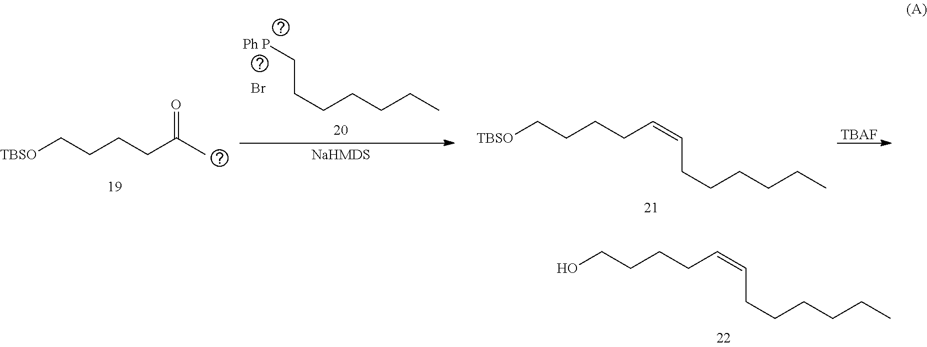

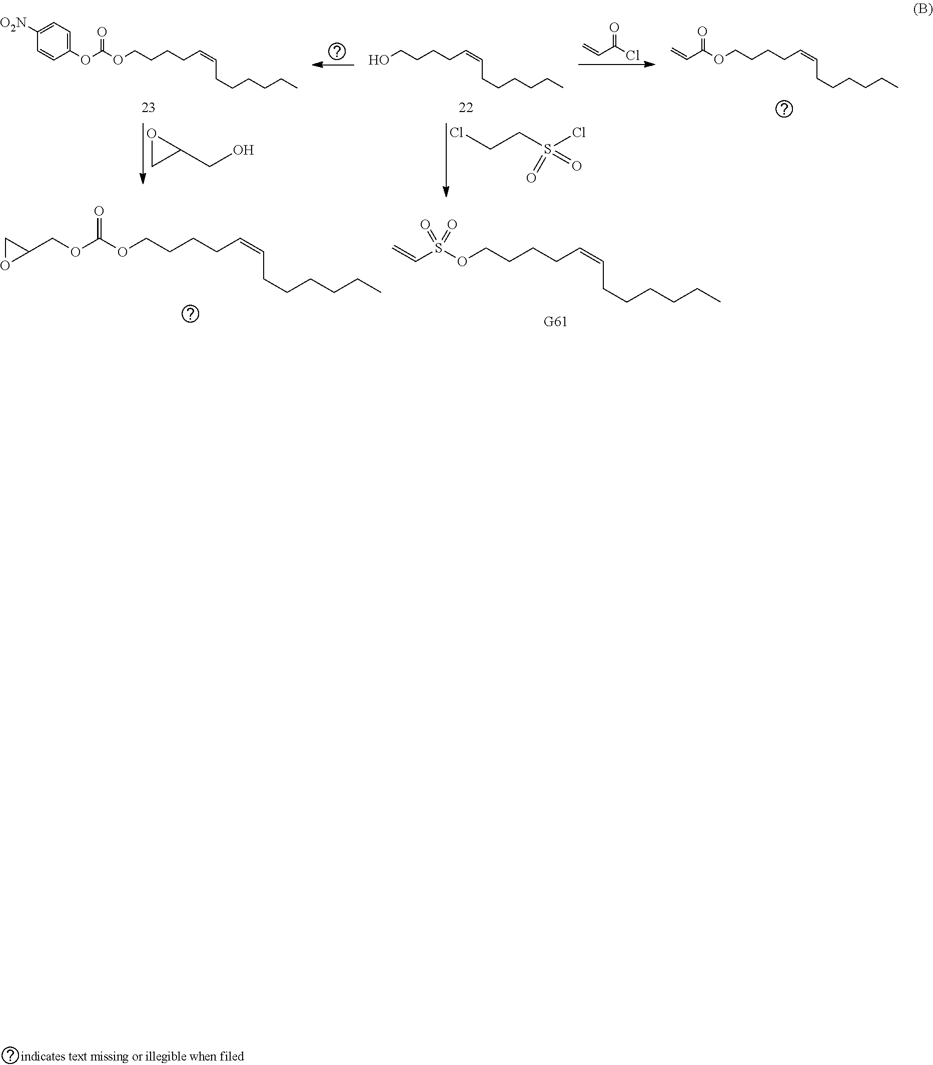

Compound 22 was synthesized using a modified version of a procedure.¹⁷ Briefly, phosphonium bromide 20 (10.2 g, 23.1 mmol, 1 equiv.) was dissolved in THF (116 ml). NaHMDS (23 ml, 1 M in THF, 1 equiv.)) was added slowly to the stirring solution of 20 at rt. The reaction mixture was stirred for 2 h after which aldehyde 19 (5 g, 23.1 mmol, 1 equiv.) was added slowly and then stirred overnight. The reaction mixture was quenched using saturated aq. NH₄C₁ (10 ml) and then washed with water (3×30 ml). The organic layer was collected, washed with brine (30 ml), dried over anhydrous Na₂SO₄, and then concentrated under low pressure. The crude material was purified using flash column chromatography using a gradient of 0→15% ethyl acetate in hexanes and carried forward to the next step.

The TBS ether (˜23.1 mmol) was dissolved in THF (46 ml). The reaction mixture was cooled to 0° C. and stirred for 10 min. Next, TBAF (46 ml, 1 M in THF, 2 equiv.) was added and stirred for 2 h at rt. The reaction mixture was quenched by adding saturated aq. NH₄Cl (10 ml) and then was washed with water (3×30 ml). The organic layer was collected, washed with brine (30 ml), dried over anhydrous Na₂SO₄, and then concentrated under low pressure. The crude material was purified using flash column chromatography using a gradient of 0->40% ethyl acetate in hexanes to give the compound 22 as pale-yellow liquid (2.26 μg, 53% yield over 2 steps).

G59. Compound 22 (500 mg, 2.7 mmol, 1 equiv.) was dissolved in DCM (13.5 ml). The mixture was then cooled to 0° C. and stirred for 10 min. Et₃N (1.13 ml, 8.14 mmol, 3 equiv.) followed by acryloyl chloride (439 μl, 5.4 mmol, 2 equiv.) was added and stirred for 3 h. The reaction mixture was quenched with saturated NaHCO₃ (5 ml), then was washed with water (3×10 ml). The organic layer was collected, washed with brine (30 ml), dried over anhydrous Na₂SO₄, and then concentrated under low pressure. The crude material was purified using flash column chromatography using a gradient of 0→5% ethyl acetate in hexanes to give the compound G59 as pale-yellow liquid (566 mg, 88% yield). ¹H NMR (500 MHz, CDCl₃) δ 6.42 (dd, J=17.3, 1.5 Hz, 1H), 6.14 (dd, J=17.3, 10.4 Hz, 1H), 5.84 (dd, J=10.4, 1.5 Hz, 1H), 5.46-5.30 (m, 2H), 4.18 (t, J=6.7 Hz, 2H), 2.07 (dq, J=28.2, 6.8 Hz, 4H), 1.71 (dt, J=15.2, 6.8 Hz, 2H), 1.61 (s, 1H), 1.46 (tt, J=10.1, 6.4 Hz, 2H), 1.40-1.24 (m, 7H), 0.94-0.87 (m, 3H). ¹³C NMR (126 MHz, CDCl₃) δ 166.35, 130.65, 130.48, 129.01, 128.63, 64.57, 31.78, 29.70, 28.99, 28.23, 27.26, 26.75, 26.04, 22.66, 14.11.

G60 precursor 23. Compound 22 (500 mg, 2.7 mmol, 1 equiv.) was dissolved in DCM (13.5 ml). The mixture was then cooled to 0° C. and stirred for 10 min. Pyridine (655 μl, 8.1 mmol, 3 equiv.) followed by p-nitrophenyl chloroformate (816 mg, 4 mmol, 1.5 equiv.) was added and stirred for 1 h. The reaction mixture was quenched with saturated NaHCO₃ (5 ml), then was washed with water (3×10 ml). The organic layer was collected, washed with brine (30 ml), dried over anhydrous Na₂SO₄, and then concentrated under low pressure. The crude material was purified using flash column chromatography using a gradient of 0->5% ethyl acetate in hexanes to give the compound 23 as pale-yellow liquid (868 mg, 92% yield). ¹H NMR (500 MHz, CDCl₃) δ 8.33-8.27 (m, 2H), 7.43-7.37 (m, 2H), 5.50-5.32 (m, 2H), 4.32 (t, J=6.6 Hz, 2H), 2.09 (dqd, J=39.9, 7.3, 1.4 Hz, 5H), 1.85-1.74 (m, 2H), 1.56-1.47 (m, 2H), 1.33 (dddd, J=27.0, 12.8, 5.8, 2.5 Hz, 9H), 0.98-0.79 (m, 3H). ¹³C NMR (126 MHz, CDCl₃) δ 155.61, 152.57, 145.35, 130.95, 128.66, 125.31, 121.80, 69.55, 31.78, 29.68, 29.00, 28.08, 27.29, 26.63, 25.72, 22.66, 14.11.

G60. Compound 23 (715 mg, 2.05 mmol, 1 equiv.) was dissolved in DCM (10 ml). DMAP (50 mg, 0.4 mmol, 0.2 equiv.) followed by glycidol (542 μl, 8.2 mmol, 4 equiv.) was added and stirred for 16 h. The reaction mixture was quenched with saturated NaHCO₃ (5 ml), then was washed with water (3×10 ml). The organic layer was collected, washed with brine (10 ml), dried over anhydrous Na₂SO₄, and then concentrated under low pressure. The crude material was purified using flash column chromatography using a gradient of 0->40% ethyl acetate in hexanes to give the compound G60 as colorless liquid (553 mg, 95% yield). 1H NMR (500 MHz, CDCl₃) δ 5.47-5.30 (m, 2H), 4.41 (dd, J=12.1, 3.3 Hz, 1H), 4.18 (t, J=6.7 Hz, 2H), 4.06 (dd, J=12.1, 6.1 Hz, 1H), 3.26 (ddt, J=6.1, 4.0, 3.0 Hz, 1H), 2.88 (dd, J=4.9, 4.1 Hz, 1H), 2.69 (dd, J=4.9, 2.6 Hz, 1H), 2.13-1.95 (m, 4H), 1.76-1.65 (m, 2H), 1.46 (tt, J=10.1, 6.5 Hz, 2H), 1.40-1.29 (m, 4H), 1.32-1.24 (m, 4H), 0.94-0.87 (m, 3H). ¹³C NMR (126 MHz, CDCl₃) δ 155.06, 130.74, 128.84, 68.43, 68.11, 49.09, 44.63, 31.78, 29.69, 28.99, 28.21, 27.26, 26.68, 25.75, 22.66, 14.11.

G61. Compound 22 (500 mg, 2.7 mmol, 1 equiv.) was dissolved in DCM (13.5 ml). The mixture was then cooled to 0° C. and stirred for 10 min. Et₃N (1.9 ml, 13.56 mmol, 5 equiv.) followed by 2-chloroethylsulfonyl chloride (570 μl, 5.4 mmol, 2 equiv.) was added and stirred for 2 h. The reaction mixture was quenched with saturated NaHCO₃ (5 ml), then was washed with water (3×10 ml). The organic layer was collected, washed with brine (30 ml), dried over anhydrous Na₂SO₄, and then concentrated under low pressure. The crude material was purified using flash column chromatography using a gradient of 0->15% ethyl acetate in hexanes to give the compound G61 as pale-yellow liquid (735 mg, 99% yield). ¹H NMR (500 MHz, CDCl₃) δ 6.55 (dd, J=16.6, 9.9 Hz, 1H), 6.43 (dd, J=16.6, 0.8 Hz, 1H), 6.14 (d, J=9.9 Hz, 1H), 5.46-5.36 (m, 1H), 5.33 (dtd, J=10.9, 7.1, 1.4 Hz, 1H), 4.14 (t, J=6.5 Hz, 2H), 2.12-1.95 (m, 4H), 1.81-1.70 (m, 2H), 1.47 (p, J=7.5 Hz, 2H), 1.39-1.27 (m, 9H), 0.93-0.87 (m, 3H). ¹³C NMR (126 MHz, CDCl₃) δ 132.57, 130.99, 129.98, 128.52, 70.91, 31.77, 29.67, 28.99, 28.52, 27.27, 26.47, 25.48, 22.65, 14.11.

General Procedure for Synthesis of Lipids. A microwave vial (0.2-0.5 mL) was charged with the epoxy ester (one equivalent in excess of the number of reactive amines) followed by a 0.42 M solution of the headgroup amine in isopropanol. The total reaction volume was made up to 250 μL. The microwave vial was then sealed and heated to 90° C. for 1 hour. Once the reaction was judged complete by LC-MS, the reaction mixture was directly loaded on a silica gel column and purified using dichloromethane (solvent A) and a mixture (solvent B) of dichloromethane, methanol, and ammonium hydroxide (25:7:1). The fractions containing the desired lipid were concentrated and dried under vacuum. Unless the glycidyl esters were synthesized from (R)- or (S)-glycidols, all lipids thus generated were obtained as diastereomeric mixtures. The lipids were characterized by ¹H and ¹³C NMR spectroscopy.

AMG1041-OHD. ¹H NMR (600 MHz, CDCl₃) δ 5.46-5.38 (m, 4H), 5.38-5.30 (m, 4H), 4.12 (t, J=6.6 Hz, 8H), 2.51 (d, J=7.7 Hz, 15H), 2.46 (t, J=7.1 Hz, 8H), 2.34-2.27 (m, 8H), 2.09 (qd, J=7.3, 1.5 Hz, 9H), 2.02 (qd, J=7.1, 1.5 Hz, 9H), 1.76 (p, J=6.8 Hz, 8H), 1.70 (p, J=7.5 Hz, 8H), 1.60 (p, J=7.0 Hz, 4H), 1.41-1.18 (m, 23H), 0.90 (t, J=6.9 Hz, 11H). ¹³C NMR (151 MHz, CDCl₃) δ 173.68, 131.14, 128.35, 62.77, 51.76, 51.51, 51.15, 33.77, 31.78, 29.67, 28.99, 27.25, 26.58, 24.98, 22.65, 14.11. HRMS (ESI) m/z 1152.9806 (calculated for C₇₀Ha₁₂₉N₄O₈ [M+H]⁺ 1153.9810).

AMG141. ¹H NMR (500 MHz, CDCl₃) δ 5.41 (dddd, J=12.4, 10.8, 6.5, 2.4 Hz, 3H), 5.37-5.25 (m, 3H), 4.15-4.06 (m, 1H), 4.06-3.98 (m, 1H), 3.94 (dpt, J=9.9, 5.0, 2.0 Hz, 1H), 3.90-3.73 (m, 1H), 3.67 (s, 5H), 3.59-3.38 (m, 2H), 3.04-2.42 (m, 8H), 2.34 (dt, J=20.4, 7.6 Hz, 5H), 2.08 (q, J=7.3 Hz, 6H), 2.01 (q, J=6.8 Hz, 6H), 1.69 (p, J=7.4 Hz, 6H), 1.44-1.12 (m, 23H), 0.89 (t, J=6.8 Hz, 9H). ¹³C NMR (126 MHz, CDCl₃) δ 174.20, 173.73, 131.19, 131.12, 128.36, 128.30, 69.34, 69.13, 68.88, 68.71, 68.53, 68.38, 68.16, 68.12, 67.12, 67.02, 66.94, 66.73, 66.62, 66.50, 66.42, 66.38, 66.34, 66.31, 66.26, 66.23, 66.14, 66.02, 65.36, 65.13, 65.09, 65.00, 64.95, 64.39, 63.98, 63.34, 62.52, 62.40, 62.15, 61.86, 61.18, 61.08, 60.94, 54.51, 54.07, 53.92, 53.79, 53.49, 53.37, 53.31, 53.17, 53.08, 52.75, 52.55, 52.25, 51.50, 51.46, 51.45, 37.89, 33.90, 33.76, 33.71, 33.61, 33.59, 33.57, 33.54, 33.46, 31.87, 31.85, 31.84, 31.82, 31.81, 31.77, 31.76, 29.80, 29.77, 29.73, 29.71, 29.70, 29.68, 29.67, 29.65, 29.62, 29.04, 28.98, 27.29, 27.25, 27.22, 26.63, 26.56, 26.52, 26.50, 26.49, 25.00, 24.88, 24.84, 24.73, 22.77, 22.64, 14.15, 14.11, 14.09. HRMS (ESI) m/z 892.6968 (calculated for C₅₁H₉₄N₃O₉[M+H]⁺ 892.6990).

AMG1141. ¹H NMR (500 MHz, CDCl₃) δ 5.46-5.37 (m, 2H), 5.32 (dtt, J=10.6, 7.2, 1.5 Hz, 2H), 4.74 (s, 3H), 4.18-3.87 (m, 2H), 3.84-3.48 (m, 1H), 3.20-2.42 (m, 15H), 2.42-2.17 (m, 6H), 2.06 (dd, J=17.8, 10.8 Hz, 4H), 2.00 (t, J=7.1 Hz, 3H), 1.69 (pd, J=8.2, 3.3 Hz, 5H), 1.44-1.13 (m, 17H), 0.89 (t, J=6.8 Hz, 6H). ¹³C NMR (126 MHz, CDCl₃) δ 173.68, 173.60, 131.16, 131.14, 128.35, 128.33, 67.96, 66.71, 66.37, 55.95, 54.89, 54.27, 51.53, 51.32, 48.22, 43.39, 43.29, 43.07, 33.90, 33.71, 33.67, 33.66, 31.77, 29.68, 29.66, 29.01, 28.99, 27.26, 27.24, 26.57, 26.55, 26.53, 24.98, 24.94, 24.91, 22.65, 14.12. HRMS (ESI) m/z 736.6107 (calculated for C₄₂H₈₀N₄O₆[M+H]⁺ 736.6078).

AMG1241. ¹H NMR (500 MHz, CDCl₃) δ 5.41 (dt, J=13.5, 7.0 Hz, 4H), 5.36-5.25 (m, 4H), 5.01 (ddtd, J=23.3, 15.3, 7.9, 7.4, 3.8 Hz, OH), 4.10 (dh, J=10.3, 5.6 Hz, 3H), 4.02 (dt, J=11.1, 4.9 Hz, 3H), 3.98-3.90 (m, 2H), 3.90-3.70 (m, 1H), 3.67 (s, OH), 3.48 (s, OH), 3.09-2.66 (m, 5H), 2.65-2.42 (m, 2H), 2.36 (td, J=7.7, 2.1 Hz, 8H), 2.29-2.12 (m, 2H), 2.08 (q, J=7.4 Hz, 7H), 2.01 (q, J=7.0 Hz, 8H), 1.69 (pd, J=7.6, 2.8 Hz, 8H), 1.39-1.18 (m, 34H), 0.89 (t, J=6.8 Hz, 11H). ¹³C NMR (126 MHz, CDCl₃) δ 173.62, 173.60, 173.58, 131.15, 131.13, 131.11, 131.09, 128.39, 128.37, 128.33, 128.32, 70.86, 68.52, 67.25, 66.76, 66.54, 66.21, 66.13, 66.09, 66.03, 65.93, 65.70, 65.55, 65.46, 58.69, 58.56, 58.33, 58.22, 54.16, 53.20, 52.43, 52.16, 51.45, 51.27, 51.13, 33.60, 33.58, 33.56, 33.53, 31.78, 29.67, 28.99, 28.95, 27.25, 27.22, 26.57, 26.52, 26.51, 24.93, 24.88, 24.82, 22.65, 22.62, 14.14, 14.12, 14.10, 14.09. HRMS (ESI) m/z 1189.9303 (calculated for C₆₈H₁₂₅N₄O₁₂[M+H]⁺ 1189.9294).

AMG1341. ¹H NMR (500 MHz, CDCl₃) δ 5.42 (dt, J=13.3, 7.1 Hz, 3H), 5.34 (h, J=7.9, 7.2 Hz, 3H), 5.03 (dtd, J=14.4, 7.3, 6.6, 3.0 Hz, OH), 4.24-4.08 (m, 2H), 4.07 (s, OH), 3.83-3.63 (m, 1H), 3.51 (dtq, J=23.5, 11.9, 5.4 Hz, 1H), 3.15 (q, J=7.9, 6.9 Hz, OH), 3.02 (dt, J=20.8, 7.2 Hz, 1H), 2.92-2.42 (m, 2H), 2.36 (dtd, J=10.8, 8.3, 7.7, 4.0 Hz, 6H), 2.05 (dq, J=35.2, 7.3 Hz, 14H), 1.87-1.48 (m, 9H), 1.42-1.11 (m, 25H), 0.90 (t, J=6.9 Hz, 9H). ¹³C NMR (126 MHz, CDCl₃) δ 173.67, 131.20, 131.18, 131.16, 128.32, 128.31, 128.29, 71.41, 66.69, 66.66, 66.59, 66.50, 66.45, 66.41, 66.19, 65.91, 65.63, 65.38, 64.57, 64.23, 58.46, 57.03, 56.28, 56.07, 51.47, 49.26, 33.62, 33.61, 33.59, 31.78, 29.70, 29.67, 29.00, 27.26, 26.56, 26.52, 24.89, 22.66, 22.64, 14.13, 14.11. HRMS (ESI) m/z 934.7434 (calculated for C₅₄H₁₀₀N₃O₉[M+H]⁺ 934.7460).

AMG1541. ¹H NMR (500 MHz, CDCl₃) δ 5.42 (dt, J=14.5, 7.3 Hz, 6H), 5.38-5.29 (m, 6H), 5.04-4.77 (m, 6H), 4.10 (dq, J=12.2, 4.1 Hz, 5H), 4.06-3.98 (m, 5H), 3.95-3.84 (m, 5H), 3.76 (ddt, J=17.6, 11.4, 6.6 Hz, 2H), 2.90-2.42 (m, 25H), 2.36 (q, J=12.0, 9.8 Hz, 12H), 2.09 (q, J=7.3 Hz, 12H), 2.02 (q, J=7.0 Hz, 11H), 1.70 (p, J=7.6 Hz, 12H), 1.40-1.21 (m, 51H), 0.90 (t, J=6.7 Hz, 16H). ¹³C NMR (126 MHz, CDCl₃) δ 173.60, 131.27, 131.19, 128.29, 128.20, 66.75, 66.20, 65.99, 57.96, 52.80, 52.45, 33.56, 31.79, 29.67, 29.00, 27.27, 26.57, 26.53, 24.90, 22.66, 14.13, 14.12. HRMS (ESI) m/z 1784.3886 (calculated for C₁₀₂H₁₈₇N₆O₁₈[M+H]⁺ 1784.3902).

AMG1541R. ¹H NMR (600 MHz, CDCl₃) δ 5.46-5.36 (m, 6H), 5.36-5.25 (m, 6H), 5.06-4.76 (m, 7H), 4.08 (dd, J=11.5, 4.3 Hz, 5H), 4.01 (ddd, J=10.9, 5.2, 2.8 Hz, 5H), 3.90 (p, J=5.3 Hz, 5H), 3.78 (ddd, J=11.9, 5.8, 3.7 Hz, 1H), 3.72 (dt, J=11.9, 4.3 Hz, 1H), 2.87-2.72 (m, 9H), 2.65 (dddd, J=23.5, 20.6, 12.0, 5.5 Hz, 2H), 2.56-2.45 (m, 11H), 2.41 (t, J=11.8 Hz, 2H), 2.39-2.25 (m, 12H), 2.07 (qd, J=7.4, 1.4 Hz, 12H), 2.00 (qd, J=7.2, 1.5 Hz, 12H), 1.68 (h, J=8.0 Hz, 12H), 1.38-1.21 (m, 49H), 0.88 (t, J=7.0 Hz, 18H). ¹³C NMR (151 MHz, CDCl₃) δ 173.60, 173.54, 131.24, 131.16, 131.15, 128.32, 128.31, 128.29, 128.27, 128.19, 77.29, 77.07, 76.86, 66.57, 66.24, 66.19, 66.10, 65.97, 57.67, 52.72, 52.67, 51.98, 51.94, 51.89, 33.80, 33.77, 33.55, 33.53, 31.76, 29.65, 28.97, 27.25, 26.55, 26.51, 24.88, 22.64, 14.11. HRMS (ESI) m/z 1784.3890 (calculated for C₁₀₂H₁₈₇N₆O₁₈ [M+H]⁺ 1784.3902).

AMG1541S. ¹H NMR (600 MHz, CDCl₃) δ 5.47-5.39 (m, 6H), 5.34 (dtt, J=10.6, 7.2, 1.5 Hz, 6H), 5.13-4.85 (m, 4H), 4.13-4.07 (m, 5H), 3.95-3.87 (m, 6H), 3.75 (dt, J=11.6, 4.2 Hz, 1H), 2.91-2.60 (m, 12H), 2.59-2.47 (m, 13H), 2.44 (ddd, J=13.1, 10.3, 2.7 Hz, 3H), 2.40-2.28 (m, 12H), 2.10 (qd, J=7.4, 1.5 Hz, 13H), 2.02 (qd, J=7.2, 1.5 Hz, 13H), 1.79-1.61 (m, 14H), 1.44-1.16 (m, 57H), 0.90 (t, J=7.0 Hz, 18H). ¹³C NMR (151 MHz, CDCl₃) δ 173.53, 131.15, 128.27, 66.19, 65.96, 57.64, 51.98, 51.92, 51.88, 33.54, 33.51, 31.81, 31.79, 31.75, 29.70, 29.68, 29.64, 29.61, 28.96, 28.91, 27.29, 27.27, 27.24, 27.20, 26.58, 26.54, 26.50, 24.87, 22.63, 22.61, 22.59, 14.14, 14.10. HRMS (ESI) m/z 1784.3911 (calculated for C₁₀₂H₁₈₇N₆O₁₈[M+H]⁺ 1784.3902).

AMG1547. ¹H NMR (600 MHz, CDCl₃) δ 5.47-5.25 (m, 12H), 4.76 (s, 7H), 4.13-4.06 (m, 6H), 4.02 (dt, J=11.4, 4.1 Hz, 5H), 3.90 (ddt, J=14.2, 10.1, 4.8 Hz, 6H), 3.83-3.69 (m, 2H), 2.79 (s, 6H), 2.55 (d, J=27.3 Hz, 12H), 2.40-2.30 (m, 12H), 2.09-1.94 (m, 24H), 1.68-1.60 (m, 13H), 1.43-1.22 (m, 45H), 0.90 (td, J=7.0, 3.2 Hz, 18H). ¹³C NMR (151 MHz, CDCl₃) δ 173.64, 173.61, 130.50, 129.04, 67.35, 66.73, 66.20, 65.97, 63.36, 58.37, 58.27, 57.97, 57.64, 53.19, 52.81, 52.45, 51.98, 34.00, 33.94, 32.56, 32.21, 31.52, 31.50, 31.42, 29.40, 29.36, 29.29, 29.25, 29.20, 29.12, 27.21, 27.19, 26.86, 26.83, 24.59, 24.54, 24.38, 22.58, 22.55, 14.09, 14.07. HRMS (ESI) m/z 1784.3873 (calculated for C₁₀₂H₁₈₇N₆O₁₈[M+H]⁺ 1784.3902).

AMG1548. ¹H NMR (600 MHz, CDCl₃) δ 5.47-5.26 (m, 12H), 4.91 (s, 6H), 4.09 (ddd, J=13.8, 7.2, 3.8 Hz, 6H), 4.02 (ddt, J=11.5, 5.6, 2.7 Hz, 6H), 3.92-3.86 (m, 6H), 3.81-3.68 (m, 2H), 2.78 (dt, J=22.1, 11.8 Hz, 6H), 2.62-2.41 (m, 12H), 2.35 (dd, J=8.9, 6.2 Hz, 12H), 2.09-1.94 (m, 24H), 1.68-1.56 (m, 14H), 1.35 (dtt, J=18.0, 7.2, 4.4 Hz, 48H), 0.96-0.87 (m, 18H). ¹³C NMR (151 MHz, CDCl₃) δ 173.69, 173.67, 130.20, 130.16, 129.43, 129.39, 70.24, 67.40, 66.75, 66.61, 66.17, 65.95, 65.18, 63.36, 34.06, 32.39, 32.26, 31.93, 31.91, 31.80, 29.41, 29.39, 29.28, 28.82, 28.77, 28.67, 27.01, 26.99, 26.96, 26.93, 26.91, 24.80, 24.77, 22.34, 22.26, 22.20, 14.01, 13.99, 13.97, 13.74. HRMS (ESI) m/z 1784.3924 (calculated for C₁₀₂H₁₈₇N₆O₁₈[M+H]⁺ 1784.3902).

AMG1549. ¹H NMR (600 MHz, CDCl₃) δ 5.41-5.32 (m, 12H), 5.09-4.75 (m, 2H), 4.13-4.05 (m, 5H), 4.02 (ddt, J=11.5, 5.8, 2.9 Hz, 5H), 3.89 (s, 4H), 3.81-3.68 (m, 2H), 2.92-2.64 (m, 10H), 2.63-2.41 (m, 19H), 2.38-2.28 (m, 12H), 2.06-1.93 (m, 22H), 1.67-1.55 (m, 12H), 1.42-1.21 (m, 47H), 0.91 (t, J=7.4 Hz, 18H). ¹³C NMR (151 MHz, CDCl₃) δ 173.74, 173.71, 129.83, 129.81, 70.25, 67.39, 66.76, 66.24, 66.10, 65.95, 65.17, 63.37, 58.28, 57.97, 57.81, 57.62, 53.25, 52.88, 52.46, 51.99, 51.72, 34.70, 34.09, 32.52, 29.61, 29.58, 29.56, 29.45, 29.43, 29.33, 29.30, 29.28, 29.26, 29.09, 29.05, 29.00, 28.94, 28.92, 28.79, 27.14, 27.11, 24.86, 22.91, 22.90, 22.88, 22.86, 22.73, 13.85, 13.82, 13.67. HRMS (ESI) m/z 1784.3919 (calculated for C₁₀₂H₁₈₇N₆O₁₈[M+H]⁺ 1784.3902).

AMG1550. ¹H NMR (600 MHz, CDCl₃) δ 5.47-5.28 (m, 12H), 5.00 (s, 5H), 4.13-4.05 (m, 5H), 4.02 (ddt, J=11.4, 5.7, 2.9 Hz, 5H), 3.94-3.85 (m, 6H), 3.82-3.69 (m, 1H), 2.86-2.63 (m, 7H), 2.62-2.42 (m, 8H), 2.38-2.28 (m, 12H), 2.08-1.94 (m, 23H), 1.62 (q, J=7.5 Hz, 13H), 1.33 (dt, J=15.1, 5.3 Hz, 50H), 0.96 (t, J=7.5 Hz, 18H). ¹³C NMR (151 MHz, CDCl₃) δ 173.75, 173.73, 131.63, 131.61, 129.18, 67.40, 66.76, 66.61, 66.24, 66.06, 65.94, 65.17, 63.36, 58.39, 58.28, 57.96, 57.62, 53.25, 52.89, 52.42, 51.99, 51.71, 51.58, 34.34, 34.09, 32.53, 29.70, 29.67, 29.66, 29.64, 29.59, 29.25, 29.20, 29.15, 29.12, 29.10, 29.08, 29.05, 29.04, 29.00, 28.98, 28.95, 27.05, 27.03, 25.60, 24.89, 20.51, 20.50, 20.48, 14.43, 14.40, 14.37, 14.35, 14.01. HRMS (ESI) m/z 1784.3931 (calculated for C₁₀₂H₁₈₇N₆O₁₈ [M+H]⁺ 1784.3902).

AMG1551. ¹H NMR (600 MHz, CDCl₃) δ 5.49-5.34 (m, 12H), 5.08-4.74 (m, 1H), 4.08 (dddd, J=9.1, 6.8, 5.0, 2.8 Hz, 4H), 4.02 (ddt, J=11.6, 5.9, 3.0 Hz, 4H), 3.94-3.86 (m, 4H), 3.82-3.67 (m, 2H), 2.86-2.63 (m, 7H), 2.60-2.42 (m, 8H), 2.38-2.28 (m, 10H), 2.03 (q, J=7.1 Hz, 9H), 1.99-1.90 (m, 1H), 1.67-1.57 (m, 24H), 1.40-1.25 (m, 52H). ¹³C NMR (151 MHz, CDCl₃) δ 173.77, 173.74, 131.59, 130.80, 124.58, 123.65, 70.25, 67.40, 67.16, 66.76, 66.62, 66.25, 66.08, 65.93, 65.17, 63.36, 58.40, 58.28, 57.96, 57.79, 57.62, 53.24, 52.89, 52.44, 51.97, 51.71, 51.59, 34.10, 32.58, 29.59, 29.54, 29.51, 29.49, 29.46, 29.43, 29.42, 29.39, 29.37, 29.35, 29.34, 29.32, 29.28, 29.27, 29.23, 29.18, 29.13, 26.82, 26.80, 26.77, 24.89, 17.93, 12.76. HRMS (ESI) m/z 1784.3912 (calculated for C₁₀₂H₁₈₇N₆O₁₈[M+H]⁺ 1784.3902).

AMG1553. ¹H NMR (500 MHz, CDCl₃) δ 5.48-5.27 (m, 24H), 4.84 (s, 7H), 4.10 (dt, J=13.3, 4.1 Hz, 6H), 4.06-3.96 (m, 6H), 3.94-3.85 (m, 6H), 3.80-3.67 (m, 2H), 2.81 (t, J=6.9 Hz, 14H), 2.78-2.63 (m, 6H), 2.60-2.46 (m, 12H), 2.45-2.28 (m, 12H), 2.10-1.98 (m, 13H), 1.33 (qd, J=7.2, 3.8 Hz, 29H), 0.95-0.86 (m, 18H). ¹³C NMR (126 MHz, CDCl₃) δ 173.06, 130.76, 130.59, 130.54, 129.94, 129.86, 129.77, 128.25, 127.56, 127.43, 127.36, 127.19, 70.18, 67.39, 66.73, 66.58, 66.20, 66.05, 65.29, 65.15, 63.33, 58.37, 57.92, 57.56, 53.21, 52.88, 52.46, 52.10, 51.94, 51.66, 51.50, 51.26, 34.24, 34.02, 33.98, 31.81, 31.80, 31.67, 30.34, 27.78, 26.95, 26.93, 26.83, 25.59, 22.68, 22.62, 22.34, 22.32, 14.07, 14.02, 14.00. HRMS (ESI) m/z 1772.2944 (calculated for C₁₀₂H₁₇₅N₆O₁₈ [M+H]⁺ 1772.2963).

AMG1554. ¹H NMR (500 MHz, CDCl₃) δ 5.44-5.29 (m, 24H), 4.94 (s, 2H), 4.09 (dt, J=12.0, 4.1 Hz, 6H), 4.05-3.97 (m, 5H), 3.94-3.84 (m, 6H), 3.82-3.65 (m, 3H), 2.87-2.66 (m, 22H), 2.64-2.42 (m, 7H), 2.41-2.29 (m, 13H), 2.12 (q, J=7.1 Hz, 12H), 2.04 (q, J=7.1 Hz, 14H), 1.71 (h, J=7.7 Hz, 13H), 1.44-1.32 (m, 14H), 0.92 (t, J=7.4 Hz, 18H). ¹³C NMR (126 MHz, CDCl₃) δ 173.59, 173.54, 173.51, 130.40, 130.20, 130.16, 129.34, 129.27, 128.64, 128.61, 128.52, 127.83, 127.79, 127.61, 67.39, 66.74, 66.19, 66.01, 65.09, 63.36, 58.41, 58.27, 57.95, 57.61, 53.24, 52.88, 52.44, 51.97, 51.68, 33.51, 33.44, 31.89, 30.40, 29.29, 29.20, 26.55, 26.51, 26.49, 25.63, 25.60, 24.78, 24.64, 22.77, 13.82, 13.80. HRMS (ESI) m/z 1772.2966 (calculated for C₁₀₂H₁₇₅N₆O₁₈ [M+H]⁺ 1772.2963).

AMG1555. ¹H NMR (500 MHz, CDCl₃) δ 5.46-5.35 (m, 17H), 5.35-5.25 (m, 6H), 4.81 (s, 6H), 4.08 (dt, J=12.3, 4.1 Hz, 7H), 4.01 (dt, J=11.7, 4.1 Hz, 6H), 3.95-3.84 (m, 6H), 3.80-3.70 (m, 3H), 2.90-2.61 (m, 36H), 2.60-2.42 (m, 6H), 2.40-2.28 (m, 16H), 2.19 (dtd, J=14.8, 7.4, 6.6, 2.7 Hz, 1H), 2.12-1.97 (m, 31H), 1.65 (h, J=6.4, 5.2 Hz, 18H), 1.40 (qp, J=7.4, 5.8, 4.2 Hz, 17H), 1.01-0.85 (m, 23H). ¹³C NMR (126 MHz, CDCl₃) δ 173.57, 132.16, 131.91, 131.89, 129.36, 129.30, 129.28, 128.61, 128.55, 127.21, 127.18, 126.99, 70.21, 68.31, 67.39, 66.74, 66.59, 66.17, 65.97, 65.19, 65.05, 63.36, 58.27, 57.94, 57.60, 53.22, 52.09, 49.37, 34.96, 34.20, 33.97, 33.92, 32.16, 30.30, 29.21, 29.13, 29.08, 29.06, 28.98, 27.33, 26.86, 26.82, 26.79, 25.53, 24.51, 24.41, 24.38, 22.55, 20.55, 20.53, 20.44, 14.30, 14.28, 14.27, 13.76. HRMS (ESI) m/z 1772.2959 (calculated for C₁₀₂H₁₇₅N₆O₁₈ [M+H]⁺ 1772.2963).

AMG1556. ¹H NMR (500 MHz, CDCl₃) δ 5.52-5.41 (m, 6H), 5.44-5.31 (m, 18H), 4.83 (s, 4H), 4.09 (dq, J=12.2, 4.2 Hz, 6H), 4.05-3.97 (m, 5H), 3.89 (t, J=9.0 Hz, 6H), 3.81-3.69 (m, 3H), 2.79 (d, J=6.4 Hz, 12H), 2.79-2.65 (m, 6H), 2.52 (dd, J=18.6, 10.7 Hz, 6H), 2.39-2.28 (m, 13H), 2.08 (s, 5H), 2.06 (d, J=6.6 Hz, 7H), 1.69-1.60 (m, 32H), 1.36 (qq, J=9.0, 4.7, 3.7 Hz, 29H). ¹³C NMR (126 MHz, CDCl₃) δ 173.67, 129.80, 129.76, 128.86, 128.14, 128.10, 124.01, 68.34, 67.40, 66.75, 66.60, 65.96, 65.05, 63.36, 58.37, 57.95, 57.61, 53.23, 52.52, 51.94, 34.04, 30.44, 30.07, 29.31, 29.28, 29.24, 29.16, 28.81, 28.75, 28.73, 28.68, 27.03, 27.01, 26.99, 26.91, 25.30, 25.28, 24.79, 17.93, 12.79. HRMS (ESI) m/z 1772.2934 (calculated for C₁₀₂H₁₇₅N₆O₁₈ [M+H]⁺ 1772.2963).

AMG1559. ¹H NMR (500 MHz, CDCl₃) δ 5.45-5.30 (m, 12H), 4.09 (t, J=6.8 Hz, 12H), 2.92 (t, J=7.2 Hz, 3H), 2.80 (t, J=7.3 Hz, 7H), 2.75 (s, 5H), 2.54 (s, 17H), 2.45 (t, J=7.3 Hz, 8H), 2.13-1.95 (m, 24H), 1.66 (p, J=7.0 Hz, 13H), 1.48-1.39 (m, 12H), 1.38-1.24 (m, 39H), 0.96-0.87 (m, 18H). ¹³C NMR (126 MHz, CDCl₃) δ 172.63, 130.67, 130.62, 129.01, 64.75, 64.45, 52.78, 51.75, 50.66, 32.77, 32.62, 31.79, 31.76, 29.70, 29.58, 29.01, 28.27, 28.24, 27.28, 26.77, 26.04, 22.67, 14.13. HRMS (ESI) m/z 1688.4221 (calculated for C₁₀₂H₁₈₇N₆O₁₂ [M+H]⁺ 1688.4207).

AMG15C12. ¹H NMR (500 MHz, CDCl₃) δ 5.12-4.77 (m, 2H), 4.20-3.99 (m, 10H), 3.90 (s, 4H), 3.77 (dtd, J=21.9, 15.2, 9.7 Hz, 3H), 3.15-2.42 (m, 40H), 2.42-2.10 (m, 13H), 1.63 (h, J=8.2 Hz, 14H), 1.39-1.09 (m, 100H), 0.90 (t, J=6.9 Hz, 18H). ¹³C NMR (126 MHz, CDCl₃) δ 173.81, 65.95, 34.12, 31.93, 29.65, 29.52, 29.50, 29.36, 29.34, 29.32, 29.21, 29.17, 24.92, 22.70, 14.14. HRMS (ESI) m/z 1796.4867 (calculated for C₁₀₂H₁₉₉N₆O₁₈ [M+H]⁺ 1796.4841).

AMG1641. ¹H NMR (500 MHz, CDCl₃) δ 5.43 (dq, J=14.3, 7.1 Hz, 5H), 5.39-5.26 (m, 5H), 4.29-4.13 (m, 8H), 4.08 (h, J=6.4, 5.6 Hz, 3H), 3.95 (p, J=5.2 Hz, 2H), 3.85 (t, J=4.5 Hz, 2H), 3.71 (dd, J=11.5, 3.9 Hz, 2H), 3.61 (dd, J=11.3, 5.6 Hz, 2H), 3.53 (dd, J=10.5, 5.0 Hz, 2H), 3.47 (dd, J=10.6, 5.9 Hz, 2H), 2.76-2.60 (m, 5H), 2.38 (td, J=7.6, 1.8 Hz, 10H), 2.10 (q, J=7.3 Hz, 11H), 2.02 (q, J=7.1 Hz, 11H), 1.71 (hept, J=7.5 Hz, 10H), 1.31 (ddt, J=18.5, 12.8, 7.6 Hz, 46H), 0.90 (t, J=6.7 Hz, 15H). ¹³C NMR (126 MHz, CDCl₃) δ 174.20, 173.76, 131.39, 131.36, 128.13, 128.11, 75.03, 73.10, 70.26, 69.29, 69.28, 65.72, 65.70, 65.21, 63.35, 62.49, 62.40, 34.91, 33.67, 33.54, 33.48, 31.77, 29.99, 29.66, 28.99, 27.26, 26.50, 26.48, 24.89, 24.87, 24.84, 24.81, 22.65, 14.13, 14.11. HRMS (ESI) m/z 1487.1618 (calculated for C₈₅H₁₅₆N₅O₁₅ [M+H]⁺ 1487.1598).

AMG1741. ¹H NMR (500 MHz, CDCl₃) δ 5.43 (dq, J=15.1, 7.4 Hz, 7H), 5.38-5.27 (m, 7H), 4.25-4.20 (m, 5H), 4.17 (dd, J=11.7, 6.1 Hz, 3H), 4.12-3.99 (m, 5H), 3.94 (tq, J=11.3, 7.0, 5.6 Hz, 3H), 3.89-3.82 (m, 2H), 3.71 (dd, J=11.4, 3.9 Hz, 3H), 3.61 (dd, J=11.4, 5.8 Hz, 3H), 3.53 (dd, J=10.5, 5.0 Hz, 2H), 3.47 (dd, J=10.6, 5.9 Hz, 1H), 2.86-2.45 (m, 17H), 2.45-2.30 (m, 14H), 2.09 (p, J=6.8, 6.4 Hz, 15H), 2.02 (q, J=7.1 Hz, 14H), 1.71 (hept, J=7.8 Hz, 14H), 1.41-1.22 (m, 63H), 0.90 (t, J=6.7 Hz, 21H). ¹³C NMR (126 MHz, CDCl₃) δ 174.19, 173.76, 131.39, 131.36, 131.20, 128.27, 128.16, 128.13, 128.11, 75.05, 73.10, 70.26, 69.30, 65.72, 65.21, 65.17, 63.35, 63.33, 62.50, 62.43, 34.92, 33.56, 33.54, 33.47, 31.79, 31.77, 29.67, 29.66, 28.99, 27.26, 26.56, 26.53, 26.50, 26.48, 24.89, 24.87, 24.84, 24.81, 22.65, 14.13, 14.11, 14.09. HRMS (ESI) m/z 1041.3133 (calculated for C₁₁₉H₂₁₉N₇O₂₁ [M+2H]²⁺/2 1041.3142, [M+H]⁺ 2082.6284).

AMG1841. ¹H NMR (500 MHz, CDCl₃) δ 7.29 (d, J=6.8 Hz, 5H), 5.42 (dt, J=14.4, 7.3 Hz, 7H), 5.33 (q, J=8.9 Hz, 7H), 4.20-3.84 (m, 8H), 3.84-3.57 (m, 5H), 3.43-2.89 (m, 4H), 2.89-2.42 (m, 9H), 2.36 (qq, J=10.8, 5.6 Hz, 13H), 2.05 (dq, J=35.0, 7.3 Hz, 29H), 1.95 (s, OH), 1.70 (pd, J=7.8, 5.2 Hz, 15H), 1.39-1.16 (m, 52H), 0.90 (t, J=6.7 Hz, 18H). ¹³C NMR (126 MHz, CDCl₃) δ 173.66, 173.63, 173.60, 131.18, 131.16, 128.34, 128.33, 128.30, 67.00, 66.34, 66.18, 65.79, 65.19, 33.66, 33.60, 31.78, 29.67, 29.00, 27.26, 26.59, 26.56, 24.97, 24.95, 24.91, 24.88, 22.66, 14.13. MALDI-MS m/z 2028.801 (calculated for C₁₁₈H₂₁₁N₈O₁₈ [M+H]⁺ 2028.5841).

AMG2041. ¹H NMR (500 MHz, CDCl₃) δ 5.51-5.39 (m, 6H), 5.33 (dt, J=11.6, 7.1 Hz, 6H), 4.12 (d, J=27.4 Hz, 15H), 3.74 (t, J=32.8 Hz, 2H), 2.93 (t, J=110.7 Hz, 18H), 2.36 (dt, J=15.2, 7.1 Hz, 13H), 2.08 (q, J=7.3 Hz, 12H), 2.02 (q, J=7.0 Hz, 12H), 1.68 (h, J=8.0 Hz, 12H), 1.42-1.04 (m, 49H), 0.90 (t, J=6.8 Hz, 17H). ¹³C NMR (126 MHz, CDCl₃) δ 173.66, 131.20, 128.28, 66.06, 33.60, 33.54, 31.79, 29.67, 29.00, 27.28, 26.57, 26.54, 26.51, 24.88, 24.84, 22.66, 14.13. HRMS (ESI) m/z 1786.4071 (calculated for C₁₀₂H₁₈₉N₆O₁₈ [M+H]⁺ 1786.4058).

AMG2141. ¹H NMR (500 MHz, CDCl₃) δ 5.42 (dtd, J=10.8, 7.1, 1.7 Hz, 7H), 5.33 (dddt, J=11.2, 7.7, 6.1, 2.1 Hz, 7H), 4.17-4.08 (m, 5H), 4.02 (dd, J=11.4, 6.1 Hz, 4H), 3.90 (s, 5H), 3.81-3.63 (m, 3H), 2.72-2.53 (m, 19H), 2.52-2.29 (m, 44H), 2.13-2.06 (m, 15H), 2.02 (q, J=6.4 Hz, 14H), 1.81-1.53 (m, 26H), 1.41-1.21 (m, 59H), 0.90 (t, J=6.8 Hz, 18H). ¹³C NMR (126 MHz, CDCl₃) δ 173.67, 131.24, 131.17, 128.32, 128.23, 74.85, 74.83, 66.43, 65.96, 63.91, 53.02, 52.41, 33.87, 33.83, 33.60, 31.79, 29.68, 29.00, 27.27, 26.58, 26.54, 24.96, 24.90, 22.66, 14.13, 14.11. HRMS (ESI) m/z 1868.4825 (calculated for C₁₀₈H₁₉₉N₆O₁₈ [M+H]⁺ 1868.4841).

AMG2241. ¹H NMR (500 MHz, CDCl₃) δ 5.42 (dtt, J=10.2, 7.1, 1.5 Hz, 7H), 5.33 (dtt, J=10.5, 7.2, 1.5 Hz, 7H), 4.13 (tq, J=7.7, 4.5 Hz, 6H), 4.02 (ddd, J=11.5, 6.1, 2.4 Hz, 5H), 3.90 (d, J=13.8 Hz, 6H), 3.77 (d, J=5.0 Hz, 2H), 2.74-2.52 (m, 18H), 2.52-2.28 (m, 42H), 2.09 (q, J=7.3 Hz, 14H), 2.02 (q, J=6.9 Hz, 13H), 1.69 (dt, J=24.3, 12.1 Hz, 21H), 1.49 (d, J=22.6 Hz, 8H), 1.35-1.20 (m, 46H), 0.90 (t, J=6.9 Hz, 18H). ¹³C NMR (126 MHz, CDCl₃) δ 173.66, 131.18, 128.31, 74.83, 66.45, 66.05, 65.85, 57.05, 54.96, 54.31, 53.02, 52.52, 33.82, 33.60, 31.78, 31.76, 29.67, 29.00, 27.26, 26.57, 26.54, 24.91, 22.66, 14.13. HRMS (ESI) m/z 1896.5175 (calculated for C₁₁₀H₂₀₃N₆O₁₈ [M+H]⁺ 1896.5154).

Representative LNP Characterization Data. The diameter, polydispersity index (PDI), and % EE were characterized for various LNPs.

| LNP | Diameter (nm) | PDI | EE % |
|---|---|---|---|
| AMG1041 | 81.79 | 0.113 | |
| AMG1042 | 79.97 | 0.306 | |
| AMG1043 | 79.27 | 0.169 | |
| AMG1044 | 94.5 | 0.191 | |
| AMG1541 | 85 | 0.107 | 86.0935 |
| AMG1547 | 100 | 0.142 | 85.33009 |
| AMG1548 | 104.7 | 0.106 | 54.6 |
| AMG1549 | 100.4 | 0.234 | 56.9 |
| AMG1550 | 93 | 0.119 | 78.08734 |
| AMG1551 | 77 | 0.145 | 67.40648 |
| AMG1553 | 94 | 0.09 | 89.0186 |
| AMG1554 | 105 | 0.1 | 91.711 |
| AMG1555 | 83 | 0.077 | 88.37399 |
| AMG1556 | 96 | 0.121 | 85.75766 |
| AMG1741 | 88 | 0.156 | 82.99264 |

### Example 3: Evaluation of the LNPs In Vivo

mRNA vaccines are a versatile platform which can be rapidly developed and deployed to combat emerging pathogens. However, the next generation of mRNA vaccines must address several limitations, including enhancing vaccine potency and accessibility. Developing effective vaccine delivery systems can significantly improve the efficacy of these platforms, creating dose-sparing effects that boost both protective efficacy and safety. In this example, epoxide ring opening chemistry was used to develope ionizable lipids via sequential combinatorial chemistry and rational design approaches. Lipid nanoparticles (LNPs) formulated with one of the top-performing ionizable lipids, AMG1541, achieved approximately three times higher in vivo mRNA delivery compared to FDA-approved counterparts and demonstrated rapid clearance (t_{1/2}=3 hours), demonstrating their potency and rapid degradation. Notably, the rapid degradation of the ionizable lipid reduced mRNA expression in the liver and spleen, potentially mitigating side effects associated with hepatic migration of LNPs following intramuscular vaccination. Furthermore, these LNPs elicited similar protective immunity to the FDA-approved ionizable lipid SM-102 at a 100-fold lower dose. This enhanced efficacy was driven by improved mRNA delivery to antigen-presenting cells in the local tissue and the draining lymph node, leading to stronger germinal center reactions. Without wishing to be bound by any particular theory, structure-activity relationship studies suggested that cyclic headgroups and R-amino alcohols facilitated interactions with the mRNA backbone and endosomal membrane, enhancing RNA delivery. This LNP platform may significantly enhance the potency and safety profile of mRNA vaccines.

Messenger RNA (mRNA) vaccines are one of the most significant global health advancements of the past decade. Their inherent adaptability and efficient manufacturing processes allows them to be rapidly updated and deployed in response to emerging pathogens. These strengths were demonstrated during the COVID-19 pandemic, with over 600 million doses administered in just one year.¹ Although mRNA vaccines have become an undeniable success story, their large-scale clinical use has revealed opportunities for improvement.² For example, increasing the immunogenicity of these vaccines is desirable to maximize the impact of future vaccination campaigns and help ensure widespread herd immunity during future pandemics.⁴ This example explores engineering new lipid nanoparticles (LNPs) to enhance the expression of antigen-encoding mRNA. More immunogenic mRNA vaccines have the potential to maintain or enhance protective immunity at lower doses, potentially reducing side effects and improving vaccine efficacy, as suggested in a clinical trial.³ Lower dosages would also reduce the cost per dose and increase manufacturing throughput, potentially facilitating better global distribution.

There are various approaches to increasing mRNA vaccine immunogenicity.^{4,5} The LNP delivery vehicle plays several roles in initiating vaccine responses. LNPs facilitate immune cell recruitment and uptake, endosomal escape, and help augment the immunogenicity of antigens. Clinically approved LNPs consist of four components: an ionizable lipid, cholesterol, a helper phospholipid, and a PEG lipid.⁶ Of these components, the ionizable lipid influences antigen production,⁷ immunogenicity,⁸ and reactogenicity (systemic or local adverse effects).⁹ Historically, ionizable lipids have been typically developed using either rational lipid design or combinatorial lipid screening. The first of these approaches utilizes structural motifs from previous top-performing ionizable lipids to iteratively generate improved lipids. Although rational design approaches have relatively high “hit” rates, they inherently generate limited structural diversity. In contrast, combinatorial chemistry allows for the exploration of a broader structural diversity by combining different building blocks, resulting in unique structural motifs in ionizable lipids.¹⁰⁻¹² However, this approach tends to have lower hit rates necessitating more extensive screening efforts. Consequently, neither approach is optimal for the development of new ionizable lipids for improved mRNA vaccine efficacy. This example discloses a hybrid rational design and combinatorial approach to develop a structurally unique series of ionizable lipids for mRNA vaccination, ultimately leading to identification of a potent ionizable lipid for mRNA vaccination.

Previously, combinatorial approaches were used to generate various ionizable lipid libraries derived from nucleophilic attack by amines on terminal epoxide ring of a hydrophobic tail which resulted in highly potent ionizable lipids such as C12-200 and cKK-E12.^{10,11} Here, epoxide ring opening chemistry was used to generate an ionizable lipid library utilizing a high-yield, reaction between “clickable” glycidyl ester functionalized hydrocarbon tails and constrained, cyclic secondary amines. Lipids from this combinatorial screen were used as a starting point for rational structure-activity development of the lipid headgroup and tail structures. AMG1541 was identified as a highly potent and rapidly biodegradable ionizable lipid which results in high levels of mRNA expression following intramuscular (IM) administration on par with the best-in-class ionizable lipids. AMG1541's potency gives remarkably stronger adaptive immune responses, yielding 13-fold higher neutralizing titers than the FDA approved ionizable lipid SM-102 at an equivalent vaccine dose. A variety of tools including ³¹P NMR were employed to evaluate the structural features which contribute to AMG1541's potency.

Design and Synthesis of AMG Lipid Library. Epoxide ring opening chemistry was used to synthesize an ionizable lipid library due to its high yield, amenability to parallel synthesis, scalability, and minimal generation of side-products, enabling simpler purification.^{10,11,13,14} Notably, epoxide ring opening chemistry also generates β-amino alcohols which enhance RNA delivery efficacy compared to their counterpart lipids bearing only tertiary amines. For example, the lipid cKK-E12 showed several-fold higher siRNA delivery activity compared to cKK-A12, which lacks β-amino alcohols.¹⁰ Both FDA-approved ionizable lipids also contain amino alcohols; SM-102 and ALC-0315 feature β- and y-amino alcohols, respectively. In addition to enhancing delivery, the lipids provided herein improve lipid clearance and tolerability by introducing one or more biodegradable ester groups into the tail structures.^{7,15} To achieve this chemical feature, glycidyl ester lipid tails were synthesized via Shiina esterification. However, these tails were more reactive and less stable compared to their nondegradable counterparts and were not compatible with the extended heating at high temperature conditions used in previous reports.^{10,11} After modifying reaction conditions, ionizable lipids could be obtained in just 1 hour at 80-90° C. by reacting these glycidyl esters with secondary amines in a microwave reactor using a polar, protic solvent such as isopropanol. These conditions consistently yielded fully substituted products with high isolated yields making it amenable to parallel lipid synthesis.^{10,11,13,16}

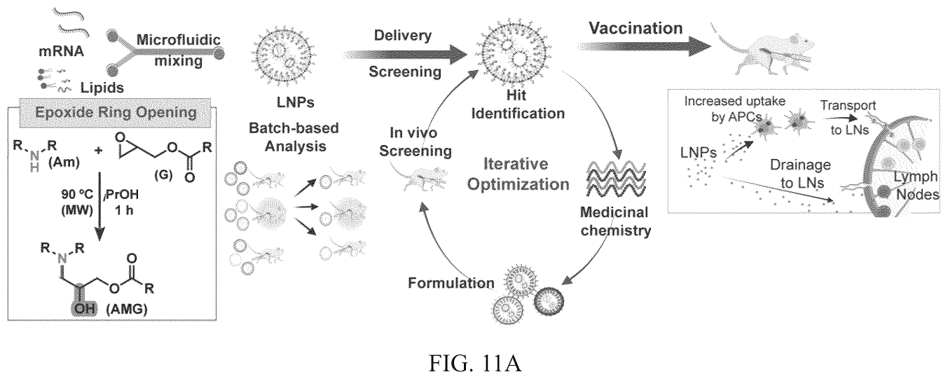

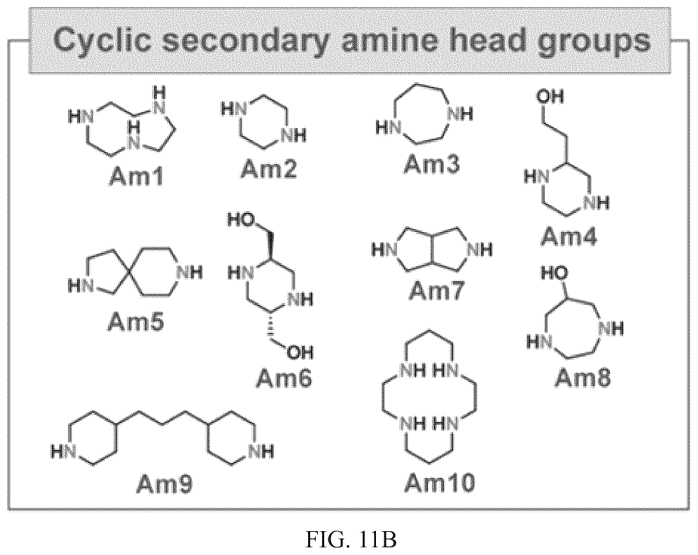

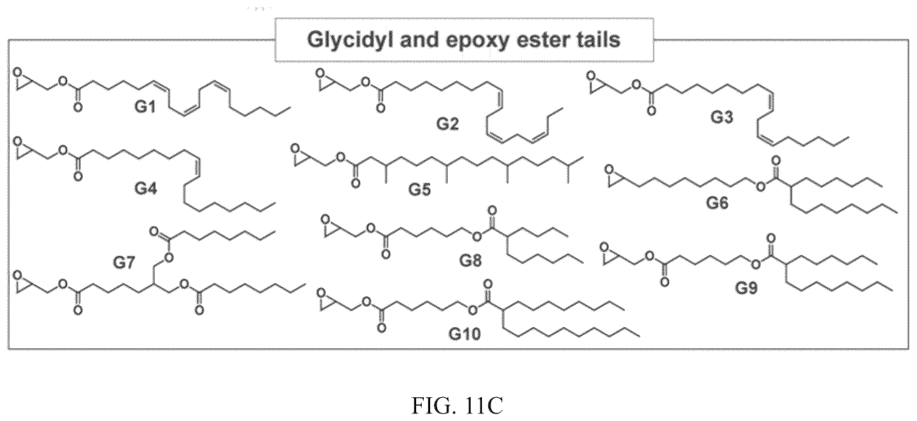

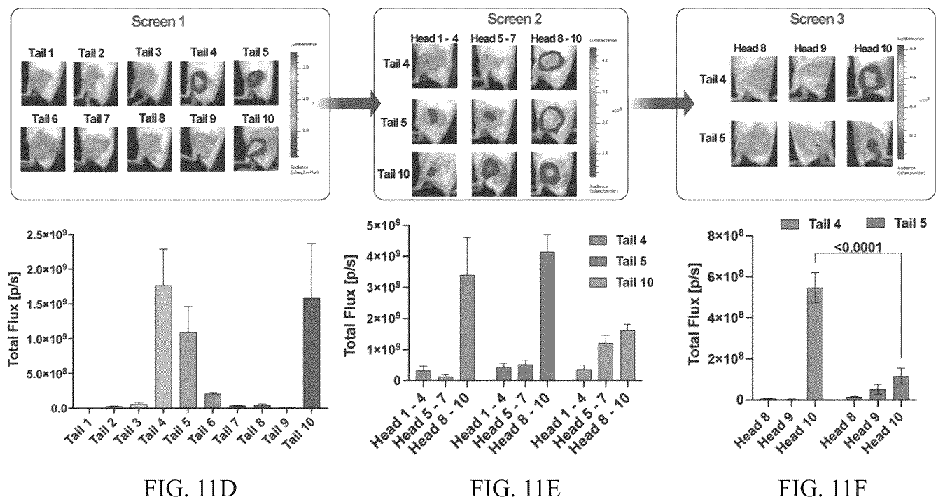

After identifying these reaction conditions, a combinatorial library was developed, containing 10 amine headgroups (Am), 9 glycidyl esters and 1 epoxy-ester tails (G) which, combined, gave 100 unique ionizable lipids (AMG). These headgroups featured both monocyclic and bicyclic amines (Am 2, 3, 4, 6, and 8) spiro (Am 5) and fused rings (Am 7), one with adjacent rings connected by flexible alkyl linkers (Am9), and aza-crown ethers (Am 1 and 10). This diversity in structural headgroups was incorporated to facilitate lipid-RNA interactions and enhance delivery efficacy. The tails included Z-alkenes with varying degrees of unsaturation or branching, to assess whether these features increase lipid fusogenicity and enhance mRNA delivery.^{13,17,18} The resultant ionizable lipids were designated by combining the headgroup number (Am #) with that of the glycidyl ester (G #) as shown in FIG. 11. For example, AMG104 refers to the compound formed by the combination of Am10 headgroup with G4 glycidyl ester.

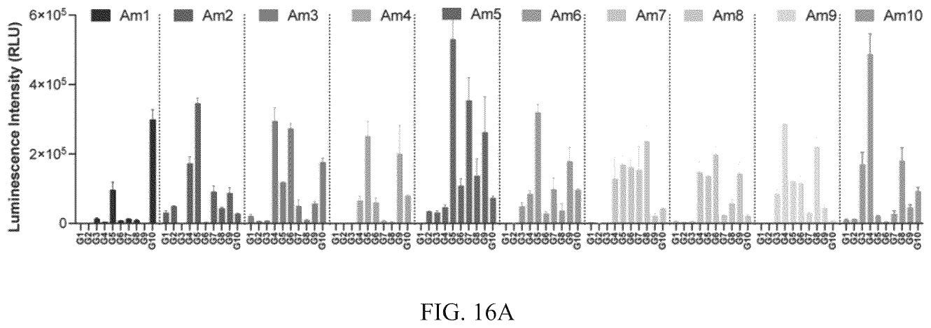

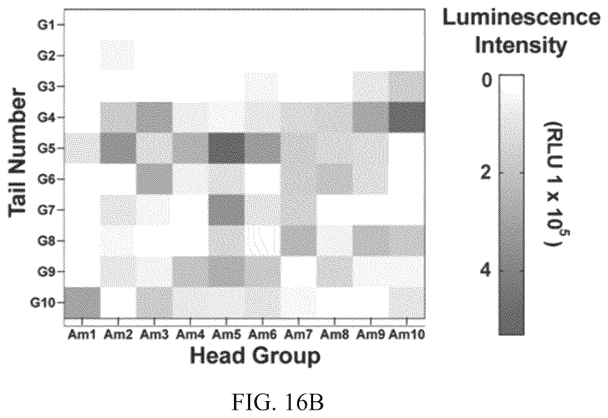

In Vitro and In Vivo Screening of AMG Lipid Library. Following synthesis, the mRNA delivery efficacy of the AMG lipids was evaluated through both in vitro and in vivo screens. To screen the first generation of AMG lipids (FIGS. 11B and 11C), firefly luciferase (FLuc) mRNA was encapsulated into LNPs using a previously reported formulation containing ionizable lipid, 1,2-dioleoyl-sn-glycero-3-phosphoethanolamine (DOPE), cholesterol, and 1,2-dimyristoyl-rac-glycero-3-methoxypolyethylene glycol-2000 (DMG-PEG).¹⁹ C2C12 myoblast cells were treated with these LNPs and FLuc expression was evaluated at 24 hrs. (FIG. 16). Many of these LNPs outperformed the benchmark LNP formulated with cKK-E12. Given the potency of these ionizable lipids in vitro, their mRNA transfection was evaluated in vivo.²⁰ To do so, a batch-based analysis approach that allows for in vivo screening of large LNP libraries was used. This screening strategy significantly reduced the number of animals, time, and cost while still accurately identifying the performance of LNPs in the library.^{8,21} In the first screening round, each AMG lipid was individually formulated into LNPs with FLuc mRNA and pooled into 10 different batches based on the structure of their tail groups. Batches were generated based on the tail structure, as this had the largest effect on mRNA delivery in vitro (FIG. 16). Each of these batches was then injected IM into the quadriceps of mice, and expression was characterized via IVIS imaging (Screen 1, FIG. 11E). Among the 10 pools, LNPs featuring tails G4, G5 and G10 demonstrated the highest mRNA expression and were therefore selected for further testing. Subsequently, the ionizable lipids with potent tails were reformulated and divided into smaller batches containing headgroups Am1-Am4, Am5-Am7, or Am8-Am10. These 9 smaller batches (Screen 2) were then injected IM to further refine and identify the most potent LNP formulations. The luminescent signal (FIG. 11F) revealed that the batches containing Am8-Am10 with tails G4 and G5 resulted in strongest mRNA transfection. In Screen 3, the 6 individual lipids from these top-performing batches were tested separately in mice (FIG. 11F). These results showed that, of the lipids tested, AMG104 yielded the highest mRNA expression and outperformed the next best lipid AMG1010 by ˜5-fold.

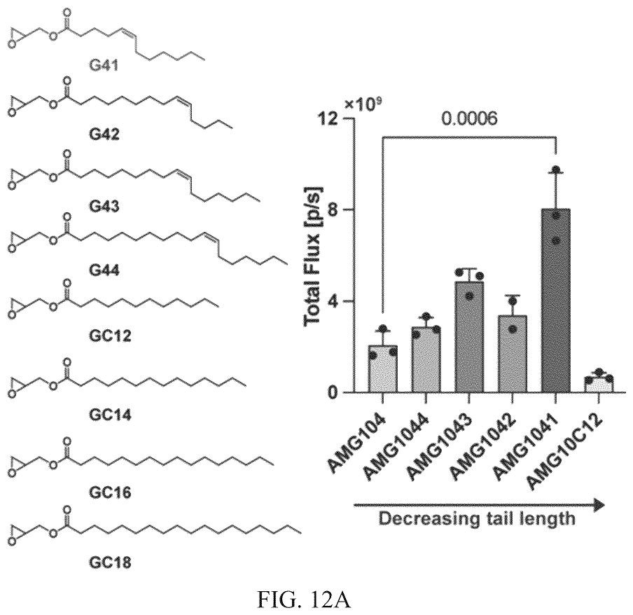

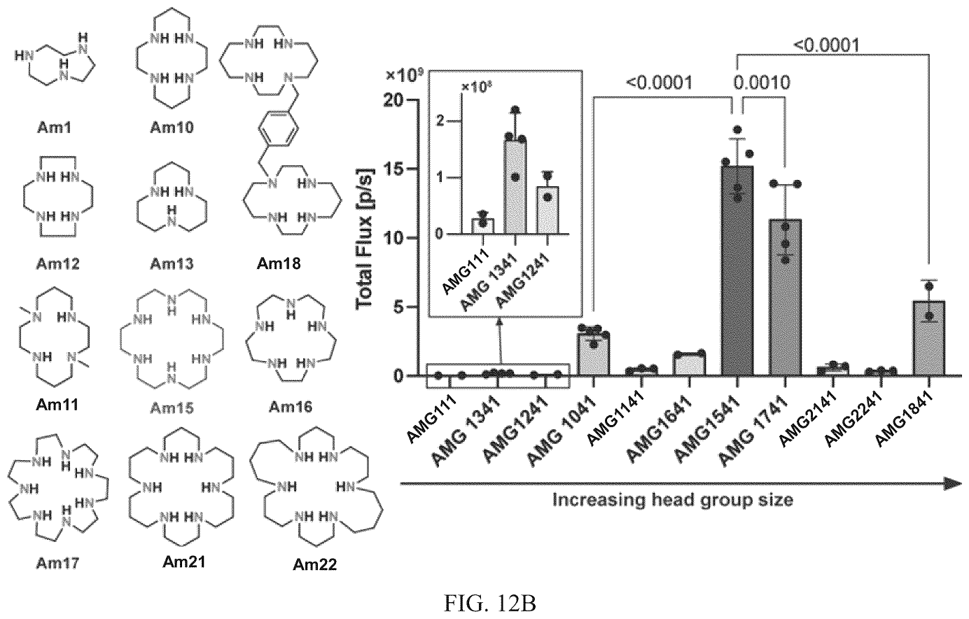

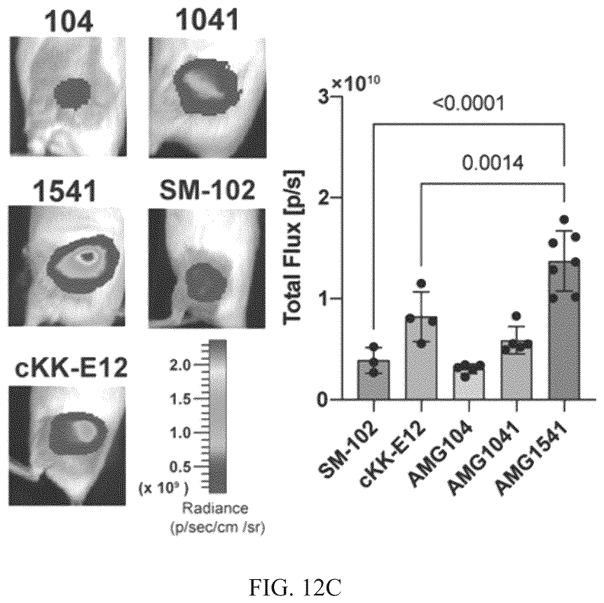

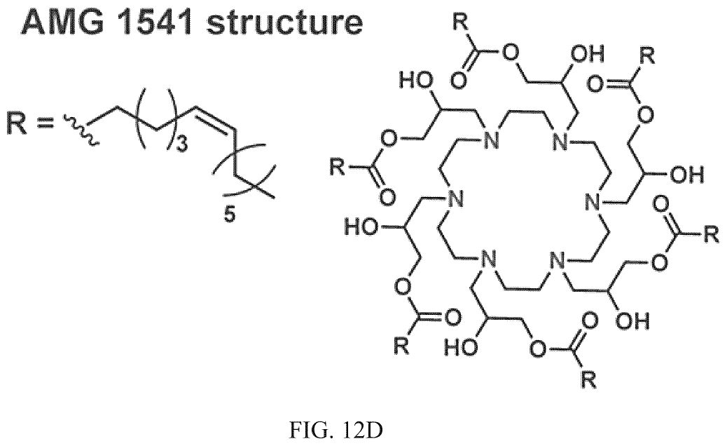

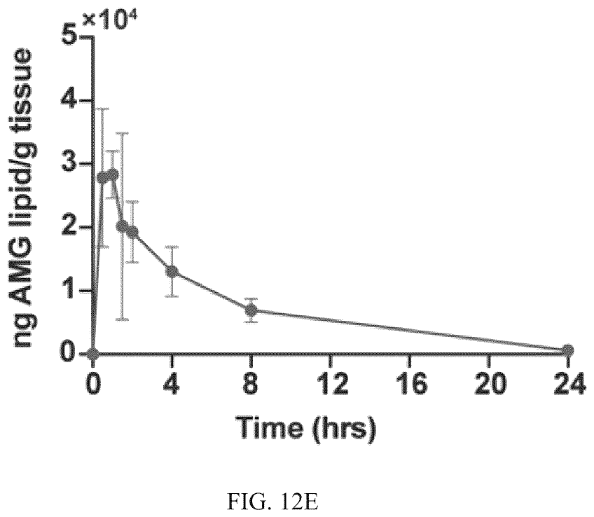

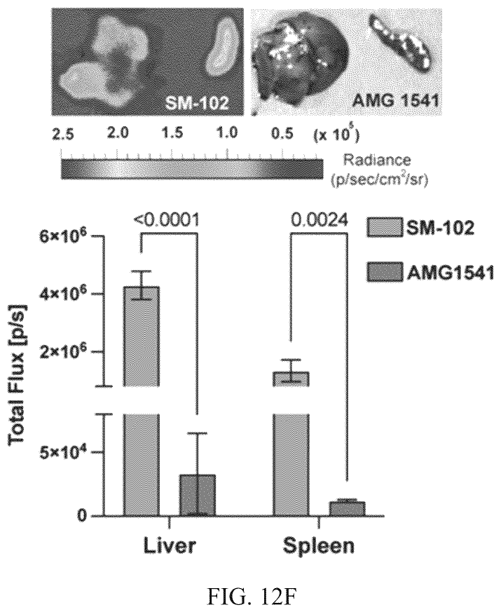

AMG lipids for enhanced mRNA delivery and improved tolerability. A rational design approach was used to further vary the ionizable lipid structure to enhance the mRNA delivery and immunogenicity. Hence, a new series of lipids was developed using the structural motifs from headgroup Am10 (1,4,8,11-tetraazacyclotetradecane) and tail G4 (glycidyl oleate) as a starting point. The lipid tail structure was varied to promote efficient LNP self-assembly while simultaneously promoting endosomal membrane disruption.¹⁸ Monounsaturated (G41-G44) or fully saturated (GC12-GC18) lipid tails were developed. When ionizable lipids were prepared by reacting these tails with AM10, AMG10C14, AMG10C16, and AMG10C18 had poor ethanol solubility and were eliminated from additional testing. The remaining five lipids were then formulated into LNPs with FLuc mRNA and injected IM (FIG. 12A). The results showed that the monounsaturated tails outperformed the GC12 saturated tail. Without wishing to be bound by any particular theory, unsaturation may promote endosomal membrane disruption.^{13,18,22} Within the unsaturated tails, there was a general trend that decreasing the tail length of the monounsaturated tails from 18 carbons to 12 carbons increased mRNA expression, with AMG1041 having the highest expression of these lipids, giving ˜3-fold higher expression than AMG104. Without wishing to be bound by any particular theory, tail length may impact membrane fusion and endosomal escape.^{10,11}

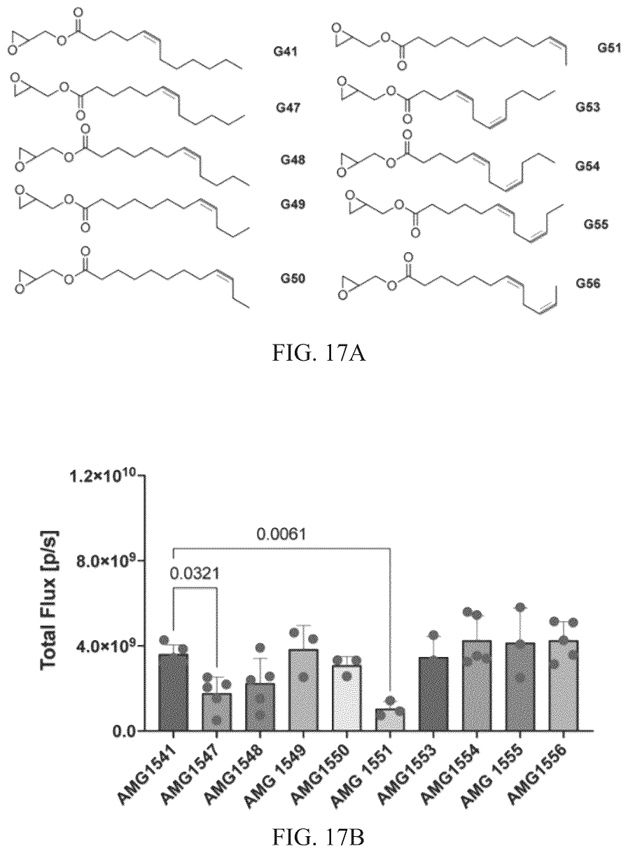

Next, the headgroup was further modified to further improve delivery. Headgroups play a multifunctional role including enhancing LNP-mRNA interactions, promoting membrane-LNP fusion and endosomal escape which all contribute to mRNA delivery.²³ A series of similar aza-crown ethers were used to further investigate structure-activity relationship and enhance mRNA delivery. These headgroups featured between three and eight nitrogens linked by varying length of methylene chains. Each of these headgroups were reacted with tail G41 to synthesize a next generation of AMG lipids, which were then formulated with FLuc mRNA and evaluated for mRNA delivery following IM administration in mice (FIG. 12B). These studies identified headgroup Am15 (1,4,7,10,13,16-hexaazacyclooctadecane or hexacyclen) as the most potent for mRNA transfection, with delivery efficacy reducing with either increased or decreased headgroup size. Without wishing to be bound by any particular theory, the potency of this headgroup may be attributed to its spatial arrangement which enables extensive RNA-lipid interactions. After identifying Am15, the position of the Z-alkene on the tail G41 was evaluated by conducting an “alkene walking” along the tail length and away from the ester by synthesizing a new series of tails G47-G55. The position of the Z-alkene did not alter in vivo mRNA delivery significantly, except when placed at the penultimate position (G51), which significantly decreased expression. Introduction of additional Z-alkenes also did not alter the potency significantly but was enough to restore the loss in potency for G51 in AMG1551 (FIG. 17). Overall, the lipids generated from the “alkene walking” experiment did not significantly outperform AMG1541 and were therefore not selected for further evaluation.

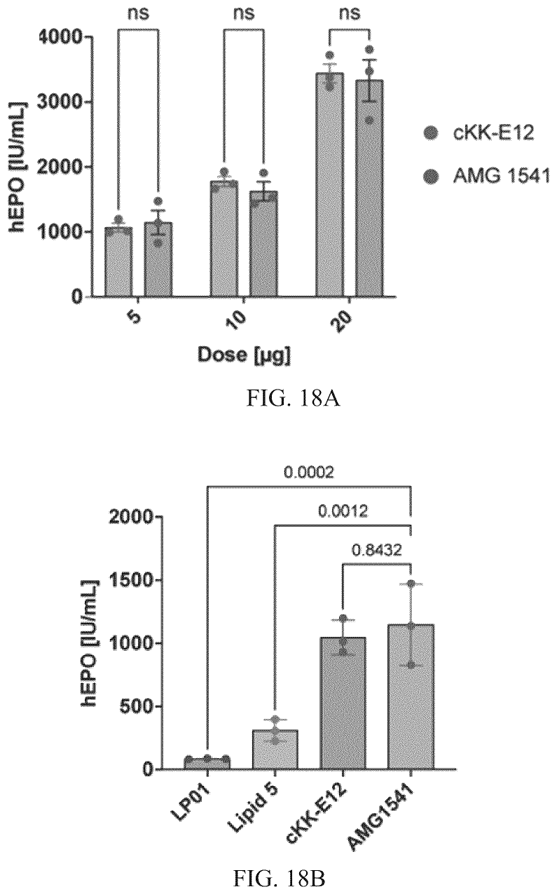

After identifying AMG1541 as the most potent lipid from these screening efforts, it was compared to lipids AMG104/AMG1041 and to lipids SM-102 and cKK-E12 (FIG. 12C). AMG1541 showed 4.5-fold and 2.3-fold higher expression compared to AMG104 and AMG1041, respectively, highlighting the utility of rational design to improve upon lipids identified using combinatorial chemistry. Moreover, AMG1541 significantly outperformed current state-of-the-art formulations, showing 1.7-fold improvement over cKK-E12 and a 3.5-fold increase compared to SM-102. AMG1541 LNPs were also tested for intravenous delivery of mRNA to the liver and found it gave comparable expression to cKK-E12, ˜3-fold higher than Lipid 5 and ˜5-fold higher than LP-01, all of which are clinically tested lipids (FIG. 18).^{24,25} These results further corroborate the potency of AMG1541 for mRNA delivery via multiple administration routes.

The clearance and biodistribution of these lipids following injection was also evaluated. In addition to mRNA delivery efficacy, clearance and biodistribution are parameters that affect translation of LNPs to the clinic, as they can impact safety and tolerability, particularly with repeat dosing.²⁶ Prolonged retention of LNPs at the injection site can also increase systemic exposure to LNP-mRNA, potentially leading to adverse side effects without enhancing the immune response.^{27,28} Lipid clearance was therefore evaluated in the blood, muscle, liver, and spleen following IM injection in mice by collecting these tissues at various time points and analyzing the lipid concentration in these tissues by LC-MS/MS (FIG. 12F). Rapid hydrolysis of the glycidyl ester tails was observed. The half-life of intact ionizable lipid was approximately three hours and dropped below detectable limits by 24 hrs. Furthermore, ionizable lipid was undetectable in the liver, spleen, and blood at all timepoints, suggesting very rapid clearance outside the site of injection (data not shown) minimizing systemic exposure. In similar studies by Hassett et al., SM-102 remained detectable at the injection site for at least 48 hours and for at least 10 hours in the liver and spleen.²⁹ This suggests that the present lipids have improved clearance kinetics relative to current clinically approved formulations. The potential for systemic exposure to LNPs and off-target antigen expression was assessed by FLuc protein expression in the liver and spleen following IM injection of AMG1541 and SM-102. Compared to AMG1541, SM-102 exhibited ˜100-fold higher mRNA transfection in both the liver and spleen (FIG. 12F). This result is consistent with the rapid lipid clearance we observed and suggests AMG1541 leads to less expression outside the muscle. Hepatic expression of antigens has been suggested to be partially responsible for COVID-19 vaccine reactogenicity, therefore it is preferable if it can be avoided.^{30,31} Together these results suggest AMG1541 has favorable PK/PD properties relative to current clinical formulations.

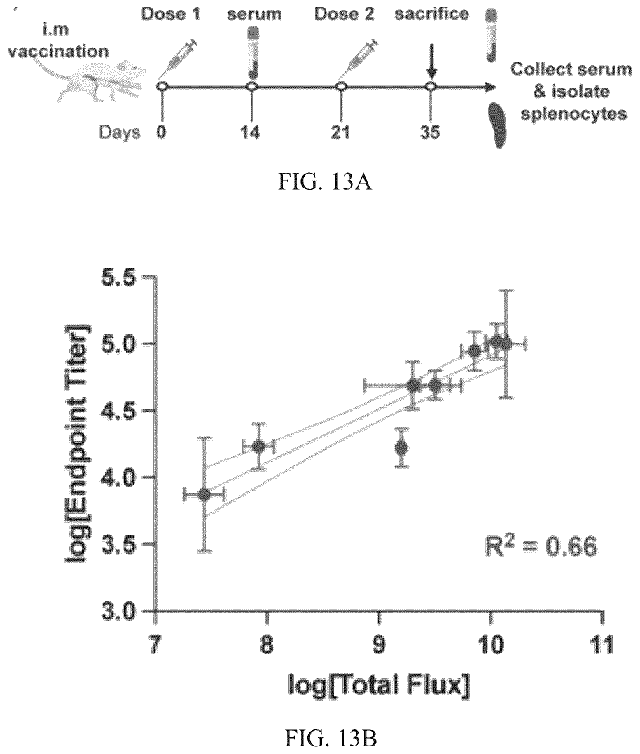

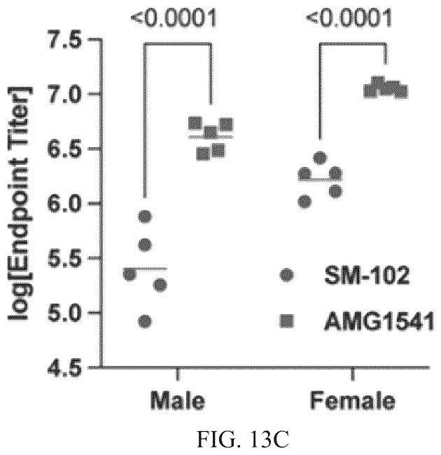

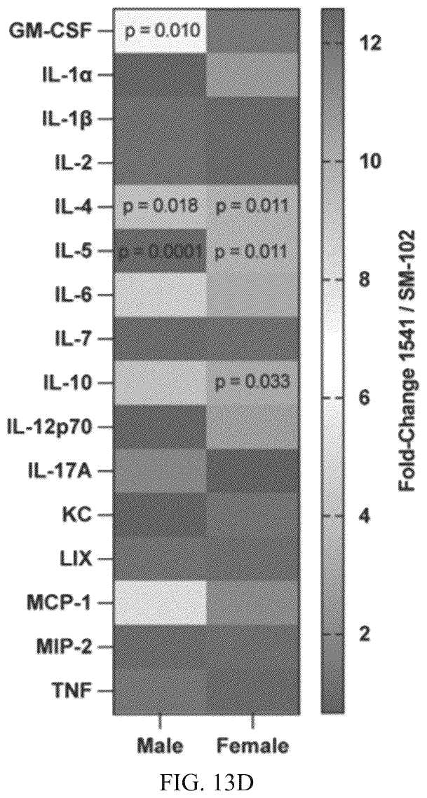

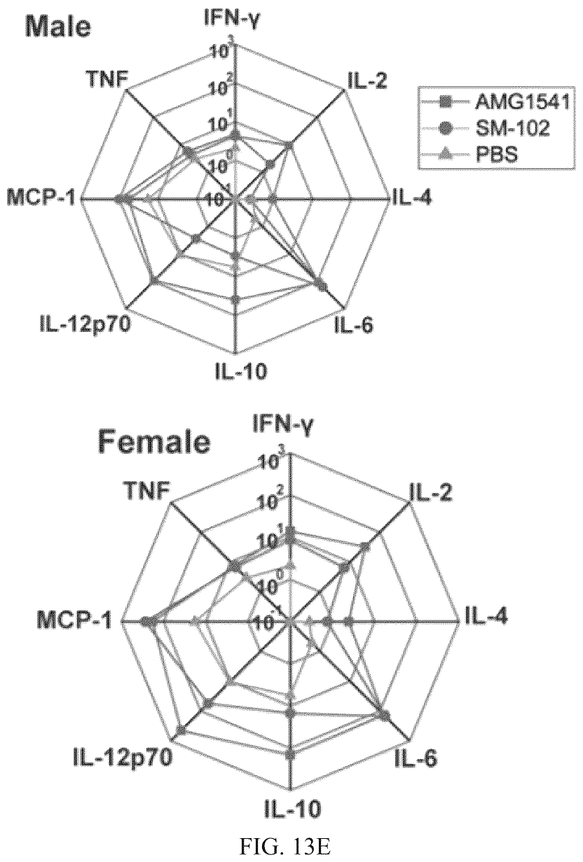

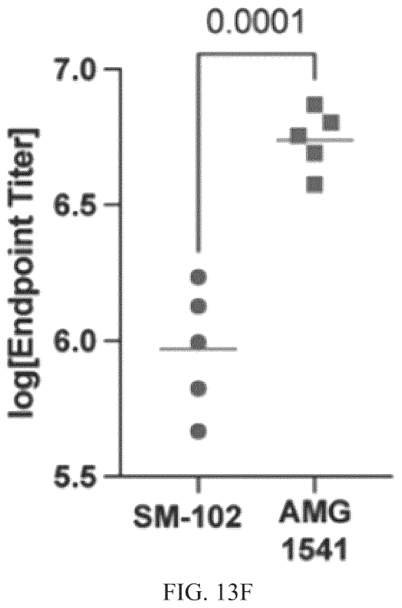

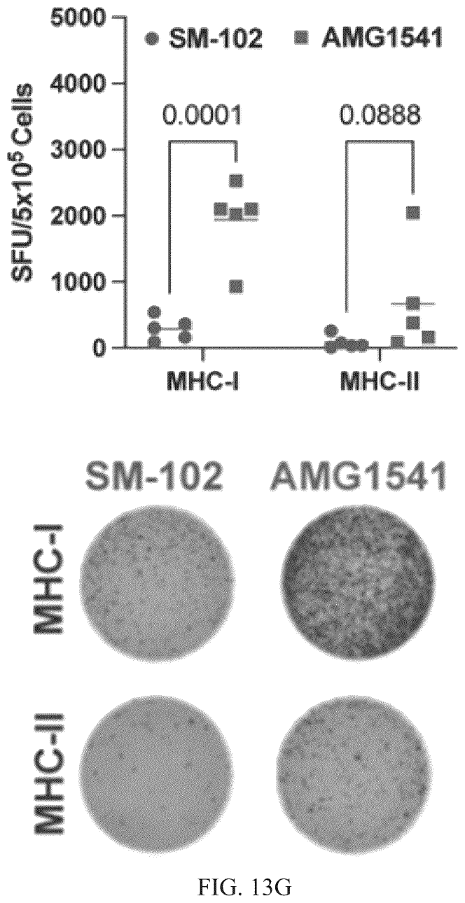

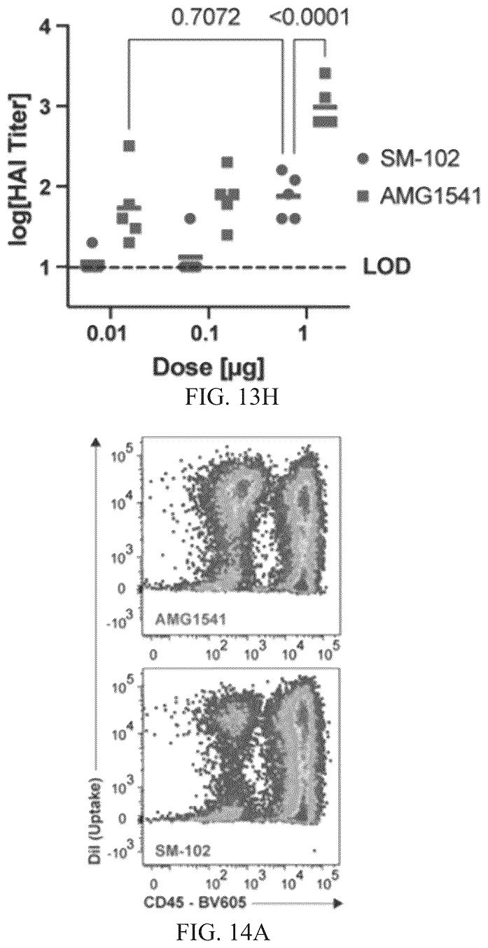

Vaccine potency of AMG ionizable lipids. Before extensively characterizing the immune response for AMG1541, it was evaluated whether increased mRNA expression would enhance immunogenicity.³² Eight different ionizable lipids (with FLuc mRNA signal spanning four orders of magnitude) were formulated with mRNA encoding the SARS-CoV-2 (B.1.617.2) spike protein. Mice were vaccinated with these LNPs; all vaccine experiments in this section followed an IM prime-boost vaccination protocol, with doses on days 0 and 21.33 Serum was collected on day 14 and serum/splenocytes on day 35 to characterize the vaccine responses (FIG. 13A). When comparing the resultant day 35 binding antibody titers to FLuc signal, we found a strong linear correlation between mRNA expression and antibody titers (FIG. 13B). Additionally, these results confirmed that AMG1541 was potent for vaccination within the subset of lipids tested. Interestingly, these results contrast with previously reported results which showed no correlation between antibody titers and FLuc signal.²⁸

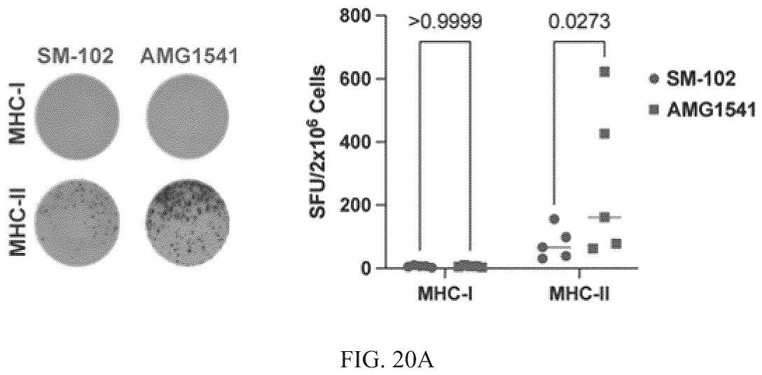

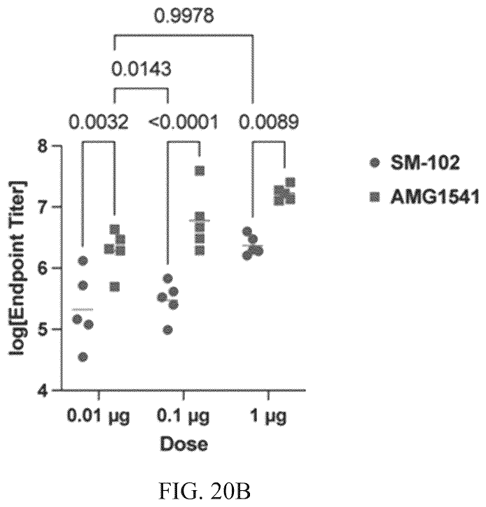

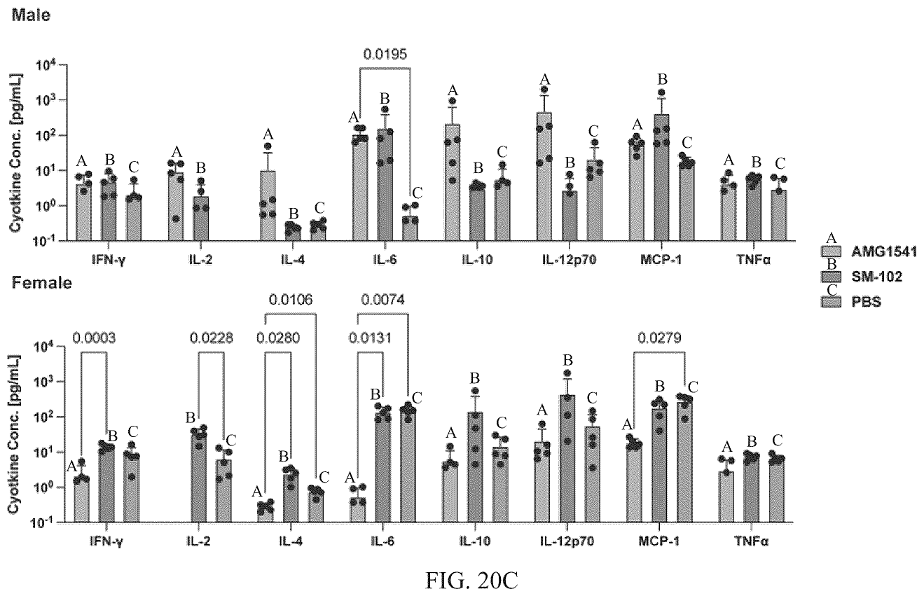

Further experiments to evaluate the dose-sparing potential of AMG1541 were performed for influenza vaccination.^{34,35} The immunogenicity of AMG1541 was compared to the clinical benchmark SM-102 by formulating each into LNPs containing hemagglutinin (HA) (A/Tasmania/503/2020 H3N2) mRNA. BALB/cJ mice were vaccinated with 1 g of these mRNA LNPs and the quality of humoral and cellular immunity following vaccination was evaluated. Enhancing each component of immunity contributes to improving vaccine protection—humoral immunity provides protection against initial infection while cellular immunity improves the clearance of breakthrough infections. AMG1541 increased humoral responses to vaccination, giving significantly higher binding antibody titers on day 35 post vaccination. Male and female mice vaccinated with AMG1541 had 15.8- and 6.8-fold higher endpoint titers, respectively (FIG. 13C). Cellular immunity was also enhanced as the media from splenocytes stimulated with MHC-binding peptides showed significant upregulation of IL-4, and IL-5 in both male and female mice and IL-10 in female mice (FIG. 13D). The upregulation of these cytokines indicates a Th2-biased immune response, consistent with previous vaccine studies using BALB/cJ mice.³³ IFN-γ ELISpots showed IFN-γ secretion was induced by MHC-II stimulation, further supporting a Th2-biased immune response (FIGS. 20A-20C). Serum cytokines were examined 6 hours after the boost dose to determine whether the enhanced immunogenicity of AMG1541 also led to increased systemic inflammation (FIGS. 20A-20C). Encouragingly, minimal cytokine upregulation was observed in male mice—only IL-2 and IL-6 were upregulated and reached similar levels in SM-102 and AMG1541 vaccinated mice. Female mice showed significantly higher cytokine levels, as expected based on their overall stronger immune response to vaccination.³⁶ However in these female mice, AMG1541 and SM-102 showed comparable cytokine levels except for IL-2 which was 5-fold higher in AMG1541 vaccinated mice. Overall, these results demonstrate that AMG1541 induced more robust cellular and humoral immunity than SM-102 in BALB/cJ mice with little to no increase in systemic inflammation following vaccination.

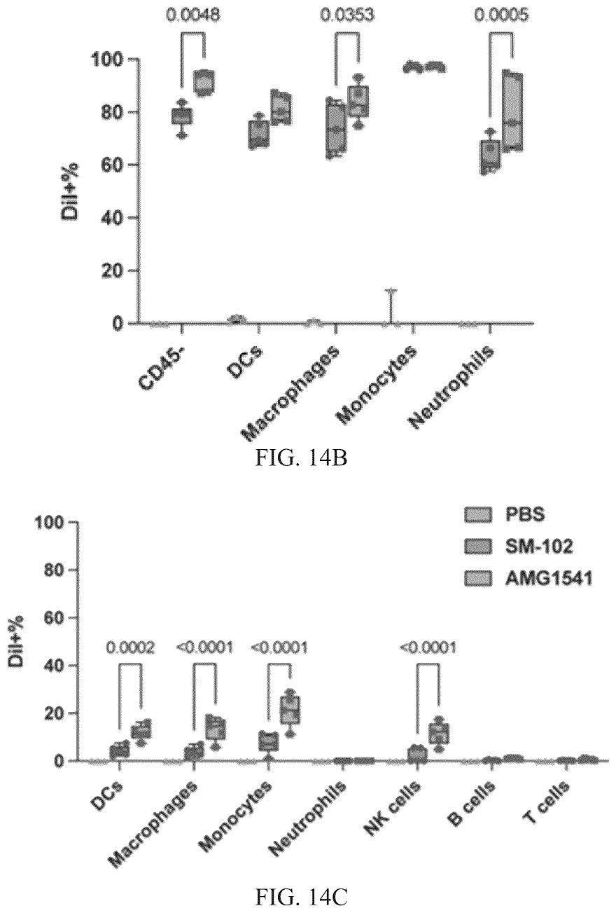

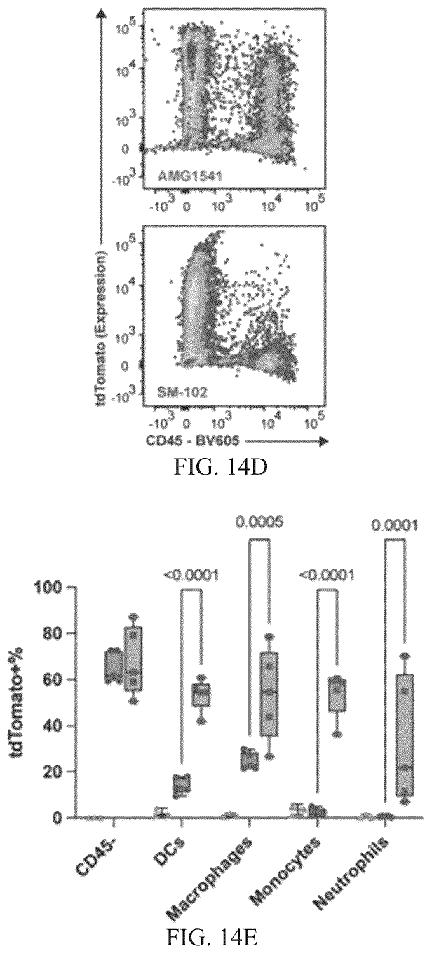

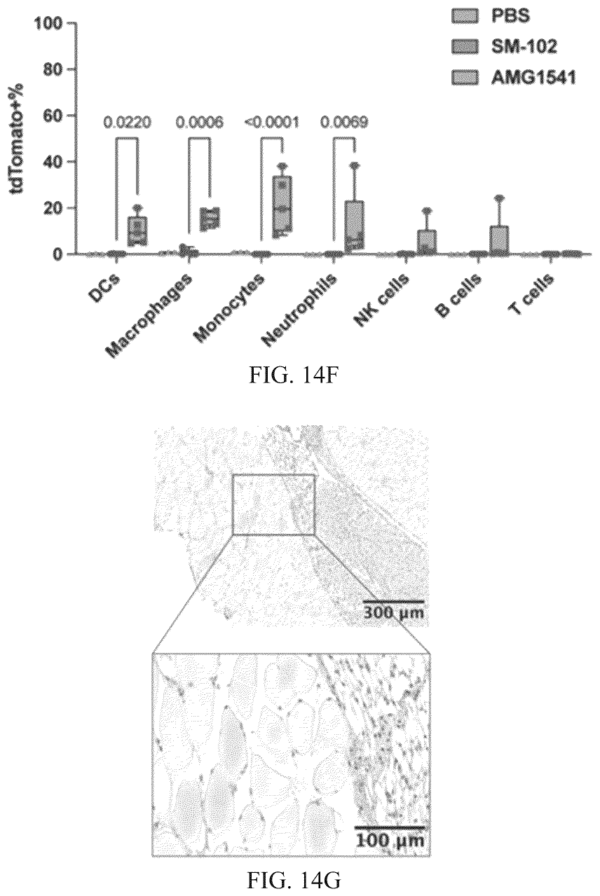

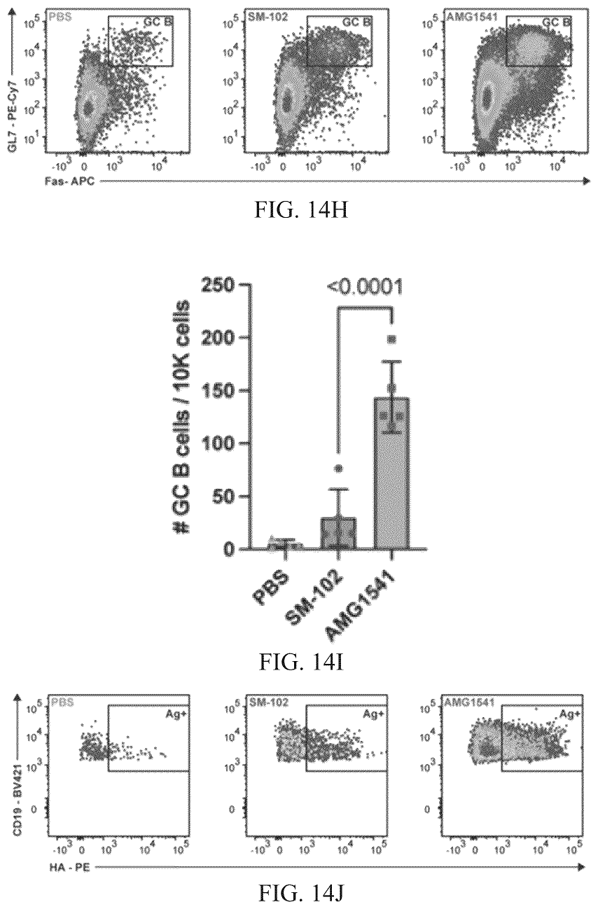

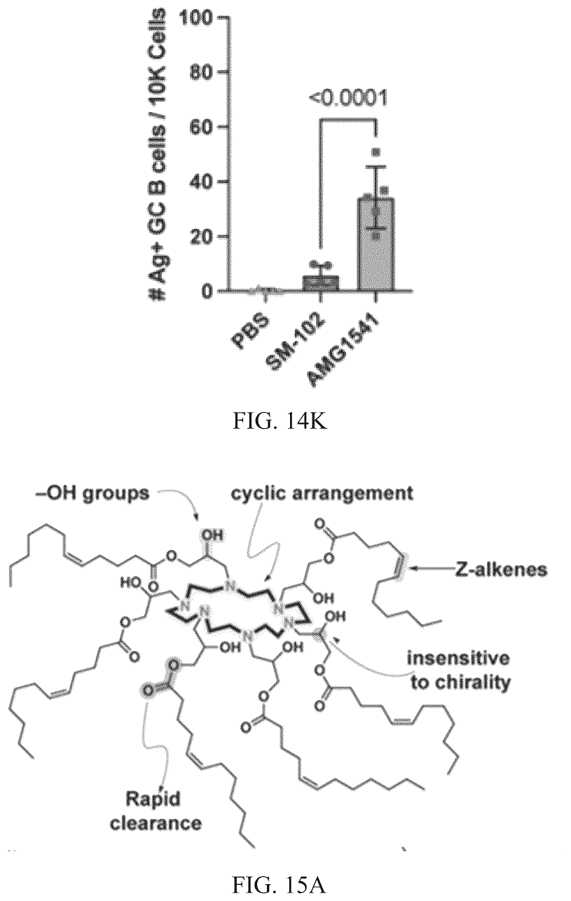

Although BALB/cJ mice are frequently used for preclinical models for studying vaccine candidates, they may exaggerate antibody responses while simultaneously failing to capture CD8+ T cell responses due to their inherent Th2 biased immune response.³⁷ Therefore, evaluation of AMG1541 was extended to female C57B1/6J mice which have a Th1-biased response. The day 35 binding antibody titers were lower than the titers in BALB/cJ mice, but AMG1541 vaccination once again outperformed SM-102, giving 5.8-fold higher antibody titers (FIG. 14F). Similarly, an IFN-γ ELISpot on peptide-stimulated splenocytes reflected the Th1 biased response, with MHC-I peptide pool stimulation yielding substantially higher IFN-γ secretion than MHC-II stimulation. When comparing lipids, AMG1541 gave an extremely robust CD8+ T cell response, with 6.6-fold more Spot Forming Units (SFU) in the MHC-I stimulated splenocytes relative to SM-102. These results further validate the potent vaccine response induced by AMG1541 LNPs. Additionally, the BALB/cJ and C57B1/6J results together indicate the Th-bias of the vaccine responses were dependent on the mouse strain and AMG1541 increases vaccine potency without affecting Th-bias.³⁷

These results demonstrated AMG1541's ability to outperform clinical LNPs at a moderate dose of 1 g. An additional goal was to maintain protective immunity at significantly lower doses. To test this, a dose-response vaccination experiment ranging from 0.01 g to 1 g was performed in female BALB/cJ mice. Neutralizing antibody titers were evaluated using the hemagglutination inhibition (HAI) assay. HAI titers are strongly correlated with vaccine protection and the assay is an FDA approved method for testing influenza vaccine efficacy^{38,39} In agreement with the binding antibody titers, higher neutralizing doses were measured across all three tested doses: SM-102 only gave detectable neutralizing immunity at a 1 μg dose, while AMG1541 gave neutralizing immunity with as little was 10 ng of mRNA (FIG. 13H). Not only was AMG1541 able to generate neutralizing immunity at a very low dose, the binding and neutralizing antibody titers were equivalent in mice vaccinated with AMG1541 at a 0.01 g dose as those with SM-102 at a 1 g dose (FIG. 13H).

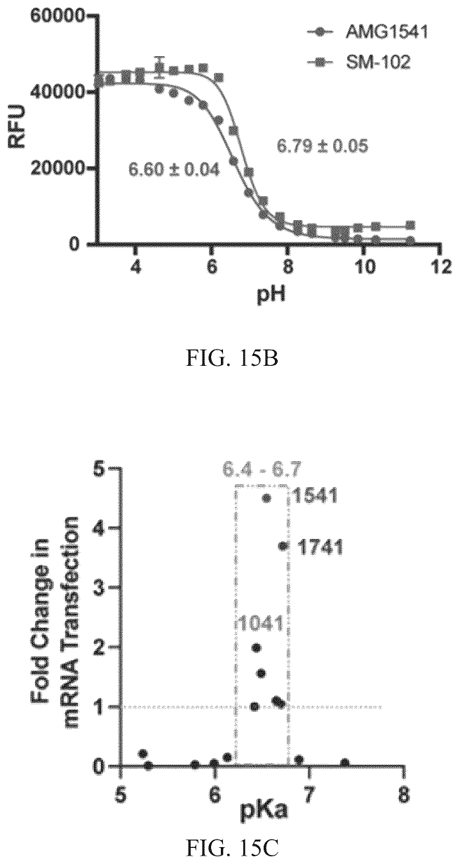

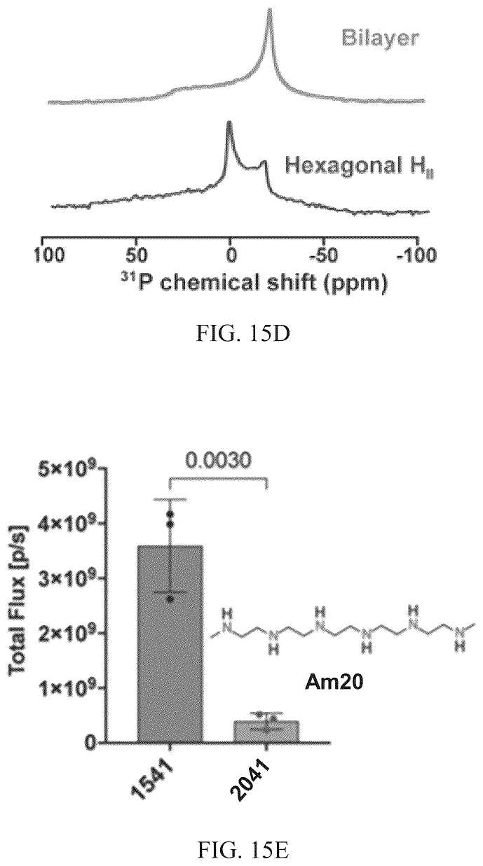

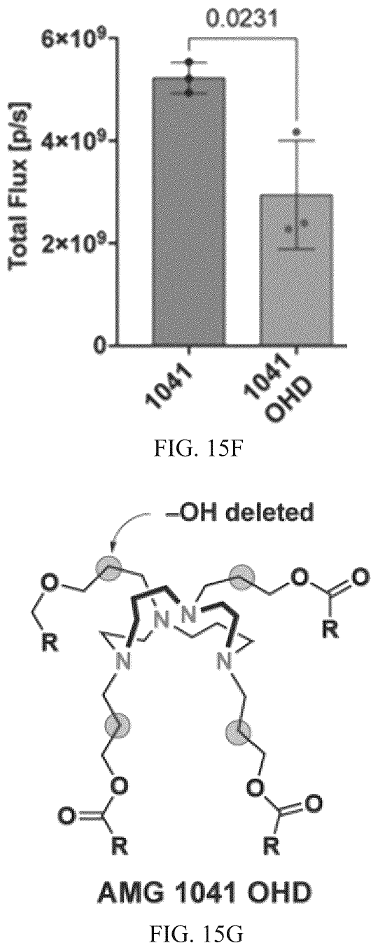

Immune cell uptake, expression, and germinal center responses. To understand the factors contributing to the enhanced vaccine response of AMG1541 LNPs, the local biodistribution and resultant protein expression following intramuscular injection was characterized. Immune cell uptake following vaccination is the first step in initiating vaccine responses as it activates innate immune pathways, facilitating further immune cell recruitment and activation.⁴⁰⁻⁴² The uptake of LNPs was characterized by dosing female BALB/cJ mice IM with fluorescently labeled AMG1541 or SM-102 LNPs and then isolating cells from the muscle and local draining lymph node (LDLN) (FIGS. 15A-15C). Within the cells isolated from muscle, the majority of cells contained LNPs, though AMG1541 showed higher uptake in CD45- cells, macrophages, and neutrophils (FIGS. 14A and 14B). These data indicate both SM-102 and AMG1541 are effective at potentiating uptake at the injection site, though AMG1541 is significantly better. Examining the LDLNs, there were larger differences between the two LNPs, with AMG1541 giving significantly higher uptake in several key APC subsets (FIG. 14C). This suggests that AMG1541 enhances lymph node trafficking, a key factor in generating a stronger adaptive immune response,⁴³ which may have contributed to the more robust vaccine efficacy observed with AMG1541 LNPs.

Although LNP uptake is an important factor in determining the immune activation of vaccines, it does not always correlate with antigen expression, which impacts generation of antigen-specific immune responses.^{44,45} Therefore, protein expression levels were characterized by injecting LNPs encapsulating tdTomato mRNA IM in female BALB/cJ mice, and cells were isolated from the muscle and LDLN (FIG. 5d-f). Compared to uptake, expression showed more significant differentials between AMG1541 and SM-102. For example, AMG1541 LNPs yielded 3.8-fold higher expression in DCs and 22-fold higher expression in monocytes in the muscle—despite similar LNP uptake levels in these cell types (FIG. 14E). In other immune cell subsets, AMG1541 LNPs consistently gave higher expression across in the muscle. In the LDLN, AMG1541 again showed significantly higher protein expression in APCs relative to SM-102 (FIG. 14F). The increased expression from AMG1541 LNPs in APCs, in conjunction with increased uptake, is likely the critical driver of its enhanced immunogenicity. Additionally, expression in muscle fibers was examined, as muscle fibers do not survive single cell dissociation for flow cytometry, using IHC. In contrast to previous reports that show minimal to no transfection of mRNA in the muscle fibers, tdTomato was expressed in the muscle fibers after IM administration of AMG1541 (FIG. 14G).²⁹ Expression in muscle fibers may further enhance vaccine immunogenicity by enhancing the recruitment of immune cells through antigen presentation or damage associated molecular patterns (DAMPs).^{46,47}

Finally, the germinal center (GC) reactions following vaccination were assessed to provide further evidence that AMG1541's enhanced uptake and expression was leading to an enhanced adaptive immune response after vaccination. GC reactions initiate shortly after vaccination and generate neutralizing antibodies through a complex affinity maturation process involving the antigen presentation to B cells and T follicular helper (Tfh) cells. Hence, stronger GC reactions are indicative of stronger neutralizing responses against pathogens. These GC reactions were characterized in female BALB/cJ mice vaccinated with either AMG1541 or SM-102 LNPs encoding HA (A/Tasmania/503/2020) by collecting the LDLNs 8 days following a prime dose. Cells within the LDLN were stained and analyzed for the development of GC B cells (FIGS. 14G-14I). Overall, AMG1541 gave much stronger GC responses than SM-102. AMG1541 treated mice had 4.8-fold more Fas+GL7+GC B cells and 6.0-fold higher Ag+ GC B cells than SM-102 treated mice (FIG. 14I). These results further support that the enhanced neutralizing responses in AMG1541 LNP vaccinated mice is the result of enhanced local uptake and expression ultimately leading to strong GC reactions.

Structural features of AMG lipids that facilitate mRNA delivery. The factors contributing to lipid potency were evaluated. The ionization properties of AMG1541 LNPs were investigated using a previously established 6-(p-toluidino)-2-napthalenesulfonic acid (TNS) assay.²² The pKa of AMG LNPs showing higher in vivo mRNA transfection than SM-102 ranged between pH 6.4-6.7. Of these LNPs, AMG1541 exhibited a pKa of ˜6.5, which has previously been shown to be effective for mRNA delivery. Interestingly, AMG1541 LNPs also exhibited a more gradual loss of ionization across the pH range examined (FIGS. 15B and 15C). Without wishing to be bound by any particular theory, this is likely due to the additional amines within the lipid headgroup possessing multiple pKas thereby broadening its buffering range. Previous studies have demonstrated that higher buffering capacities enhance endosomal escape⁴⁸⁻⁵¹. In addition to endosomal buffering, it is hypothesized that the protonated ionizable lipids within the acidic endosome bind with anionic phospholipids within the endosomal membrane, destabilizing the membrane's bilayer and promoting endosomal pore formation.^{17,52} The membrane fusion properties of the ionizable lipids was evaluated via ³¹P NMR. When bilayer forming DPPS vesicles were mixed with equimolar AMG lipid under acidic conditions, a bilayer to hexagonal phase transition was observed, indicating their membrane-destabilizing nature (FIG. 15D). Previous ³¹P NMR studies have demonstrated that membrane destabilization by ionizable lipids is correlated with the mRNA delivery efficacy of the LNPs. Together these results suggest that these novel ionizable lipids promote endosomal disruption through their buffering and membrane destabilizing properties.

How specific structural features of the lipid headgroup impacted RNA delivery was evaluated. First, the cyclic Am15 headgroup was compared with its linear analog Am20. The linear lipid AMG2041 gave nearly 7-fold lower FLuc expression upon IM administration, demonstrating the importance of cyclic structure (FIG. 15E). Additionally, modifying the length of the methylene bridges between the amines, as with Am21 and Am22, significantly reduced mRNA expression demonstrating the efficacy of the two carbon spacer in headgroup Am15 (FIG. 12B). These results suggest Am15 adopts a spatial orientation within lipid nanoparticles that may facilitate lipid-RNA or lipid-membrane interactions. Previous reports have demonstrated, for example, that controlling the nitrogen spacer lengths in polyamines facilitate interactions with phospholipid membranes.

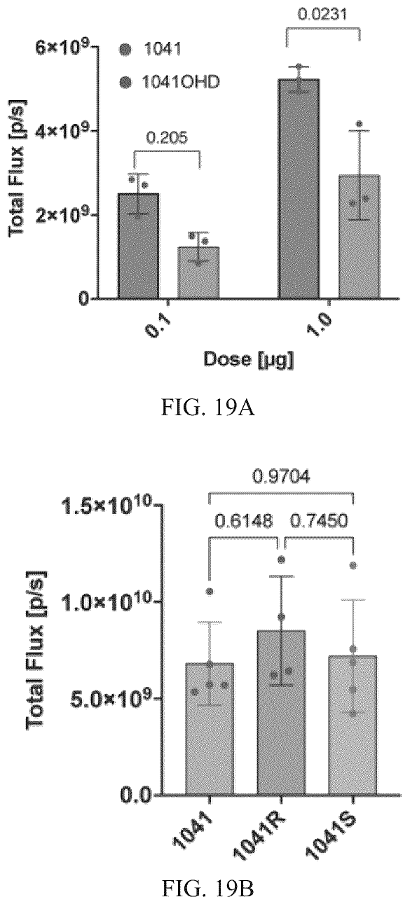

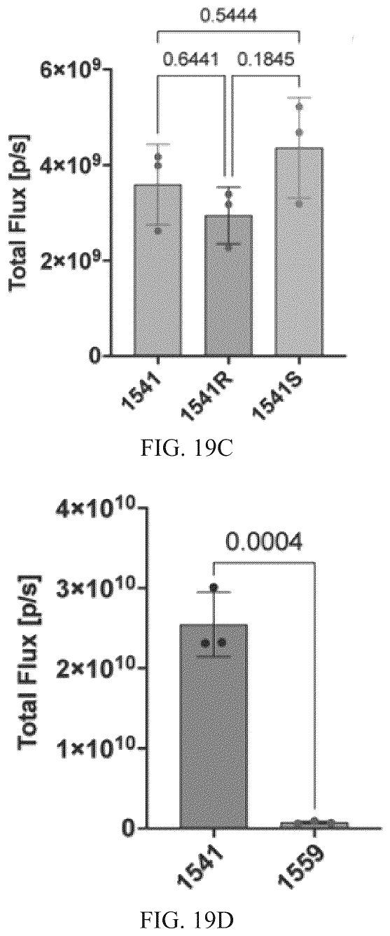

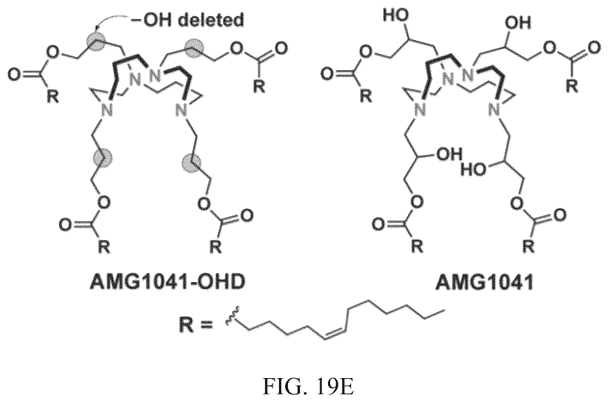

Finally, the role of the P-hydroxyls formed by the epoxide ring opening reaction in the facilitating mRNA delivery was evaluated. As discussed previously, epoxide ring opening chemistry was used to generate resultant P-hydroxyls that could enhance mRNA delivery.^{10,29} The in vivo efficacy of mRNA delivery for AMG1041 was compared to that of its —OH deleted version (AMG1041-OHD). LNPs formulated with AMG1041-OHD exhibited ˜50% loss in IM FLuc expression at both a 0.1 and 1 μg dose (FIG. 15, FIG. 19). Additionally, AMG1559, which was synthesized via Michael addition and lacks P-hydroxyls, showed a dramatic 34-fold drop in transfection. Enantiomerically pure versions of AMG1041 and AMG1541 showed no differences in potency (FIG. 19). These data confirmed the importance of proximal hydroxyls for the potency of these lipids, while demonstrating that the specific orientation imposed by stereochemistry was not important.

This example discloses aza-crown ether based ionizable lipids developed using combinatorial epoxy ring-opening chemistry and iterative development via rational design. One of the lipids features hexacyclen headgroup (Am15) bearing R-amino alcohols, a structure not previously used for ionizable lipids. Structure-activity studies revealed this structure achieved potent RNA delivery through increased lipid-RNA interactions. Additionally, the incorporation of biodegradable glycidyl ester tails facilitated rapid clearance from the injection site, positioning these as some of the most degradable LNPs reported to date. AMG1541 demonstrated ˜3.5-fold higher in vivo mRNA transfection intramuscularly (IM) compared to state-of-the-art SM-102 LNPs in SpikeVax mRNA vaccines.

The immunogenicity of these mRNA-LNPs directly correlated with the delivery efficacy of the LNPs. In depth vaccine characterization showed that these novel ionizable lipids generate significantly higher humoral and cellular response across different strains and sexes of mice. Notably, AMG1541 generated similar protective immunity as compared to SM-102 LNPs at a 100-fold lower dose, suggesting that this more potent LNP formulation can induce dose-sparing while maintaining protective immunity. Mechanistic characterization of the vaccine response revealed that AMG1541 LNPs exhibited increased drainage to the lymph nodes and increased mRNA expression in APCs in both the muscles and local draining lymph nodes. This increased antigen expression in APCs generated stronger germinal centers in the draining lymph nodes that resulted in stronger Ab titers. These findings highlight the potential for developing more potent mRNA vaccines by engineering LNPs to enhance mRNA delivery efficacy in innate immune cells to amplify the ensuing adaptive immune response.

### Example 4: Lipid Nanoparticle Formulation and Administration Methods

Animal studies. All animal studies were approved by the MIT IACUC and were consistent with local, state, and federal regulations as applicable. Animals were housed in a pathogen-free environment with a 12-hour day/night cycle with access to food and water ad libitum. All mice were 6-8 weeks old at the start of experiments. BALB/cJ mice were used for lipid screening studies, and vaccination studies as indicated. C57B1/6J mice were used for the germinal center response studies, biodistribution, expression studies, and vaccine studies as indicated.

Lipid nanoparticle formulation. 1,2-dioleoyl-sn-glycero-3-phosphoethanolamine (DOPE) (Avanti), 1,2-distearoyl-sn-glycero-3-phosphocholine (DSPC) (Avanti), Cholesterol (Millipore-Sigma), and 1,2-dimyristoyl-rac-glycero-3-methoxypolyethylene glycol-2000 (DMG-PEG2k) (Avanti) stocks were prepared by dissolving at 10 mg/mL in ethanol. SM-102 (BroadPharm) and other ionizable lipids were dissolved at 40 mg/mL in ethanol. The lipid phases were prepared using previously reported formulations summarized in the following table. Separately, the aqueous phases for formulations were made by preparing a 133 ng/L solution of mRNA in 10 mM citrate, pH 3.0. The LNPs were then formulated using a herringbone microfluidic mixing channel mixing the aqueous and lipid phases in a 3:1 volume ratio (CITE) at a total flow rate of 1.2 mL/min.

| Component | AMG lipids | SM-102 |
|---|---|---|
| Ionizable lipid:RNA weight ratio | 10:1 | 10:1 |
| Ionizable lipid (mol %) | 35 | 50 |
| Helper lipid (mol %) | DOPE, 16 | DSPC, 10 |
| Cholesterol (mol %) | 46.5 | 38.5 |
| DMG-PEG2k | 2.5 | 1.5 |

Following formulation, LNPs were exchanged into PBS using 100 kDa cutoff Amicon centrifugal filters (Millipore-Sigma). Briefly, LNPs were diluted at least 4× with PBS then concentrated, repeating 3 times before concentrating to the final desired volume such that the final pH was 7-7.5. In some cases, LNPs were frozen at −80° C. in 7.5% w/v sucrose in PBS before use.

LNP characterization. Concentration of mRNA was determined using a Quant-IT Ribogreen assay. LNPs were diluted in either native TE buffer (ThermoFisher) or denaturing TE buffer+0.5% Triton-X 100 (Millipore-Sigma) to measure the “free” and total mRNA, respectively. 100 μL of these unknowns were added to a black 96-well plate. Separately, standard curves ranging from 2 ng/μL-0 ng/μL were prepared in each of these buffers and 100 μL were added to the same 96-well plate. Finally, Quant-IT Ribogreen reagent (ThermoFisher) was diluted 1:200 in TE buffer and 100 μL of this solution was added to each of the wells. The well plate was then mixed at 400 rpm with a plate shaker for 5 mins. and the fluorescence was read with an excitation/emission of 485/535. LNPs were measured in triplicate and standards were measured in duplicate. RNA concentration in each of the unknown wells was calculated by fitting the standard curves with a linear trendline. Encapsulation efficiency (% EE) was calculated according to:

$$\% EE = \frac{( total R NA ) - ( free RNA )}{total RNA} \times 100 .$$

All dosing was performed based on the total RNA concentration.

Dynamic light scattering and zeta potential measurements were performed using a Malvern Zetasizer system. The reported diameter and PDI were obtained from the cumulants fit of the autocorrelation function. Zeta potential measurements were performed in 0.1×PBS, pH 7.4 and calculated using the Smoluchowski approximation.

Lipid pharmacokinetics (PK) study. Six to 8 weeks old male C57B1/6 mice were obtained from Charles River Laboratories (Wilmington, MA). Mice were administered with mRNA-LNP containing nontargeting (luciferase) control mRNA (FLuc). Mice (n=3/timepoint/group) were administered at 0.3 mg/kg by intravenous bolus injection via the lateral tail vein. Blood, liver, and spleen were collected at 0.5, 1, 1.5, 2, 4, 8, and 24 hours postdose. Mice were perfused with saline before tissues collection. Blood samples were processed to obtain plasma. All samples were processed and analyzed by liquid chromatography with tandem LC-MS/MS.

| Time (hrs) | [AMG1041] ng/g tissue | | |
|---|---|---|---|
| 0 | 0 | 0 | 0 |
| 0.5 | 36400 | 15600 | 31600 |
| 1 | 32200 | 24800 | 28000 |
| 1.5 | 9800 | 30600 | |
| 2 | 14000 | 23400 | 20400 |
| 4 | 17200 | 12600 | 9400 |
| 8 | 4800 | 8000 | 8000 |
| 24 | 600 | 600 | 600 |

LC-MS method for lipid PK study. Reversed-phase C8 chromatography/positive ion mode MS detection was used to quantify lipids. Analyses of polar and non-polar plasma lipids were conducted using an LC-MS system comprised of a Shimadzu Nexera X2 U-HPLC (Shimadzu Corp.) coupled to an Exactive Plus orbitrap mass spectrometer (Thermo Fisher Scientific). Plasma samples (10 μL) were extracted for lipid analyses using 190 μL of isopropanol containing 1,2-didodecanoyl-sn-glycero-3-phosphocholine (Avanti Polar Lipids) as an internal standard. After centrifugation, supernatants were injected directly onto a 100×2.1 mm, 1.7 μm ACQUITY BEH C8 column (Waters). The column was eluted isocratically with 80% mobile phase A (95:5:0.1 v/v/v 10 mM NH₄OAc/MeOH/HCO₂H) for 1 minute followed by a linear gradient to 80% mobile-phase B (99.9:0.1 vol/vol MeOH/HCO₂H) over 2 minutes, a linear gradient to 100% mobile phase B over 7 minutes, then 3 minutes at 100% mobile-phase B. MS analyses were carried out using electrospray ionization in the positive ion mode using full scan analysis over 200-1100 m/z at 70,000 resolution and 3 Hz data acquisition rate. Other MS settings were as follows: sheath gas 50, in source CID 5 eV, sweep gas 5, spray voltage 3 kV, capillary temperature 300° C., S-lens RF 60, heater temperature 300° C., microscans 1, automatic gain control target 1′10⁶, and maximum ion time 100 ms. Raw data were processed using TraceFinder software (Thermo Fisher Scientific) for targeted peak integration and manual review of a subset of identified lipids and using Progenesis QI (Nonlinear Dynamics) for peak detection and integration of both lipids of known identity and unknowns. Lipid identities were determined based on comparison to reference plasma extracts and are denoted by total number of carbons in the lipid acyl chain(s) and total number of double bonds in the lipid acyl chain(s).

Endotoxin testing. All LNPs used for vaccine experiments were tested for endotoxin prior to injection using the PyroGene Recombinant Factor C Endpoint Fluorescent Assay (Lonza) according to the manufacturer's instructions.

RNA synthesis. Hemagglutinin (A/Tasmania/503/2020) RNA and the firefly luciferase RNA used for in vivo experiments were generously provided by Sanofi. The SARS-CoV-2 spike (B.1.617.2) ORF was obtained as a linear gene block (Integrated DNA Technologies). The tdTomato ORF was obtained from Addgene plasmid #54642 using PCR. The firefly luciferase ORF used for the smFISH endosomal escape assay was obtained from pGL3 (Promega) using PCR. These ORFs were cloned into a custom mRNA plasmid containing a T7 promotor with a CleanCap AG site, 5′/3′ UTRs, and a 100-nucleotide poly A tail. Plasmids were transformed into NEB 5-alpha or NEB Stable competent E. Coli (New England Biolabs) according to the manufacturer's instructions. Plasmids were purified using mini or midi-preps (Qiagen) according to the manufacturer's instructions. Sequences were verified using nanopore sequencing. Plasmids were then linearized using 10 U of NsiI-HF (New England Biolabs) per 1 g of DNA in rCutSmart buffer (New England Biolabs) for 1 hr. at 37° C. IVT was then performed using the HiScribe T7 kit (New England Biolabs) using the manufacturer's instructions for synthesis of CleanCapped mRNA. N1-methylpseudouridine triphosphate (TriLink) was substituted for UTP in all reactions to produce fully modified mRNA. Following IVT, template DNA was removed by adding 4 U of DNAse I (New England Biolabs) per 1 g of template to the IVT reaction and incubating at 37° C. for 15 mins. The mRNA was then purified using the Monarch RNA cleanup kit (500 g) (New England Biolabs). The molecular weight of the RNA was verified using a Fragment Analyzer (Agilent).

Immunization studies. LNPs were formulated as described above with hemagglutinin (A/Tasmania/503/2020) or spike (B.1.617.2) encoding mRNA. All LNPs were injected IM in the quadricep with an injection volume of 50 μL with the doses specified in the main text. All vaccination experiments followed a prime-boost dosing regimen with doses given on day 0 and day 21. On day 35, mice were sacrificed, and blood was collected via cardiac puncture and their spleens were collected for further characterization.

Binding antibody ELISAs. Clear, flat-bottomed, high-binding 96 well plates were coated with either spike (B.1.617.2) (Bio-Techne) or hemagglutinin (provided as a gift from Sanofi). Plates were coated overnight at 4° C. by adding 100 μL of a 1 μg/mL antigen solution in 100 mM carbonate buffer, pH 9.6 to each well. The coated plates were then washed with ELISA wash buffer (Biolegend) and blocked for 1 hr. at room temperature (RT) with 150 μL of 1% BSA in PBS. The plate was then washed again, and 100 μL of serially diluted serum samples were then added to the plate and incubated at RT for 1 hr. Next, the plate was washed and the secondary goat anti-mouse IgG, HRP antibody (ThermoFisher) was diluted 1:3,000. 100 μL of this solution added to each well an incubated at RT for 1 hr. After incubation, the plate was washed and 100 μL of TMB chromogen solution (ThermoFisher) was added to each well and incubated for 30 mins. Finally, the HRP reaction was stopped by the addition of 100 μL of 0.5 M H₂SO₄. The absorbance of each well was immediately read at 450 nm with 600 nm as a reference using an Infinite M200 Pro (Tecan) plate reader. The resultant data were then fit with a 4-PL sigmodal curve and the endpoint titers were taken as the point at which this curve crossed four times the absorbance of the blank wells.

IFN-γ ELISpot. Spleens were dissociated into single cell suspensions by mashing through a 70 m strainer (Coming). Cells were pelleted and red blood cells lysed using RBC lysis buffer (Biolegend) for 5 mins. on ice. The RBC lysis reaction was stopped by adding 4 volumes of PBS+1% BSA. Cells were then pelleted, resuspended in PBS+1% BSA, and counted using a countess 3 automated cell counter (ThermoFisher).

Mouse IFN-γ ELISpots (BD Biosciences) were performed according to the manufacturer's instructions. Briefly, ELISpot plates were coated with the capture antibody overnight at 4° C., washed, then blocked for 2 hrs. at RT using complete media: 10% FBS (ThermoFisher) in RPMI 1640+GlutaMAX (ThermoFisher). After blocking, splenocytes were plated at the indicated density in 100 μL of complete media. Separately, Hemagglutinin (A/Tasmania/503/2020) MHC-I and MHC-II restricted peptide pools were obtained as a gift from Sanofi and were dissolved in DMSO at 0.2 mg/mL. These peptide pools were diluted 1:100 in complete media and 100 μL of this solution was added to the cells in the ELISpot plate (final peptide concentration 1 g/mL). Cells were stimulated for 16 hrs. the supernatant was then collected for further analysis, and spots were developed on the plate according to the manufacturer's instructions. Plates were allowed to dry overnight and then imaged on an ImmunoSpot Analyzer (C.T.L.).

LNP biodistribution and expression characterization. For the biodistribution studies, LNPs were formulated as above with the addition of 1,1′-Dioctadecyl-3,3,3′,3′-tetramethylindocarbocyanine perchlorate (DiI) (Millipore-Sigma) at a 1:10 DiI:mRNA weight ratio. For the expression studies, LNPs were formulated with tdTomato encoding mRNA. In both studies, 5 g of mRNA LNPs in 50 μL of PBS were injected IM into the quadricep muscle. 24 hrs. after injection, mice were sacrificed and the quadricep and local draining inguinal lymph nodes harvested. Tissues were then processed and stained according to the protocol below.

Germinal center response characterization. LNPs were formulated as described above with hemagglutinin (A/Tasmania/503/2020) encoding mRNA. 5 μg of mRNA LNPs in 50 μL of PBS were injected IM into the quadricep muscle. 8 days after injection the inguinal lymph nodes were collected and processed for flow cytometry using the protocols below. To stain for antigen-positive B cells, hemagglutinin (A/Tasmania/503/2020) protein was obtained as a gift from Sanofi. The protein was conjugated with phycoerythrin (PE) using a PE lightning link conjugation kit (Abcam) according to the manufacturer's instructions.

Flow cytometry. Muscle tissues were dissociated into a single cell suspension using a mouse skeletal muscle dissociation kit (Miltenyi) according to the manufacturer's instructions. Following dissociation, the dissociation mixture was passed through a 70 m cell strainer (Coming) and debris was removed from the cell suspensions using debris removal solution (Miltenyi) according to the manufacturer's instructions. Red blood cells (RBCs) were then removed using RBC lysis solution (Biolegend). Lymph nodes were dissociated by using a syringe plunger to mash the tissue through a 70 m strainer. Cells were then counted used a Countess 3 automated cell counter (ThermoFisher).

Following dissociation, 10⁵ muscle cells and 10⁶ lymph node cells were stained for 30 mins. at 4° C. with Zombie-NIR viability dye (Biolegend), then Fc blocked (Miltenyi), and stained with the antibodies of interest at 4° C. for 30 mins. Following staining, cells were washed twice and then fixed with 4.2% PFA (BD Biosciences) at 4° C. for 15 mins. Finally, cells were stored in PBS+1% BSA (Miltenyi) prior to analysis and analyzed within 2 days on a BD FACS Symphony A3 (BD Biosciences). Compensation was performed using UltraComp eBeads compensation beads (ThermoFisher).

Immunohistochemistry (IHC). Quadricep muscles were harvested 24 hrs. after injection of 5 μg of tdTomato mRNA LNPs and fixed for 24 hrs. in 10% neutral buffered formalin at 4° C. Following fixing, tissues were washed with deionized water and stored in 70% ethanol. Tissues were then cut in the transverse direction along their midplane, dehydrated and paraffin embedded. 4 m thick sections were then deparaffinized and subjected to heat-mediated antigen retrieval in pH 6 at 97° C. for 20 mins. Sections then underwent endogenous peroxidase deactivation for 10 mins. followed by blocking for 30 mins. After blocking, the primary antibody (Abcam, ab62341, 1:100) was added for 60 mins. slides were washed and the secondary antibody (R&D Systems, VC003-025) was added for 30 mins. After washing, staining was performed with DAB substrate for 5 mins. followed by counterstaining with hematoxylin. Slides were then mounted and scanned using a slide scanner with a 40× objective.

HAI Assay. HAI assays were performed by IBT bioservices. Viral titers were standardized by defining one HA unit as the amount of inactivated influenza A/Tasmania/503/2020 virus required to completely hemagglutinate an equal volume of 1% turkey red blood cells (TRBCs) (Lampire). Serum was heat inactivated at 56° C. then receptor destroying enzyme (RDE) (DENKA) was added to the serum and incubated at 37° C. for 20 hrs. RDE was then deactivated at 56° C. for 60 mins. Serum samples were then serially diluted in DPBS and 25 μL of diluted serum was added to a 96-well V bottom plate. Subsequently, four HA units of inactivated virus in 25 μL of DPBS were added to the diluted serum and incubated for 30 mins. at RT. 50 μL of a 1% (4×10⁷ cells/mL) solution of turkey red blood cells (Lampire) was then added to the serum and virus mixture. The HAI titer was then calculated as the highest dilution which completely inhibited hemagglutination.

Luminex. The multiplexing analysis was performed using the Luminex™ 200 system (Luminex) by Eve Technologies Corp. All samples were diluted 1:1 with PBS+1% BSA (Miltenyi). The supernatants from the stimulated splenocytes used for the ELISpot assay were analyzed using Eve Technologies' Mouse High Sensitivity 18-Plex Discovery Assay® (MilliporeSigma, Burlington, Massachusetts, USA) according to the manufacturer's protocol. The 18-plex consisted of GM-CSF, IFNγ, IL-1α, IL-1β, IL-2, IL-4, IL-5, IL-6, IL-7, IL-10, IL-12(p70), IL-13, IL-17A, KC/CXCL1, LIX, MCP-1, MIP-2 and TNFα. Assay sensitivities of these markers range from 0.06-9.06 pg/mL for the 18-plex. Serum collected 6 hrs. after vaccination was analyzed using Eve Technologies' Mouse Focused 10-Plex Discovery Assay® (MilliporeSigma, Burlington, Massachusetts, USA) according to the manufacturer's protocol. The 10-plex consisted of GM-CSF, IFNγ, IL-1β, IL-2, IL-4, IL-6, IL-10, IL-12p70, MCP-1, and TNFα. Assay sensitivities of these markers range from 0.4-10.9 pg/mL for the 10-plex. Individual analyte sensitivity values are available in the MilliporeSigma MILLIPLEX® MAP protocol.

### REFERENCES

1. Mathieu, E. et al. A global database of COVID-19 vaccinations. Nature Human Behaviour 2021 5:7 5, 947-953 (2021).
2. Chaudhary, N., Weissman, D. & Whitehead, K. A. mRNA vaccines for infectious diseases: principles, delivery and clinical translation. Nat Rev Drug Discov 20, 817-838 (2021).
3. Yassini, P. et al. Interim analysis of a phase 1 randomized clinical trial on the safety and immunogenicity of the mRNA-1283 SARS-CoV-2 vaccine in adults. Hum Vaccin Immunother 19, (2023).
4. Gupta, A., Rudra, A., R^{ee}d, K., Langer, R. & Anderson, D. G. Advanced technologies for the development of infectious disease vaccines. Nat Rev Drug Discov (2024) doi:10.1038/s41573-024-01041-z.
5. Chaudhary, N., Weissman, D. & Whitehead, K. A. mRNA vaccines for infectious diseases: principles, delivery and clinical translation. Nat Rev Drug Discov 20, 817-838 (2021).
6. Verbeke, R., Hogan, M. J., Loré, K. & Pardi, N. Innate immune mechanisms of mRNA vaccines. Immunity 55, 1993-2005 (2022).
7. Sabnis, S. et al. A Novel Amino Lipid Series for mRNA Delivery: Improved Endosomal Escape and Sustained Pharmacology and Safety in Non-human Primates. Molecular Therapy 26, 1509-1519 (2018).
8. Miao, L. et al. Delivery of mRNA vaccines with heterocyclic lipids increases anti-tumor efficacy by STING-mediated immune cell activation. Nat Biotechnol 37, 1174-1185 (2019).
9. Tahtinen, S. et al. IL-1 and IL-1ra are key regulators of the inflammatory response to RNA vaccines. Nat Immunol 23, 532-542 (2022).
10. Dong, Y. et al. Lipopeptide nanoparticles for potent and selective siRNA delivery in rodents and nonhuman primates. Proc Natl Acad Sci USA 111, 3955-3960 (2014).
11. Love, K. T. et al. Lipid-like materials for low-dose, in vivo gene silencing. Proc Natl Acad Sci USA 107, 1864-1869 (2010).
12. Whitehead, K. A. et al. Degradable lipid nanoparticles with predictable in vivo siRNA delivery activity. Nature Communications 2014 5:1 5, 1-10 (2014).
13. Fenton, O. S. et al. Bioinspired Alkenyl Amino Alcohol Ionizable Lipid Materials for Highly Potent In Vivo mRNA Delivery. Advanced Materials 28, 2939-2943 (2016).
14. Goldman, R. L. et al. Understanding structure activity relationships of Good HEPES lipids for lipid nanoparticle mRNA vaccine applications. Biomaterials 301, 122243 (2023).
15. Fukami, T. & Yokoi, T. The Emerging Role of Human Esterases. Drug Metab Pharmacokinet 27, 466-477 (2012).
16. Brotzel, F., Ying, C. C. & Mayr, H. Nucleophilicities of primary and secondary amines in water. Journal of Organic Chemistry 72, 3679-3688 (2007).
17. Heyes, J., Palmer, L., Bremner, K. & MacLachlan, I. Cationic lipid saturation influences intracellular delivery of encapsulated nucleic acids. Journal of Controlled Release 107, 276-287 (2005).
18. Semple, S. C. et al. Rational design of cationic lipids for siRNA delivery. Nat Biotechnol 28, 172-176 (2010).
19. Kauffman, K. J. et al. Optimization of Lipid Nanoparticle Formulations for mRNA Delivery in Vivo with Fractional Factorial and Definitive Screening Designs. Nano Lett 15, 7300-7306 (2015).
20. Whitehead, K. A. et al. In Vitro-In Vivo Translation of Lipid Nanoparticles for Hepatocellular siRNA Delivery. ACS Nano 6, 6922-6929 (2012).
21. Li, B. et al. Enhancing the immunogenicity of lipid-nanoparticle mRNA vaccines by adjuvanting the ionizable lipid and the mRNA. Nat Biomed Eng (2023) doi:10.1038/s41551-023-01082-6.
22. Jayaraman, M. et al. Maximizing the Potency of siRNA Lipid Nanoparticles for Hepatic Gene Silencing In Vivo. Angewandte Chemie International Edition 51, 8529-8533 (2012).
23. Cornebise, M. et al. Discovery of a Novel Amino Lipid That Improves Lipid Nanoparticle Performance through Specific Interactions with mRNA. Adv Funct Mater 32, 2106727 (2022).
24. Sabnis, S. et al. A Novel Amino Lipid Series for mRNA Delivery: Improved Endosomal Escape and Sustained Pharmacology and Safety in Non-human Primates. Molecular Therapy 26, 1509-1519 (2018).
25. Finn, J. D. et al. A Single Administration of CRISPR/Cas9 Lipid Nanoparticles Achieves Robust and Persistent In Vivo Genome Editing. Cell Rep 22, 2227-2235 (2018).
26. Maier, M. A. et al. Biodegradable Lipids Enabling Rapidly Eliminated Lipid Nanoparticles for Systemic Delivery of RNAi Therapeutics. Molecular Therapy 21, 1570-1578 (2013).
27. Ndeupen, S. et al. The mRNA-LNP platform's lipid nanoparticle component used in preclinical vaccine studies is highly inflammatory. iScience 24, (2021).
28. Hassett, K. J. et al. Optimization of Lipid Nanoparticles for Intramuscular Administration of mRNA Vaccines. Mol Ther Nucleic Acids 15, 1-11 (2019).
29. Hassett, K. J. et al. mRNA vaccine trafficking and resulting protein expression after intramuscular administration. Mol Ther Nucleic Acids 35, (2024).
30. Trougakos, I. P. et al. Adverse effects of COVID-19 mRNA vaccines: the spike hypothesis. Trends Mol Med 28, 542-554 (2022).
31. Efe, C. et al. Liver injury after SARS-CoV-2 vaccination: Features of immune-mediated hepatitis, role of corticosteroid therapy and outcome. Hepatology 76, (2022).
32. Broudic, K. et al. Nonclinical safety evaluation of a novel ionizable lipid for mRNA delivery. Toxicol Appl Pharmacol 451, 116143 (2022).
33. Corbett, K. S. et al. SARS-CoV-2 mRNA vaccine design enabled by prototype pathogen preparedness. Nature | 586, (2020).
34. Pecetta, S. & Rappuoli, R. mRNA, the beginning of a new influenza vaccine game. Proceedings of the National Academy of Sciences 119, e2217533119 (2022).
35. Russell, C. A. et al. Seasonal influenza vaccine performance and the potential benefits of mRNA vaccines. Hum Vaccin Immunother 20, 2336357 (2024).
36. Fink, A. L., Engle, K., Ursin, R. L., Tang, W.-Y. & Klein, S. L. Biological sex affects vaccine efficacy and protection against influenza in mice. Proceedings of the National Academy of Sciences 115, 12477-12482 (2018).
37. Watanabe, H., Numata, K., Ito, T., Takagi, K. & Matsukawa, A. INNATE IMMUNE RESPONSE IN TH1- AND TH2-DOMINANT MOUSE STRAINS. Shock 22, (2004).
38. A, P. S. Correlates of Protection Induced by Vaccination. Clinical and Vaccine Immunology 17, 1055-1065 (2010).
39. Khoury, D. S. et al. Neutralizing antibody levels are highly predictive of immune protection from symptomatic SARS-CoV-2 infection. Nat Med 27, 1205-1211 (2021).
40. Hervé, C., Laupeze, B., Del Giudice, G., Didierlaurent, A. M. & Tavares Da Silva, F. The how's and what's of vaccine reactogenicity. NPJ Vaccines 4, 39 (2019).
41. Arunachalam, P. S. et al. Systems vaccinology of the BNT162b2 mRNA vaccine in humans. Nature 596, 410-416 (2021).
42. Schramm, C. A. et al. Interaction dynamics between innate and adaptive immune cells responding to SARS-CoV-2 vaccination in non-human primates. Nat Commun 14, 7961 (2023).
43. Liu, H. et al. Structure-based programming of lymph-node targeting in molecular vaccines. Nature 507, 519-522 (2014).
44. Fenton, O. S. et al. Customizable Lipid Nanoparticle Materials for the Delivery of siRNAs and mRNAs. Angewandte Chemie International Edition 57, 13582-13586 (2018).
45. Liang, F. et al. Efficient Targeting and Activation of Antigen-Presenting Cells In Vivo after Modified mRNA Vaccine Administration in Rhesus Macaques. Molecular Therapy 25, 2635-2647 (2017).
46. Vono, M. et al. The adjuvant MF59 induces ATP release from muscle that potentiates response to vaccination. Proceedings of the National Academy of Sciences 110, 21095-21100 (2013).
47. Thurston, T. L. M., Wandel, M. P., von Muhlinen, N., Foeglein, Á. & Randow, F. Galectin 8 targets damaged vesicles for autophagy to defend cells against bacterial invasion. Nature 482, 414-418 (2012).
48. Haensler, J. & Szoka, F. C. Polyamidoamine Cascade Polymers Mediate Efficient Transfection of Cells in Culture. Bioconjug Chem 4, 372-379 (1993).
49. Bus, T., Traeger, A. & Schubert, U. S. The great escape: how cationic polyplexes overcome the endosomal barrier. J Mater Chem B 6, 6904-6918 (2018).
50. Freeman, E. C., Weiland, L. M. & Meng, W. S. Modeling the proton sponge hypothesis: examining proton sponge effectiveness for enhancing intracellular gene delivery through multiscale modeling. J Biomater Sci Polym Ed 24, 398-416 (2013).
51. Nguyen, J. & Szoka, F. C. Nucleic Acid Delivery: The Missing Pieces of the Puzzle?Acc Chem Res 45, 1153-1162 (2012).
52. Hafez, I. M., Maurer, N. & Cullis, P. R. On the mechanism whereby cationic lipids promote intracellular delivery of polynucleic acids. Gene Therapy 2001 8:15 8, 1188-1196 (2001).
53. Finger, S., Schwieger, C., Arouri, A., Kerth, A. & Blume, A. Interaction of linear polyamines with negatively charged phospholipids: The effect of polyamine charge distance. Biol Chem 395, 769-778 (2014).
54. Cornebise, M. et al. Discovery of a Novel Amino Lipid That Improves Lipid Nanoparticle Performance through Specific Interactions with mRNA. Adv Funct Mater 32, 2106727 (2022).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 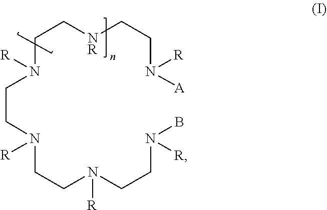
or a pharmaceutically acceptable salt, solvate, tautomer, stereoisomer, or isotopically labeled derivative thereof, wherein:
n is 1, 2, or 3;
A and B are independently optionally substituted, saturated or unsaturated aliphatic; or
A and B combine to form optionally substituted, C₂-C₆ alkylene;
each R is independently hydrogen, a nitrogen protecting group, or a group of the Formula (i), (ii), (iii), (iv), or (v):
(i) 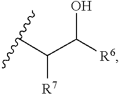
wherein one of R⁶ and R⁷ is hydrogen, and the other is 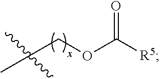
(ii) 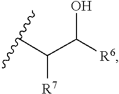
wherein one of R⁶ and R⁷ is hydrogen, and the other is 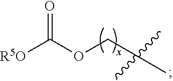
(iii) 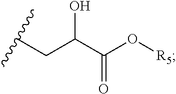
(iv) 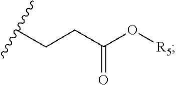
and
(v) 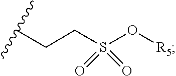
wherein each instance of R⁵ is independently optionally substituted, -saturated or unsaturated, C₄-C₃₅ aliphatic or optionally substituted, -saturated or unsaturated, C₄-C₃₅ heteroaliphatic; and
each x independently is an integer from 1 to 10, inclusive;
provided that at least one R is of Formula (i), (ii), (iii), (iv), or (v).

2. (canceled)

3. The compound of claim 1, of Formula (III), Formula (IV), or Formula (V): 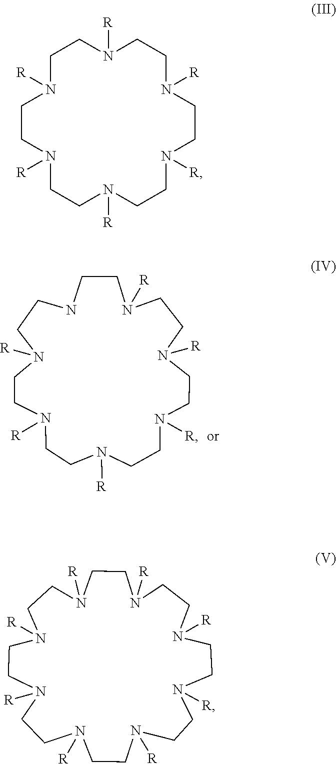
or a pharmaceutically acceptable salt thereof.

4. A compound of Formula (XI): 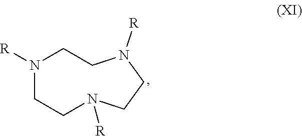
or a pharmaceutically acceptable salt, solvate, tautomer, stereoisomer, or isotopically labeled derivative thereof, wherein:
each R is independently hydrogen, a nitrogen protecting group, or a group of the Formula (i), (ii), (iii), (iv), or (v):
(i) 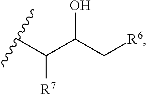
wherein one of R⁶ and R⁷ is hydrogen, and the other is 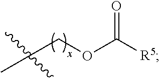
(ii) 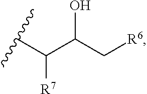
wherein one of R⁶ and R⁷ is hydrogen, and the other is 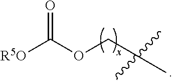
(iii) 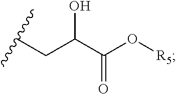
(iv) 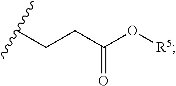
and
(v) 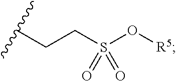
wherein each instance of R⁵ is independently optionally substituted, saturated or unsaturated, C₄-C₃₅ aliphatic or optionally substituted, saturated or unsaturated, C₄-C₃₅ heteroaliphatic; and
each x independently is an integer from 1 to 10, inclusive;
provided that at least one R is of Formula (i), (ii), (iii), (iv), or (v).

5. A compound of Formula (XII): 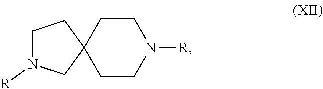
or a pharmaceutically acceptable salt, solvate, tautomer, stereoisomer, or isotopically labeled derivative thereof, wherein:
each R is independently hydrogen, a nitrogen protecting group, or a group of the Formula (i), (ii), (iii), (iv), or (v):
(i) 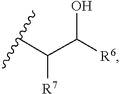
wherein one of R⁶ and R⁷ is hydrogen, and the other is 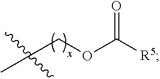
(ii) 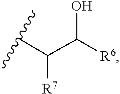
wherein one of R⁶ and R⁷ is hydrogen, and the other is 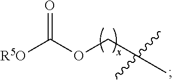
(iii) 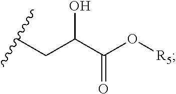
(iv) 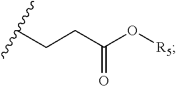
and
(v) 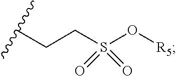
wherein each instance of R⁵ is independently optionally substituted, saturated or unsaturated, C₄-C₃₅ aliphatic or optionally substituted, saturated or unsaturated, C₄-C₃₅ heteroaliphatic; and
each x independently is an integer from 1 to 10, inclusive;
provided that at least one R is of Formula (i), (ii), (iii), (iv), or (v).

6. A compound of Formula (XIII): 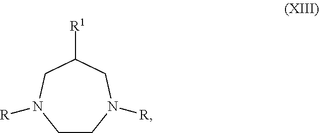
or a pharmaceutically acceptable salt, solvate, tautomer, stereoisomer, or isotopically labeled derivative thereof, wherein:
R¹ is hydrogen, —OR^{A}, —SR^{A}, or —N(R^{A})₂,
each instance of R^{A} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, an oxygen protecting group when attached to an oxygen atom, a nitrogen protecting group when attached to a nitrogen atom, or a sulfur protecting group when attached to a sulfur atom, or wherein two instances of R^{A} attached to the same nitrogen atom are joined together with the intervening atoms to form optionally substituted heterocyclyl or optionally substituted heteroaryl;
each R is independently hydrogen, a nitrogen protecting group, or a group of the Formula (i), (ii), (iii), (iv), or (v):
(i) 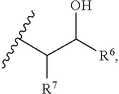
wherein one of R⁶ and R⁷ is hydrogen, and the other is 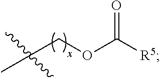
(ii) 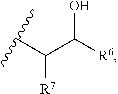
wherein one of R⁶ and R⁷ is hydrogen, and the other is 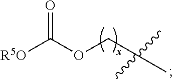
(iii) 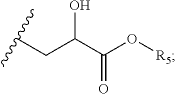
(iv) 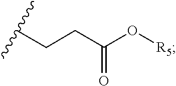
and
(v) 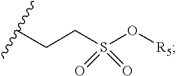
wherein each instance of R is independently optionally substituted, saturated or unsaturated, C₄-C₃₅ aliphatic or optionally substituted, saturated or unsaturated, C₄-C₃₅ heteroaliphatic; and
each x independently is an integer from 1 to 10, inclusive;
provided that at least one R is of Formula (i), (ii), (iii), (iv), or (v).

7. (canceled)

8. The compound of claim 1, or a pharmaceutically acceptable salt thereof, wherein each R is 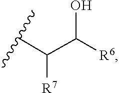
wherein one of R⁶ and R⁷ is hydrogen, and the other is

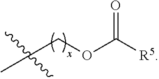

9. The compound of claim 1, or a pharmaceutically acceptable salt thereof, wherein each R is 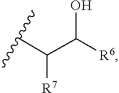
wherein one of R⁶ and R⁷ is hydrogen, and the other is

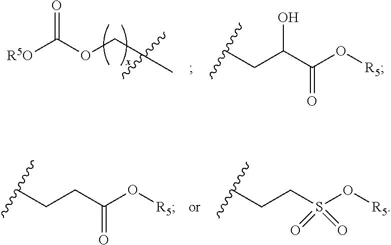

10. (canceled)

11. The compound of claim 1, or a pharmaceutically acceptable salt thereof, wherein each R⁷ is hydrogen.

12. The compound of claim 1, or a pharmaceutically acceptable salt thereof, wherein each x is 1 or 8.

13-16. (canceled)

17. The compound of claim 8, or a pharmaceutically acceptable salt thereof, wherein each R⁵ is independently optionally substituted, C₄-C₃₅ alkyl or optionally substituted, C₄-C₃₅ alkenyl.

18-19. (canceled)

20. The compound of claim 1, or a pharmaceutically acceptable salt thereof, wherein each R⁵ is independently optionally substituted, C₄-C₃₅ heteroalkyl or optionally substituted, C₄-C₃₅ heteroalkenyl.

21. (canceled)

22. The compound of claim 1, or a pharmaceutically acceptable salt thereof, wherein each R⁵ is independently selected from the group consisting of:

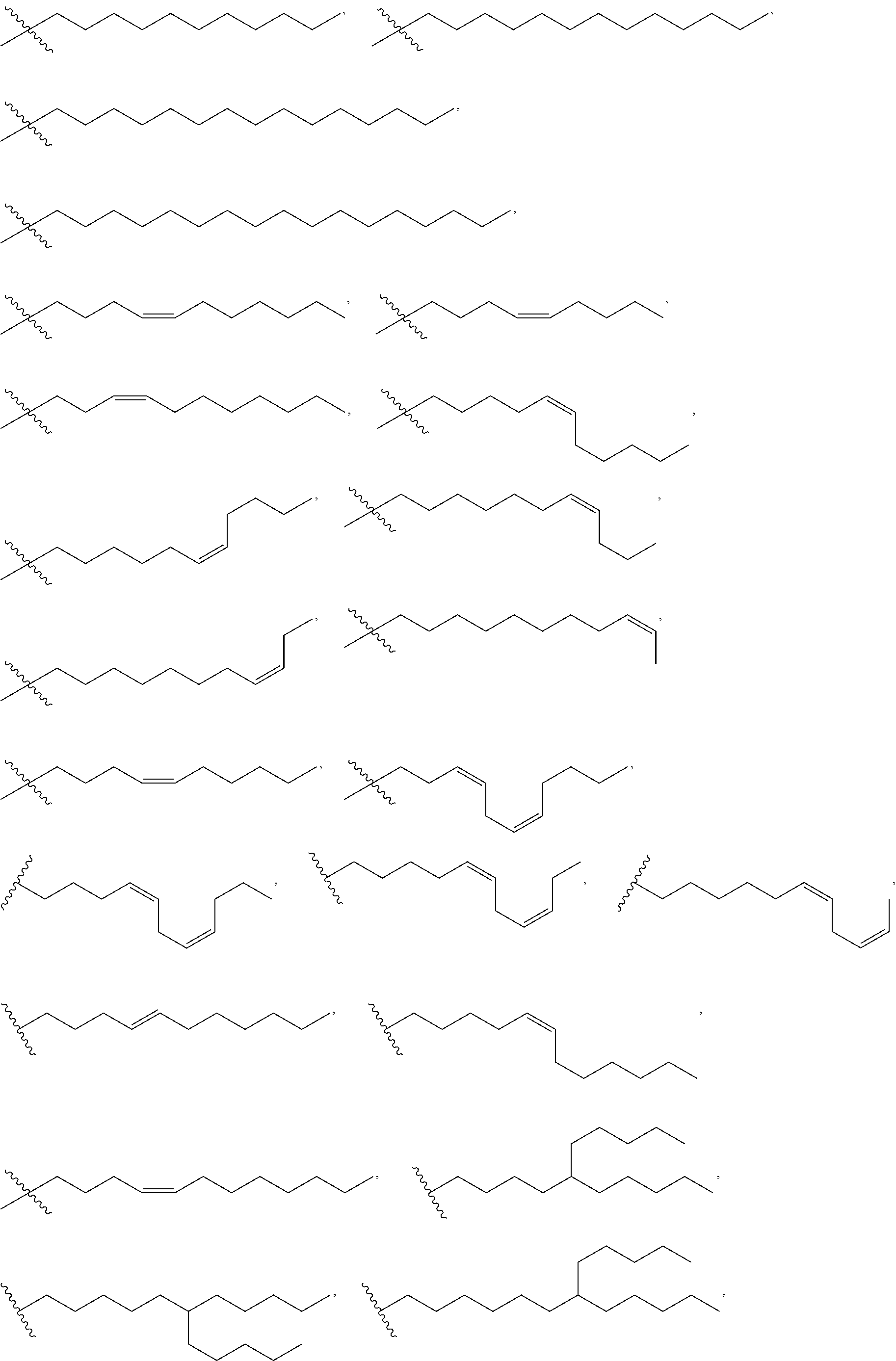

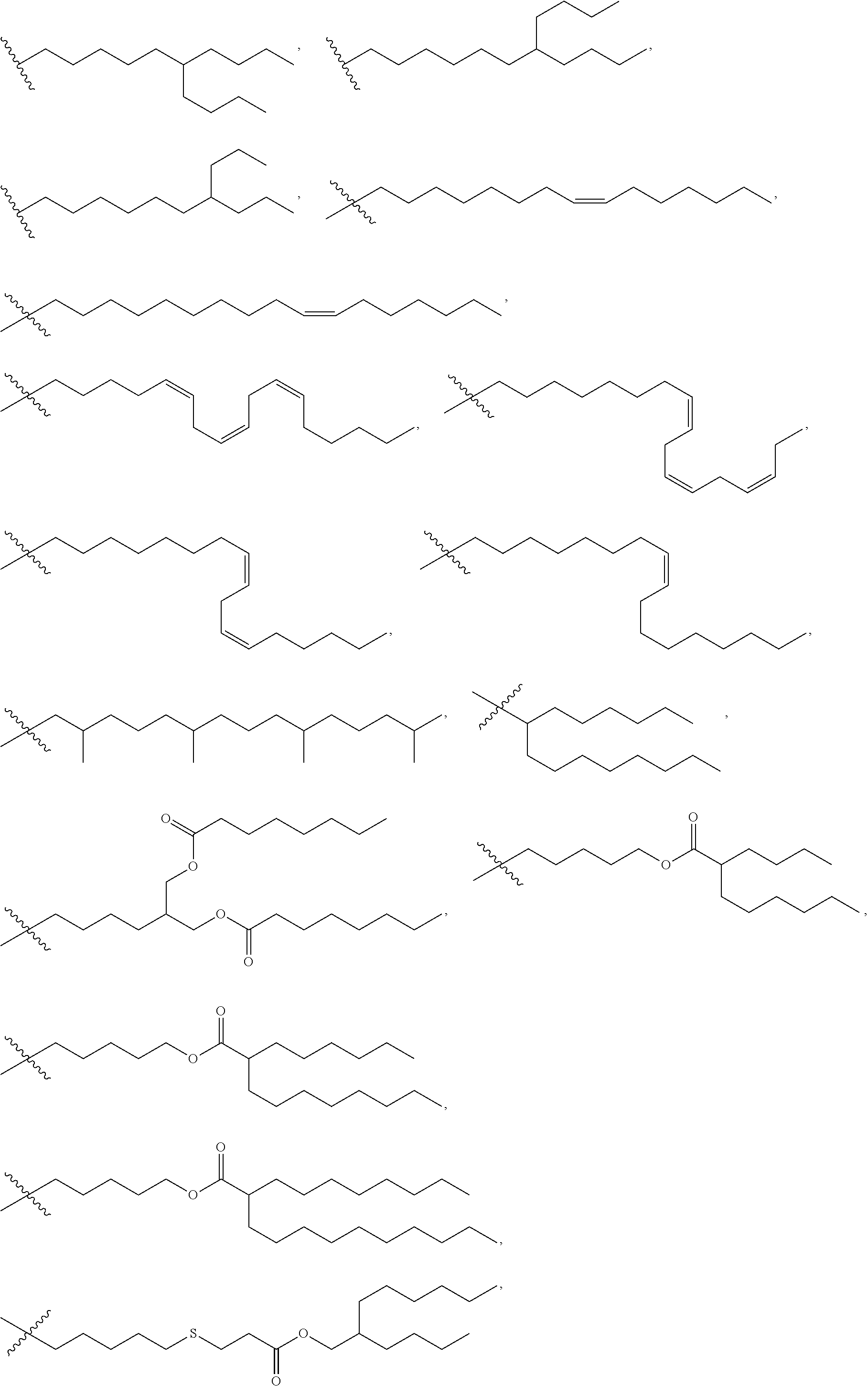

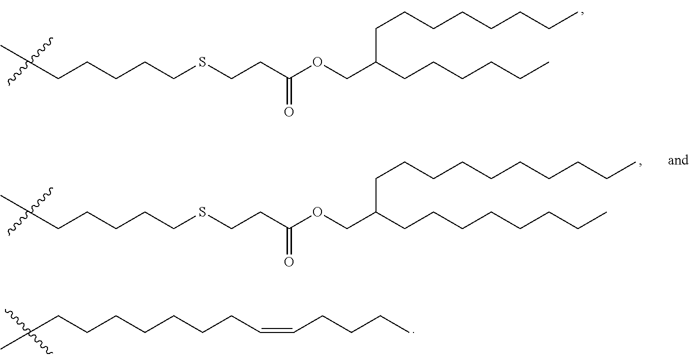 [SMILES: *CCCCCSCCC(=O)OCC(CCCCCC)CCCCCCCC ; *CCCCCSCCC(=O)OCC(CCCCCCCC)CCCCCCCCCC ; *CCCCCCC/C=C\CCCC]

23-27. (canceled)

28. The compound of claim 3, or a pharmaceutically acceptable salt thereof, wherein the compound is of Formula (IV), and each R is 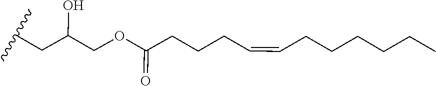 [SMILES: *CC(O)COC(=O)CCC/C=C\CCCCCC]
each R is 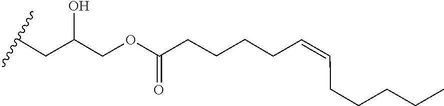 [SMILES: *CC(O)COC(=O)CCCC/C=C\CCCCC]
each R is 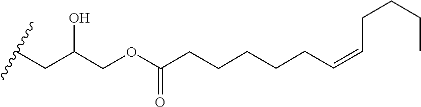 [SMILES: *CC(O)COC(=O)CCCCC/C=C\CCCC]
each R is 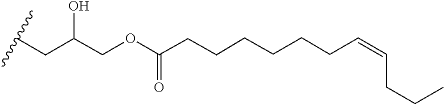 [SMILES: *CC(O)COC(=O)CCCCCC/C=C\CCC]
each R is 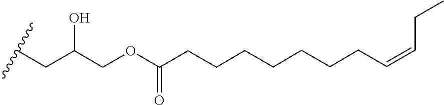 [SMILES: *CC(O)COC(=O)CCCCCCC/C=C\CC]
each R is 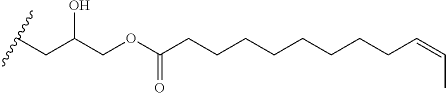 [SMILES: *CC(O)COC(=O)CCCCCCCC/C=C\C]
each R is 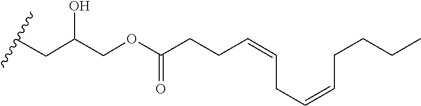 [SMILES: *CC(O)COC(=O)CC/C=C\C/C=C\CCCC]
each R is 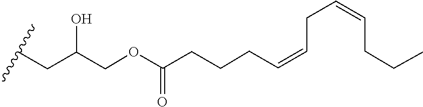 [SMILES: *CC(O)COC(=O)CCC/C=C\C/C=C\CCC]
each R is 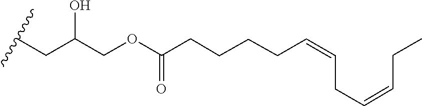 [SMILES: *CC(O)COC(=O)CCCC/C=C\C/C=C\CC]
or each R is

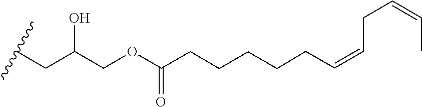 [SMILES: *CC(O)COC(=O)CCCCC/C=C\C/C=C\C]

29. (canceled)

30. The compound of claim 3, or a pharmaceutically acceptable salt thereof, the compound is of Formula (V), and wherein each R is 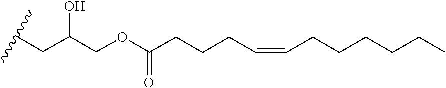 [SMILES: *CC(O)COC(=O)CCC/C=C\CCCCCC]
each R is 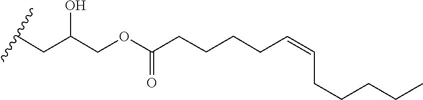 [SMILES: *CC(O)COC(=O)CCCC/C=C\CCCCC]
each R is 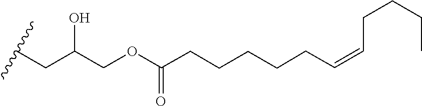 [SMILES: *CC(O)COC(=O)CCCCC/C=C\CCCC]
each R is 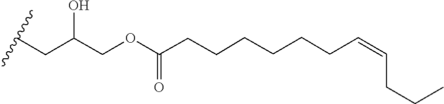 [SMILES: *CC(O)COC(=O)CCCCCC/C=C\CCC]
each R is 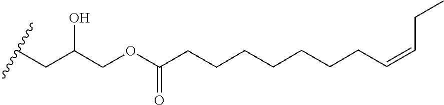 [SMILES: *CC(O)COC(=O)CCCCCCC/C=C\CC]
each R is 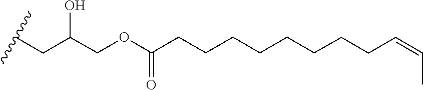 [SMILES: *CC(O)COC(=O)CCCCCCCC/C=C\C]
each R is 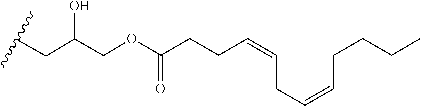 [SMILES: *CC(O)COC(=O)CC/C=C\C/C=C\CCCC]
each R is 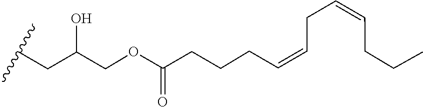 [SMILES: *CC(O)COC(=O)CCC/C=C\C/C=C\CCC]
each R is 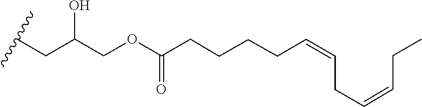 [SMILES: *CC(O)COC(=O)CCCC/C=C\C/C=C\CC]
or each R is

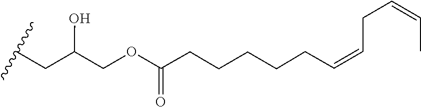 [SMILES: *CC(O)COC(=O)CCCCC/C=C\C/C=C\C]

31-32. (canceled)

33. The compound of claim 1, of Formula (VI): 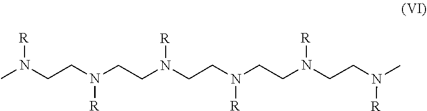
or a pharmaceutically acceptable salt thereof.

34. The compound of claim 33, or a pharmaceutically acceptable salt thereof, wherein each R is 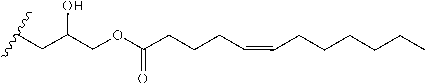 [SMILES: *CC(O)COC(=O)CCC/C=C\CCCCCC]
each R is 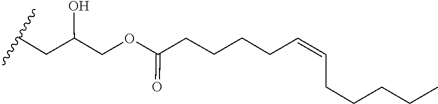 [SMILES: *CC(O)COC(=O)CCCC/C=C\CCCCC]
each R is 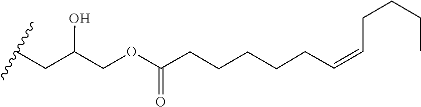 [SMILES: *CC(O)COC(=O)CCCCC/C=C\CCCC]
each R is 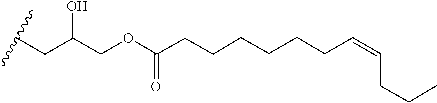 [SMILES: *CC(O)COC(=O)CCCCCC/C=C\CCC]
each R is 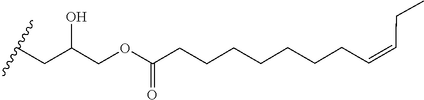 [SMILES: *CC(O)COC(=O)CCCCCCC/C=C\CC]
each R is 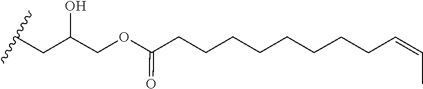 [SMILES: *CC(O)COC(=O)CCCCCCCC/C=C\C]
each R is 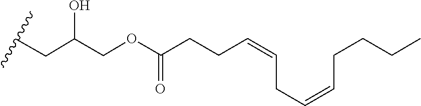 [SMILES: *CC(O)COC(=O)CC/C=C\C/C=C\CCCC]
each R is 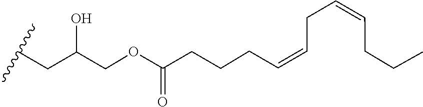 [SMILES: *CC(O)COC(=O)CCC/C=C\C/C=C\CCC]
each R is 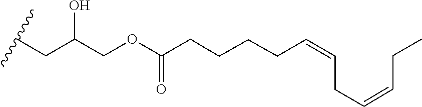 [SMILES: *CC(O)COC(=O)CCCC/C=C\C/C=C\CC]
or each R is

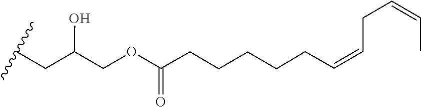 [SMILES: *CC(O)COC(=O)CCCCC/C=C\C/C=C\C]

35-38. (canceled)

39. The compound of claim 1, wherein the compound is: 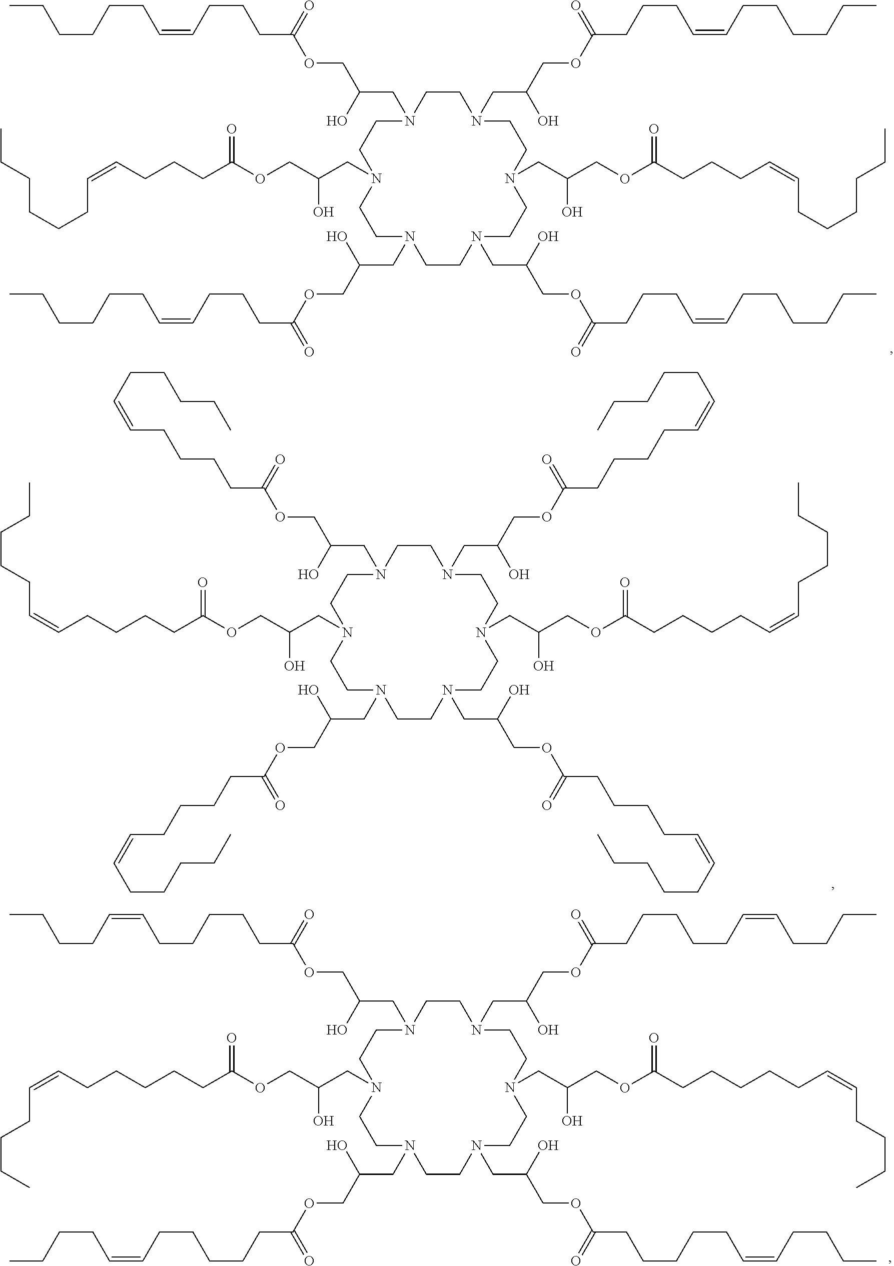 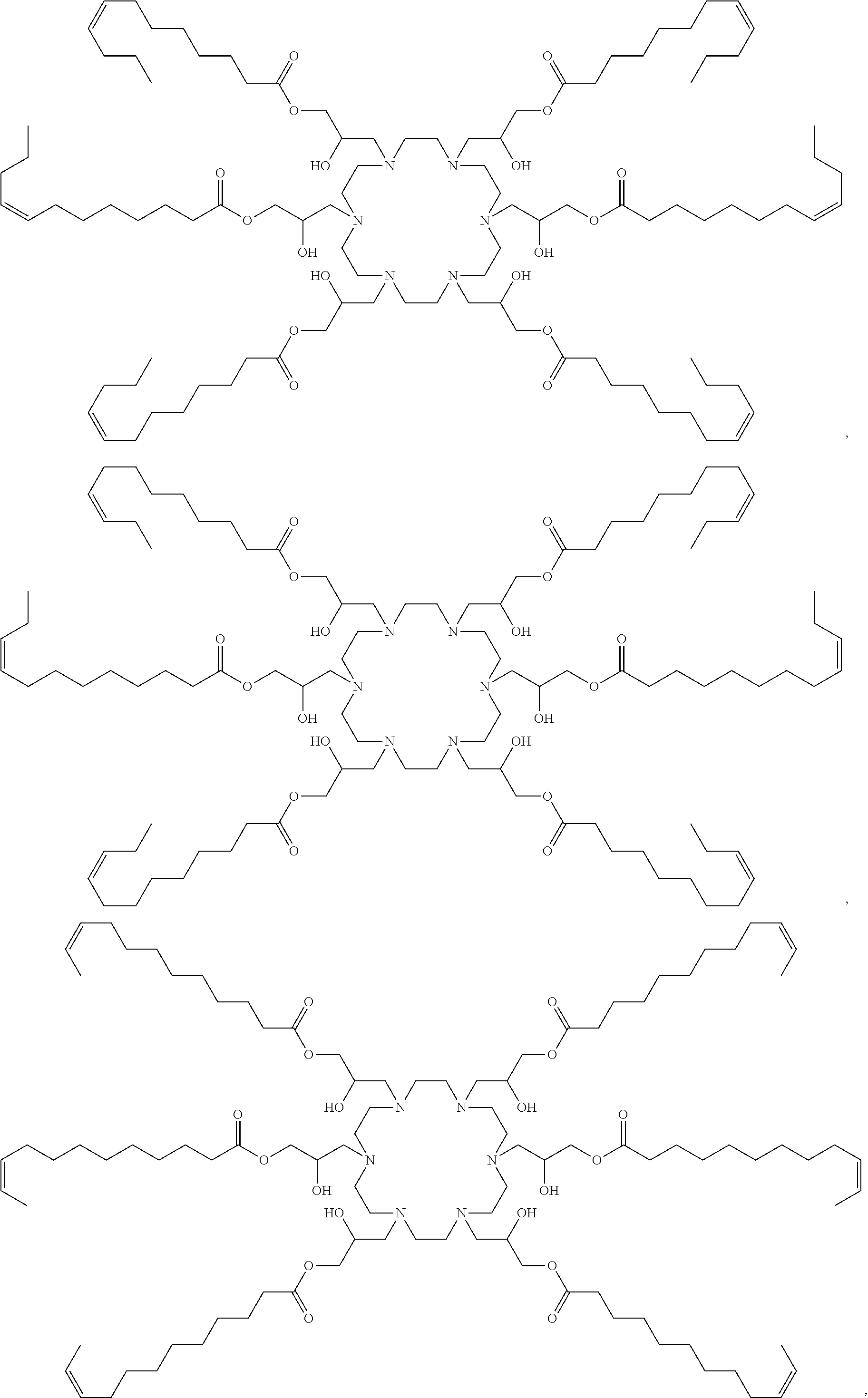 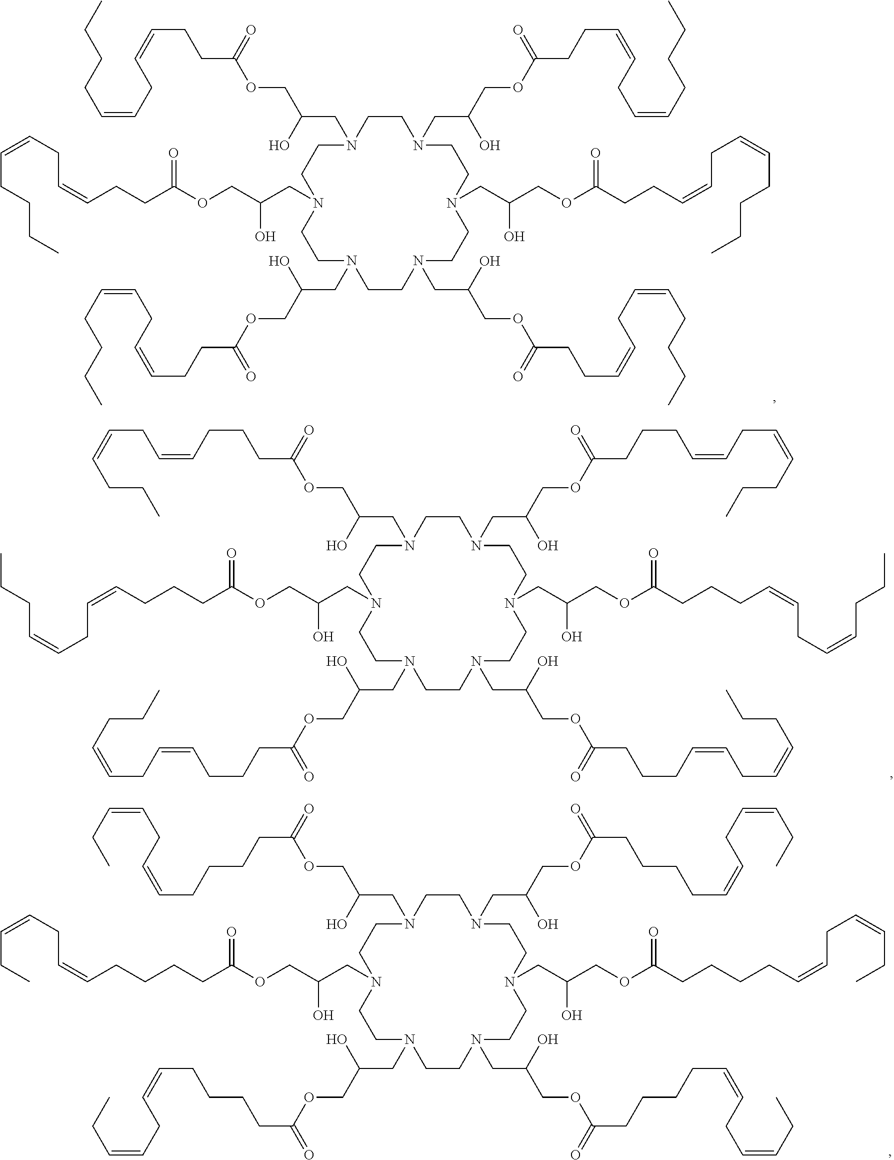 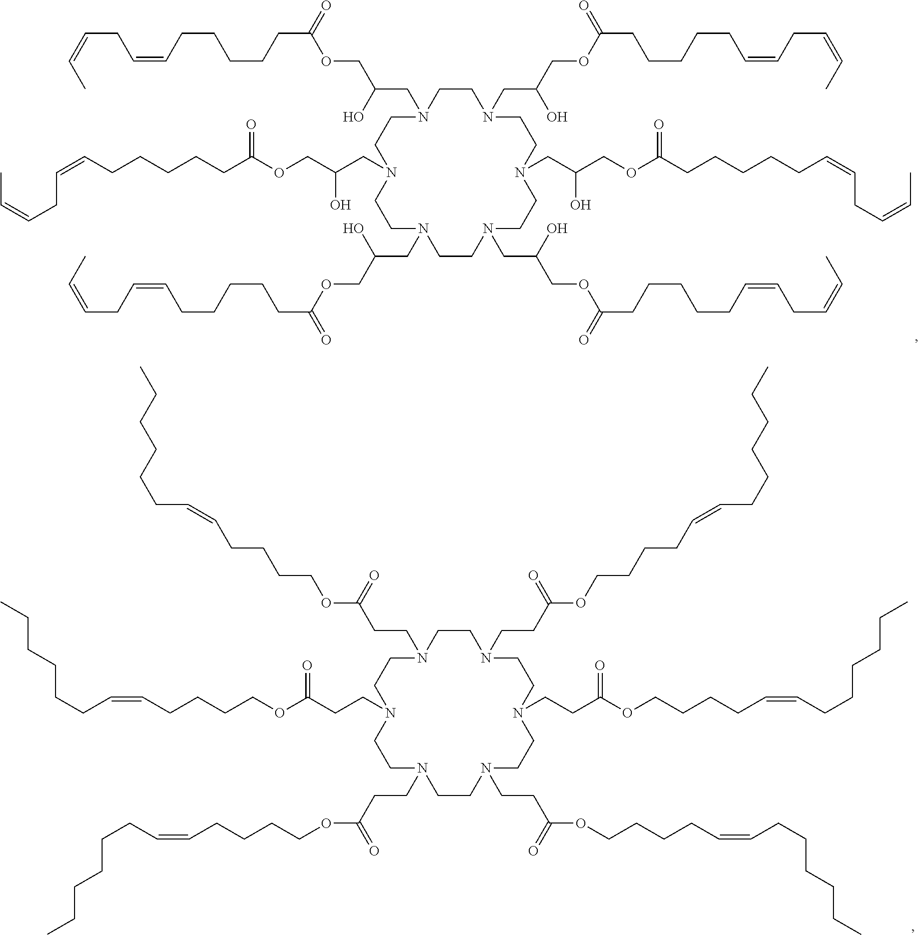 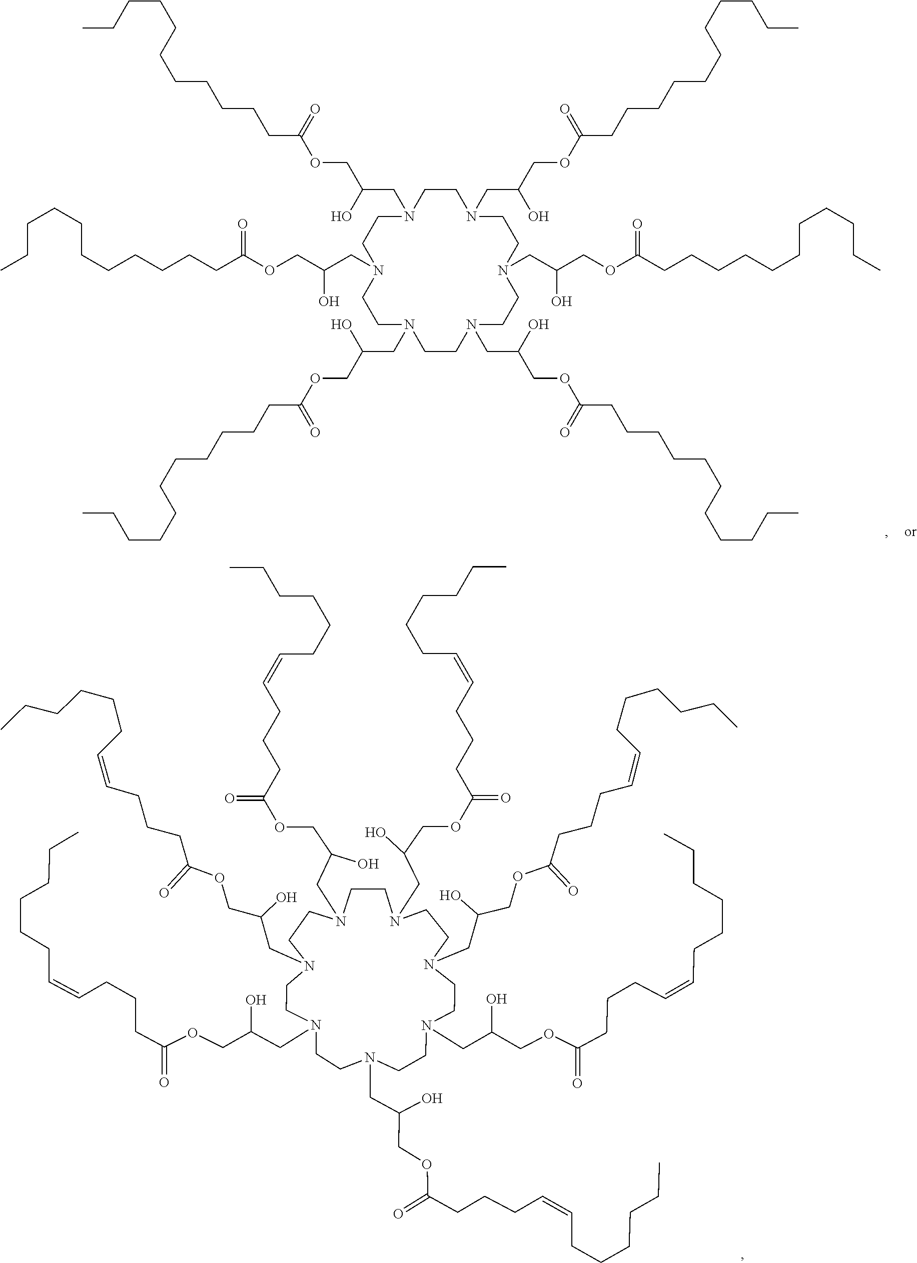
or a pharmaceutically acceptable salt thereof.

40. (canceled)

41. A composition comprising a compound of claim 1, or a pharmaceutically acceptable salt thereof, and an agent.

42-66. (canceled)

67. A method of delivering an agent to a subject, tissue, or a cell, comprising administering to the subject or contacting the tissue or cell with a composition of claim 41.

68-87. (canceled)

88. A method of treating and/or preventing a disease, disorder, or condition in a subject comprising administering to the subject composition of claim 41.

89-119. (canceled)
